(19) United States
(12) Patent Application Publication
(10) Pub. No.: US 2023/0190951 A1
(43) Pub. Date: Jun. 22, 2023

(54) CONJUGATES OF ANTIBODIES AN IMMUNE CELL ENGAGERS

(71) Applicant: SYNAFFIX B.V., Oss (NL)

(72) Inventors: Remon VAN GEEL, Lithoijen (NL); Willem Johannes Petrus VUGS, Oss (NL); Sander Sebastiaan VAN BERKEL, Oss (NL); Floris Louis VAN DELFT, Nijmegen (NL)

(21) Appl. No.: 17/812,160

(22) Filed: Jul. 12, 2022

Related U.S. Application Data
(63) Continuation of application No. PCT/EP2021/050599, filed on Jan. 13, 2021.

(30) Foreign Application Priority Data
Jan. 13, 2020 (EP) 20151544.2

(51) Int. Cl. A61K 47/68

(52) U.S. Cl. CPC A61K 47/6813; A61K 47/6855; A61K 47/6889

(57) ABSTRACT

The present invention concerns a process for preparing a multispecific antibody construct, comprising conjugating a functionalized antibody Ab(F)ₓ containing x reactive moieties F, wherein x is an integer in the range 1 -10, and an immune cell-engaging polypeptide containing one or two reactive moieties Q, wherein the antibody is specific for a tumour cell and the immune cell-engaging polypeptide is specific for an immune cell, wherein the reaction forms a covalent linkage between the functionalized antibody and the immune cell-engaging polypeptide by reaction of Q with F. The invention further concerns the multispecific antibody constructs obtainable by the process according to the invention and medical uses thereof.

### FIELD OF THE INVENTION

The present invention relates to immune cell engagers generated from antibodies and other polypeptides. More specifically the invention relates to conjugates, compositions and methods suitable for the attachment of an immune cell-binding polypeptide of interest to an antibody without requiring genetic engineering of the antibody before such attachment. The resulting antibody-immune cell engager conjugates as compounds, compositions, and methods can be useful, for example, in immunotherapy for cancer patients.

### BACKGROUND OF THE INVENTION

Antibody-drug conjugates (ADC), considered as magic bullets in therapy, are comprised of an antibody to which is attached a pharmaceutical agent. The antibodies (also known as ligands) can be small protein formats (scFv’s, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

An alternative strategy to employ monoclonal antibodies for targeted delivery of a specific protein agent is by genetic fusion of the latter protein to one (or more) of the antibody’s termini, which can be the N-terminus or the C-terminus on the light chain or the heavy chain (or both). In this case, the biologically active protein of interest, e.g. a protein toxin like Pseudomonas exotoxin A (PE38) or an anti-CD3 single chain variable fragment (scFv), is genetically encoded as a fusion to the antibody, possibly but not necessarily via a peptide spacer, so the antibody is expressed as a fusion protein. The peptide spacer may contain a protease-sensitive cleavage site, or not.

A monoclonal antibody may also be genetically modified in the protein sequence itself to modify its structure and thereby introduce (or remove) specific properties. For example, mutations can be made in the antibody Fc-fragment in order to nihilate binding to Fc-gamma receptors, binding to the FcRn receptor or binding to a specific cancer target may be modulated, or antibodies can be engineered to lower the pl and control the clearance rate from circulation.

An emerging strategy in therapeutic treatment involves the use of an antibody that is able to bind simultaneously to multiple antigens or epitopes, a so-called bispecific antibody (simultaneously addressing two different antigens or epitopes), or a trispecific antibody (addressing three different antigens of epitopes), and so forth, as summarized in Kontermann and Brinkmann, Drug Discov. Today 2015, 20, 838-847, incorporated by reference. A bispecific antibody with ‘two-target’ functionality can interfere with multiple surface receptors or ligands associated, for example with cancer, proliferation or inflammatory processes. Bispecific antibodies can also place targets into close proximity, either to support protein complex formation on one cell, or to trigger contacts between cells. Examples of ‘forced-connection’ functionalities are bispecific antibodies that support protein complexation in the clotting cascade, or tumor-targeted immune cell recruiters and/or activators. Depending on the production method and structure, bispecific antibodies vary in the number of antigen-binding sites, geometry, half-life in the blood serum, and effector function.

A wide range of different formats for multispecific antibodies has been developed over the years, which can be roughly divided into IgG-like (bearing a Fc-fragment) and non-IgG-like (lacking a Fc-fragment) formats, as summarized by Kontermann and Brinkmann, Drug Discov. Today 2015, 20, 838-847 and Yu and Wang, J. Cancer Res. Clin. Oncol. 2019, 145, 941-956, incorporated by reference. Most bispecific antibodies are generated by one of three methods by somatic fusion of two hybridoma lines (quadroma), by genetic (protein/cell) engineering, or by chemical conjugation with cross-linkers, totalling more than 60 different technological platforms today.

IgG-like formats based on full IgG molecular architectures include but are not limited to IgG with dual-variable domain (DVD-Ig), Duobody technology, knob-in-hole (KIH) technology, common light chain technology and cross-mAb technology, while truncated IgG versions include ADAPTIR, XmAb and BEAT technologies. Non-IgG-like approaches include but are not limited BITE, DART, TandAb and ImmTAC technologies. Bispecific antibodies can also be generated by fusing different antigen-binding moieties (e.g., scFv or Fab) to other protein domains, which enables further functionalities to be included. For example, two scFv fragments have been fused to albumin, which endows the antibody fragments with the long circulation time of serum albumin, as demonstrated by Müller et al., J. Biol. Chem. 2007, 282, 12650-12660, incorporated by reference. Another example is the ‘dock-and-lock’ approach based on heterodimerization of cAMP-dependent protein kinase A and protein A kinase-anchoring protein, as reported by Rossi et al., Proc. Nat. Acad. Sci. 2006, 103, 6841-6846, incorporated by reference. These domains can be linked to Fab fragments and entire antibodies to form multivalent bispecific antibodies, as shown by Rossi et al., Bioconj. Chem. 2012, 23, 309-323. The dock-and-lock strategy requires the generation of a fusion protein between the targeting antibody and a peptide fragment for docking onto the protein A kinase-anchoring protein. Therapeutic Ab fragments (scFv, diabody) may also be fused with albumin or proteins that bind albumin, which increases the half-life of the drug in the blood up to five to six times. The construction of such molecules gives unpredictable results, thereby bispecific antibodies generated as the result of different Ab-fragment fusion or binding of Abs to other proteins have limited application in research and development of new therapeutic molecules.

Chemical conjugation to generate a non-IgG-type bispecific antibody was used for the first time by Brennan et al., Science 1985, 229, 81-83, incorporated by reference: two Fab₂ fragments obtained by pepsinolysis of rabbit IgG were reduced and then oxidized, resulting in bispecific Fab₂. Similarly, homo- and heterobifunctional reagents interacting with cysteine residues was reported by Glennie et al. 1987, 139, 2367-2375, incorporated by reference. Chemical conjugation of Abs against CD3 and CD20 (rituximab) was used to obtain T cells with bispecific antibody-coated surfaces, as shown by Gall et al., Exp. Hematol. 2005, 33, 452-459, incorporated by reference. Generation of the bispecific CD20 × CD3 was ensured by treatment of OKT3 (anti-CD3) with Traut’s reagent, followed by mixing with maleimide-functionalized rituximab (obtained by pretreament of rituximab with sulfosuccinimidyl 4-(N-maleimidomethyl)cyclohexane-1-carboxylate (sulfo-SMCC). By virtue of the random chemical conjugation of both antibodies, followed by random heterodimerization, the bispecific antibody is inevitable obtained as a highly heterogeneous mixture (also containing multimers). The only chemical method reported to date that is also site-specific is the CovX-Body technology, as reported by Doppalapudi et al., Bioorg. Med. Chem. Lett. 2007, 17, 501-506, incorporated by reference, based on the instalment of an aldolase catalytic antibody site into the the targeting antibody, followed by treatment with peptide fragment chemically modified with a azetidinone-motif, leading to spontaneous ligation. Bispecific antibodies were produced by the addition of two short peptides that inhibited VEGF or angiopoietin 2 with a branched linker and then with the Abs, as reported by Doppalapudi et al., Proc. Nat. Acad. Sci. 2010, 107, 22611-22616, incorporated by reference.

Formats of bispecific antibody generation based on chemical Ab or Ab-fragment conjugation today are not in use, in particular due to the low yield of product (of low purity) and high cost-of-goods. Besides, the advance in recombinant DNA technologies enabled the efficient generation of fusion proteins and positive clinical results were obtained therewith. Regardless, a non-genetic chemical modification approach could significantly accelerate time-to-clinic, in case proper control of site-specificity of stoichiometry can be ensured.

Examples of bispecific antibodies that have been or are currently under clinic development are catumaxomab (EpCAM × CD3), blinatumomab (CD19 × CD3), GBR1302 (Her2 × CD3), MEDI-565 (CEA × CD3), BAY2010112 (PSMA × CD3), RG7221 (angiopoietin × VEGF), RG6013 (FIX × FX), RG7597 (Her1 × Her3), MCLA128 (Her2 × Her3), MM111 (Her2 × Her3), MM141 (IGF1R × Her3), ABT122 (TNFalpha × IL17), ABT981 (IL1a × II1b), ALX0761 (IL17A × IL17F), SAR156597 (IL4 × IL13), AFM13 (CD30 × CD16) and LY3164530 (Her1 × cMET).

A popular strategy in the field of cancer therapy employs a bispecific antibody binding to an upregulated tumor-associated antigen (TAA or simply target) as well as to a receptor present on a cancer-destroying immune cell. e.g. a T cell or an NK cell. Such bispecific antibodies are also known as T cell or NK cell-redirecting antibodies, respectively. Although the approach of immune cell redirecting is already more than 30 years old, new technologies are overcoming the limitations of the 1^{st} generation immune cell-redirecting antibodies, especially extending half-life to allow intermittent dosing, reducing immunogenicity and improving the safety profile. Currently, there is one approved drug (blinatumomab or Blincyto®) and more than 30 other bispecific formats in various stages of clinical development. The basis for the approval of blinatumomab (2014) resulted from a single-arm trial with a 32% complete remission rate and a minimal residual disease (MRD) response (31%) in all patients treated. Currently, 51 clinical trials of blinatumomab are being carried out for ALL (39 trials), NHL (10 trials), multiple myeloma (1 trial) and lymphoid cancer with Richter’s transformation (1 trial). However, Blinatumomab suffers from a main drawback because of its short serum half-life (2.11 h, due to the relatively small molecule and simple structure), and patients require continuous intravenous infusion.

Like other methods of therapy for severe diseases, therapeutic bispecific antibodies cause different side effects, the most common of which are nausea, vomiting, abdominal pain, fatigue, leukopenia, neutropenia, and thrombopenia. In many patients, Abs against therapeutic bispecific antibodies appear in the blood during treatment. Most adverse events occur during the beginning of therapy, and in most cases side effects normalize under continued treatment. The majority of data on therapeutic BsAb adverse effects are available on blinatumomab and catumaxomab, since these drugs have undergone numerous clinical trials. A common side effect of blinatumomab and catumaxomab therapy is “cytokine storm”, elevation of cytokine levels and some neurological events. Cytokine release-related symptoms are general side effects of many therapeutic mAbs and occur due to specific mechanisms of action: use of cytotoxic T cells as effectors. Minimizing cytokine-release syndrome is possible with a low initial dose of the drug in combination with subsequent high doses, as well as corticosteroid (dexamethasone) and antihistamine premedication.

One way to mitigate the adverse events associated with immune cell engagement therapy, in particular cytokine release syndrome, and to avoid the use of step-up-dosing regimens, was reported by Bacac et al., Clin. Cancer Res. 2018, 24, 4785-4797, incorporated by reference. It was shown that with significantly higher potency and safer administration could be achieved by generating a CD20 × CD3 T cell engager with a 2:1 molecular format, i.e. bivalent binding to CD20 and monovalent binding to CD3, which is achieved by insertion of the anti-CD3 fragment in one of the Fab arms of the full-IgG anti-CD20 antibody. The resulting bispecific antibody is associated with a long half-life and high potency enabled by high-avidity bivalent binding to CD20 and head-to-tail orientation of B- and T cell-binding domains in a 2:1 molecular format. A heterodimeric human IgG1 Fc region carrying the “PG LALA” mutations was incorporated to abolish binding to Fcg receptors and to complement component C1q while maintaining neonatal Fc receptor (FcRn) binding, enabling a long circulatory half-life. The bispecific CD20-T cell engagers displays considerably higher potency than other CD20-TCB antibodies in clinical development and is efficacious on tumor cells expressing low levels of CD20. CD20-TCB also displays potent activity in primary tumor samples with low effector:target ratios.

By far the most investigated receptor for the purpose of T cell-engagement involves the CD3 receptor on activated T cells. T cell-redirecting bispecific antibodies are amongst the most used approaches in cancer treatment and the first report in which bispecific antibodies specifically engaged CD3 on T cells on one side and the antigens of cancer cells independent of their T cell receptor (TCR) on the other side, was published 30 years ago. T cell-redirecting antibodies have made considerable progress in hematological malignancies and solid tumour treatments in the past 10 years. Catumaxomab is the first bispecific antibody of its kind targeting epithelial cell adhesion molecule (EpCAM) and CD3, which was approved in Europe (2009) for the treatment of malignant ascites (but withdrawn in 2017 for commercial reasons). This discovery was followed by another successful bispecific targeting CD19 and CD3 (blinatumomab), which was given marketing permission by the FDA for relapsed or refractory precursor B-cell acute lymphoblastic leukemia (ALL) treatment in 2014. At present, although many patients benefit from blinatumomab, there are a number of T cell-redirecting antibodies with different formats and characteristics showing potential anti-tumour efficacy in clinical studies.

The concept of redirecting T cells to the tumor is currently expanded to other receptors, which are at the same time costimulatory, such as CD137 (4-1 BB), CD134 (OX40), CD27 or ICOS.

In the field of CD137 targeting, agonistic monoclonal antibodies (so not bispecific) have shown much preclinical promise but their clinical development has been slow due to a poor therapeutic index, in particular liver toxicity. CD137 is expressed on T cells that are already primed to recognize tumor antigen through MHC/TCR interaction. It is a TNFRSF (tumor necrosis factor receptor super family) member which requires clustering to deliver an activating signal to T cells. Monospecific monoclonal antibodies that can agonise CD137 are in the clinic and known to be potent T cell activators but suffer from treatment-limiting hepatotoxicity due to Fc-receptor and multivalent format-driven clustering. Bispecific tumor-targeted antibodies that are monovalent for CD137, are unable to cause CD137 clustering in normal tissue. Only upon binding of the bispecific antibody to a tumor-associated antigen on tumor cells, clustering of co-engaged CD137 on tumor-associated T cells is induced. This drives a highly potent but tumor-specific T cell activation. The tumor-targeted cross-linking of Cd137/4-1BB might provide a safe and effective way for co-stimulation of T cells for cancer immunotherapy and its combination with T cell bispecific antibodies may provide a convenient “off-the-shelf,” systemic cancer immunotherapy approach for many tumor types. Examples of anti-CD137-based bispecific antibodies in clinical development include MP0310 (FAP × CD137), RG7827 (FAP × CD137), ALG.APV-527 (5T4 × CD137), MCLA145 (PD-1 × CD137), PRS342 (glypican-3 × CD137), PRS-343 (Her2 × CD137), CB307 (PSMA × CD137). Various of the above bispecifics are deliberately chosen as monovalent for CD137 and as such is unable to cause CD137 clustering in normal tissue. For example, only after binding of the bispecific CB307 to PSMA on tumor cells, it causes clustering of co-engaged CD137 on tumour-associated T cells, thereby driving a highly potent but tumor-specific T cell activation.

Antibodies known to bind T cells are known in the art, highlighted by Martin et al., Clin. Immunol. 2013, 148, 136-147 and Rossi et al., Int. Immunol. 2008, 20, 1247-1258, both incorporated by reference, for example OKT3, UCHT3, BMA031 and humanized versions thereof. Antibodies known to bind to Vy9V82 T cells are also known, see for example de Bruin et al., J. Immunol. 2017, 198, 308-317, incorporated by reference.

Similar to T cell engagement, NK cell recruitment to the tumor microenvironment is under broad investigation. NK cell engagement is typically based on binding CD16, CD56, NKp46, or other NK cell-specific receptors, as summarized in Konjevic et al., 2017, http://dx.doi.org/10.5772/intechopen.69729, incorporated by reference. NK cell engagers can be generated by fusion or insertion of an NK-binding antibody (fragment) to a full IgG binding to a tumor-associated antigen. Alternatively, specific cytokines can also be employed, given that NK cell antitumor activity is regulated by numerous activating and inhibitory NK cell receptors, alterations in NK cell receptor expression and signaling underlie diminished cytotoxic NK cell function. Based on this and on predictive in vitro findings, cytokines including IFNα, IL-2, IL-12, IL-15, and IL-18 have been used systemically or for ex vivo activation and expansion of NK cells and have led to improved NK cells antitumor activity by increasing the expression of NK cell activating receptors and by inducing cytotoxic effector molecules. Moreover, this cytokine-based therapy enhances NK cell proliferation and regulatory function, and it has been shown that it induces NK cells exhibiting cytokine induced memory-like properties that represent a newly defined NK cell subset with improved NK cell activity and longevity. Both for cancer therapy as well as for the treatment of chronic inflammation, several cytokine payloads have been developed and tested in preclinical trials. Proinflammatory cytokines such as IL-2, TNF and IL-12 have been investigated for tumor therapy, as they have been found to increase and activate the local infiltrate of leukocytes at the tumor site. For example, IL-2 monotherapy has been approved as aldesleukin (Proleukin®) and is in phase III clinical trials in combination with nivolumab (NKTR-214). Similarly, various recombinant versions of IL-15 are under clinical evaluation (rhIL-15 or ALT-803). Specific mutants of IL-15 have been reported, for example by Behar et al., Prof. Engin. Des. Sel. 2011, 24, 283-290 and Silva et al., Nature 2019, 565, 186-191, both incorporated by reference, and the complex of IL-15 with IL-15 receptor (IL-15R), as reported by Rubinstein et al., Proc. Nat. Acad. Sci. 2006, 103, 9166-9171, incorporated by reference and fusion constructs of IL-15 and IL-15R (Sushi domain) have also been evaluated for antitumor activity, see for example Bessard et al., Mol. Canc. Ther. 2009, 8, 2736-2745, incorporated by reference. In addition, antibodies have been developed, as for example reported by Boyman et al., Science 2006, 311, 1924-1927, Arenas-Ramirez et al., Sci. Transl. Med. 2016, 8, DOI: 10.1126/scitranslmed.aag3187, Lee et al, Oncoimmunology 2020, 9, e1681869, DOI: 10.1080/2162402X.2019.1681869, WO 2017070561, WO2018217058, WO2016005950, all incorporated by reference, for recruitment of endogenous IL-2, most favorably by binding to a IL-2 domain that normally binds to IL-2Rα, thereby leading to selective activation of CD8+ T cells without activation of Treg. By contrast immunosuppressive cytokines such as IL-10 may be considered as payloads for the treatment of chronic inflammatory conditions or of other diseases (e.g., endometriosis).

Systemic administration of pro-inflammatory cytokines can lead to severe off-target-related adverse effects, which may limit the dose and prevent escalation to therapeutically active regimens. Certain cytokine products (e.g., IL-2, TNF, IL-12) have exhibited recommended doses in the single-digit milligram range (per person) or even below. Adverse effects associated with the intravenous administration of pro-inflammatory cytokines may include hypotension, fever, nausea or flu-like symptoms, and may occasionally also cause serious haematologic, endocrine, autoimmune or neurologic events. In view of these considerations, there is a clear biomedical need for the development of ‘next-generation’ cytokine products, which are better tolerated and which display a preferential action at the site of disease, helping to spare normal tissues, as summarized in Murer and Neri, New Biotechnol. 2019, 52, 42-53, incorporated by reference. Thus, the targeted delivery of cytokines to the tumor aims at inducing a local pro-inflammatory environment, which may activate and recruit immune cells. A list of antibody-cytokine fusions described in the literature has been reported by Hutmacher and Neri, Adv. Drug Deliv. Rev. 2018, 141, 67-91, incorporated by reference. A list of clinical cytokine fusions is provided in Murer and Neri, New Biotechnol. 2019, 52, 42-53, incorporated by reference. Various IL-15 fusions proteins are under preclinical evaluation, as summarized in “T-cell & NK-Cell Engaging Bispecific Antibodies 2019: A Business, Stakeholder, Technology and Pipeline Analysis”, 2019, released by La Merie publishing, incorporated by reference, for example OXS-3550 (CD33-IL-15-CD16 fusion) prepared by Trike technology is currently in phase I.

A common strategy in the field of immune cell engagement employs nihilation or removal of binding capacity of the antibody to Fc-gamma receptors, which has multiple pharmaceutical implications. The first consequence of removal of binding to Fc-gamma receptors is the reduction of Fc-gamma receptor-mediated uptake of antibodies by e.g. macrophages or megakaryocytes, which may lead to dose-limiting toxicity as for example reported for Kadcyla® (trastuzumab-DM1) and LOP628. Selective deglycosylation of antibodies in vivo affords opportunities to treat patients with antibody-mediated autoimmunity. Removal of high-mannose glycoform in a recombinant therapeutic glycoprotein may be beneficial, since high-mannose glycoforms are known to compromise therapeutic efficacy by aspecific uptake by endogenous mannose receptors and leading to rapid clearance, as for example described by Gorovits and Krinos-Fiorotti, Cancer Immunol. Immunother. 2013, 62, 217-223 and Goetze et al, Glycobiology 2011, 21, 949-959 (both incorporated by reference). In addition, Van de Bovenkamp et al, J. Immunol. 2016, 196, 1435-1441 (incorporated by reference) describe how high mannose glycans can influence immunity. It was described by Reusch and Tejada, Glycobiology 2015, 25, 1325-1334 (incorporated by reference), that inappropriate glycosylation in monoclonal antibodies could contribute to ineffective production from expressed Ig genes.

In the field of immune therapy, binding of glycosylated antibodies to Fc-gamma receptors on immune cells may induce systemic activation of the immune system, prior to binding of the antibody to the tumor-associated antigen, leading to cytokine storm (cytokine release syndrome, CRS). Therefore, in order to reduce the risk of CRS, the vast majority of immune cell engagers in the clinic are based on Fc-silenced antibodies, lacking the capacity to bind to Fc-gamma receptors. In addition, various companies in the field of bispecific antibodies are tailoring molecular architectures with defined ratios with regard to target-binding versus immune cell-engaging antibody domains. For example, Roche is developing T cell-engagers based on asymmetric monoclonal antibodies that retain bivalent binding capacity to the TAA (for example CD20 or CEA) by both CDRs, but with an additional anti-CD3 fragment engineered into one of the two heavy chains only (2:1 ratio of target-binding:CD3-binding). Similar strategies can be employed for engagement/activation of T cells with anti-CD137 (4-1BB), anti-OX40, anti-CD27 or NK cell-engagement/activation with anti-CD16, CD56, NKp46, or other NK cell specific receptors.

Abrogation of binding to Fc-gamma receptor can be achieved in various ways, for example by specific mutations in the antibody (specifically the Fc-fragment) or by removal of the glycan that is naturally present in the Fc-fragment (C_{H}2 domain, around N297). Glycan removal can be achieved by genetic modification in the Fc-domain, e.g. a N297Q mutation or T299A mutation, or by enzymatic removal of the glycan after recombinant expression of the antibody, using for example PNGase F or an endoglycosidase. For example, endoglycosidase H is known to trim high-mannose and hybrid glycoforms, while endoglycosidase S is able to trim complex type glycans and to some extent hybrid glycan. Endoglycosidase S2 is able to trim both complex, hybrid and high-mannose glycoforms. Endoglycosidase F2 is able to trim complex glycans (but not hybrid), while endoglycosidase F3 can only trim complex glycans that are also 1,6-fucosylated. Another endoglycosidase, endoglycosidase D is able to hydrolyze Man5 (M5) glycan only. An overview of specific activities of different endoglycosidases is disclosed in Freeze et al. in Curr. Prot. Mol. Biol., 2010, 89:17.13A.1-17, incorporated by reference herein. An additional advantage of deglycosylation of proteins for therapeutic use is the facilitated batch-to-batch consistency and significantly improved homogeneity.

Inspiration may be taken from the field of ADC technologies to prepare antibody-protein conjugates for the generation of bispecific antibodies or antibody-cytokine fusions.

Many technologies are known for bioconjugation, as summarized in G.T. Hermanson, “Bioconjugate Techniques”, Elsevier, 3^{rd} Ed. 2013, incorporated by reference. Two main technologies can be recognized for the preparation of ADCs by random conjugation, either based on acylation of lysine side-chain or based on alkylation of cysteine side-chain. Acylation of the ε-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in the manufacuting of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Another important technology for conjugation to cysteine side-chain is by means of disulfide bond, a bioactivatable connection that has been utilized for reversibly connecting protein toxins, chemotherapeutic drugs, and probes to carrier molecules (see for example Pillow et al., Chem. Sci. 2017, 8, 366-370. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on Michael addition on unsaturated bonds, such as reaction with acrylate reagents, see for example Bernardim et al., Nat. Commun. 2016, 7, DOI: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/ (accessed Jan. 7^{th}, 2020). Reaction with methylsulfonylphenyloxadiazole has also been reported for cysteine conjugation by Toda et al., Angew. Chem. Int. Ed. 2013, 52, 12592-12596, incorporated by reference.

A number of processes have been developed that enable the generation of an antibody-drug conjugate with defined drug-to-antibody ratio (DAR), by site-specific conjugation to a (or more) predetermined site(s) in the antibody. Site-specific conjugation is typically achieved by engineering of a specific amino acid (or sequence) into an antibody, serving as the anchor point for payload attachment, see for example Aggerwal and Bertozzi, Bioconj. Chem. 2014, 53, 176-192, incorporated by reference, most typically engineering of cysteine. Besides, a range of other site-specific conjugation technologies has been explored in the past decade, most prominently genetic encoding of a non-natural amino acid, e.g. ρ-acetophenylalanine suitable for oxime ligation, or ρ-azidomethylphenylalanine suitable for click chemistry conjugation. The majority of approaches based on genetic reengineering of an antibody lead to ADCs with a DAR of ~2. An alternative approach to antibody conjugation without reengineering of antibody involves the reduction of interchain disulfide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ~4 (DAR4).

Ruddle et al., ChemMedChem 2019, 14, 1185-1195 have recently shown that DAR1 conjugates can be prepared from antibody Fab fragments (prepared by papain digestion of full antibody or recombinant expression) by selective reduction of the C_{H}1 and C_{L} interchain disulfide chain, followed by rebridging the fragment by treatment with a symmetrical PDB dimer containing two maleimide units. The resulting DAR1-type Fab fragments were shown to be highly homogeneous, stable in serum and show excellent cytotoxicity. In a follow-up publication, White et al., MAbs 2019, 11, 500-515, and also in WO2019034764, incorporated by reference, it was shown that DAR1 conjugates can also be prepared from full IgG antibodies, after prior engineering of the antibody: either an antibody is used which has only one intrachain disulfide bridge in the hinge region (Flexmab technology, reported in Dimasi et al., J. Mol. Biol. 2009, 393, 672-692, incorporated by reference) or an antibody is used which has an additional free cysteine, which may be obtained by mutation of a natural amino acid (e.g. HC-S239C) or by insertion into the sequence (e.g. HC-i239C, reported by Dimasi et al., Mol. Pharmaceut. 2017, 14, 1501-1516). Either engineered antibody was shown to enable the generation of DAR1 ADCs by reaction of the resulting cysteine-engineered ADC with a bis-maleimide derived PBD dimer. It was shown that the Flexmab-derived DAR1 ADCs was highly resistant to payload loss in serum and exhibited potent antitumor activity in a HER2-positive gastric carcinoma xenograft model. Moreover, this ADC was tolerated in rats at twice the dose compared to a site-specific DAR2 ADC prepared using a single maleimide-containing PBD dimer. However, no improvement in therapeutic window was noted, since the minimal effective dose (MED) of the DAR1 ADC versus the DAR2 ADC increased with the same factor 2.

It has been shown in WO2014065661, and van Geel et al., Bioconj. Chem. 2015, 26, 2233-2242, both incorporated by reference, that antibodies can be site-specifically conjugated based on enzymatic remodeling of the native antibody glycan at N297 (trimming by endoglycosidase and introduction of azido-modified GaINAc derivative under the action of a glycosyltransferase) followed by attachment of a cytotoxic payload using click chemistry. It was demonstrated by and Verkade et al., Antibodies 2018, 7, 12, that the introduction of an acylated sulfamide further improves the glycan remodeling technology in terms of therapeutic index and the DAR of the resulting antibody-drug conjugates could be tailored towards DAR2 or DAR4 by choice of specific linker. It was also demonstrated that glycan trimming before conjugation leads to nihilation of binding of the resulting antibody-drug conjugates (ADCs) to Fc-gamma receptors (Fc-silencing). ADCs prepared by this technology were found to display a significantly expanded therapeutic index versus a range of other conjugation technologies and the technology of glycan-remodeling conjugation currently clinically applied in for example ADCT-601 (ADC Therapeutics).

A similar enzymatic approach to convert an antibody into an azido-modified antibody with concomitant Fc-silencing, reported by Lhospice et al., Mol. Pharmaceut. 2015, 12, 1863-1871, incorporated by reference, employs the bacterial enzyme transglutaminase (BTG or TGase). It was shown that deglycosylation of the native glycosylation site N297 with PNGase F liberates the neighbouring N295 to become a substrate for TGase-mediated introduction, which converts the deglycosylated antibody into a bis-azido antibody upon subjection to an azide-bearing molecule in the presence of TGase. Subsequently, the bis-azido antibody was reacted with DBCO-modified cytotoxins to produce ADCs with DAR2. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

Besides the attachment of small molecules, it has also been amply demonstrated that various click chemistries are suitable for the generation of protein-protein conjugates. For example, Witte et al., Proc. Nat. Acad. Sci. 2012, 109, 11993-11998, incorporated by reference, have shown the unnatural N-to-N and C-to-C protein dimers can be obtained by a combination of sortase-mediated introduction of two complementary click probes (azide and DBCO) into two different proteins, followed by seamless ligation based on metal-free click chemistry (strain-promoted azide-alkyne cycloaddition or SPAAC). Wagner et al., Proc. Nat. Acad. Sci. 2014, 111, 16820-16825, incorporated by reference, have applied this approach to prepare a bispecific antibody based on C-terminal sortagging with an anti-influenza scFv, which was further extended to metal-free click chemistry based on inverse eletron-demand Diels-Alder cycloaddition with tetrazines by Bartels et al., Methods 2019, 154, 93-101, incorporated by reference. Tetrazine ligation had been applied earlier also by for example Devaraj et al., Angew. Chem. Int. Ed. 2009, 48, 7013-7016 and Robillard et al., Angew. Chem. Ed. Engl. 2010, 49, 3375-3378, both incorporated by reference, for antibody modification by first (random) chemical installment of a trans-cyclooctene (TCO) onto an antibody. In contrast, site-specific introduction of TCO (or tetrazine or cyclopropene other click moieties for tetrazine ligation) onto antibodies can be achieved by a multitude of methods based on prior genetic modification of the antibody as described above and for example reported by Lang et al., J. Am. Chem. Soc. 2012, 134, 10317-10320, Seitchik et al., J. Am. Chem. Soc. 2012, 134, 2898-2901 and Oller-Salvia, Angew. Chem. Int. Ed. 2018, 57, 2831-2834, all incorporated by reference.

Sortase is a suitable enzyme for site-specific modification of proteins after prior introduction of a sortase recognition sequence, as first reported by Popp et al., Nat. Chem. Biol. 2007, 3, 707-708). Many other enzyme-enzyme recognition sequence combinations are also known for site-specific protein modification, as for example summarized by Milczek, Chem. Rev. 2018, 118, 119-141, incorporated by reference, and specifically applied to antibodies as summarized by Falck and Müller, Antibodies 2018, 7, 4 (doi:10.3390/antib7010004) and van Berkel and van Delft, Drug Discov. Today: Technol. 2018, 30, 3-10, both incorporated by reference. Besides, a wide array of methods is available for non-genetic modification of native proteins, as summarized by Koniev and Wagner, Chem. Soc. Rev. 2015, 44, 5495-5551, incorporated by reference and for N-terminal modification by Rosen and Francis, Nat. Chem. Biol. 2017, 13, 697-705 and Chen et al., Chem. Sci. 2017, 8, 27172722, both incorporated by reference. Any of the above approaches could be employed to install a proper click probe into a polypeptide/protein, as for example summarized by Chen et al., Acc. Chem. Res. 2011, 44, 762-773 and Jung and Kwon, Polymer Chem. 2016, 7, 4585-4598, both incorporated by reference, and applied to an immune cell engager or a cytokine. Upon installation of the complementary click probe into the antibody targeting the tumor-associated antigen, an immune cell engager can be readily generated while the stoichiometry of tumor-binding antibody to immune cell binder can be tailored by proper choice of technology.

It has also been shown by Bruins et al., Bioconjugate Chem. 2017, 28, 1189-1193, incorporated by references, that antibodies can be site-specifically conjugated to cytotoxic payload by tyrosinase-mediated oxidation of a suitably positioned tyrosine through an intermediate 1,2-quinone that subsequently can undergo cycloaddition with a strained alkyne or alkene. The technology is referred to as strain-promoted oxidation-controlledy quinone-alkyne cycloaddition (SPOCQ).

Chemical approaches have also been developed for site-specific modification of antibodies without prior genetic modification, as for example highlighted by Yamada and Ito, ChemBioChem. 2019, 20, 2729-2737.

Chemical conjugation by affinity peptide (CCAP) for site-specific modification has been developed by Kishimoto et al., Bioconj. Chem. 2019, by using a peptide that binds with high affinity to human IgG-Fc, thereby enabling selective modification of a single lysine in the Fc-fragment with a biotin moiety or a cytotoxic payload. Similarly, Yamada et al., Angew. Chem. Int. Ed. 2019, 58, 5592-5597 and Matsuda et al., ACS Omega 2019, 4, 20564-20570, both incorporated by reference, have demonstrated that a similar approach (AJICAP™ technology) can be applied for the site-specific introduction of thiol groups on a single lysine in the antibody heavy chain. CCAP or AJICAP™ technology may also be employed for the site-specific introduction of azide groups or other functionalities.

As is clear, genetic fusion of an immune cell engager or cytokine to an IgG leads to homogenous products. Chemical conjugation of immune cell engagers to antibodies has been applied but leads to heterogenous mixtures. To date, no methods have been reported for the preparation of homogenous bispecific antibodies or antibody-cytokine fusions that do not require prior reengineering of the full-length IgG and/or enables tailoring of the number of immune cell-engaging polypeptides as well as the spacer length and structure between IgG and polypeptide. In addition, no non-genetic methods have been reported to convert an IgG into a bispecific antibody that is Fc-silent.

### SUMMARY OF THE INVENTION

A method is described suitable for conversion of a full-length IgG into an immune cell engaging bispecific (or trispecific or multispecific antibody) without requiring genetic modification of the IgG. The method enables tailoring of the molecular format of the immune cell-engaging bispecific antibody to defined 2:1 or 2:2 ratio, i.e. the ratio of complement-dependent regions in full IgG CDR (2) versus immune cell-engaging polypeptide (1 or 2). Further, the method presented is also suitable for application to an IgG that is already bispecific (i.e. with two different CDRs, for example a Duobody or a bispecific IgG obtained by knob-in-hole technology or controlled Fab-exchange technology), thereby generating a trispecific immune cell-engaging antibody of 1:1:1 or 1:1:2 format, i.e. ratio of complement-dependent regions in full IgG CDR (1:1) versus immune cell-engaging polypeptide (1 or 2). The molecular format may be further tailored by installation of more than two immune cell-engaging polypeptides, for example to give a 2:4 or a 1:1:4 or a 2:8 molecular format. Thirdly, enzymatic or chemical modification of the polypeptide fragment, i.e. the immune cell-engaging antibody or the cytokine, prior to conjugation to IgG, enables straightforward optimization of distance between IgG and polypeptide by tailoring of the spacer structure between click probe and polypeptide fragment, whereby the spacer can have any chemical structure and may consist for example of a chain of amino acids or any chemical spacer, e.g. a polyethyleneglycol-based spacer. Finally, in case the first click probe is installed onto the IgG antibody by enzymatic remodelling of the glycan structure including an endoglycosidase trimming step, the resulting bi- or multispecific antibody construct will no longer be able to bind to Fc-gamma receptors (Fc-silent), without reengineering of the antibody.

The process according to the invention is for preparing a multispecific antibody construct, and comprises conjugating a functionalized antibody Ab(F)ₓ containing x reactive moieties F, wherein x is an integer in the range 1 - 10, and an immune cell-engaging polypeptide containing one or two reactive moieties Q, wherein the antibody is specific for a tumour cell and the immune cell-engaging polypeptide is specific for an immune cell, wherein the reaction forms a covalent linkage between the functionalized antibody and the immune cell-engaging polypeptide by reaction of Q with F. The invention further concerns the multispecific antibody constructs obtainable by the process according to the invention and medical uses thereof.

### DESCRIPTION OF THE FIGURES

FIG. 1 shows a representative (but not comprehensive) set of functional groups (F) in a biomolecule, either naturally present or introduced by engineering, which upon reaction with a reactive group lead to connecting group Z. Functional group F may be artificially introduced (engineered) into a biomolecule at any position of choice. The pyridazine connecting group (bottom line) is the product of the rearrangement of the tetrazabicyclo[2.2.2]octane connecting group, formed upon reaction of tetrazine with alkyne, with loss of N₂. Connecting groups Z of structure (10a) - (10j) are preferred connecting groups to be used in the present invention.

FIG. 2 shows cyclooctynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring.

FIG. 3 shows several structures of derivatives of UDP sugars of galactosamine, which may be modified with e.g. a 3-mercaptopropionyl group (11a), an azidoacetyl group (11b), or an azidodifluoroacetyl group (11c) at the 2-position, or with an azido group at the 6-position of N-acetyl galactosamine (11d) or with a thiol group at the 6-position of N-acetyl galactosamine (11e). The monosaccharide (i.e. with UDP removed) are preferred moieties Su to be used in the present invention.

FIG. 4 shows the general process for non-genetic conversion of a monoclonal antibody into an antibody containing probes for click conjugation (F). The click probe may be on various positions in the antibody, depending on the technology employed. For example, the antibody may be converted into an antibody containing two click probes (structure on the left) or four click probes (bottom structure) or eight probes (structure on the right) for click conjugation.

FIG. 5 depicts how an IgG antibody modified with two click probes (F) can react with a polypeptide modified with the complementary click probe (Q) to form a stable bond (Q) upon reaction, where the polypeptide is elected from any polypeptide that is able to bind to an immune cell, thereby forming a bispecific antibody. Modification of the polypeptide with a single click probe Q may be achieved by any selective genetic or non-genetic method. Probes for click conjugation may be elected from any suitable combination depicted in FIG. 1. Stoichiometry of the resulting bispecific antibody depends on the number of click probes F installed in the first modification of the antibody. A symmetrical, bivalent IgG may be employed (CDR1 = CDR2), thus leading to a bispecific antibody with a 2:2 molecular format (2x attachment of polypeptide). A non-symmetrical antibody may also be employed (CDR1 ≠ CDR2), thus leading to a trispecific antibody with a 1:1:2 molecular format. If more than 2 click probes F are installed, the molecular format may be further varied, leading to for example a 2:4 molecular format (4x F installed on a symmetrical antibody) or 1:1:8 molecular format (8x F installed on a non-symmetrical antibody).

FIG. 6 shows three alternative methods to install a single immune cell-engaging polypeptide onto a full-length antibody (2:1 molecular format). The full-length antibody therefore has has first been modified with two click probes F. In one approach (arrow down), the IgG(F₂) is subjected to a polypeptide that has been modified with two complementary click probes Q, connected via a suitable spacer, both of which will react with one occurrence of F on the antibody. In the second approach (arrow right), the IgG(F₂) is subjected to a trivalent construct containing three complementary probes Q of which two will react with IgG(F₂), leaving one unit of Q free for subsequent reaction with F-modified polypeptide. In the third approach (diagonal arrow), the IgG(F₂) is subjected to a trivalent construct containing two complementary probes Q and one non-reactive click probe F₂ (which is also different from F). The two click probes Q will react with IgG(F₂), leaving F₂ for subsequent reaction with Q2-modified polypeptide.

FIG. 7 depicts a specific example of forming a bispecific antibody of 2:2 molecular format based on glycan remodeling of a full-length IgG and azide-cyclooctyne click chemistry. The IgG is first enzymatically remodeled by endoglycosidase-mediated trimming of all different glycoforms, followed by glycosyltransferase-mediated transfer of azido-sugar onto the core GIcNAc liberated by endoglycosidase. In the next step, the azido-remodeled IgG is subjected to an immune cell-engaging polypeptide, which has been modified with a single cyclooctyne for metal-free click chemistry (SPAAC), leading to a bispecific antibody of 2:2 molecular format. It is also depicted that the cyclooctyne-polypeptide construct will have a specific spacer between cyclooctyne and polypeptide, which enables tailoring of IgG-polypeptide distance or impart other properties onto the resulting bispecific antibody.

FIG. 8 is an illustration of how a azido-sugar remodeled antibody can be converted into a bispecific with a 2:1 molecular format by subjecting first to trivalent cyclooctyne construct suitable for clipping onto bis-azido antibody, leaving one cyclooctyne free for subsequent SPAAC with azido-modified polypeptide, effectively installing only one polypeptide onto the IgG. The latter polypeptide may also be modified with other complement click probes for reaction with cyclooctyne, e.g. a tetrazine moiety for inverse electron-demand Diels-Alder cycloaddition. Any combinations of F and Q (FIG. 1) can be envisaged here.

FIG. 9 shows various options for trivalent constructs for reaction with a bis-azidosugar modified mAb. The trivalent construct may be homotrivalent or heterotrivalent (2+1 format). A homotrivalent contract (X = Y) may consist of 3x cyclooctyne or 3x acetylene or 3x maleimide or 3x other thiol-reactive group. A heterotrivalent construct (X ≠ Y) may for example consist of two cyclooctyne groups and one maleimide group or two maleimides groups and one trans-cyclooctene group. The heterotrivalent construct may exist of any combination of X and Y unless X and Y and reactive with each other (e.g. maleimide + thiol).

FIG. 10 shows the general concept of sortase-mediated ligation of proteins (capital letters for common amino acid abbreviations) for C-terminal (top) or N-terminal (bottom) ligation to a protein of interest. For C-terminal ligation, a LPXTGG sequence recombinantly fused to the C-terminus of a protein of interest, where X can be any amino acid except proline and GG may be further fused to other amino acids (sequences), and sortase-mediated ligation is achieved by treatment with substrate GGG-R (with R is functionality of interest) to form a new peptide bond. Similarly, for N-terminal ligation, a GGG sequence is fused to the N-terminus of a protein of interest, for ligation with an LPXTGG sequence, where the leucine is modified with functionality of interest R, X can be any amino acid except proline and GG may be further fused to other amino acids (sequences).

FIG. 11 shows a range of bivalent BCN reagents (105, 107, 118, 125, 129, 134), trivalent BCN reagents (143, 145, 150), and monovalent BCN reagents for sortagging (154, 157, 161, 163, 168).

FIG. 12 shows a range of bivalent or trivalent cross-linkers (XL01-XL13).

FIG. 13 shows a range of antibody variants as starting materials for subsequent conversion to antibody conjugates

FIG. 14 shows a range of metal-free click reagents equipped with N-terminal GGG (169-171 and 176) or C-terminal LPETGG (172-175), suitable for sortagging of proteins.

FIG. 15 shows structures of scFv’s hOKT3 (200), mOKT3 (PF04) and α-4-1BB (PF31) equipped wth C-terminal LPETGG, C-terminal G₄SY, N-terminal SLR (or both), possibly also G₄S spacer. Structures 201-204 and PF01, PF02, PF04-PF09 are derivatives of 200, PF04 or PF31, equipped with a suitable click probe (BCN, tetrazine or azide) obtained by enzymatic or chemical derivatization.

FIG. 16 shows bivalent, bis-BCN-modified derivatives of 200.

FIG. 17 shows structures of various mutants of IL-15 (PF18) or IL-15R-IL-15 fusion protein (207, 208 and PF26, IL-15R = Sushi domain of IL-15 receptor) and derivatives thereof equipped with a suitable click probe (BCN, tetrazine or azide) or maleimide, in each case modified at its N-terminus to enable site-specific modification.

FIG. 18 shows bivalent derivatives of PF26, equipped with bis-BCN (PF27 and PF29) or bis-maleimide (PF28), as well as bis-BCN-modified IL-15 (PF30), derived from PF18.

FIG. 19 shows SDS-PAGE analysis: Lane 1 - rituximab; Lane 2 - rit-v1a; Lane 3 - rit-v1a-145; Lane 4 - rit-v1a-(201)₂; Lane 5 - rit-v1a-145-204; Lane 6 - rit-v1a-145-PF01; Lane 7 -rit-v1a-145-PF02. Gels were stained with coomassie to visualize total protein. Samples were analyzed on a 6% SDS-PAGE under non-reducing conditions (left) and 12% SDS-PAGE under reducing conditions (right).

FIG. 20 shows RP-HPLC traces of B12-v1a (upper trace) and B12-v1a-145 (lowertrace). Samples have been digested with IdeS prior to RP-HPLC analysis.

FIG. 21 shows the RP-HPLC trace under reducing conditions for the crosslinking of trastuzumab GaINProSH trast-v5b with bis-maleimide-BCN XL01 and subsequent labelling with azido-MMAF LD12 (=313).

FIG. 22 shows the RP-HPLC analysis under reducing conditions for the crosslinking of trastuzumab S239C mutant trast-v6 with bis-maleimide-BCN XL01 subsequent labelling with azido-MMAF LD12 (=313).

FIG. 23 shows the SDS-page analysis under reducing conditions for the crosslinking of trastuzumab trast-v7 with bis-maleimide-BCN XL01 subsequent labelling with azido-MMAF LD12 (=313), azido-IL15 PF19, hOkt3-tetrazine PF02 and anti-4-1BB-azide PF09.

FIG. 24 shows the RP-HPLC trace under reducing conditions for the crosslinking of trastuzumab GaINProSH trast-v5b with bis-maleimide-azide XL02 and the subsequent labelling with BCN-MMAE LD11 (=312) and BCN-IL15Rα-IL15 PF15.

FIG. 25 shows the RP-HPLC trace under reducing conditions for the crosslinking of trastuzumab S239C mutant trast-v6 with bis-maleimide-azide XL02 and the subsequent labelling with BCN-MMAE LD11 (=312) and BCN-IL15Rα-IL15 PF15.

FIG. 26 shows the SDS-page analysis under reducing conditions for the crosslinking of trastuzumab S239C mutant trast-v6 with bis-maleimide-azide XL02 and the subsequent labelling with BCN-MMAE LD11 (=312) and BCN-IL15Rα-IL15 PF15.

FIG. 27 shows the SDS-page analysis under reducing conditions for the crosslinking of trastuzumab trast-v7 with bis-maleimide-azide XL02 and C-lock-azide XL03 and the subsequent labelling with BCN-MMAE LD11 (=312).

FIG. 28 shows the RP-HPLC trace under reducing conditions for the crosslinking of trastuzumab S239C mutant trast-v6 with C-lock-azide XL03 and the subsequent labelling with BCN-MMAE LD11 (=312)

FIG. 29 shows the SDS-page analysis under reducing conditions for the crosslinking of trastuzumab S239C mutant trast-v6 with C-lock-azide XL03 and the subsequent labelling with BCN-MMAE LD11 (=312) and BCN-IL15Rα-IL15 PF15.

FIG. 30 shows the SDS-page analysis under reducing conditions for the crosslinking of trast-v8 with bis-hydroxylamine-BCN XL06 and subsequent labelling with anti-4-1BB-azide PF09 or hOkt3-tetrazine PF02

FIG. 31 shows SDS-PAGE analysis: Lane 1 -trast-v1a; Lane 2 -trast-v1a-XL11; Lane 3 and 4 - trast-v1a-XL11-PF01; Lane 5 - rit-v1a; Lane 6 - rit-v1a-XL11; Lane 7 and 8 - rit-v1a-XL11-PF01. Gels were stained with coomassie to visualize total protein. Samples were analyzed on a 6% SDS-PAGE under non-reducing conditions (left) and 12% SDS-PAGE under reducing conditions (right).

FIG. 32 shows the RP-HPLC trace under reducing conditions for the crosslinking of trastuzumab S239C mutant trast-v6 with bis-bromoacetamide-BCN XL12 and subsequent labelling with azido-MMAF LD12 (=313).

FIG. 33 shows the native SDS page analysis for the trast-v2-(PF15)₂ conjugate.

FIG. 34 shows the SDS-page analysis under reducing conditions for the crosslinking of trastuzumab GaINProSH trast-v5b with bis-maleimide-azide XL02 and the subsequent labelling with BCN-MMAE LD11 (=312) and BCN-IL15Rα-IL15 PF15.

FIG. 35 shows the SDS-page analysis under reducing conditions for the crosslinking of trastuzumab trast-v9 with bis-azide-MMAF LD10 (=310) and azido-IL15 PF19 via CuAAC.

FIG. 36 shows the RP-HPLC trace and SDS-page analysis under reducing conditions for the crosslinking of trastuzumab GaINProSH trast-v5b with bis-maleimide-BCN XL01 subsequent labelling with azido-MMAF LD12 (=313), azido-IL15 PF19, hOkt3-tetrazine PF02 and anti-4-1BB-azide PF09.

FIG. 37 shows the SDS-page analysis under reducing conditions for the crosslinking of trastuzumab S239C mutant trast-v6 with bis-maleimide-BCN XL01 subsequent labelling with azido-MMAF LD12 (=313), azido-IL15 PF19, hOkt3-tetrazine PF02 and anti-4-1BB-azide PF09.

FIG. 38 shows the SDS-page analysis under reducing conditions for the crosslinking of trastuzumab GaINProSH trast-v5b and trastuzumab S239C mutant trast-v6 with bis-maleimide-MMAE LD09 (=309), bis-maleimide-IL15Rα-IL15 PF28 and maleimide-IL15Rα-IL15 PF16.

FIG. 39 shows the SDS-page analysis under reducing conditions for the crosslinking of trastuzumab S239C mutant trast-v6 with bis-bromoacetamide-BCN XL12 subsequent labelling with azido-MMAF LD12 (=313) and hOkt3-tetrazine PF02.

FIG. 40 shows SDS-PAGE analysis on a 6% gel under non-reducing conditions: Lane 1 - rituximab; Lane 2- rit-v1a-(201)₂; Lane 3 - rit-v1a-145-PF08; Lane 4- B12-v1a-145-PF01; Lane 5 - B12-v1a-145-PF08. Gels were stained with coomassie to visualize total protein. Lanes 1 and 2 are included as a reference for non-conjugated mAb and 2:2 molecular format.

FIG. 41 shows SDS-PAGE analysis on a 6% gel under non-reducing conditions: Lane 1 - rit-v1a-(201)₂; Lane 2 - rit-v1a-145-PF01; Lane 3 - rit-v1a; Lane 4 - rit-v1a-PF22; Lane 5 -trast-v1a-PF22. Gels were stained with coomassie to visualize total protein. Lanes 1 and 2 are included as a reference for non-conjugated mAb and 2:2 molecular format.

FIG. 42 shows SDS-PAGE analysis on a 6% gel under non-reducing conditions: Lane 1 - trast-v1a; Lane 2 - trast-v1a-PF23. Gels were stained with coomassie to visualize total protein. Lanes 1 is included as a reference for non-conjugated mAb.

FIG. 43 shows SDS-PAGE analysis on a 6% gel under non-reducing conditions: Lane 1 - rit-v1a; Lane 2 - rit-v1a-(201)₂; Lane 3 - rit-v1a-145-PF01; Lane 4 - rit-v1a-PF22; Lane 5 - rit-v1a-PF23. Gels were stained with coomassie to visualize total protein. Lanes 1-4 are included as a reference for non-conjugated mAb, 2:1 and 2:2 molecular format.

FIG. 44 shows SDS-PAGE analysis on a 6% gel under non-reducing conditions: Lane 1 - rit-v1a-145; Lane 2 - rit-v1a-145-PF09; Lane 3 - trast-v1a-145; Lane 4 - trast-v1a-145-PF09; Lane 5 - rit-v1a; Lane 6 - rit-v1a-(PF07)₂; Lane 7 - trast-v1a; Lane 8 - trast-v1a-(PF07)₂. Gels were stained with coomassie to visualize total protein.

FIG. 45 shows non-reducing SDS-page analysis: lane 1 - Trast-v1a-(PF. )₁₋₂; lane 2 -trast-v1a-(209)₁₋₂; lane 3 - trast-v1a-(PF11)₁₋₂; lane 4 - trast-v1a; lane 5 - trast-v1a-145-PF12; lane 6 - trast-v1a-145. Gels were stained with coomassie to visualize total protein.

FIG. 46 shows SDS-PAGE analysis on a 6% gel under non-reducing conditions: Lane 1 - rit-v1a-145; Lane 2 - rit-v1a-145-PF17; Lane 3 - trast-v1a-145; Lane 4 - trast-v1a-145-PF17. Gels were stained with coomassie to visualize total protein.

FIG. 47 shows SDS-PAGE analysis on a 6% gel under non-reducing conditions: Lane 1 - trast-v1a; Lane 2 - trast-v1a-PF29; Lane 3 - rit-v1a; Lane 4 - rit-v1a-PF29. Gels were stained with coomassie to visualize total protein.

FIG. 48 shows effect of bispecifics based on hOKT3 200 on RajiB Tumor cell killing with human PBMCs. Bispecifics and calculated EC₅₀ values are shown in the legend. B12-v1a-145-PF01 was included as a negative control.

FIG. 49 shows effect of bispecifics based on anti-4-1BB PF31 on RajiB Tumor cell killing with human PBMCs. Bispecifics and calculated EC₅₀ values are shown in the legend. B12-v1a-145-PF31 was included as a negative control.

FIG. 50 shows cytokine levels in supernatants of a RajiB-PBMC co-culture after incubation with bispecifics based on hOKT3 200. The murine OKT3 mlgG2a antibody (Invitrogen 16-0037-81) was included as a positive control.

FIG. 51 shows cytokine levels in supernatants of a RajiB-PBMC co-culture after incubation with bispecifics based on anti-4-1BB PF31. The murine OKT3 mlgG2a antibody (Invitrogen 16-0037-81) was included as a positive control.

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application. When the structure of a compound is depicted as a specific cis or trans isomer, it is to be understood that the invention of the present application is not limited to that specific cis or trans isomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “monosaccharide” is herein used in its normal scientific meaning and refers to an oxygen-containing heterocycle resulting from intramolecular hemiacetal formation upon cyclisation of a chain of 5-9 (hydroxylated) carbon atoms, most commonly containing five carbon atoms (pentoses), six carbon atoms (hexose) or nine carbon atoms (sialic acid). Typical monosaccharides are ribose (Rib), xylose (Xyl), arabinose (Ara), glucose (Glu), galactose (Gal), mannose (Man), glucuronic acid (GIcA), N-acetylglucosamine (GIcNAc), N-acetylgalactosamine (GaINAc) and N-acetylneuraminic acid (NeuAc).

The term “cytokine” is herein used in its normal scientific meaning and are small molecule proteins (5-20 kDa) that modulate the activity of immune cells by binding to their cognate receptors and by triggering subsequent cell signalling. Cytokines include chemokines, interferons (IFN), interleukins, monokines, lymphokines, colony-stimulating factors (CSF) and tumour necrosis factors (TNF). Examples of cytokines are IL-1 alpha (IL1a), IL-1 beta (IL1b), IL-2 (IL2), IL-4 (IL4), IL-5 (IL5), IL-6 (IL6) , IL8 (IL-8), IL-10 (IL10), IL-12 (IL12), IL-15 (IL15), IFN-alpha (IFNA), IFN-gamma (IFN- G), and TNF-alpha (TNFA).

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art. Typical examples of antibodies include, amongst others, abciximab, rituximab, basiliximab, palivizumab, infliximab, trastuzumab, efalizumab, alemtuzumab, adalimumab, tositumomab-I131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

The term “antibody construct” is herein defined as the covalently linked combination of two or more different proteins, wherein one protein is an antibody or an antibody fragment and the other protein (or proteins) is an immune cell-engaging polypeptide, such as an antibody, an antibody fragment or a cytokine. Typically, one of the proteins is an antibody or antibody fragments with high affinity for a tumor-associated receptor or antigen, while one (or more) of the other proteins is an antibody, antibody fragment or polypeptide with high affinity for a receptor or antigen on an immune cell.

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “bispecific” is herein defined as an antibody construct with affinity for two different receptors or antigens, which may be present on a tumour cell or an immune cell, wherein the bispecific may be of various molecular formats and may have different valencies.

The term “trispecific” is herein defined as an antibody construct with affinity for three different receptors or antigens, which may be present on a tumour cell or an immune cell, wherein the trispecific may be of various molecular formats and may have different valencies.

The term “multispecific” is herein defined as an antibody construct with affinity for at least two different receptors or antigens, which may be present on a tumour cell or an immune cell, wherein the multispecific may be of various molecular formats and may have different valencies.

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “polar linker” is herein defined as a linker that contains structural elements with the specific aim to increase polarity of the linker, thereby improving aqueous solubility. A polar linker may for example comprise one or more units, or combinations thereof, selected from ethylene glycol, a carboxylic acid moiety, a sulfonate moiety, a sulfone moiety, an acylated sulfamide moiety, a phosphate moiety, a phosphinate moiety, an amino group or an ammonium group.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “bioconjugate” is herein defined as a compound wherein a biomolecule is covalently connected to a payload via a linker. A bioconjugate comprises one or more biomolecules and/or one or more target molecules.

A “biomolecule” is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “payload” refers to the moiety that is covalently attached to a targeting moiety such as an antibody. Payload thus refers to the monovalent moiety having one open end which is covalently attached to the targeting moiety via a linker. A payload may be small molecule or a biomolecule.

The term “molecular format” refers to the number and relative stoichiometry of different binding elements in a bispecific, trispecific or multispecific antibody, with 2:2 molecular format denoting a bispecific with two polypeptide fragments able to bind one target and polypeptide fragment able to bind another target, and with 1:1:2 molecular format denoting a trispecific with one polypeptide fragment able to bind one target, another polypeptide fragment able to bind another target and a third polypeptide fragment able to bind a third target, where in all cases the targets are different. The term “2:1 molecular format” refer to a protein conjugate consisting of a bivalent monoclonal antibody (IgG-type) conjugated to a single functional payload.

The term “complement-dependent region” or “CDR” refers to the variable fragment of an antibody that is able to bind a specific receptor or antigen.

The present inventors have developed an improved process for the manufacture of multispecific antibody constructs, which are on one hand specific for a tumour cell and on the other hand specific for an immune cell, such as a T cell, an NK cell, a monocyte, a macrophage, a granulocyte. For the first time, it has become possible to prepare such bispecific or multispecific constructs with full control of molecular format and without the need of genetic engineering. The process according to the invention specifically couples an x number of immune cell-engaging polypeptides to a tumour-specific antibody, such that final constructs have a predetermined molecular format and a ratio of tumour-binding domains versus immune cell-binding domain of for example 2:1 or 2:2 or even 2:8.

The present invention concerns the process for preparing the multispecific antibody constructs as well as the multispecific antibody constructs obtainable thereby. The invention further concerns the (medical) use of the multispecific antibody constructs according to the invention. The invention further concerns the intermediary immune cell-engaging polypeptide containing one or two reactive moieties Q.

Here below, molecular moieties are defined in starting materials, intermediates and final products. The skilled person understands that any definition of preferred embodiment of either one of those equally applies to the other compounds, as long as that part of the molecule is not affected during the conversion. Likewise, anything structurally defined for the process according to the invention equally applies to the compounds according to the invention.

### Process for Preparing a Multispecific Antibody Construct

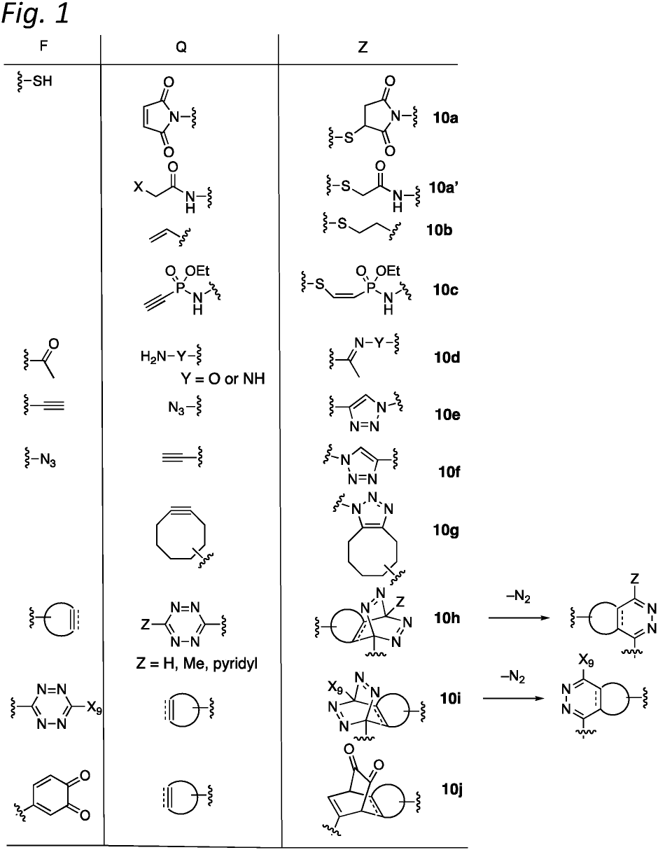

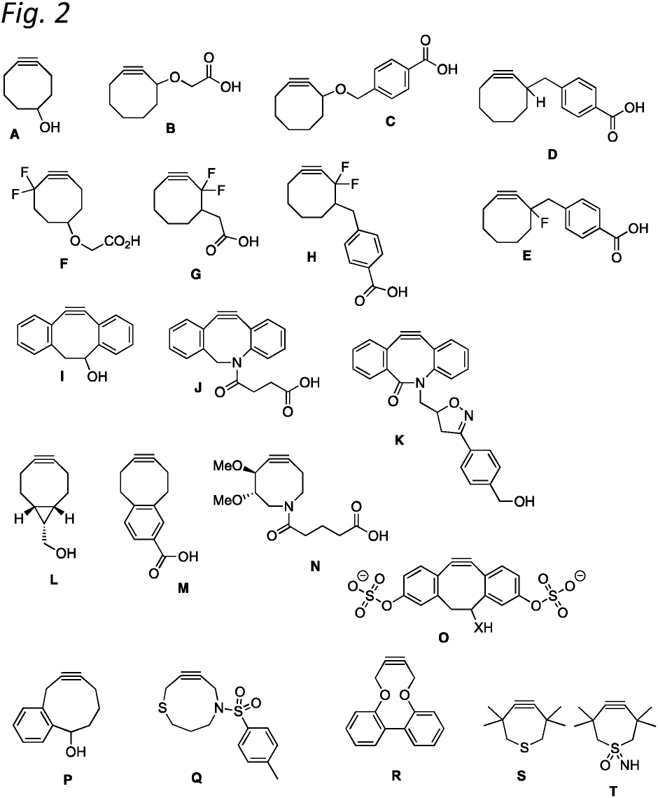

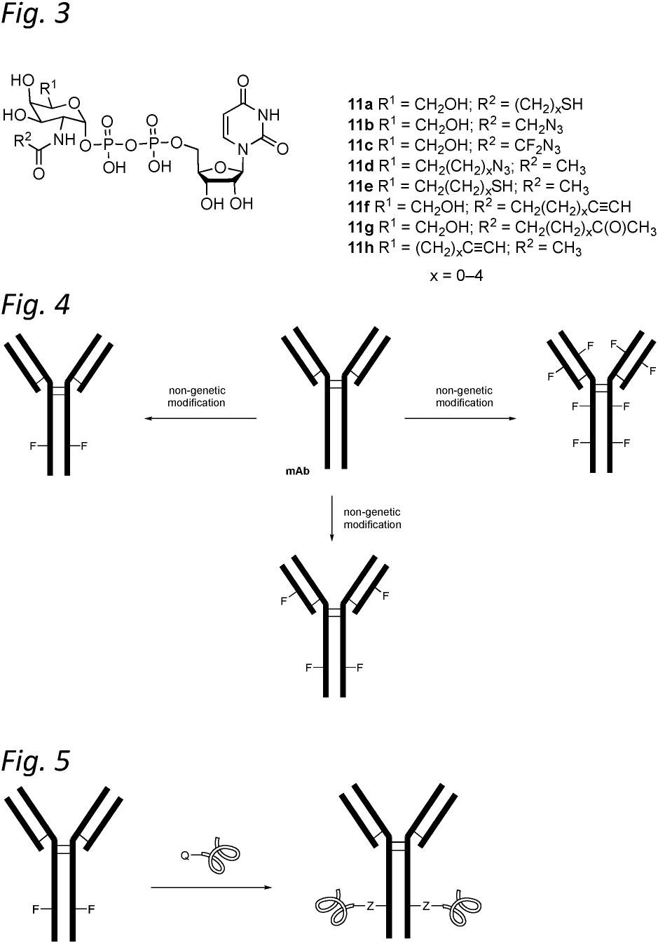

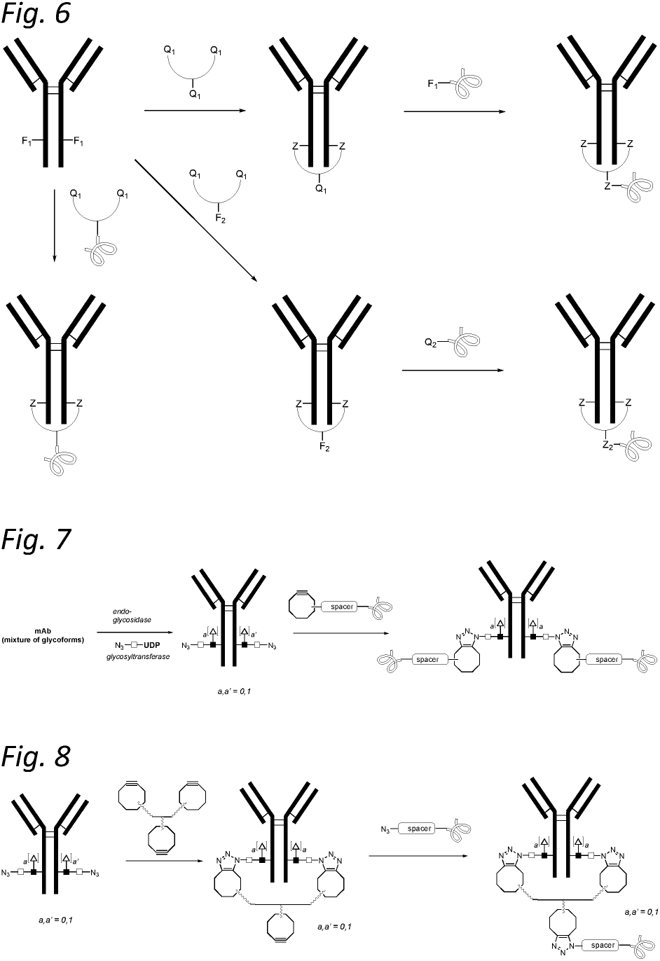

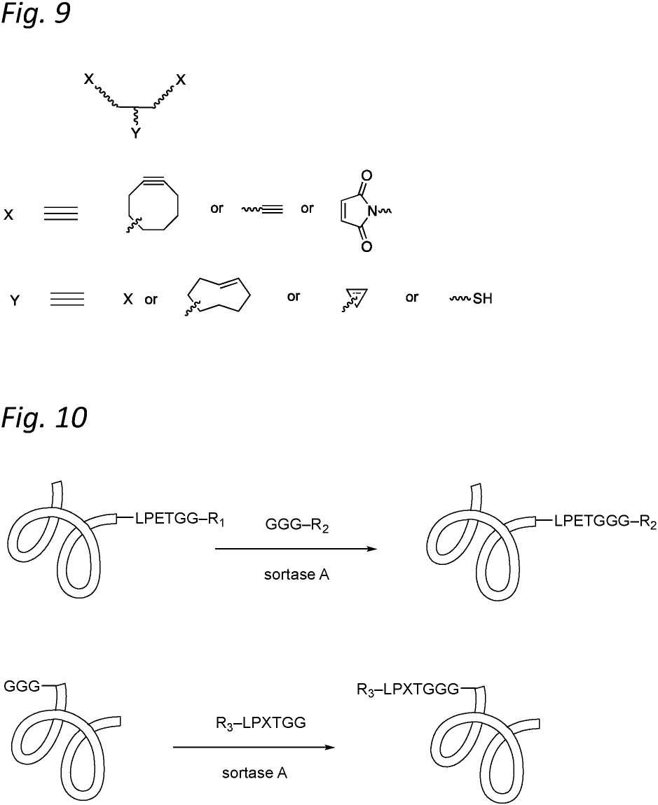

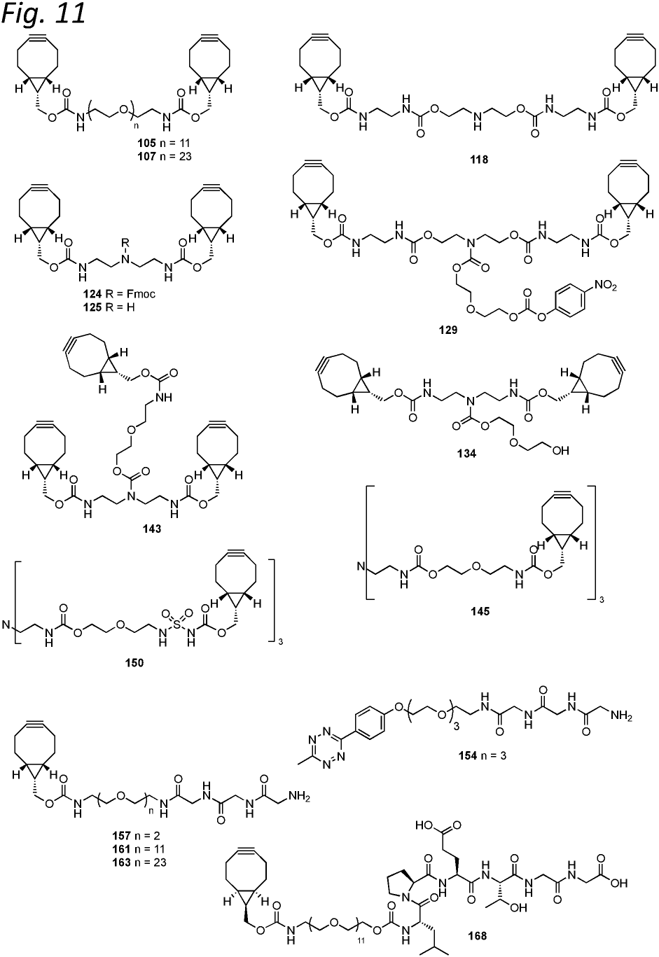

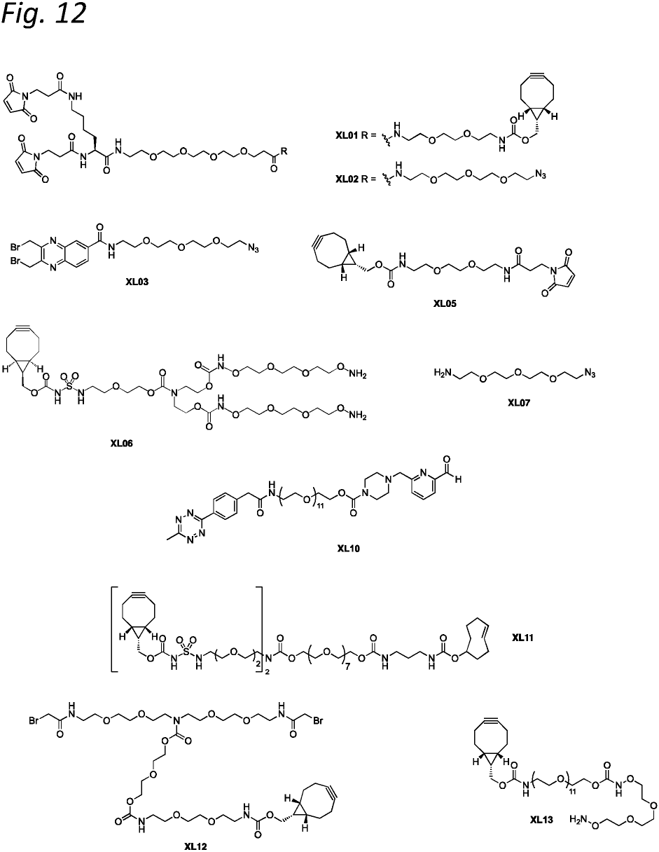

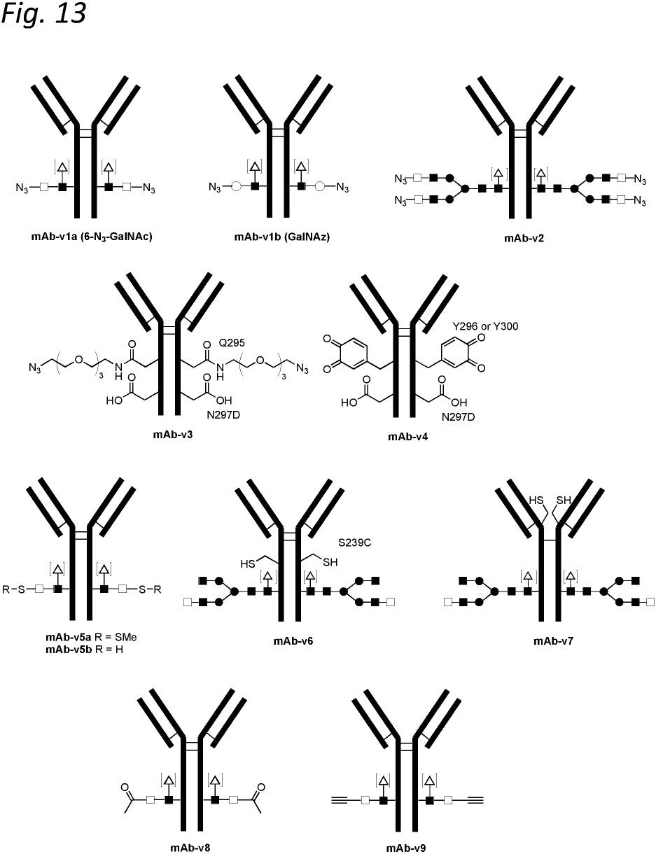

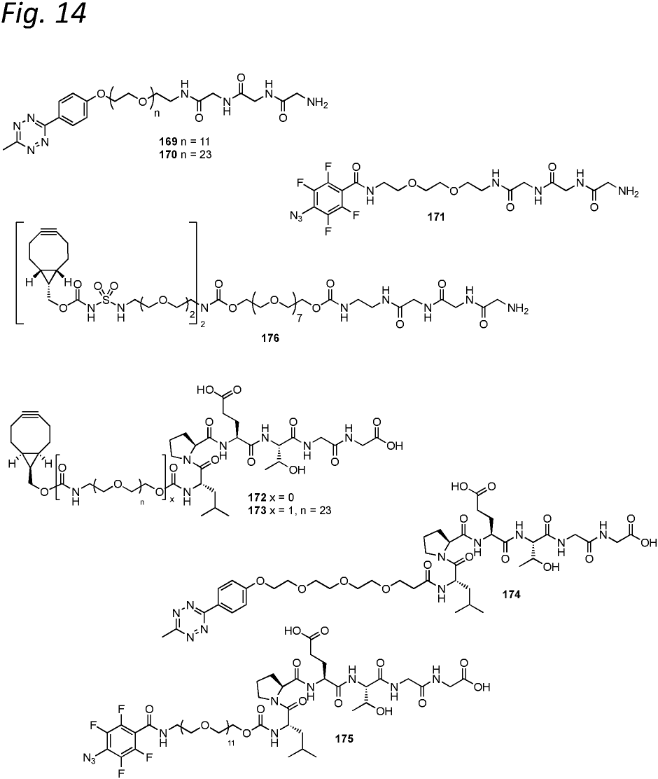

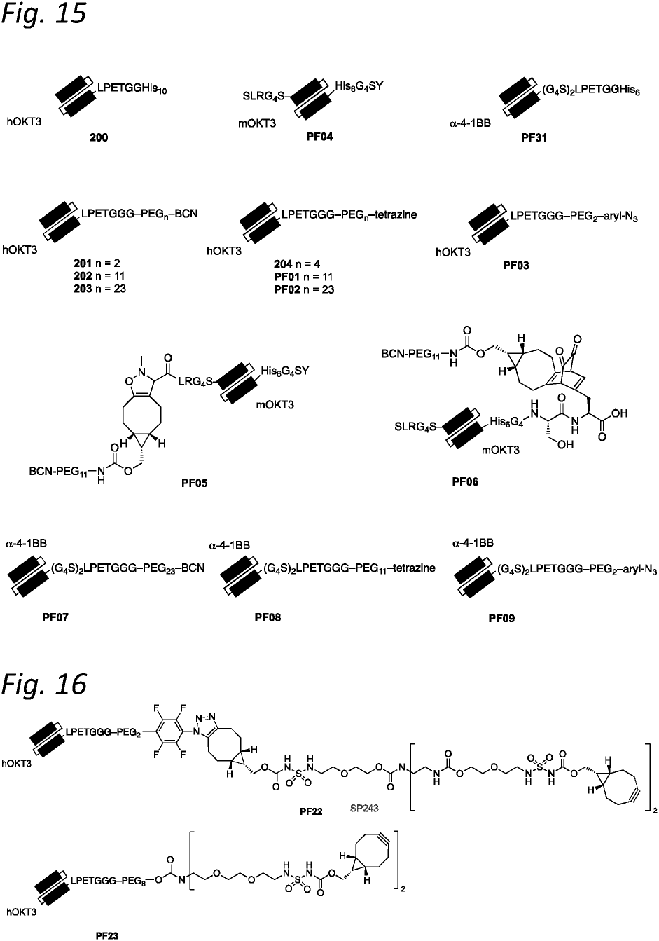

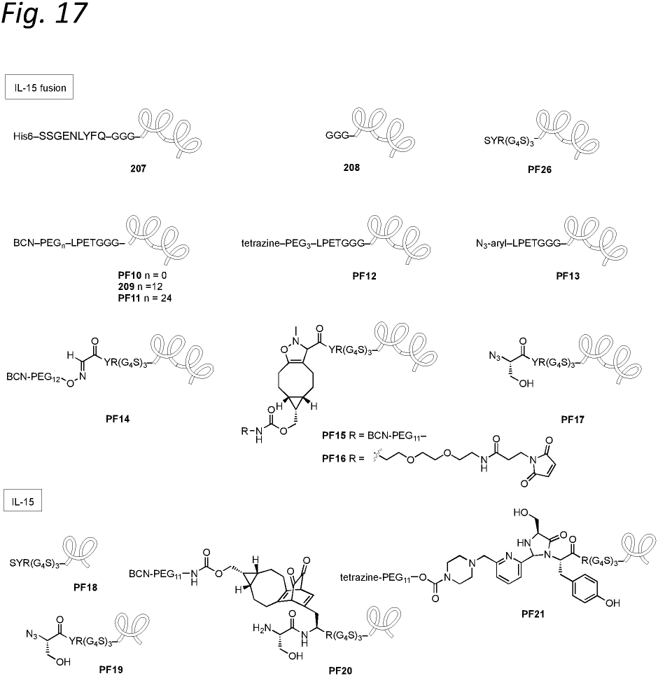

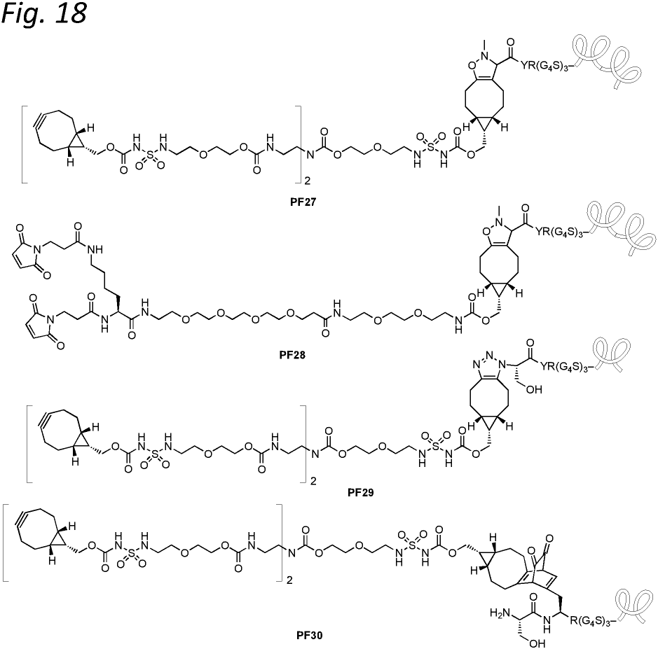

In a first aspect, the invention concerns a process for preparing a multispecific antibody construct. The process according to the invention involves a reaction between an appropriately functionalized antibody and an appropriately functionalized immune cell-engaging polypeptide. The reaction affords the conjugation of both fragments, i.e. a covalent linkage between the functionalized antibody and the immune cell-engaging polypeptide is formed. For that to happen, the immune cell-engaging polypeptide contains or is functionalized with one or two reactive moieties Q and the functionalized antibody contains or is functionalized with 1 - 10 reactive moieties F, wherein Q and F are reactive towards each other such that the conjugation reaction forms a covalent linkage between the functionalized antibody and the immune cell-engaging polypeptide by reaction of Q with F (a list of potential Q and F moieties is provided in FIG. 1).

Generally, the process according to the invention is represented according to Scheme 1.

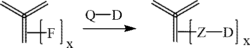

Herein, the immune cell-engaging polypeptide is represented by D. The conjugation reaction between reactive moieties F and Q affords connecting group Z. There may be a linker (L^{A}) present in between Q and D, giving Scheme 1b.

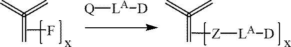

Herein, L^{A} is a linker that covalently links Q and D or, after reaction of Q with F, covalently links Z and D. Herein, D represents the immune cell-engaging polypeptide.

The multispecific antibody constructs obtained by the process according to the invention can be represented by structure (1a) or (1b):

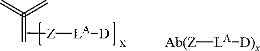

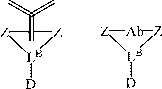

Herein, L^{A} is a bivalent linker that connects Z to D, and L^{B} is a trivalent linker that connects two occurrences of Z to D.

In a preferred embodiment, x = 2. The process according to this embodiment can be represented according to Scheme 2 or 3.

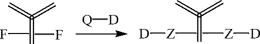

Herein, the immune cell-engaging polypeptide is represented by D. The conjugation reaction between reactive moieties F and Q affords connecting group Z.

In an especially preferred embodiment, x = 2 and the functionalized antibody is reacted with an immune cell-engaging polypeptide having two reactive groups Q. The process according to this preferred embodiment can be represented according to Scheme 3.

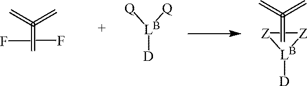

(2) (1b)

Herein, the immune cell-engaging polypeptide is represented by structure (2). The polypeptide D is connected two both reactive moieties Q via a trivalent linker L^{B}. The same linker is present in the final multispecific antibody construct, where it links both occurrences of Z with the polypeptide D.

In a preferred embodiment, x = 1. The process according to this embodiment can be represented according to Scheme 4.

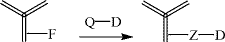

Herein, the immune cell-engaging polypeptide is represented by D. The conjugation reaction between reactive moieties F and Q affords connecting group Z.

The functionalized antibody containing one reactive moiety F in a preferred embodiment has structure (3) as shown below. Herein, L^{C} is a trivalent linker that links F to the antibody via two instances of Z. Performing the process according to the invention with the functionalized antibody according to structure (3) affords the multispecific antibody construct according to structure (1b). Herein, linker L^{B} that connects to D contains the connecting group that is formed when F and Q react and covalently attach.

The functionalized antibody according to structure (3) can be prepared by reacting a linker compound comprising two reactive moieties Q¹ and one reactive moiety F with a functionalized antibody comprising two reactive moieties F¹, wherein Q¹ and F¹ react to form a covalent connection between the antibody and F, as depicted in Scheme 5 below. The linker compound contains the same linker L^{C}, which links F to both occurrences of Q¹.

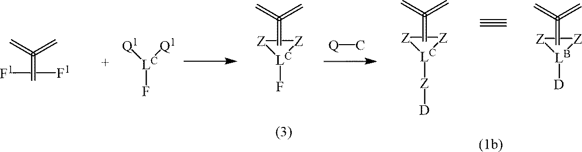

Herein, Q¹ and F¹ are reactive moieties just as Q and F, and the definition and preferred 5 embodiments of Q and F equally apply to Q¹ and F¹. The presence of F in the linker compound should not interfere with the reaction, which can be accomplished with the inertness of F in the reaction between Q¹ and F¹. The inventors have found that a trivalent linker compound wherein both Q¹ and F are the same reactive moiety, the reaction with Ab(F¹)₂ only occurs for two combinations Q¹/F¹, and the third reactive moiety remains unreacted. Further reduction of a third reaction taking place at the linker compound is accomplished by performing the reaction in dilute conditions.

The present invention makes use of linkers. Linkers, also referred to as linking units, are well-known to a person skilled in the art and may be any chain of potentially substituted aliphatic carbon atoms or (hetero)aromatic moieties or a combination thereof. Suitable examples of suitable linkers include (poly)ethylene glycol diamines (e.g. 1,8-diamino-3,6-dioxaoctane or equivalents comprising longer ethylene glycol chains), polyethylene glycol chains or polyethylene oxide chains, polypropylene glycol chains or polypropylene oxide chains and 1,h-diaminoalkanes wherein h is the number of carbon atoms in the alkane. A preferred class of suitable linkers comprises polar linkers. Polar linkers for better aqueous solubility are also known in the art and contains structural elements with the specific aim to increase polarity. A polar linker may for example comprise one or more units, or combinations thereof, selected from ethylene glycol, a carboxylic acid moiety, a sulfonate moiety, a sulfone moiety, an acylated sulfamide moiety, a phosphate moiety, a phosphinate moiety, an amino group or an ammonium group. The linkers defined here are suitable candidates for any of the linkers defined in the context of the present invention, including L^{A}, L^{B}, L^{C}, L¹, L² and L³.

The process according to the invention affords a multispecific antibody construct. Thus, the specificity of the functionalized antibody and the immune cell-engaging polypeptide is towards cell types. The antibody is preferably a monoclonal antibody, more preferably selected from the group consisting of IgA, IgD, IgE, IgG and IgM antibodies. Even more preferably AB is an IgG antibody. The IgG antibody may be of any IgG isotype, such as IgG1, IgG2, Igl3 or IgG4. Preferably, the antibody is a full-length antibody, but AB may also be a Fc fragment.

Typically, the functionalized antibody is specific for an extracellular receptor on a tumour cell. In a preferred embodiment, the extracellular receptor is selected from the group of consisting of CD30, nectin-4 (PVRL4), folate receptor alpha (FOLR1), CEACAM5 (CD66e), CD37, TF (CD142, thromoplastin), ENPP3, CD203c (AGS-16), EGFR, CD138/syndecan-1, Axl, DKL-1, IL13R, HER3, CD166, LIV-1 (SLC39A6, ZIP6), c-Met, CD25 (IL-2R-α), PTK7 (CCK4), CD71 (transferrin R), FLT3, GD3, ASCT2, IGF-1R, CD123 (IL-3Rα), CD74, guanyl cyclase C (GCC), CD205 (Ly75), ROR1, ROR2, CD46, CD228 (P79, SEMF), CD70, Globo H, Lewis Y (CD174), MUC1 (PEM), CA-IX (CA9, MN), PSMA, CanAg, EphA2, Cripto, av-integrin (ITGAV, CD51), CD56 (NCAM), SLITRK6 (SLC44A4), 5T4 (TPBG), c-KIT (CD117), FGFR2, Notch3, CS1 (SLAMF7, CD319), gpNMB, TIM- 1, CD19, CD20, Cadherin-6 (CDH6), P-cadherin (pCAD, CDH3), C4.4a, DPEP3, MFI2 (TAA), CD48a (SLAMF2), LRRC15, PRLR (prolactin), DLL3 (delta-like 3), CD324, RNF43, ADAM-9, AMHRII (anti-Müllerian), CD13, CD38, CD45, claudin (CLDN18.2), Gal-3BP (Mac-2 bp), GFRA1, MICA/B, RON, TM4SF, TWEAKR, TROP-2 (EGP-1), BCMA (CD269), B7-H3 (CD276), BMPR1B (bone morphogenetic protein receptor-type IB), E16 (LAT1, SLC7A5), STEAP1 (six transmembrane epithelial antigen of prostate), MUC16 (0772P, CA125), MPF (MPF, mesothelin (MSLN), SMR, megakaryocyte potentiating factor, mesothelin), NaPi2b (NAPI-3B, NPTIIb, SLC34A2, solute carrier family 34 (sodium phosphate), member 2, type II sodium-dependent phosphate transporter 3b), Sema 5b (FLJ10372, KIAA1445, Mm.42015, SEMASB, SEMAG, Semaphorin 5b Hlog, sema domain, seven thrombospondin repeats (type 1 and type 1-like), transmembrane domain (TM) and short cytoplasmic domain, (semaphorin) 5B), PSCA hlg (2700050C12Rik, C530008O16Rik, RIKEN cDNA 2700050C12, RIKEN cDNA 2700050C12 gene), ETBR (Endothelin type B receptor), MSG783 (RNF24, hypothetical protein FLJ20315), STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein), TrpM4 (BR22450, F120041, TRPM4, TRPM4B, transient receptor potential cation channel, subfamily M, member 4), CRIPTO (CR, CR1, CRGF, CRIPTO, TDGF, teratocarcinoma-derived growth factor), CD21 (CR2 (Complement receptor 2) or C3DR (C3d/Epstein Barr virus receptor) or Hs 73792), CD79b (CD79B, CD7913, IGb (immunoglobulin-associated beta), B29), FcRH2 (IFGP4, IRTA4, SPAP1A (SH2 domain containing phosphatase anchor protein 1a) , SPAP1B, SPAP1C), HER2, NCA, MDP, IL20Rα, Brevican, EphB2R, ASLG659, PSCA, GEDA, BAFF-R (B cell-activating factor receptor, BLyS receptor 3, BR3), CD22 (B-cell receptor CD22-B isoform), CD79a (CD79A, CD79α, immunoglobulin-associated alpha), CXCR5 (Burkitt’s lymphoma receptor 1), HLA-DOB (Beta subunit of MHC class II molecule (Ia antigen)), P2X5 (Purinergic receptor P2× ligand-gated ion channel 5), CD72 (B-cell differentiation antigen CD72, Lyb-2), LY64 (Lymphocyte antigen 64 (RP105), type I membrane protein of the leucine rich repeat (LRR) family), FcRH1 (Fc receptor-like protein 1), FcRH5 (IRTA2, Immunoglobulin superfamily receptor translocation associated 2), TENB2 (putative transmembrane proteoglycan), PMEL17 (silver homolog; SILV; D12S53E; PMEL17; SI; SIL), TMEFF (transmembrane protein with EGF-like and two follistatin-like domains 1; Tomoregulin-1), GDNF-Ra1 (GDNF family receptor alpha 1, GFRA1, GDNFR, GDNFRA, RETL1, TRNR1, RET1L, GDNFR-alpha1, GFR-ALPHA-1), Ly6E (lymphocyte antigen 6 complex, locus E; Ly67,RIG-E,SCA-2,TSA-1), TMEM46 (shisa homolog 2 (Xenopus laevis); SHISA2), Ly6G6D (lymphocyte antigen 6 complex, locus G6D; Ly6-D, MEGT1), LGR5 (leucine-rich repeat-containing G protein-coupled receptor 5; GPR49, GPR67), RET (ret proto-oncogene; MEN2A; HSCR1; MEN2B; MTC1; PTC; CDHF2; Hs.168114; RET51; RET-ELE1), LY6K (lymphocyte antigen 6 complex, locus K; LY6K; HSJ001348; FLJ35226), GPR19 (G protein-coupled receptor 19; Mm.4787), GPR54 (KISS1 receptor; KISS1R; GPR54; HOT7T175; AXOR12), ASPHD1 (aspartate beta-hydroxylase domain containing 1; LOC253982), Tyrosinase (TYR; OCAIA; OCA1A; tyrosinase; SHEP3), TMEM118 (ring finger protein, transmembrane 2; RNFT2; FLJ14627), GPR172A (G protein-coupled receptor 172A; GPCR41; FLJ11856; D15Ertd747e), CD33, TAG72 (tumour-associated glycoprotein-72), CLL-1, CLEC12A, MOSPD2, EpCAM, CD133, FAP, PD-L1 and SSTR2.

The immune cell-engaging polypeptide is preferably selected from the group consisting of Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides. Typically, the immune cell-engaging polypeptide is specific for an extracellular receptor on an immune cell. Associating tumour cells with immune cells such as T-cells, NK-cells, monocytes, macrophages and granulocyte, has been identified as a particularly interesting approach towards the treatment of cancer. Thus, in a preferred embodiment, the immune cell towards which the polypeptide is specific is for an cellular receptor on a T-cell, an NK-cell, a monocyte, a macrophage or a granulocyte, preferably on a T-cell or an NK-cell. In one embodiment, the immune cell-engaging polypeptide is specific for a cellular receptor on a T cell, preferably wherein the cellular receptor on a T cell is selected from the group consisting of CD3, CD28, CD137 (4-1BB), CD134 (OX40), CD27, Vγ9Vδ2 and ICOS. Especially preferred T cell-engaging peptides are selected from OKT3, UCHT1, BMA031 and VHH 6H4, most preferably OKT3 is used. In an alternative embodiment, the cell-engaging polypeptide is specific for a cellular receptor on a NK cell, preferably wherein the cellular receptor on a NK cell is selected from the group consisting of CD16, CD56, CD335 (NKp46), CD336 (NKp44), CD337 (NKp30), CD28, NKG2A, NKG2D (CD94), KIR, DNAM-1 and CD161. Especially preferred NK cell-engaging peptides are selected from IL-2, IL-15, IL-15/IL-15R complex and IL-15/IL-15R fusion, most preferably IL-15/IL-15R fusion. In one embodiment, the immune cell-engaging polypeptide is specific for a cellular receptor on a monocyte or a macrophage, preferably wherein the cellular receptor on the monocyte or macrophage is CD64. In one embodiment, the immune cell-engaging polypeptide is specific for a cellular receptor on a granulocyte, preferably wherein the cellular receptor on the granulocyte is CD89. In one embodiment, the immune cell-engaging polypeptide is an antibody specific for IL-2 or IL-15.

In especially preferred embodiment, the immune cell-engaging peptide is selected from OKT3, UCHT1, BMA031, VHH 6H4, IL-2, IL-15, IL-15/IL-15R complex, IL-15/IL-15R fusion, an antibody specific for IL-2 and an antibody specific for IL-15, more preferably selected from OKT3, IL-15/IL-15R fusion, IL-15, mAb602, Nara1 or TCB2. In especially preferred embodiment, the immune cell-engaging peptide is OKT3 or IL-15/IL-15R fusion. In another especially preferred embodiment, the immune cell-engaging peptide is OKT3 or IL-15. Most preferably, the immune cell-engaging polypeptide is OKT3.

In one distinct embodiment, the invention also pertains to multispecific antibody constructs according to the invention, wherein the D is not an immune cell-engaging polypeptide as defined herein, but is an antibody as defined here above for the functionalized antibody, wherein both the antibody Ab and D are different antibodies, directed to different targets. Preferably, both targets are selected from the list provided in paragraph [0099] above. Preferred combinations of targets are those of the prior art conjugates disclosed in paragraph [0010] above. In an especially preferred embodiment, one of the antibody targets HER1 and the other one targets cMET.

The process according to the invention is versatile in that various constructs can be obtained, depending on the number (x) of reactive groups F present on the functionalized antibody and the number (y) of reactive groups Q present on the immune cell-engaging polypeptide. For example, when x = 1 and y = 1, a 1:1 construct can be obtained. For example, when x = 2 and y = 1, a 2:1 construct can be obtained. When an immune cell-engaging polypeptide having two reactive groups Q (y = 2) is used, a 1:1 construct can be obtained when x = 2.

The number of functional groups introduced in the functionalized antibody can be governed by the preparation of the functionalized antibody. For example, random conjugation of an antibody with a chemical construct consisting of reactive moiety F connected to an active ester can be achieved to result in an average number of acylation events per antibody, which can be tailored by adjusting the stoichiometry of the reactive moiety F-active ester construct versus antibody. Similarly, reduction of interchain disulfide bonds of an antibody followed by reaction with a defined number of reactive moiety F containing maleimide constructs (or other thiol-reactive constructs) leads to a loading of groups F that can be tailored by stoichiometry. A more controlled, site-specific process of antibody conjugation can be achieved for example by genetic engineering of the antibody to contain two unpaired cysteines (one per heavy chain or one per light chain), to provide exactly two reactive moieties F onto the antibody upon subjection of the antibody to F containing maleimide constructs. Genetic encoding enables the direct expression of an antibody to contain a predefined number of reactive moieties F at specific sites by applying the AMBER stop codon. A range of enzymatic approaches have been also been reported to install a defined number of reactive moieties F onto an antibody, for example based on transglutaminase (TGase), sortase, formyl-glycine generating enzyme (FGE) and others. Thus, in one embodiment, the functionalized antibody is prepared by random conjugation, reduction of interchain disulfide bonds followed by reaction with F-containing thiol-reactive constructs, introduction of unpaired cysteine residues followed by reaction with F-containing thiol-reactive constructs, enzymatic introduction of reactive moieties F, and introduction of reactive moieties by genetic engineering. The use of genetic engineering is least preferred in the context of the present application, while enzymatic introduction of reactive moieties F is most preferred.

For example, GlycoConnect technology (see e.g. WO 2014/065661 and van Geel et al., Bioconj. Chem. 2015, 26, 2233-2242, incorporated by reference) utilizes the naturally present glycans at the heavy chain of monoclonal antibodies to introduce a fixed number of click probes, in particular azides. Thus, in a preferred embodiment, the functionalized antibody is prepared by (i) optionally trimming of the native glycan with a suitable endoglycosidase, thereby liberating the core GlcNAc, which is typically present on Asn-297, followed by (ii) transfer of an unnatural, azido-bearing sugar substrate from the corresponding UDP-sugar under the action of a suitable glycosyltransferase, for example transfer of GalNAz with galactosyltransferase mutant Gal-T(Y289L) or 6-azidoGalNAc with GalNAc-transferase (GalNAc-T). Alternatively, GalNAc-T can also be applied to install onto the core GIcNAc GalNAc derivatives harbouring aromatic moieties or thiol function on the Ac group. The functionalized antibody according to structure (4) can be obtained with this technology, wherein trimming step (i) may be employed to obtained functionalized antibodies having e = 0, or can be omitted to obtain functionalized antibodies having e = 1 - 10. Preferably, step (i) is performed and e = 0.

Thus, in a preferred embodiment, the functionalized antibody is according to structure (4)

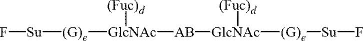

Herein:
AB is the antibody;
D is 0 or 1;
e is an integer in the range of 0 10;
G is a monosaccharide moiety;
GlcNAc is an N-acetylglucosamine moiety;
Su is a monosaccharide derivative;
Fuc is a fucose moiety;
F are reactive groups capable of undergoing a conjugation reaction with Q, wherein they are joined in connecting group Z.

Each of the two GlcNAc moieties in (4) are preferably present at a native N-glycosylation site in the Fc-fragment of antibody AB. Preferably, said GIcNAc moieties are attached to an asparagine amino acid in the region 290-305 of AB. In a further preferred embodiment, the antibody is an IgG type antibody, and, depending on the particular IgG type antibody, said GIcNAc moieties are present on amino acid asparagine 297 (Asn297 or N297) of the antibody.

G is a monosaccharide moiety and e is an integer in the range of 0 - 10. G is preferably selected from the group consisting of glucose (Glc), galactose (Gal), mannose (Man), fucose (Fuc), N-acetylglucosamine (GIcNAc), N-acetylgalactosamine (GalNAc), N-acetylneuraminic acid (NeuNAc) and sialic acid and xylose (Xyl). More preferably, G is selected from the group consisting of glucose (Glc), galactose (Gal), mannose (Man), fucose (Fuc), N-acetylglucosamine (GlcNAc) and N-acetylgalactosamine (GalNAc).

In a preferred embodiment, e is 0 and G is absent. G is typically absent when the glycan of the antibody is trimmed. Trimming refers to treatment with endoglycosidase, such that only the core GlcNAc moiety of the glycan remains.

In another preferred embodiment, e is an integer in the range of 1 - 10. In this embodiment it is further preferred that G is selected from the group consisting of glucose (Glc), galactose (Gal), mannose (Man), fucose (Fuc), N-acetylglucosamine (GIcNAc), N-acetylgalactosamine (GalNAc), N-acetylneuraminic acid (NeuNAc) or sialic acid and xylose (Xyl), more preferably from the group consisting of glucose (Glc), galactose (Gal), mannose (Man), fucose (Fuc), N-acetylglucosamine (GIcNAc) and N-acetylgalactosamine (GalNAc).

When e is 3 - 10, (G)ₑ may be linear or branched. Preferred examples of branched oligosaccharides (G)ₑ are (a), (b), (c), (d), (e), (f), (g) and (h) as shown below.

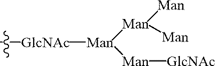

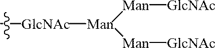

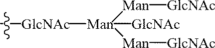

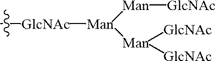

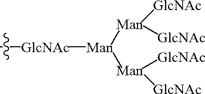

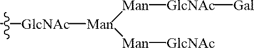

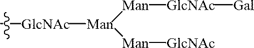

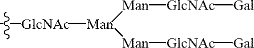

In case G is present, it is preferred that it ends in GlcNAc. In other words, the monosaccharide residue directly connected to Su is GlcNAc. The presence of a GlcNAc moiety facilitates the synthesis of the functionalized antibody, as monosaccharide derivative Su can readily be introduced by glycosyltransfer onto a terminal GlcNAc residue. In the above preferred embodiments for (G)ₑ, having structure (a) - (h), moiety Su may be connected to any of the terminal GlcNAc residues, i.e. not the one with the wavy bond, which is connected to the core GIcNAc residue on the antibody.

It is particularly preferred that G is absent, i.e. that e = 0. An advantage of an antibody-payload-conjugate (1) wherein e = 0 is that when such conjugate is used clinically, binding to Fc gamma receptors CD16, CD32 and CD64 is significantly reduced or fully abrogated.

Su is a monosaccharide derivative, also referred to as sugar derivative. Preferably, the sugar derivative is able to be incorporated into the functionalized antibody by means of glycosyltransfer. More preferably, Su is Gal, Glc, GalNAc or GlcNAc, more preferably Gal or GalNAc, most preferably GalNAc. The term derivative refers to the monosaccharide being appropriately functionalized in order to connect to (G)ₑ and F.

### The Immune Cell-engaging Polypeptide

Immune cell-engaging polypeptides are known in the art, and include Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides. The immune cell-engaging polypeptide comprising one or two functional moieties Q can be obtained by procedures known in the art, such as by chemical or enzymatic modification of the immune cell-engaging polypeptide.

The immune cell-engaging polypeptide in the context of the present invention can be represented by (Q)_{y}-L-D, wherein y is 1 or 2 and D represents the immune cell-engaging polypeptide. L is a bivalent linker (L^{A}) in case y = 1, which covalently links reactive moiety Q and D, or L is trivalent linker (L^{B}) in case y = 2, which covalently links both reactive moieties Q and D.

In a preferred embodiment, linker L is a trivalent linker L^{B} according to the structure (9). Similarly, a preferred embodiment of the immune cell-engaging polypeptide comprising two reactive moieties Q is according to structure (12a). In case the multispecific antibody fragments according to the invention are prepared according to Scheme 5, via structure (3), trivalent linker L^{C} may also be represented by structure (9).

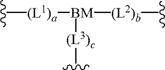

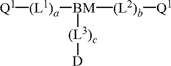

Herein, L¹, L², L³ and BM together make up linker L. BM represents a branching moiety, L¹, L² and L³ are each individually linkers and a, b and c are each individually 0 or 1. The wavy bonds represent the connection points with both reactive moieties Q and Z or D.

### The Branching Moiety BM

A “branching moiety” in the context of the present invention refers to a moiety that is embedded in a linker connecting three moieties. In other words, the branching moiety comprises at least three bonds to other moieties, one bond to reactive group F, connecting group Z or payload D, one bond to reactive group Q or connecting group Z, and one bond to reactive group Q or connecting group Z.

Any moiety that contains at least three bonds to other moieties is suitable as branching moiety in the context of the present invention. Suitable branching moieties include a carbon atom (BM-1), a nitrogen atom (BM-3), a phosphorus atom (phosphine (BM-5) and phosphine oxide (BM-6)), aromatic rings such as a phenyl ring (e.g. BM-7) or a pyridyl ring (e.g. BM-9), a (hetero)cycle (e.g. BM-11 and BM-12) and polycyclic moieties (e.g. BM-13, BM-14 and BM-15). Preferred branching moieties are selected from carbon atoms and phenyl rings, most preferably BM is a carbon atom. Structures (BM-1) to (BM-15) are depicted here below, wherein the three branches, i.e. bonds to other moieties as defined above, are indicated by *-(a bond labelled with *).

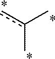

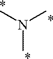

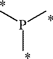

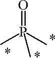

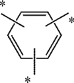

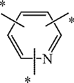

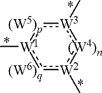

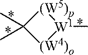

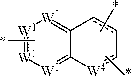

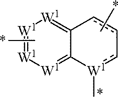

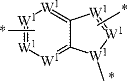

In (BM-1), one of the branches labelled with * may be a single or a double bond, indicated with

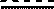

In (BM-11) to (BM-15), the following applies:
each of n, p, q and q is individually an integer in the range of 0 5, preferably 0 or 1, most preferably 1;
each of W, W and W³ is independently selected from C(R²¹)_{w} and N;
each of W, W⁵ and W⁶ is independently selected from C(R²¹)_{w+1}, N(R²²)_{w}, O and S;
each

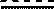

represents a single or double bond;
w is 0 or 1 or 2, preferably 0 or 1;
each R²¹ is independently selected from the group consisting of hydrogen, OH, C₁ - C₂₄ alkyl groups, C₁ - C₂₄ alkoxy groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups, wherein the C₁ - C₂₄ alkyl groups, C₁ - C₂₄ alkoxy groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups are optionally substituted and optionally interrupted by one or more heteroatoms selected from O, S and NR³ wherein R³ is independently selected from the group consisting of hydrogen and C₁ - C₄ alkyl groups; and
each R²² is independently selected from the group consisting of hydrogen, C₁ - C₂₄ alkyl groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups, wherein the C₁ - C₂₄ alkyl groups, C₁ - C₂₄ alkoxy groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups are optionally substituted and optionally interrupted by one or more heteroatoms selected from O, S and NR³ wherein R³ is independently selected from the group consisting of hydrogen and C₁ - C₄ alkyl groups.

The skilled person appreciates that the values of w and the bond order of the bonds represented by

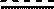

are interdependent. Thus, whenever an occurrence of W is bonded to an endocyclic double bond, w = 1 for that occurrence of W, while whenever an occurrence of W is bonded to two endocyclic single bonds, w = 0 for that occurrence of W. For BM-12, at least one of o and p is not 0.

Representative examples of branching moieties according to structure (BM-11) and (BM-12) include cyclopropyl, cyclobutyl, cyclopentyl, cyclohexyl, cycloheptyl, cyclooctyl, cyclopropenyl, cyclobutenyl, cyclopentenyl, cyclohexenyl, cycloheptenyl, cyclooctenyl, aziridine, azetidine, diazetidine, oxetane, thietane, pyrrolidine, dihydropyrrolyl, tetrahydrofuranyl, dihydrofuranyl, thiolanyl, imidazolinyl, pyrazolidinyl, oxazolidinyl, isoxazolidinyl, thiazolidinyl, isothiazolidinyl, dioxolanyl, dithiolanyl, piperidinyl, oxanyl, thianyl, piperazinyl, morpholino, thiomorpholino, dioxanyl, trioxanyl, dithyanyl, trithianyl, azepanyl, oxepanyl and thiepanyl. Preferred cyclic moieties for use as branching moiety include cyclopropenyl, cyclohexyl, oxanyl (tetrahydropyran) and dioxanyl. The substitution pattern of the three branches determines whether the branching moiety is of structure (BM-11) or of structure (BM-12).

Representative examples of branching moieties according to structure (BM-13) to (BM-15) include decalin, tetralin, dialin, naphthalene, indene, indane, isoindene, indole, isoindole, indoline, isoindoline, and the like.

In a preferred embodiment, BM is a carbon atom. In case the carbon atom is according to structure (BM-1) and has all four bonds to distinct moieties, the carbon atom is chiral. The stereochemistry of the carbon atom is not crucial for the present invention, and may be S or R. The same holds for the phosphine (BM-6). Most preferably, the carbon atom is according to structure (BM-1). One of the branches indicated with * in the carbon atom according to structure (BM-1) may be a double bond, in which case the carbon atom may be part of an alkene or imine. In case BM is a carbon atom, the carbon atom may be part of a larger functional group, such as an acetal, a ketal, a hemiketal, an orthoester, an orthocarbonate ester, an amino acid and the like. This also holds in case BM is a nitrogen or phosphorus atom, in which case it may be part of an amide, an imide, an imine, a phosphine oxide (as in BM-6) or a phosphotriester.

In a preferred embodiment, BM is a phenyl ring. Most preferably, the phenyl ring is according to structure (BM-7). The substitution pattern of the phenyl ring may be of any regiochemistry, such as 1,2,3-substituted phenyl rings, 1,2,4-substituted phenyl rings, or 1,3,5-substituted phenyl rings. To allow optimal flexibility and conformational freedom, it is preferred that the phenyl ring is according to structure (BM-7), most preferably the phenyl ring is 1,3,5-substituted. The same holds for the pyridine ring of (BM-9).

In a preferred embodiment, the branching moiety BM is selected from a carbon atom, a nitrogen atom, a phosphorus atom, a (hetero)aromatic ring, a (hetero)cycle or a polycyclic moiety.

### Linkers

L^{A}, L^{B} and L^{C} may be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

Each of L¹, L² and L³ may be absent or present, but preferably all three linking units are present. In a preferred embodiment, each of L¹, L² and L³, if present, is independently a chain of at least 2, preferably 5 to 100, atoms selected from C, N, O, S and P. Herein, the chain of atoms refers to the shortest chain of atoms going from the extremities of the linking unit. The atoms within the chain may also be referred to as backbone atoms. As the skilled person will appreciate, atoms having more than two valencies, such as C, N and P, may be appropriately functionalized in order to complete the valency of these atoms. In other words, the backbone atoms are optionally functionalized. In a preferred embodiment, each of L¹, L² and L³, if present, is independently a chain of at least 5 to 50, preferably 6 to 25 atoms selected from C, N, O, S and P. The backbone atoms are preferably selected from C, N and O.

Linkers L¹ and L² connect BM with reactive moieties Q (before reaction) or with connecting groups Z (after reaction). It is preferred that L¹ and L² are both present, i.e. a = b = 1, more preferably they are the same. In an especially preferred embodiment, (L¹)ₐ-Q is identical to (L²)_{b}-Q. Linker connects BM with reactive moiety F¹ (before reaction) or with payload D (after reaction). In one embodiment, L³is absent and c = 0. In an alternative and more preferred embodiment, L³ is present and c = 1. If L³ is present, it may be the same as L¹ and L² or different.

L¹, L² and L³ may be independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, L¹, L² and L³, if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C1₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

Even more preferably, L¹, L² and L³, if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

Yet even more preferably, L¹, L² and L³, if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR³, preferably O, wherein R³ is independently selected from the group consisting of hydrogen and C₁ - C₄ alkyl groups, preferably hydrogen or methyl.

Most preferably, L¹, L² and L³, if present, are independently selected from the group consisting of linear or branched C₁-C₂₀ alkylene groups, the alkylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR³, preferably O and/or or S—S, wherein R³ is independently selected from the group consisting of hydrogen and C₁ - C₄ alkyl groups, preferably hydrogen or methyl.

Preferred linkers L¹, L² and L³ include -(CH₂)ₙ₁-, -(CH₂CH₂)ₙ₁-, -(CH₂CH₂O)ₙ₁-, -(OCH₂CH₂)ₙ₁-, -(CH₂CH₂O)ₙ₁CH₂CH₂-, -CH₂CH₂(OCH₂CH₂)ₙ₁-, -(CH₂CH₂CH₂O)ₙ₁-, -(OCH₂CH₂CH₂)ₙ₁-, -(CH₂CH₂CH₂o)ₙ₁CH₂CH₂CH₂- and -CH₂CH₂CH₂(OCH₂CH₂CH₂)ₙ₁-, wherein n1 is an integer in the range of 1 to 50, preferably in the range of 1 to 40, more preferably in the range of 1 to 30, even more preferably in the range of 1 to 20 and yet even more preferably in the range of 1 to 15. More preferably n1 is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

In a preferred embodiment, at least one of L¹, L² and L³ contains a peptide spacer as known in the art, preferably comprising 2 - 5 amino acids, more preferably a dipeptide or tripeptide spacer, most preferably a dipeptide spacer. Although any peptide spacer may be used, preferably the peptide spacer is selected from Val-Cit, Val-Ala, Val-Lys, Val-Arg, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, more preferably Val-Cit, Val-Ala, Ala-Ala-Asn. In one embodiment, the peptide spacer is Val-Cit. In one embodiment, the peptide spacer is Val-Ala.

In a preferred embodiment, the peptide spacer is represented by general structure (27):

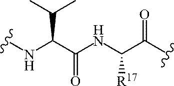

Herein, R¹⁷ = CH₃ or CH₂CH₂CH₂NHC(O)NH₂. The wavy lines indicate the connection to (L₄)ₙ and (L⁶)ₚ, preferably L⁵ according to structure (27) is connected to (L⁴)ₙ via NH and to (L⁶)ₚ via C(O).

In case linker L³ is part of linker L^{B}, i.e. when it provides a link between BM and D, it typically contains a connecting group Z that is formed when D is connected to both reactive moieties Q. The connecting group within linker L³ may be part of the moieties defined above, or may separately be present within linker L³. Thus, L³ as part of linker L^{B} may contain a moiety Z, which may take any form, and is preferably as defined further below for the connecting group obtained by the reaction of Q and F.

### Reactive Moieties Q and F

In the context of the present invention, the term “reactive moiety” may refer to a chemical moiety that comprises a functional group, but also to a functional group itself. For example, a cyclooctynyl group is a reactive group comprising a functional group, namely a C—C triple bond. Similarly, an N-maleimidyl group is a reactive group, comprising a C—C double bond as a functional group. However, a functional group, for example an azido functional group, a thiol functional group or an alkynyl functional group, may herein also be referred to as a reactive group.

In order to be reactive in the process according to the invention, reactive moiety Q should be capable of reacting with reactive moiety F present on the functionalized antibody. In other words, reactive moiety Q is reactive towards reactive moiety F present on the functionalized antibody. Herein, a reactive moiety is defined as being “reactive towards” another reactive moiety when said first reactive moiety reacts with said second reactive moiety selectively, optionally in the presence of other functional groups. Complementary reactive moiety are known to a person skilled in the art, and are described in more detail below and are exemplified in FIG. 1. As such, the conjugation reaction is a chemical reaction between Q and F forming a conjugate comprising a covalent connection between the antibody and the polypeptide. The definition of the reactive moiety Q provided here equally applies to F, Q¹ and F¹, except where denoted otherwise.

In a preferred embodiment, reactive moiety Q is selected from the group consisting of, optionally substituted, N-maleimidyl groups, ester groups, carbonate groups, protected thiol groups, alkenyl groups, alkynyl groups, tetrazinyl groups, azido groups, phosphine groups, nitrile oxide groups, nitrone groups, nitrile imine groups, diazo groups, ketone groups, (O-alkyl)hydroxylamino groups, hydrazine groups, allenamide groups, triazine groups, phosphonamidite groups. In an especially preferred embodiment, reactive moiety Q is an N-maleimidyl group, a phosphonamidite group, an azide group or an alkynyl group, most preferably reactive moiety Q is an alkynyl group. In case Q is an alkynyl group, it is preferred that Q is selected from terminal alkyne groups, (hetero)cycloalkynyl groups and bicyclo[6.1.0]non-4-yn-9-yl] groups.

In a preferred embodiment, Q comprises or is an N-maleimidyl group, preferably Q is a N-maleimidyl group. In case Q is an N-maleimidyl group, Q is preferably unsubstituted. Q is thus preferably according to structure (Q1), as shown below.

In another preferred embodiment, Q comprises or is an alkenyl group, including cycloalkenyl groups, preferably Q is an alkenyl group. The alkenyl group may be linear or branched, and is optionally substituted. The alkenyl group may be a terminal or an internal alkenyl group. The alkenyl group may comprise more than one C—C double bond, and preferably comprises one or two C-C double bonds. When the alkenyl group is a dienyl group, it is further preferred that the two C- C double bonds are separated by one C—C single bond (i.e. it is preferred that the dienyl group is a conjugated dienyl group). Preferably said alkenyl group is a C₂ - C₂₄ alkenyl group, more preferably a C₂ - C₁₂ alkenyl group, and even more preferably a C₂ - C₆ alkenyl group. It is further preferred that the alkenyl group is a terminal alkenyl group. More preferably, the alkenyl group is according to structure (Q8) as shown below, wherein I is an integer in the range of 0 to 10, preferably in the range of 0 to 6, and p is an integer in the range of 0 to 10, preferably 0 to 6. More preferably, I is 0, 1, 2, 3 or 4, more preferably I is 0, 1 or 2 and most preferably I is 0 or 1. More preferably, p is 0, 1, 2, 3 or 4, more preferably p is 0, 1 or 2 and most preferably p is 0 or 1. It is particularly preferred that p is 0 and I is 0 or 1, or that p is 1 and I is 0 or 1.

A particularly preferred alkenyl group is a cycloalkenyl group, including heterocycloalkenyl groups, wherein the cycloalkenyl group is optionally substituted. Preferably said cycloalkenyl group is a C₃ - C₂₄ cycloalkenyl group, more preferably a C₃ - C₁₂ cycloalkenyl group, and even more preferably a C₃ - C₈ cycloalkenyl group. In a preferred embodiment, the cycloalkenyl group is a trans-cycloalkenyl group, more preferably a trans-cyclooctenyl group (also referred to as a TCO group) and most preferably a trans-cyclooctenyl group according to structure (Q9) or (Q10) as shown below. In another preferred embodiment, the cycloalkenyl group is a cyclopropenyl group, wherein the cyclopropenyl group is optionally substituted. In another preferred embodiment, the cycloalkenyl group is a norbornenyl group, an oxanorbornenyl group, a norbornadienyl group or an oxanorbornadienyl group, wherein the norbornenyl group, oxanorbornenyl group, norbornadienyl group or an oxanorbornadienyl group is optionally substituted. In a further preferred embodiment, the cycloalkenyl group is according to structure (Q11), (Q12), (Q13) or (Q14) as shown below, wherein X₄ is CH₂ or O, R²⁷ is independently selected from the group consisting of hydrogen, a linear or branched C₁ - C₁₂ alkyl group or a C₄ - C₁₂ (hetero)aryl group, and R¹⁴ is selected from the group consisting of hydrogen and fluorinated hydrocarbons. Preferably, R²⁷ is independently hydrogen or a C₁ - C₆ alkyl group, more preferably R²⁷ is independently hydrogen or a C₁ - C₄ alkyl group. Even more preferably R²⁷ is independently hydrogen or methyl, ethyl, n-propyl, i-propyl, n-butyl, s-butyl or t-butyl. Yet even more preferably R²⁷ is independently hydrogen or methyl. In a further preferred embodiment, R¹⁴ is selected from the group of hydrogen and —CF₃, —C₂F₅, —C₃F₇ and —C₄F₉, more preferably hydrogen and —CF₃. In a further preferred embodiment, the cycloalkenyl group is according to structure (Q11), wherein one R²⁷ is hydrogen and the other R²⁷ is a methyl group. In another further preferred embodiment, the cycloalkenyl group is according to structure (Q12), wherein both R²⁷ are hydrogen. In these embodiments it is further preferred that I is 0 or 1. In another further preferred embodiment, the cycloalkenyl group is a norbornenyl (X⁴ is CH₂) or an oxanorbornenyl (X⁴ is O) group according to structure (Q13), or a norbornadienyl (X⁴ is CH₂) or an oxanorbornadienyl (X⁴ is O) group according to structure (Q14), wherein R²⁷ is hydrogen and R¹⁴ is hydrogen or —CF₃, preferably —CF₃.

In another preferred embodiment, Q comprises or is an alkynyl group, including cycloalkynyl groups, preferably Q comprises an alkynyl group. The alkynyl group may be linear or branched, and is optionally substituted. The alkynyl group may be a terminal or an internal alkynyl group. Preferably said alkynyl group is a C₂ - C₂₄ alkynyl group, more preferably a C₂ - C₁₂ alkynyl group, and even more preferably a C₂ - C₆ alkynyl group. It is further preferred that the alkynyl group is a terminal alkynyl group. More preferably, the alkynyl group is according to structure (Q15) as shown below, wherein I is an integer in the range of 0 to 10, preferably in the range of 0 to 6. More preferably, I is 0, 1, 2, 3 or 4, more preferably I is 0, 1 or 2 and most preferably I is 0 or 1.

A particularly preferred alkynyl group is a cycloalkynyl group, wherein the cycloalkynyl group is heterocycloalkynyl group or cycloalkynyl group, and is optionally substituted. Preferably, the (hetero)cycloalkynyl group is a (hetero)cyclooctynyl group, i.e. a heterocyclooctynyl group or a cyclooctynyl group, wherein the (hetero)cyclooctynyl group is optionally substituted. In a further preferred embodiment, the (hetero)cyclooctynyl group is according to structure (Q36) and defined further below. Preferred examples of the (hetero)cyclooctynyl group include structure (Q16), also referred to as a DIBO group, (Q17), also referred to as a DIBAC group, or (Q18), also referred to as a BARAC group, (Q19), also referred to as a COMBO group, and (Q20), also referred to as a BCN group, all as shown below, wherein X⁵ is O or N R²⁷, and preferred embodiments of R²⁷ are as defined above. The aromatic rings in (Q16) are optionally O-sulfonylated at one or more positions, preferably at two positions, most preferably as in (Q40) (sulfonylated dibenzocyclooctyne (s-DIBO)), whereas the rings of (Q17) and (Q18) may be halogenated at one or more positions. A particularly preferred cycloalkynyl group is a bicyclo[6.1.0]non-4-yn-9-yl] group (BCN group), which is optionally substituted. Preferably, the bicyclo[6.1.0]non-4-yn-9-yl] group is according to structure (Q20) as shown below.

In another preferred embodiment, Q comprises or is a conjugated (hetero)diene group, preferably Q is a conjugated (hetero)diene group capable of reacting in a Diels-Alder reaction. Preferred (hetero)diene groups include optionally substituted tetrazinyl groups, optionally substituted 1,2-quinone groups and optionally substituted triazine groups. More preferably, said tetrazinyl group is according to structure (Q21), as shown below, wherein R²⁷ is selected from the group consisting of hydrogen, a linear or branched C₁ - C₁₂ alkyl group or a C₄ - C₁₂ (hetero)aryl group. Preferably, R²⁷ is hydrogen, a C₁ - C₆ alkyl group or a C₄ - C₁₀ (hetero)aryl group, more preferably R²⁷ is hydrogen, a C₁ - C₄ alkyl group or a C₄ - C₆ (hetero)aryl group. Even more preferably R²⁷ is hydrogen, methyl, ethyl, n-propyl, i-propyl, n-butyl, s-butyl, t-butyl or pyridyl. Yet even more preferably R²⁷ is hydrogen, methyl or pyridyl. More preferably, said 1,2-quinone group is according to structure (Q22) or (Q23). Said triazine group may be any regioisomer. More preferably, said triazine group is a 1,2,3-triazine group or a 1,2,4-triazine group, which may be attached via any possible location, such as indicated in structure (Q24). The 1,2,3-triazine is most preferred as triazine group.

In another preferred embodiment, Q comprises or is an azido group, preferably Q is an azido group. Preferably, the azide group is according to structure (Q25) as shown below.

In another preferred embodiment, Q comprises or is an allenamide group, preferably Q is an allenamide group. Preferably, the allenamide group is according to structure (Q35).

In another preferred embodiment, Q comprises or is an phosphonamidate group, preferably Q is an phosphonamidate group. Preferably, the phosphonamidate group is according to structure (Q36).

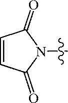

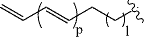

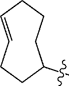

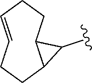

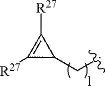

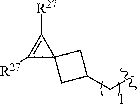

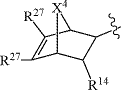

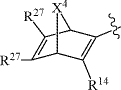

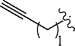

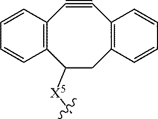

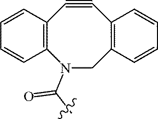

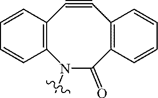

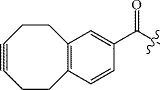

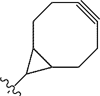

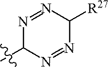

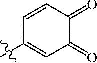

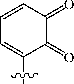

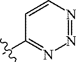

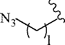

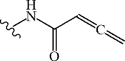

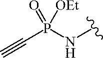

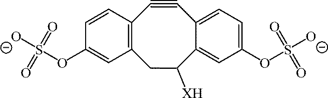

Herein, the aromatic rings in (Q16) are optionally O-sulfonylated at one or more positions, 5 whereas the rings of (Q17) and (Q18) may be halogenated at one or more positions.

In case Q is a cycloalkynyl group, it is preferred to Q is selected from the group consisting of (Q42) - (Q60):

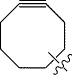

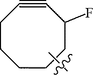

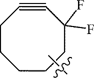

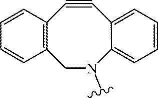

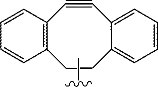

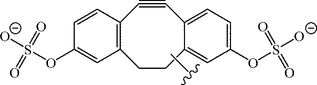

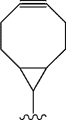

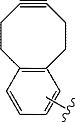

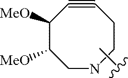

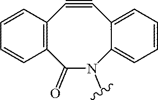

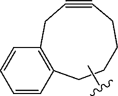

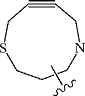

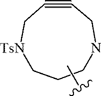

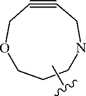

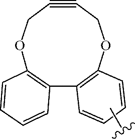

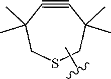

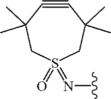

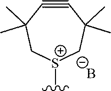

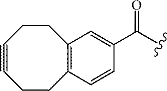

Herein, the connection to the remainder of the molecule, depicted with the wavy bond, may be to any available carbon or nitrogen atom of Q. The nitrogen atom of (Q50), (Q53), (Q54) and (Q55) may bear the connection, or may contain a hydrogen atom or be optionally functionalized. B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf. In the conjugation reaction, B⁽⁻⁾ does not need to be a pharmaceutically acceptable anion, since B⁽⁻⁾ will exchange with the anions present in the reaction mixture anyway. In case (Q59) is used for Q, the negatively charged counter-ion is preferably pharmaceutically acceptable upon isolation of the antibody construct according to the invention, such that the antibody construct is readily useable as medicament.

Q is capable of reacting with a reactive moiety F that is present on an antibody. Complementary reactive groups F for reactive group Q are known to a person skilled in the art, and are described in more detail below. Some representative examples of reaction between F and Q and their corresponding products (connecting group Z) are depicted in FIG. 1.

In a preferred embodiment, the conjugation is achieved by cycloaddition or nucleophilic reaction, preferably wherein the cycloaddition is a [4+2] cycloaddition or a 1,3-dipolar cycloaddition and the nucleophilic reaction is a Michael addition or a nucleophilic substitution.

Thus, in a preferred embodiment of the conjugation process according to the invention, conjugation is accomplished via a nucleophilic reaction, such as a nucleophilic substitution or a Michael reaction. A preferred Michael reaction is the maleimide-thiol reaction, which is widely employed in bioconjugation. Thus, in a preferred embodiment, Q is reactive in a nucleophilic reaction, preferably in a nucleophilic substitution or a Michael reaction. Herein, it is preferred that Q comprises a maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety, most preferably a maleimide moiety.

In case a nucleophilic reaction is used for the conjugation, it is preferred that the structural moiety Q—(L¹)ₐ—BM—(L²)_{b}—Q is selected from bromomaleimide, bis-bromomaleimide, bis(phenylthiol)maleimide, bis-bromopyridazinedione, bis(halomethyl)benzene, bis(halomethyl)pyridazine, bis(halomethyl)pyridine or bis(halomethyl)triazole.

Thus, in a preferred embodiment of the conjugation process according to the invention, conjugation is accomplished via a cycloaddition, such as a [4+2] cycloaddition or a 1,3-dipolar cycloaddition, preferably the 1,3-dipolar cycloaddition. According to this embodiment, the reactive group Q is selected from groups reactive in a cycloaddition reaction. Herein, reactive groups Q and F are complementary, i.e. they are capable of reacting with each other in a cycloaddition reaction.

A typical [4+2] cycloaddition is the Diels-Alder reaction, wherein Q is a diene or a dienophile. As appreciated by the skilled person, the term “diene” in the context of the Diels-Alder reaction refers to 1,3-(hetero)dienes, and includes conjugated dienes (R₂C═CR—CR═CR₂), imines (e.g. R₂C═CR—N═CR₂ or R₂C═CR—CR═NR, R₂C═N—N═CR₂) and carbonyls (e.g. R₂C═CR—CR═O or O═CR—CR═O). Hetero-Diels-Alder reactions with N— and O-containing dienes are known in the art. Any diene known in the art to be suitable for [4+2] cycloadditions may be used as reactive group Q. Preferred dienes include tetrazines as described above, 1,2-quinones as described above and triazines as described above. Although any dienophile known in the art to be suitable for [4+2] cycloadditions may be used as reactive group Q, the dienophile is preferably an alkene or alkyne group as described above, most preferably an alkyne group. For conjugation via a [4+2] cycloaddition, it is preferred that Q is a dienophile (and F is a diene), more preferably Q is or comprises an alkynyl group.

For a 1,3-dipolar cycloaddition, Q is a 1,3-dipole or a dipolarophile. Any 1,3-dipole known in the art to be suitable for 1,3-dipolar cycloadditions may be used as reactive group Q. Preferred 1,3-dipoles include azido groups, nitrone groups, nitrile oxide groups, nitrile imine groups and diazo groups. Although any dipolarophile known in the art to be suitable for 1,3-dipolar cycloadditions may be used as reactive groups Q, the dipolarophile is preferably an alkene or alkyne group, most preferably an alkyne group. For conjugation via a 1,3-dipolar cycloaddition, it is preferred that Q is a dipolarophile (and F is a 1,3-dipole), more preferably Q is or comprises an alkynyl group.

Thus, in a preferred embodiment, Q is selected from dipolarophiles and dienophiles. Preferably, Q is an alkene or an alkyne group. In an especially preferred embodiment, Q comprises an alkyne group, preferably selected from the alkynyl group as described above, the cycloalkenyl group as described above, the (hetero)cycloalkynyl group as described above and a bicyclo[6.1.0]non-4-yn-9-yl] group. More preferably Q comprises a terminal alkyne or a cyclooctyne moiety, preferably bicyclononyne (BCN), azadibenzocyclooctyne (DIBAC/DBCO), dibenzocyclooctyne (DIBO) or sulfonylated dibenzocyclooctyne (s-DIBO), more preferably BCN or DIBAC/DBCO, most preferably BCN. In alternative preferred embodiment, Q is selected from the formulae (Q5), (Q6), (Q7), (Q8), (Q20) and (Q9), more preferably selected from the formulae (Q6), (Q7), (Q8), (Q20) and (Q9). Most preferably, Q is a bicyclo[6.1.0]non-4-yn-9-yl] group, preferably of formula (Q20). These groups are known to be highly effective in the conjugation with azido-functionalized antibodies.

In an especially preferred embodiment, reactive group Q comprises an alkynyl group and is according to structure (Q36):

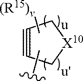

Herein:
R¹⁵ is independently selected from the group consisting of hydrogen, halogen, —OR¹⁶, —NO₂, —CN, —S(O)₂R¹⁶, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an annelated cycloalkyl or an annelated (hetero)arene substituent, and wherein R¹⁶ is independently selected from the group consisting of hydrogen, halogen, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups;
X₁₀ is C(R¹⁷)₂, O, S or NR¹⁷, wherein R¹⁷ is R¹⁵;
u is 0, 1, 2, 3, 4 or 5;
u′ is 0, 1, 2, 3, 4 or 5;
wherein u + u′ = 5;
v = 9 or 10.

Preferred embodiments of the reactive group according to structure (Q36) are reactive groups according to structure (Q37), (Q6), (Q7), (Q8), (Q9) and (Q20).

In an especially preferred embodiment, reactive group Q comprises an alkynyl group and is according to structure (Q37):

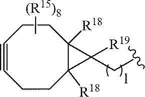

Herein:
R¹⁵ is independently selected from the group consisting of hydrogen, halogen, —OR¹⁶, —NO₂, —CN, —S(O)₂R¹⁶, C₁ - C₂₄ alkyl groups, C₅ - C₂₄ (hetero)aryl groups, C₇ - C₂₄ alkyl(hetero)aryl groups and C₇ - C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an annelated cycloalkyl or an annelated (hetero)arene substituent, and wherein R¹⁶ is independently selected from the group consisting of hydrogen, halogen, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups;
R¹⁸ is independently selected from the group consisting of hydrogen, halogen, C₁ - C₂₄ 25 alkyl groups, C₆ - C₂₄ (hetero)aryl groups, C₇ - C₂₄ alkyl(hetero)aryl groups and C₇ - C₂₄ (hetero)arylalkyl groups;
R¹⁹ is selected from the group consisting of hydrogen, halogen, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
I is an integer in the range 0 to 10.

In a preferred embodiment of the reactive group according to structure (Q37), R¹⁵ is independently selected from the group consisting of hydrogen, halogen, —OR¹⁶, C₁ - C₆ alkyl groups, C₅ - C₆ (hetero)aryl groups, wherein R¹⁶ is hydrogen or C₁ - C₆ alkyl, more preferably R¹⁵ is independently selected from the group consisting of hydrogen and C₁ - C₆ alkyl, most preferably all R¹⁵ are H. In a preferred embodiment of the reactive group according to structure (Q37), R¹⁸ is independently selected from the group consisting of hydrogen, C₁ - C₆ alkyl groups, most preferably both R¹⁸ are H. In a preferred embodiment of the reactive group according to structure (Q37), R¹⁹ is H. In a preferred embodiment of the reactive group according to structure (Q37), I is 0 or 1, more preferably I is 1. An especially preferred embodiment of the reactive group according to structure (Q37) is the reactive group according to structure (Q20).

### Connecting Group Z

Z is a connecting group, that covalently connects both parts of the conjugate according to the invention. The term “connecting group” herein refers to the structural element, resulting from the reaction between Q and F, connecting one part of a compound and another part of the same compound. As will be understood by the person skilled in the art, the nature of a connecting group depends on the type of reaction with which the connection between the parts of said compound was obtained. As an example, when the carboxyl group of R—C(O)—OH is reacted with the amino group of H₂N—R′ to form R—C(O)—N(H)—R′, R is connected to R′ via connecting group Z, and Z is represented by the group —C(O)—N(H)—. Since connecting group Z originates from the reaction between Q and F, it can take any form. Moreover, for the working of the present invention, the nature of connecting group Z is not crucial at all.

Since up to 10 reactive moiety F can be present or introduced in an antibody, the conjugate according to the present invention may contain per antibody 10 polypeptides D. This is denoted by the label x, which may be an integer in the range 1 - 10, preferably in the range 1 - 8, more preferably x = 1, 2 or 4 or 8, more preferably x = 1 or 2.

In the context of the present invention, connecting group Z connects the antibody, optionally via a spacer, to linker L. Numerous reactions are known in the art for the attachment of a reactive group Q to a reactive group F. Consequently, a wide variety of connecting groups Z may be present in the conjugate according to the invention. In one embodiment, the connecting group Z is selected from the options described above, preferably as depicted in FIG. 1.

For example, when F comprises or is a thiol group, complementary groups Q include N-maleimidyl groups and alkenyl groups, and the corresponding connecting groups Z are as shown in FIG. 1. When F comprises or is a thiol group, complementary groups Q also include allenamide groups and phosphonamidate groups.

For example, when F comprises or is a ketone group, complementary groups Q include (O-alkyl)hydroxylamino groups and hydrazine groups, and the corresponding connecting groups Z are as shown in FIG. 1.

For example, when F comprises or is an alkynyl group, complementary groups Q include azido groups, and the corresponding connecting group Z is as shown in FIG. 1.

For example, when F comprises or is an azido group, complementary groups Q include alkynyl groups, and the corresponding connecting group Z is as shown in FIG. 1.

For example, when F comprises or is a cyclopropenyl group, a trans-cyclooctene group or a cycloalkyne group, complementary groups Q include tetrazinyl groups, and the corresponding connecting group Z is as shown in FIG. 1. In particular cases, Z is only an intermediate structure and will expel N₂, thereby generating a dihydropyridazine (from the reaction with alkene) or pyridazine (from the reaction with alkyne).

For example, when F comprises or is a tetrazinyl group, complementary groups Q include a cyclopropenyl group, a trans-cyclooctene group or a cycloalkyne group, and the corresponding connecting group Z is as shown in FIG. 1. In particular cases, Z is only an intermediate structure and will expel N₂, thereby generating a dihydropyridazine (from the reaction with alkene) or pyridazine (from the reaction with alkyne).

Additional suitable combinations of F and Q, and the nature of resulting connecting group Z are known to a person skilled in the art, and are e.g. described in G.T. Hermanson, “Bioconjugate Techniques”, Elsevier, 3rd Ed. 2013 (ISBN:978-0-12-382239-0), in particular in Chapter 3, pages 229 - 258, incorporated by reference. A list of complementary reactive groups suitable for bioconjugation processes is disclosed in Table 3.1, pages 230 - 232 of Chapter 3 of G.T. Hermanson, “Bioconjugate Techniques”, Elsevier, 3rd Ed. 2013 (ISBN:978-0-12-382239-0), and the content of this Table is expressly incorporated by reference herein.

In a preferred embodiment, connecting group Z is according to any one of structures (Za) to (Zk), as defined below. Preferably, Z is according to structures (Za), (Ze) or (Zj):

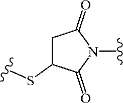

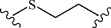

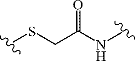

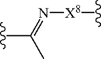

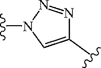

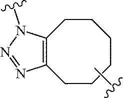

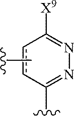

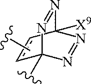

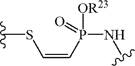

Herein,
X⁸ is O or NH.
X⁹ is selected from H, C₁₋₁₂ alkyl and pyridyl, wherein the C₁₋₁₂ alkyl preferably is C₁₋₄ alkyl, most preferably methyl.
R²³ is C₁₋₁₂ alkyl, preferably C₁₋₄ alkyl, most preferably ethyl.
In structure (Zg) and (Zh), the bond represents either a single or a double bond, and may be connected via either side of this bond to linkers L.

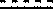

The wavy lines indicate the connection to linkers L. The connectivity depends on the specific nature of Q and F. Although either site of the connecting groups according to (Za) to (Zg) may be connected to L, it is preferred that the left-most of these groups as depicted is connected to (L¹)a/(L²)b.

Connecting group (Zh) typically rearranges to (Zg) with the liberation of N₂.

In a preferred embodiment, each Z is independently selected from the group consisting of —O—, —S—, —S—S—, —NR²—, —N═N—, —C(O)—, —C(O)—NR²—, —O—C(O)—, —O—C(O)—O—, —O—C(O)—NR², —NR₂—C(O)—,—NR²—C(O)—O—, —NR²—C(O)—NR²—, —S—C(O)—, —S—C(O)—O—, —S—C(O)—NR²—, —S(O)—, —S(O)²—, —O—S(O)₂—, —O—S(O)₂—O—, —O—S(O)₂—NR²—, —O—S(O)—, —O—S(O)—O—, —O—S(O)—NR²—, —O—NR²—C(O)—, —O—NR²—C(O)—O—, —O—NR²—C(O)—NR²—, —NR²—O—C(O)—, —NR²—O—C(O)—O—, —NR²—O—C(O)—NR²—, —O—NR²—C(S)—, —O—NR²—C(S)—O—, —O—NR²—C(S)—NR²—, —NR²—O—C(S)—, —NR²—O—C(S)—O—, —NR²—O—C(S)—NR²—, —O—C(S)—, —O—C(S)—O—, —O—C(S)—NR²—, —NR²—C(S)—, —NR²—C(S)—O—, —NR²—C(S)—NR²—, —S—S(O)₂—, —S—S(O)₂—O—, —S—S(O)₂—NR²—, —NR₂—O—S(O)—, —NR²—O—S(O)—O—, —NR²—O—S(O)—NR²—, —NR²—O—S(O)₂—, —NR²—O—S(O)₂—O—, —NR²—O—S(O)₂—NR²—, —O—NR²—S(O)—, —O—NR²—S(O)—O—,—O—NR²—S(O)—NR²—, —O—NR²—S(O)²—O—, —O—NR²—S(O)₂—NR²—, —O—NR²—S(O)₂—, —O—P(O)(R²)₂—, —S—P(O)(R²)₂—, —NR₂—P(O)(R²)₂— and the moieties represented by any one of (Za) -(Zi). Herein, R² is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

More preferably, each Z contains a moiety selected from the group consisting of a succinimide, a triazole, a cyclohexene, a cyclohexadiene, an isoxazoline, an isoxazolidine, a pyrazoline, a piperazine, a thioether, an amide or an imide group. Preferably, Z comprises a moiety selected from selected from the group consisting of a triazole, a cyclohexene, a cyclohexadiene, an isoxazoline, an isoxazolidine, a pyrazoline, a piperazine, a thioether, an amide or an imide group. In an especially preferred embodiment, Z comprises a triazole moiety or a succinimide moiety. Triazole moieties are especially preferred to be present in Z.

In an especially preferred embodiment, connecting group Z comprises a triazole moiety and is according to structure (Zj):

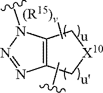

Herein, R¹⁵, X¹⁰, u, u′ and v are as defined for (Q36), and all preferred embodiments thereof equally apply to (Zj). The wavy lines indicate the connection to adjacent moieties (Su and (L¹)ₐ or (L²)_{b}), and the connectivity depends on the specific nature of Q and F. Although either site of the connecting group according to (Zj) may be connected to (L¹)a/(L²)b, it is preferred that the upperwavy bond as depicted represents the connectivity to Su. The connecting groups according to structure (Zf) and (Zk) are preferred embodiments of the connecting group according to (Zj).

In an especially preferred embodiment, connecting group Z comprises a triazole moiety and is according to structure (Zk):

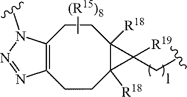

Herein, R¹⁵, R¹⁸, R¹⁹, and I are as defined for (Q37), and all preferred embodiments thereof equally apply to (Zj). The wavy lines indicate the connection to adjacent moieties (Su and (L¹)a or (L²)b), and the connectivity depends on the specific nature of Q and F. Although either site of the connecting group according to (Zj) may be connected to (L¹)a, it is preferred that the left wavy bond as depicted represents the connectivity to Su.

In a preferred embodiment, Q comprises or is an alkyne moiety and F is an azido moiety, such that connecting group Z comprises an triazole moiety. Preferred connecting groups comprising a triazole moiety are the connecting groups according to structure (Ze) or (Zj), wherein the connecting groups according to structure (Zj) is preferably according to structure (Zk) or (Zf). In a preferred embodiment, the connecting groups is according to structure (Zj), more preferably according to structure (Zk) or (Zf).

### Immune Cell-engaging Polypeptides

In a further aspect, the invention concerns immune cell-engaging polypeptides comprising one or two reactive moieties Q. Preferably, the immune cell-engaging polypeptide according to the invention has two moieties Q. The immune cell-engaging polypeptide according to the invention has structure (Q)₂—L—D. Herein, D is an immune cell-engaging polypeptide, which is defined above; L is a linker, which is defined above; and Q is a reactive group as defined above. The definitions, including preferred embodiments, recited above are equally applicable to the immune cell-engaging polypeptide according to the invention.

In a preferred embodiment, the immune cell-engaging polypeptide according to the invention has structure (12a):

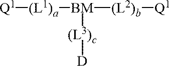

The immune cell-engaging polypeptide according to the invention is especially suitable as intermediate in the preparation of multispecific antibody constructs according to the invention.

### Multispecific Antibody Construct

The invention further concerns the multispecific antibody construct obtainable by the process according to the invention. In one embodiment, the multispecific antibody construct according to the invention has structure (13a) or (13b). The multispecific antibody construct of structure (13b) preferably has the structure (13c).

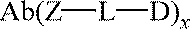

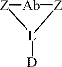

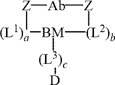

Herein, Ab, Z, L, D, x, L¹, L², L³, a, b, c and BM are as defined above, including preferred embodiments thereof.

### Application

The multispecific antibody constructs according to the invention, or the multispecific antibody constructs obtainable by the process according to the invention, are especially suitable in the treatment of cancer. The invention thus further concerns the use of the multispecific antibody construct according to the invention in medicine. In a further aspect, the invention also concerns a method of treating a subject in need thereof, comprising administering the multispecific antibody construct according to the invention to the subject. The method according to this aspect can also be worded as the multispecific antibody construct according to the invention for use in treatment. The method according to this aspect can also be worded as use of the multispecific antibody construct according to the invention for the manufacture of a medicament. Herein, administration typically occurs with a therapeutically effective amount of the multispecific antibody construct according to the invention.

The invention further concerns a method for the treatment of a specific disease in a subject in need thereof, comprising the administration of the multispecific antibody construct according to the invention as defined above. The specific disease may be selected from cancer, a viral infection, a bacterial infection, a neurological disease, an autoimmune disease, an eye disease, hypercholesterolaemia and amyloidosis, more preferable from cancer and a viral infection, most preferably the disease is cancer. The subject in need thereof is typically a cancer patient. The use of multispecific antibody construct according to the invention is well-known in such treatments, especially in the field of cancer treatment, and the multispecific antibody constructs according to the invention are especially suited in this respect. In the method according to this aspect, the multispecific antibody construct is typically administered in a therapeutically effective amount. The present aspect of the invention can also be worded as a multispecific antibody construct according to the invention for use in the treatment of a specific disease in a subject in need thereof, preferably for the treatment of cancer. In other words, this aspect concerns the use of a multispecific antibody construct according to the invention for the preparation of a medicament or pharmaceutical composition for use in the treatment of a specific disease in a subject in need thereof, preferably for use in the treatment of cancer.

It is preferred that the multispecific antibody construct according to the invention is Fc-silent, i.e. does not significantly bind to Fc gamma receptors CD16 when used in clinically. This is the case when G is absent, i.e. that e = 0. Preferably, also the binding towards CD32 and CD64 is significantly reduced.

The invention further concerns a method for associating an immune cell with a tumour cell. A sample comprising the immune cell and the tumour cell is contacted with the multispecific antibody construct according to the invention. The immune cell binds to the immune cell-engaging peptide and the tumour cell to the antibody, as such a complex association of tumour cell, immune cell and multispecific antibody construct. This contacting may take place in a sample in vitro, e.g. taking from a subject, or in vivo within a subject, in which case the multispecific antibody construct according to the invention is administered to the subject.

Administration in the context of the present invention refers to systemic administration. Hence, in one embodiment, the methods defined herein are for systemic administration of the multispecific antibody construct. In view of the specificity of the multispecific antibody constructs, they can be systemically administered, and yet exert their activity in or near the tissue of interest (e.g. a tumour). Systemic administration has a great advantage over local administration, as the drug may also reach tumour metastasis not detectable with imaging techniques and it may be applicable to hematological tumours.

The invention further concerns a pharmaceutical composition comprising the antibody-payload conjugate according to the invention and a pharmaceutically acceptable carrier.

### EXAMPLES

The invention is illustrated by the following examples.

### General Procedures

Chemicals were purchased from commonly used suppliers (Sigma-Aldrich, Acros, Alfa Aesar, Fluorochem, Apollo Scientific Ltd and TCI) and were used without further purification. Solvents (including dry solvents) for chemical transformations, work-up and chromatography were purchased from Aldrich (Dorset, UK) at HPLC grade, and used without further distillation. Silica gel 60 F254 analytical thin layer chromatography (TLC) plates were from Merck (Darmstadt, Germany) and visualized under UV light, with potassium permanganate stain or anisaldehyde stain. Chromatographic purifications were performed using Acros silica gel (0.06-0.200, 60A) or prepacked columns (Screening Devices) in combination with a Buchi Sepacore C660 fraction collector (Flawil, Switzerland). Reversed phase HPLC purifications were performed using an Agilent 1200 system equipped with a Waters Xbridge C18 column (5 µm OBD, 30 x 100 mm, PN186002982). Deuterated solvents used for NMR spectroscopy were obtained from Cambridge Isotope Laboratories. Bis-mal-Lys-PEG₄-TFP ester (177) was obtained from Quanta Biodesign, O-(2-aminoethyl)-O′-(2-azidoethyl)diethylene glycol (XL07) and compounds 344 and 179 were obtained from Broadpharm, 2,3-bis(bromomethyl)-6-quinoxalinecarboxylic acid (178) was obtained from ChemScene and 32-azido-5-oxo-3,9,12,15,18,21,24,27,30-nonaoxa-6-azadotriacontanoic acid (348) was obtained from Carbosynth.

### General Procedure for Mass Spectral Analysis of Monoclonal Antibodies and ADCs

Prior to mass spectral analysis, IgG was treated with IdeS (Fabricator™) for analysis of the Fc/2 fragment. A solution of 20 µg (modified) IgG was incubated for 1 hour at 37° C. with 0.5 µL IdeS (50 U/µL) in phosphate-buffered saline (PBS) pH 6.6 in a total volume of 10 µL. Samples were diluted to 40 µL followed by electrospray ionization time-of-flight (ESI-TOF) analysis on a JEOL AccuTOF. Deconvoluted spectra were obtained using Magtran software.

### General Procedure for Analytical RP-HPLC

Prior to RP-HPLC analysis, IgG was treated with IdeS, which allows analysis of the Fc/2 fragment. A solution of (modified) IgG (100 µL, 1 mg/mL in PBS pH 7.4) was incubated for 1 hour at 37° C. with 1.5 µL IdeSfFabricator™ (50 U/µL) in phosphate-buffered saline (PBS) pH 6.6. The reaction was quenched by adding 49% acetonitrile, 49% water, 2% formic acid (100 µL). RP-HPLC analysis was performed on an Agilent 1100 series (Hewlett Packard). The sample (10 µL) was injected with 0.5 mL/min onto a ZORBAX Poroshell 300SB-C8 column (1x75 mm, 5 µm, Agilent) with a column temperature of 70° C. A linear gradient was applied in 25 minutes from 30 to 54% acetonitrile and water in 0.1% TFA.

### General Procedure for Analytical HPLC-SEC

HPLC-SEC analysis was performed on an Agilent 1100 series (Hewlett Packard). The sample (4 µL, 1 mg/mL) was injected with 0.86 mL/min onto a Xbridge BEH200A (3.5 µM, 7.8x300 mm, PN186007640 Waters) column. Isocratic elution using 0.1 M sodium phosphate buffer pH 6.9 (NaH₂PO₄/Na₂HPO₄) was performed for 16 minutes.

### Example 1. Synthesis of Compound 102

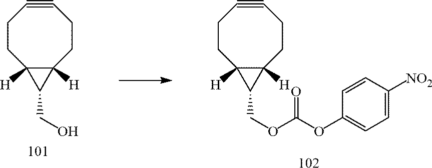

To a cooled (0° C.) solution of 4-nitrophenyl chloroformate (30.5 g, 151 mmol) in DCM (500 mL) was added pyridine (24.2 mL, 23.7 g, 299 mmol). A solution of BCN-OH (101, 18.0 g, 120 mmol) in DCM (200 mL) was added dropwise to the reaction mixture. After the addition was completed, a saturated aqueous solution of NH₄Cl (500 mL) and water (200 mL) were added. After separation, the aqueous phase was extracted with DCM (2 × 500 mL). The combined organic phases were dried (Na₂SO₄) and concentrated. The crude material was purified by silica gel chromatography and the desired product 102 was obtained as an off-white solid (18.7 g, 59 mmol, 39%). ¹H NMR (400 MHz, CDCl₃) δ (ppm) 8.32-8.23 (m, 2H), 7.45-7.34 (m, 2H), 4.40 (d, J = 8.3 Hz, 2H), 2.40-2.18 (m, 6H), 1.69-1.54 (m, 2H), 1.51 (quintet, J= 9.0 Hz, 1H), 1.12-1.00 (m, 2H)

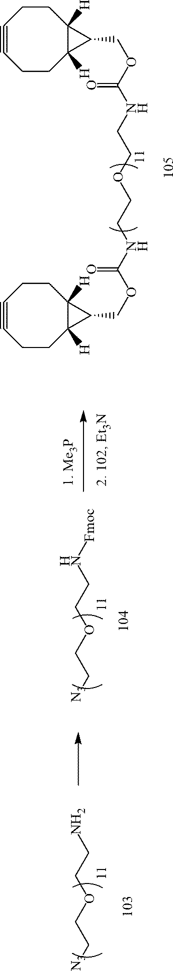

### Example 2. Synthesis of Compound 104

To a cooled solution (-5° C.) of azido-PEG₁₁-amine (103) (182 mg, 0.319 mmol) in THF (3 mL) were added a 10% aqueous NaHCO₃ solution (1.5 mL) and 9-fluorenylmethoxycarbonyl chloride (99 mg, 0.34 mmol) dissolved in THF (2 mL). After 2 h, EtOAc (20 mL) was added and the mixture was washed with brine (2 × 6 mL), dried over MgSO₄, and concentrated. Purification by silica gel column chromatography (0 → 11% MeOH in DCM) gave 104 as a clear oil in 98% yield (251 mg, 0.316 mmol). LCMS (ESI+) calculated for C₃₉H₆₀N₄O₁₃₊(M+Na+) 815.42 found 815.53.

### Example 3. Synthesis of Compound 105

A solution of 104 (48 mg, 0.060 mmol) in THF (3 mL) and water (0.2 mL) was prepared and cooled down to 0° C. Trimethylphosphine (1 M in toluene, 0.24 mL, 0.24 mmol) was added and the mixture was left stirring for 23 h. The water was removed via extraction with DCM (6 mL). To this solution, (1R,8S,9 s)-bicyclo[6.1.0]non-4-yn-9-ylmethyl (4-nitrophenyl) carbonate (102) (25 mg, 0.079 mmol) and triethylamine (10 µL, 0.070 mmol) were added. After 27 h, the mixture was concentrated and the residue was dissolved in DMF (3 mL), followed by the addition of piperidine (400 µL). After 1 h, the mixture was concentrated and the residue was purified by silica gel column chromatography (0 → 21% MeOH in DCM), which gave 105 as a colorless oil (8.3 mg, 0.0092 mmol). LCMS (ESI+) calculated for C₄₆H₇₆N₂O₁₅₊(M+NH₄₊) 914.52 found 914.73.

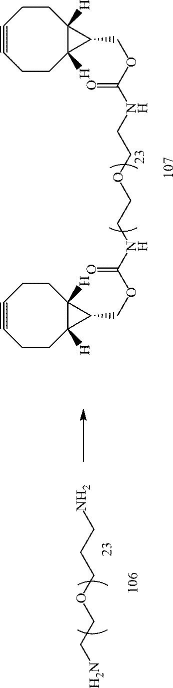

### Example 4. Synthesis of Compound 107

A solution of (1R,8S,9 s)-bicyclo[6.1,0]non-4-yn-9-ylmethyl (4-nitrophenyl) carbonate (102) (4.1 mg, 0.013 mmol) in dry DCM (500 µL) was slowly added to a solution of amino-PEG23-amine (106) (12.3 mg, 0.0114 mmol) in dry DCM (500 µL). After 20 h, the mixture was concentrated and the residue was purified by silica gel column chromatography (0 → 25% MeOH in DCM) which gave the desired compound 107 in 73% yield (12 mg, 0.0080 mmol). LCMS (ESI+) calculated for C₇₀H₁₂₄N₂O₂₇₊(M+ NH₄₊) 1443.73 found 1444.08.

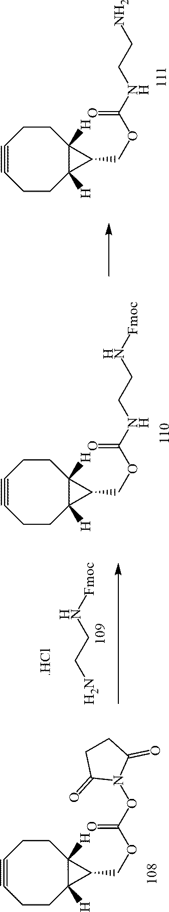

### Example 5. Synthesis of Compound 108

To a solution of BCN-OH (101, 21.0 g, 0.14 mol) in MeCN (450 mL) were added disuccinimidyl carbonate (53.8 g, 0.21 mol) and triethylamine (58.5 mL, 0.42 mol). After the mixture was stirred for 140 minutes, it was concentrated in vacuo and the residue was co-evaporated once with MeCN (400 mL). The residue was dissolved in EtOAc (600 mL) and washed with H₂O (3 × 200 mL). The organic layer was dried over Na₂SO₄ and concentrated in vacuo. The residue was purified by silica gel column chromatography (0 → 4% EtOAc in DCM) and gave 108 (11.2 g, 38.4 mmol, 27% yield) as a white solid. ¹H NMR (400 MHz, CDCl₃): δ (ppm) 4.45 (d, 2H, J = 8.4 Hz), 2.85 (s, 4H), 2.38-2.18 (m, 6H), 1.65- 1.44 (m, 3H), 1.12-1.00 (m, 2H).

### Example 6. Synthesis of Compound 110

To a solution of (1R,8 S,9 s)-bicyclo[6.1,0]non-4-yn-9-ylmethyl N-succinimidyl carbonate (108) (500 mg, 1.71 mmol) in DCM (15 mL) were added triethylamine (718 uL, 5.14 mmol) and mono-Fmoc ethylenediamine hydrochloride (109) (657 mg, 2.06 mmol). The mixture was stirred for 45 min,diluted with EtOAc (150 mL) and washed with a 50% saturated aqueous NH₄Cl solution (50 mL). The aqueous layer was extracted with EtOAc (50 mL) and the combined organic layers were washed with H₂O (10 mL). The combined organic extracts were concentrated in vacuo and the half of the residue was purified by silica gel column chromatography (0 → 3% MeOH in DCM) which gave the desired compound 110 in 42% yield (332 mg, 0.72 mmol). ¹H NMR (400 MHz, CDCl₃) δ (ppm) 7.77 (d, J = 7.5 Hz, 2H), 7.59 (d, J = 7.4 Hz, 2H), 7.44-7.37 (m, 2H), 7.36-7.28 (m, 2H), 5.12 (br s, 1H), 4.97 (br s, 1H), 44.41 (d, J = 6.8 Hz, 2H), 4.21 (t, J = 6.7 Hz, 1H), 4.13 (d, J = 8.0 Hz, 2H), 3.33 (br s, 4H), 2.36-2.09 (m, 6H), 1.67-1.45 (m, 2H), 1.33 (quintet, J = 8.6 Hz, 1H), 1.01-0.85 (m, 2H). LCMS (ESI+) calculated for C₂₈H₃₁N₂O₄₊(M+ H+) 459.23 found 459.52.

### Example 7. Synthesis of Compound 111

Compound 110 (327 mg, 0.713 mmol) was dissolved in DMF (6 mL) and piperidine (0.5 mL) was added. After 2 h, the mixture was concentrated and the residue was purified by silica gel column chromatography (0 → 32% 0.7 N NH₃ MeOH in DCM), which gave the desired compound 111 as a yellow oil (128 mg, 0.542 mmol, 76%). ¹H-NMR (400 MHz, CDCl₃) δ (ppm, rotamers) 5.2 (bs, 1H), 4.15 (d, J = 8.0 Hz, 2H), 3.48-3.40 (m, ⅔H), 3.33-3.27 (m, ⅔H), 3.27-3.19 (m, 1⅓H), 2.85-2.80 (m, 1 ⅓H), 2.36-2.17 (m, 6H), 1.67-1.50 (m, 2H), 1.36 (quintet, J= 8.5 Hz, 1H), 1.01-0.89 (m, 2H).

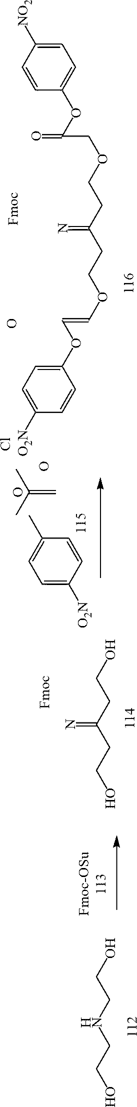

### Example 8. Synthesis of Compound 114

To a solution of diethanolamine (112) (208 mg, 1.98 mmol) in water (20 mL) were added MeCN (20 mL), NaHCOs (250 mg, 2.97 mmol) and a solution of Fmoc—OSu (113) (601 mg, 1.78 mmol) in MeCN (20 mL). The mixture was stirred for 2 h and DCM (50 mL) was added. After separation, the organic phase was washed with water (20 mL), dried (Na₂SO₄) and concentrated. The desired product 114 was obtained as a colorless thick oil (573 mg, 1.75 mmol, 98%). ¹H NMR (400 MHz, CDCl₃) δ (ppm) 7.79-7.74 (m, 2H), 7.60-7.54 (m, 2H), 7.44-7.37 (m, 2H), 7.36-7.30 (m, 2H), 4.58 (d, J = 5.4 Hz, 2H), 4.23 (t, J = 5.3 Hz, 1H), 3.82-3.72 (m, 2H), 3.48-3.33 (m, 4H), 3.25-3.11 (m, 2H).

### Example 9. Synthesis of Compound 116

To a solution of 114 (567 mg, 1.73 mmol) in DCM (50 mL) were added 4-nitrophenyl chloroformate (115) (768 mg, 3.81 mmol) and Et₃N (1.2 mL, 875 mg). The mixture was stirred for 18h and concentrated. The residue was purified by silica gel chromatography (0% → 10% MeOH in DCM, then 20% → 70% EtOAc in heptane, which afforded 32 mg (49 µmol, 2.8%) of the desired product 116. ¹H NMR (400 MHz, CDCl₃) δ (ppm) 8.31-8.20 (m, 4H), 7.80-7.74 (m, 2H), 7.59-7.54 (m, 2H), 7.44-7.37 (m, 2H), 7.37-7.29 (m, 6H), 4.61 (d, J = 5.4 Hz, 2H), 4.39 (t, J = 5.1 Hz, 2H), 4.25 (t, J = 5.5 Hz, 1H), 4.02 (t, J = 5.0 Hz, 2H), 3.67 (t, J = 4.8 Hz, 2H), 3.45 (t, J = 5.2 Hz, 2H).

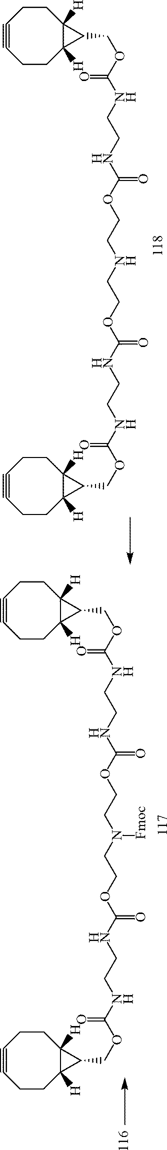

### Example 10. Synthesis of Compound 117

To a solution of 116 (34 mg, 0.050 mmol) in DCM (2 mL) were added 111 (49 mg, 0.21 mmol) and triethylamine (20 µL, 0.14 mmol). The mixture was left stirring overnight at room temperature. After 23 h, the mixture was concentrated. Purification by silica gel column chromatography (0 → 40% MeOH in DCM) gave 117 as a white solid in 61% yield (27 mg, 0.031 mmol). LCMS (ESI+) calculated for C₄₇H₅₇N₅O₁₀₊(M+H⁺) 851.41 found 852.49.

### Example 11. Synthesis of Compound 118

Compound 118 was obtained during the preparation of 117 (3.8 mg, 0.0060 mmol). LCMS (ESI+) calculated for C₃₂H₄₇N₅O₈⁺(M+H⁺) 629.34 found 630.54.

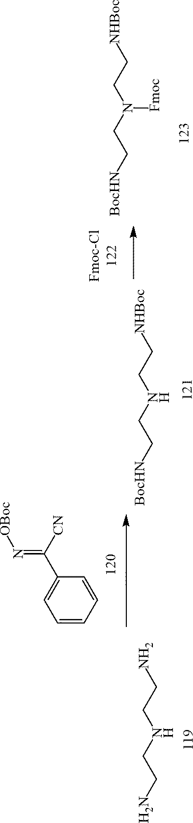

### Example 12. Synthesis of Compound 121

A solution of diethylenetriamine (119) (73 µL, 0.67 mmol) and triethylamine (283 µL, 2.03 mmol) in THF (6 mL) was cooled down to -5° C. and placed under a nitrogen atmosphere. 2-(Boc-oxyimino)-2-phenylacetonitrile (120) (334 mg, 1.35 mmol) was dissolved in THF (4 mL) and slowly added to the cooled solution. After 2.5 h, the ice bath was removed and the mixture was stirred for an additional of 2.5 h at room temperature, and concentrated in vacuo. The residue was redissolved in DCM (15 mL) and washed with a 5% aqueous NaOH solution (2 × 5 mL), brine (2 × 5 mL) and dried over MgSO₄. Purification by silica gel column chromatography (0 → 14% MeOH in DCM) gave 121 as a colorless oil in 91% yield (185 mg, 0.610 mmol). ¹H-NMR (400 MHz, CDCl₃) δ (ppm) 5.08 (s, 2H), 3.30-3.12 (m, 4H), 2.74 (t, J = 5.9 Hz, 4H), 1.45 (s, 18H).

### Example 13. Synthesis of Compound 123

To a cooled solution (-10° C.) of 121 (33.5 mg, 0.110 mmol) in THF (2 mL) were added a 10% aqueous NaHCO₃ solution (500 µL) and 9-fluorenylmethoxycarbonyl chloride (122) (34 mg, 0.13 mmol) dissolved in THF (1 mL). After 1 h, the mixture was concentrated and the residue was redissolved in EtOAc (10 mL), washed with brine (2 × 5 mL), dried over Na₂SO₄, and concentrated. Purification by silica gel column chromatography (0 → 50% MeOH in DCM) gave 123 in 86% yield (50 mg, 0.090 mmol). ¹H-NMR (400 MHz, CDCl₃) δ (ppm) 7.77 (d, J = 7.4 Hz, 2H), 7.57 (d, J = 7.4 Hz, 2H), 7.43-7.38 (m, 2H), 7.36-7.31 (m, 2H), 5.57 (d, J = 5.2 Hz, 2H), 4.23 (t, J = 5.1 Hz, 1H), 3.40-2.83 (m, 8H), 1.41 (s, 18H).

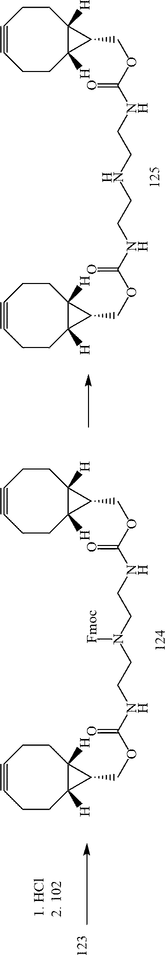

### Example 14. Synthesis of Compound 124

To a solution of 123 (50 mg, 0.095 mmol) in DCM (3 mL) was added 4 M HCl in dioxane (200 µL). The mixture was stirred for 19 h, concentrated and a white solid was obtained (35 mg). without purification, the deprotected intermediate and (1R,8 S,9 s)-bicyclo[6.1.0]non-4-yn-9-ylmethyl (4-nitrophenyl) carbonate (102) (70 mg, 0.22 mmol) were dissolved in DMF (3 mL) and triethylamine (34 µL, 0.24 mmol) was added. After 2 h, the mixture was concentrated and the residue was purified by silica gel column chromatography (0 → 25% MeOH in DCM) to yield 124 in 48% (31 mg, 0.045 mmol). LCMS (ESI+) calculated for C₄₁H₄₇N₃O₆⁺ (M+H⁺) 677.35 found 678.57.

### Example 15. Synthesis of Compound 125

To a solution of 124 (10 mg, 0.014 mmol) in DMF (500 µL) was added piperidine (20 µL). After 3.5 h, the mixture was concentrated. Purification by silica gel column chromatography (0 → 20% MeOH in DCM) gave 125 in 58% yield (3.7 mg, 0.0080 mmol). LCMS (ESI+) calculated for C₂₆H₃₇N₃O₄⁺ (M+H⁺) 455.28 found 456.41.

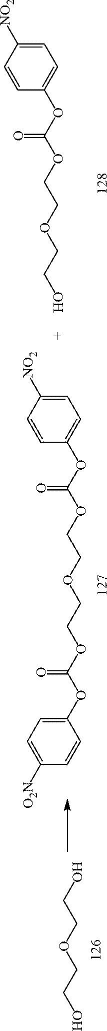

### Example 16. Synthesis of Compound 127 and 128

To a solution of diethyleneglycol (126) (446 µL, 0.50 g, 4.71 mmol) in DCM (20 mL) were added 4-nitrophenol chloroformate (115) (1.4 g, 7.07 mmol) and Et₃N (3.3. mL, 2.4 g, 23.6 mmol). The mixture was stirred, filtered and concentrated in vacuo (at 55° C.). The residue was purified by silica gel chromatography (15% → 75% EtOAc in heptane) and two products were isolated. Product 127 was obtained as a white solid (511 mg, 1.17 mmol, 25%).¹H NMR (400 MHz, CDCl₃) δ (ppm) 8.31-8.23 (m, 4H), 7.43-7.34 (m, 4H), 4.54-4.44 (m, 4H), 3.91-3.83 (m, 4H). Product 128 was obtained as a colorless oil (321 mg, 1.18 mmol, 25%).¹H NMR (400 MHz, CDCl₃) δ (ppm) 8.32-8.24 (m, 2H), 7.43-7.36 (m, 2H), 4.50-4.44 (m, 2H), 3.86-3.80 (m, 2H), 3.81-3.74 (m, 2H), 3.69-3.64 (m, 2H).

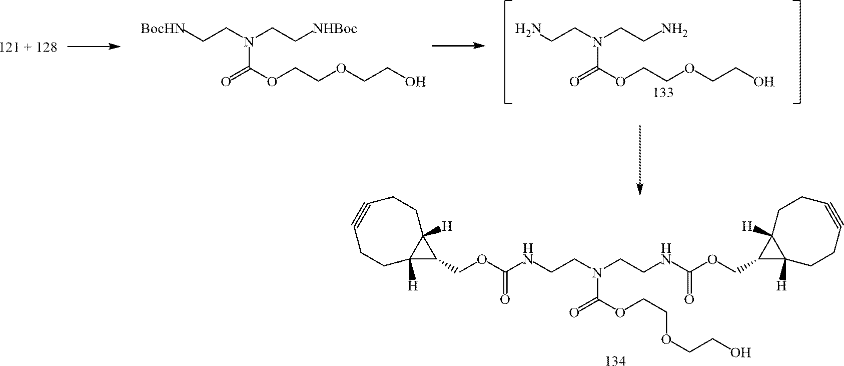

### Example 17. Synthesis of Compound 132

To a solution of 121 (168 mg, 0.554 mmol) in DCM (2 mL), were added a solution of 128 (240 mg, 0.89 mmol) in DCM (1 mL), DCM (1 mL) and Et₃N (169 mg, 233 µL). The mixture was stirred for 17 h, concentrated and purified by silica gel chromatography (gradient of EtOAc in heptane). The desired product 132 was obtained as a slightly yellow oil (85 mg, 0.20 mmol, 35%). ¹H NMR (400 MHz, CDCl₃) δ (ppm) 5.24-5.02 (m, 2H), 4.36-4.20 (m, 3H), 3.84-3.67 (m, 4H), 3.65-3.58 (m, 2H), 3.47-3.34 (m, 4H), 3.34-3.18 (m, 4H), 1.44 (bs, 18H).

### Example 18. Synthesis of Compound 134

To a solution of 132 (81 mg, 0.19 mmol) in DCM (3 mL) was added 4 N HCl in dioxane (700 µL). The mixture was stirred for 19 h, concentrated and the residue was taken up in DMF (0.5 mL). Et₃N (132 µL, 96 mg, 0.95 mmol), DMF (0.5 mL) and (1R,8 S,9 s)-bicyclo[6.1,0]non-4-yn-9-ylmethyl (4-nitrophenyl) carbonate (102) (132 mg, 0.42 mmol) were added and the resulting mixture was stirred for 2 h. The mixture was concentrated and the residue was purified by silica gel chromatography (0% → 3% MeOH in DCM). The desired product 134 was obtained as a colorless film (64 mg, 0.11 mmol, 57%). ¹H NMR (400 MHz, CDCl₃) δ (ppm) 4.31-4.23 (m, 2H), 4.22-4.08 (m, 4H), 3.80-3.68 (m, 4H), 3.66-3.58 (m, 2H), 3.50-3.28 (m, 8H), 2.80-2.65 (m, 1H), 2.40-2.10 (m, 12H), 1.68-1.48 (m, 4H), 1.35 (quintet, J = 8.1 Hz, 1H), 1.02-0.87 (m, 2H). LCMS (ESI+) calculated for C₃₁H₄₆N₃O₈⁺ (M+H⁺) 588.33 found 588.43.

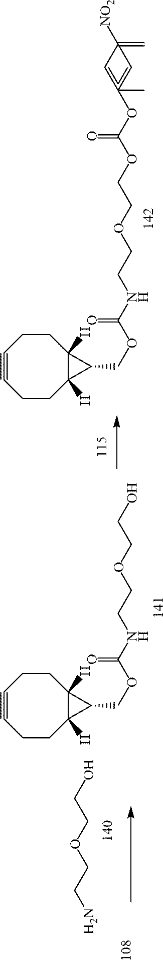

### Example 19. Synthesis of Compound 141

To a solution of (1R,8S,9S)-bicyclo[6.1.0]non-4-yn-9-ylmethyl N-succinimidyl carbonate (108) (16.35 g, 56.13 mmol) in DCM (400 ml) were added 2-(2-aminoethoxy)ethanol (140) (6.76 ml, 67.35 mmol) and triethylamine (23.47 ml, 168.39 mmol). The resulting pale yellow solution was stirred at rt for 90 min. The mixture was concentrated in vacuo and the residue was co-evaporated once with acetonitrile (400 mL). The resulting oil was dissolved in EtOAc (400 mL) and washed with H₂O (3 × 200 mL). The organic layer was concentrated in vacuo. The residue was purified by silica gel column chromatography (50% → 88% EtOAc in heptane) and gave 141 (11.2 g, 39.81 mmol, 71% yield) as a pale yellow oil. ¹H-NMR (400 MHz, CDCl₃): δ (ppm) 5.01 (br s, 1H), 4.17 (d, 2H, J= 12.0 Hz), 3.79-3.68 (m, 2H), 3.64-3.50 (m, 4H), 3.47-3.30 (m, 2H), 2.36-2.14 (m, 6H), 1.93 (br s, 1H), 1.68-1.49 (m, 2H), 1.37 (quintet, 1H, J= 8.0 Hz), 1.01-0.89 (m, 2H).

### Example 20. Synthesis of Compound 142

To a solution of 141 (663 mg, 2.36 mmol) in DCM (15 mL) were added triethylamine (986 uL, 7.07 mmol) and 4-nitrophenyl chloroformate (115) (712 mg, 3.53 mmol). The mixture was stirred for 4 h and concentrated in vacuo. Purification by silica gel column chromatography (0 → 20% EtOAc in heptane) gave 142 (400 mg, 0.9 mmol, yield 38%) as a pale yellow oil. ¹H-NMR (400 MHz, CDCl₃) δ (ppm) 8.29 (d, J = 9.4 Hz, 2H), 7.40 (d, J = 9.3 Hz, 2H), 5.05 (br s, 1H), 4.48-4.41 (m, 2H), 4.16 (d, J = 8.0 Hz, 2H), 3.81-3.75 (m, 2H), 3.61 (t, J = 5.0 Hz, 2H), 3.42 (q, J = 5.4 Hz, 2H), 2.35-2.16 (m, 6H), 1.66-1.50 (m, 2H), 1.35 (quintet, J = 8.6 Hz, 1H), 1.02-0.88 (m, 2H). LCMS (ESI+) calculated for C₂₂H₂₆N₂NaO₈⁺(M+Na⁺) 469.16 found 469.36.

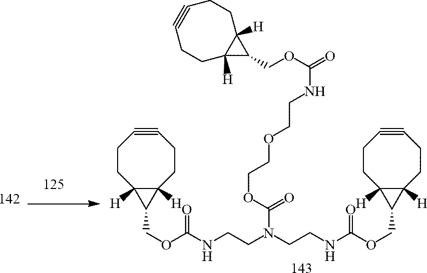

### Example 21. Synthesis of Compound 143

A solution of 142 (2.7 mg, 6.0 µmol) in DMF (48 µL) and Et₃N (2.1 µL, 1.5 mg, 15 µmol) were added to a solution of 125 (2.3 mg, 5.0 µmol) in DMF (0.32 mL). The mixture was left standing for 4 d, diluted with DMF (100 µL) and purified by RP HPLC (C18, 30% → 100% MeCN (1% AcOH) in water (1% AcOH). The product 143 was obtained as a colorless film (2.8 mg, 3.7 µmol, 74%). LCMS (ESI+) calculated for C₄₂H₅₉N₄O₉⁺ (M+H⁺) 763.43 found 763.53.

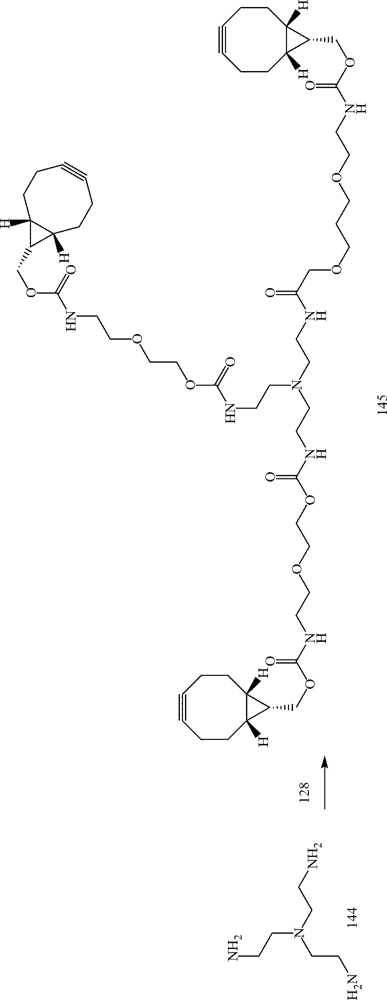

### Example 22. Synthesis of Compound 145

To a solution of 128 (200 mg, 0.45 mmol) in DCM (1 mL) were added triethylamine (41.6 uL, 0.30 mmol) and tris(2-aminoethyl)amine 144 (14.9 uL, 0.10 mmol). After stirring the mixture for 150 minutes, it was concentrated in vacuo. The residue was purified by silica gel column chromatography (25% → 100% EtOAc in DCM then 0% → 10% MeOH in DCM) and gave 145 in 43% yield (45.4 mg, 42.5 umol) as a yellow oil. ¹H NMR (400 MHz, CDCl₃)— δ (ppm) 5.68-5.18 (m, 6H), 4.32-4.18 (m, 6H), 4.18-4.11 (d, J= 7.9 Hz, 6H), 3.74-3.61 (m, 6H), 3.61-3.51 (m, 6H), 3.43-3.29 (m, 6H), 3.29-3.15 (m, 6H), 2.65-2.47 (m, 6H), 2.37-2.16 (m, 18H), 1.69-1.49 (m, 6H), 1.35 (quintet, J= 8.9 Hz, 3H), 1.03-0.87 (m, 6H).

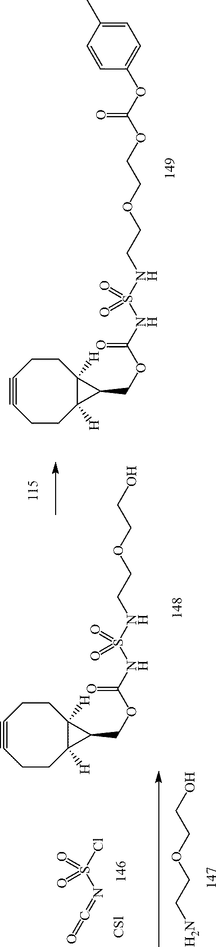

### Example 23. Synthesis of Compound 148

To a solution of BCN-OH (101) (3.0 g, 20 mmol) in DCM (300 mL) was added CSI (146) (1.74 mL, 2.83 g, 20 mmol). After the mixture was stirred for 15 min, Et₃N (5.6 mL, 4.0 g, 40 mmol) was added. The mixture was stirred for 5 min and 2-(2-aminoethoxy)ethanol (147) (2.2 mL, 2.3 g, 22 mmol) was added. The resulting mixture was stirred for 15 min and saturated aqueous NH₄Cl (300 mL) was added. The layers were separated, and the aqueous phase was extracted with DCM (200 mL). The combined organic layers were dried (Na₂SO₄) and concentrated. The residue was purified by silica gel chromatography (0% to 10% MeOH in DCM). The fractions, containing the desired product, were concentrated. The residue was taken up in EtOAc (100 mL) and concentrated. The desired product 148 was obtained as a slightly yellow oil (4.24 g, 11.8 mmol, 59%). ¹H NMR (400 MHz, CDCl₃) δ (ppm) 5.99-5.79 (bs, 1H), 4.29 (d, J= 8.3 Hz, 2H), 3.78-3.74 (m, 2H), 3.66-3.56 (m, 4H), 3.37-3.30 (m, 2H), 2.36-2.16 (m, 6H), 1.63-1.49 (m, 2H), 1.40 (quintet, J= 8.7 Hz, 1H), 1.05-0.94 (m, 2H).

### Example 24. Synthesis of Compound 149

To a solution of 148 (3.62 g, 10.0 mmol) in DCM (200 mL) were added 4-nitrophenyl chloroformate (15) (2.02 g, 10.0 mmol) and Et₃N (4.2 mL, 3.04 g, 30.0 mmol). The mixture was stirred for 1.5 h and concentrated. The residue was purified by silica gel chromatography (20% → 70% EtOAc (1% AcOH) in heptane (1% AcOH). The product 149 was obtained as a white foam (4.07 g, 7.74 mmol, 74%). ¹H NMR (400 MHz, CDCl₃) δ (ppm) 8.32-8.26 (m, 2H), 7.45-7.40 (m, 2H), 5.62-5.52 (m, 1H), 4.48-4.42 (m, 2H), 4.28 (d, J = 8.2 Hz, 2H), 3.81-3.76 (m, 2H), 3.70-3.65 (m, 2H), 3.38-3.30 (m, 2H), 2.35-2.16 (m, 6H), 1.62-1.46 (m, 2H), 1.38 (quintet, J= 8.7 Hz, 1H), 1.04-0.93 (m, 2H).

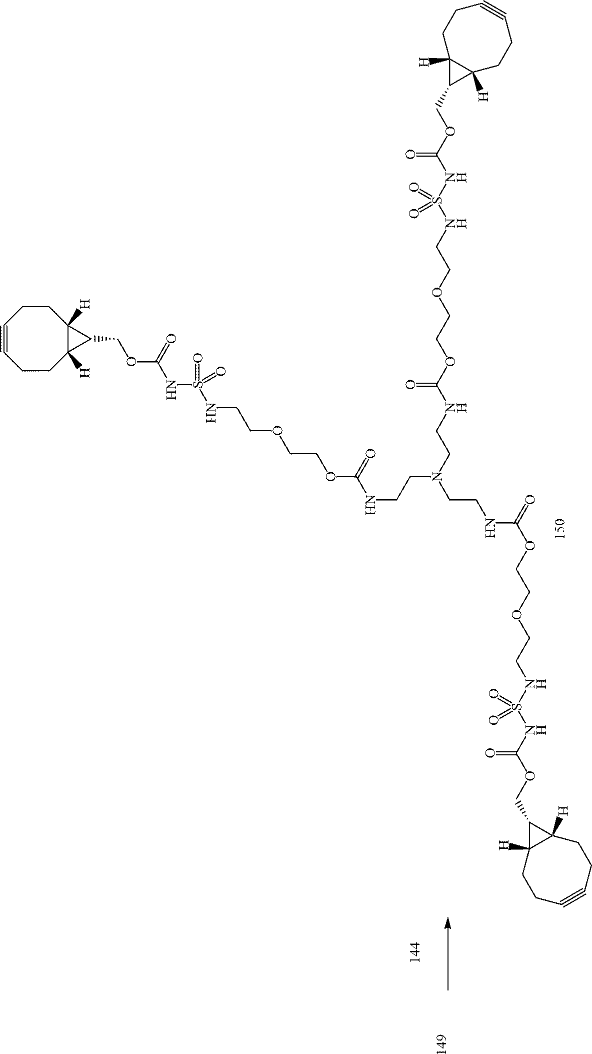

### Example 25. Synthesis of Compound 150

To a solution of 149 (200 mg, 0.38 mmol) in DCM (1 mL) were added triethylamine (35.4 uL, 0.24 mmol) and tris(2-aminoethyl)amine (144) (12.6 uL, 84.6 umol). The mixture was stirred for 120 min and concentrated in vacuo. The residue was purified by silica gel column chromatography (25% → 100% EtOAc in DCM then 0% → 10% MeOH in DCM) and gave 150 in 36% yield (40.0 mg, 30.6 umol) as a white foam. 1H NMR (400 MHz, CDCI₃): δ (ppm) 6.34-5.72 (m, 6H), 4.34-4.18 (m, 12H), 3.76-3.58 (m, 12H), 3.43-3.30 (m, 6H), 3.30-3.18 (m, 6H), 2.64-2.49 (m, 6H), 2.38-2.14 (m, 18H), 1.65-1.47 (m, 6H), 1.39 (quintet, J = 9.1 Hz, 3H), 1.06-0.90 (m, 6H).

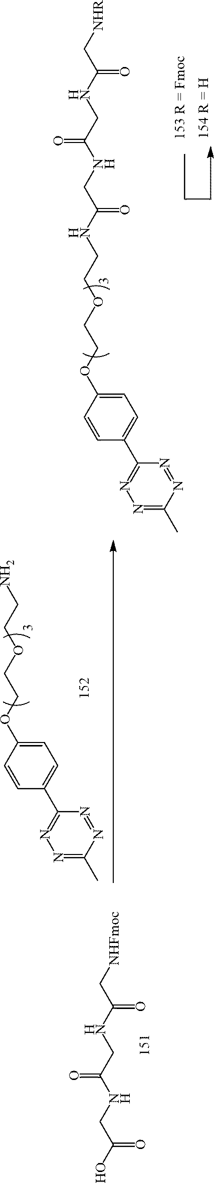

### Example 26. Synthesis of Compound 153

To a mixture of Fmoc—Gly—Gly—Gly—OH (151) (31.2 mg, 75.8 µmol) in anhydrous DMF (1 mL) were added N,N-diisopropylethylamine (40 µL, 29 mg, 0.23 mmol) and HATU (30.3 mg, 79.6 µmol). After 10 min tetrazine-PEG3-ethylamine (152) (30.3 mg, 75.8 µmol) was added and the mixture was vortexed. After 2 h, the mixture was purified by RP HPLC (C18, 30% → 90% MeCN (1% AcOH) in water (1% AcOH). The desired product was obtained as a pink film (24.1 mg, 31.8 µmol, 42%). LCMS (ESI+) calculated for C₃₈H₄₅N₈O₉+(M+H+) 757.33 found 757.46.

### Example 27. Synthesis of Compound 154

To a solution of 153 (24.1 mg, 31.8 µmol) in DMF (500 µL) was added diethylamine (20 µL, 14 mg, 191 µmol). The mixture was left standing for 2 h and purified by RP HPLC (C18, 5% → 90% MeCN (1% AcOH) in water (1% AcOH). The desired product 154 was obtained as a pink film (17.5 mg, 32.7 µmol, quant). LCMS (ESI+) calculated for C₂₃H₃₅N₈O₇+ (M+H+) 535.26 found 535.37.

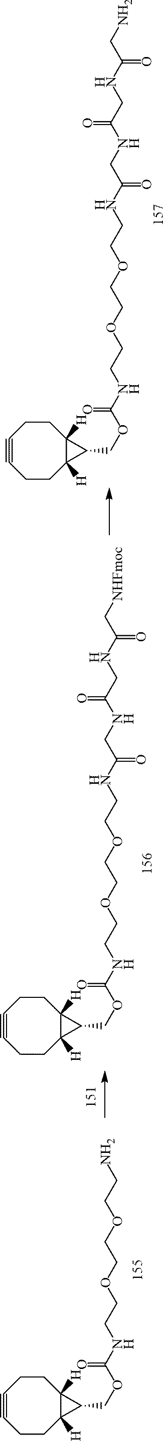

### Example 28. Synthesis of Compound 156

A solution of N-[(1R,8S,9s)-bicyclo[6.1.0]non-4-yn-9-ylmethyloxycarbonyl]-1,8-diamino-3,6-dioxaoctane (155) (68 mg, 0.21 mmol) in dry DMF (2 mL) was transferred to a solution of Fmoc—Gly—Gly—Gly—OH (151) (86 mg, 0.21 mmol) in dry DMF (2 mL). DIPEA (100 µL, 0.630 mmol) and HATU (79 mg, 0.21 mmol) were added. After 1.5 h, the mixture was concentrated and the residue was purified by silica gel column chromatography (0 → 11% MeOH in DCM) which gave the desired compound 156 in 34% yield (52 mg, 0.072 mmol). LCMS (ESI+) calculated for C₃₅H₄₇N₅O₉+ (M+ H+) 717.34 found 718.39.

### Example 29. Synthesis of Compound 157

Compound 156 (21 mg, 0.029 mmol) was dissolved in DMF (2.4 mL) and piperidine (600 µL) was added. After 20 minutes, the mixture was concentrated and the residue was purified by preparative HPLC, which gave the desired compound 157 as a white solid (9.3 mg, 0.018 mmol, 64%). LCMS (ESI+) calculated for C₂₃H₃₇N₅O₇+(M+ H+) 495.27 found 496.56.

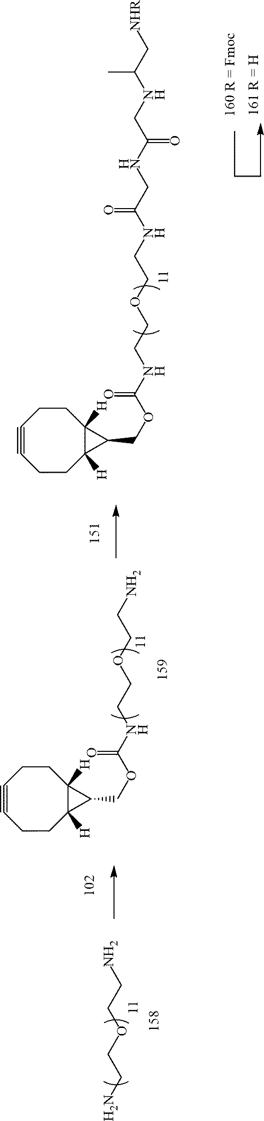

### Example 30. Synthesis of Compound 159

To a solution of amino-PEG₁₁-amine (158) (143 mg, 0.260 mmol) in DCM (5 mL) was slowly added (1R,8S,9s)-bicyclo[6.1.0]non-4-yn-9-ylmethyl (4-nitrophenyl) carbonate (102) (41 mg, 0.13 mmol) dissolved in DCM (5 mL). After 1.5 h, the mixture was reduced and the residue was purified by silica gel column chromatography (0 → 20% 0.7 N NH₃ MeOH in DCM) which gave the desired compound 159 as a clear oil (62 mg, 0.086 mmol, 66%). LCMS (ESI+) calculated for C₃₅H₄₆N₂O₁₃+(M+ H+) 720.44 found 721.56.

### Example 31. Synthesis of Compound 160

A solution of 159 (62 mg, 0.086 mmol) in dry DMF (2 mL) was transferred to a solution of Fmoc— Gly—Gly—Gly—OH (151) (36 mg, 0.086 mmol) in dry DMF (2 mL). DIPEA (43 µL, 0.25 mmol) and HATU (33 mg, 0.086 mmol) were added. After 18 h, the mixture was concentrated and the residue was purified by silica gel column chromatography (0 → 20% MeOH in DCM) which gave the desired compound 160 in 62% yield (60 mg, 0.054 mmol). LCMS (ESI+) calculated for C56H83N5O18+ (M+ H+) 1113.57 found 1114.93.

### Example 32. Synthesis of Compound 161

Compound 160 (36 mg, 0.032 mmol) was dissolved in DMF (2 mL) and piperidine (200 µL) was added. After 2 h, the mixture was concentrated and the residue was purified by silica gel column chromatography (0 → 40% 0.7 N NH₃ MeOH in DCM) which gave the desired compound 161 as a yellow oil (16.7 mg, 0.0187 mmol, 58%). LCMS (ESI+) calculated for C₄₁H₇₃N₅O₁₆+ (M+H+) 891.51 found 892.82.

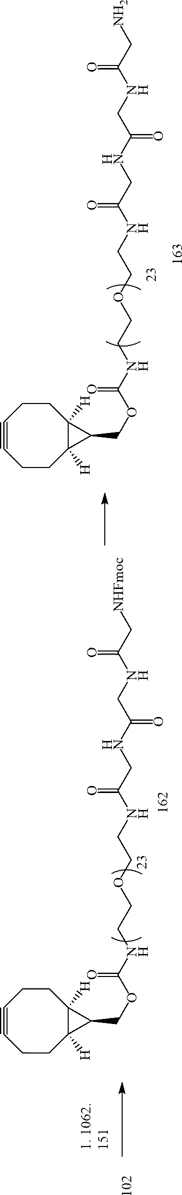

### Example 33. Synthesis of Compound 162

To a solution of amino-PEG23-amine (106) (60 mg, 0.056 mmol) in DCM (3 mL) was slowly added (1R,8S,9 s)-bicyclo[6.1.0]non-4-yn-9-ylmethyl (4-nitrophenyl) carbonate (102) (12 mg, 0.037 mmol) dissolved in DCM (5 mL). After 4 h, the mixture was concentrated and redissolved in DMF (2 mL), after which Fmoc—Gly—Gly—Gly—OH (51) (23 mg, 0.056 mmol), HATU (21 mg, 0.056 mmol), and DIPEA (27 µL, 0.16 mmol) were added. After 20 h, the mixture was concentrated and the residue was purified by silica gel column chromatography (0 → 27% MeOH in DCM) which gave the desired compound 162 in 93% (57 mg, 0.043 mmol). LCMS (ESI+) calculated for C₈₀H₁₃₁ N₅O₃₀+ (M+NH₄+) 1641.89 found 1659.92.

### Example 34. Synthesis of Compound 163

Compound 162 (57 mg, 0.034 mmol) was dissolved in DMF (1 mL) and piperidine (120 µL) was added. After 2 h, the mixture was concentrated, redissolved in water and the Fmoc-piperidine byproduct was removed with extraction with diethyl ether (3 x 10 mL). After freeze dry, 163 was obtained as an yellow oil (46.1 mg, 0.032 mmol, 95%). LCMS (ESI+) calculated for C₆₅H₁₂₁NsO₂₈+ (M+H+) 1419.82 found 1420.91.

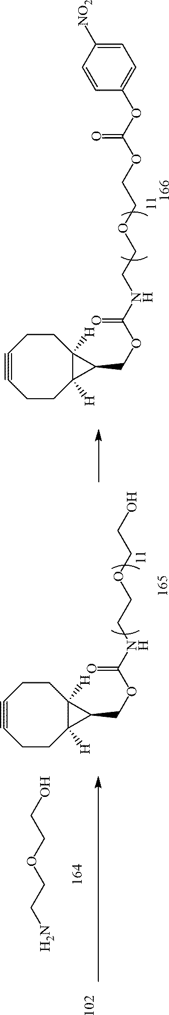

### Example 35. Synthesis of Compound 165

To a solution of (1R,8S,9s)-bicyclo[6.1.0]non-4-yn-9-ylmethyl (4-nitrophenyl) carbonate (102) (204 mg, 0.650 mmol) were added amino-PEG12-alcohol (164) (496 mg, 0.908 mmol) and triethyl amine (350 µL, 2.27 mmol). After 19 h, the mixture was concentrated and the residue was purified by silica gel column chromatography (2 → 20% MeOH in DCM) which gave 165 as a clear yellow oil (410 mg, 0.560 mmol, 87%). LCMS (ESI+) calculated for C₃₅H₆₃NO₁₄+ (M+ Na+) 721.42 found 744.43.

### Example 36. Synthesis of Compound 166

To a solution of 165 (410 mg, 0.560 mmol) in DCM (6 mL) were added 4-nitrophenyl chloroformate (171, 0.848 mmol) and triethyl amine (260 µL, 1.89 mmol). After 18 h, the mixture was concentrated and the residue was purified by silica gel column chromatography (0 → 7% MeOH in DCM) which gave the desired compound 166 as a clear oil (350 mg, 0.394 mmol, 70%). LCMS (ESI+) calculated for C₄₂H₆₆N₂O₁₈+(M+ Na+) 886.43 found 909.61.

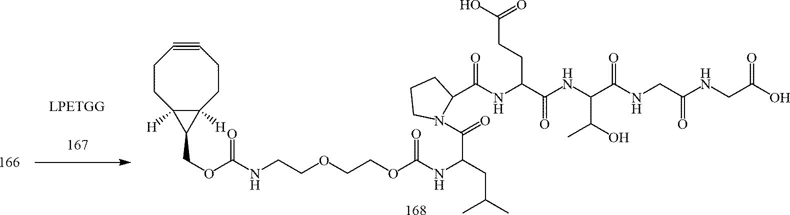

### Example 37. Synthesis of Compound 168

To a solution of 166 (15 mg, 0.017 mmol) in DMF (2 mL) were added peptide LPETGG (167) (9.7 mg, 0.017 mmol) and triethylamine (7 µL, 0.05 mmol). After 46 h, the mixture was concentrated and the residue was purified by preparative HPLC, which gave the desired compound 168 in 63% (14 mg, 0.010 mmol). LCMS (ESI+) calculated for C₆₀H₁₀₁N₇O₂₅+ (M+H+) 1319.68 found 1320.92.

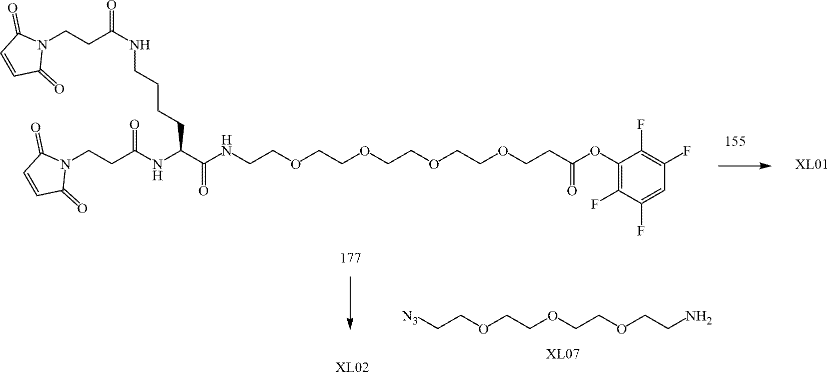

### Example 38. Synthesis of XL01

To a solution of 155 (9.7 mg, 0.03 mmol) in anhydrous DMF (170 µL) were added 177 (bis-maleimide-lysine-PEG₄-TFP, Broadpharm) (20 mg, 0.024 mmol) and Et₃N (9.9 µL, 0.071 mmol). After stirring at room temperature for 42 h, the mixture was diluted with DCM (0.4 mL) and purified by flash column chromatography over silicagel (0% → 18% MeOH in DCM) to give XL01 as a clear oil (10.2 mg, 0.010 mmol, 43%). LCMS (ESI+) calculated for C₄₉H₇₂N₇O₁₆+ (M+H+) 1003.12 found 1003.62.

### Example 39. Synthesis of Bis-maleimide Azide XL02

To a vial containing 177 (32.9 mg, 39.0 µmol, 1.0 equiv.) in dry DMF (400 µL) was added XL07 (9.2 mg, 42.1 µmol, 1.08 equiv.) and the solution was mixed and left at rt for circa 50 min. Next, DiPEA was added and the resulting solution was mixed and left at rt for circa 2 hours. The reaction mixture was then purified directly by silica gel chromatography (DCM → 14% MeOH in DCM). The desired product XL02 was obtained as a colorless oil (28.9 mg, 32.2 µmol, 83% yield). LCMS (ESI+) calculated for C₃₉H₆₂N₉O₁₅+ (M+H+) 896.97. found 896.52.

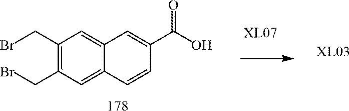

### Example 40. Synthesis of XL03

To a vial containing 2,3-bis(bromomethyl)-6-quinoxalinecarboxylic acid 178 (51.4 mg, 142.8 µmol, 1.00 equiv.) in dry DCM (7.5 mL) was added DIC (9.0 mg, 71.4 µmol, 0.5 equiv.). The resulting mixture was left at rt for 30 minutes, followed by the addition of a solution of XL07 (17.7 mg, 78.5 µmol, 0.55 equiv.) in dry DCM (0.5 mL). The reaction mixture was stirred at rt for circa 35 minutes and then purified directly by silica gel chromatography (DCM → 10% MeOH in DCM) to give impure product (72 mg) as a white solid. The impure product was taken up in 1.0 mL DMF and 50% of this solution was co-evaporated with toluene (2x). The residue was purified by silica gel chromatography (12 → 30% acetone in toluene). The desired product XL03 was obtained as a colorless oil (20.1 mg, 35.9 µmol). LCMS (ESI+) calculated for C₁₉H₂₅Br₂N₆o₄+ (M+H+) 561.03. found 561.12

### Example 41. Synthesis of XL05

To a solution of 178 (30 mg, 0.09 mmol), in DCM (0.3 mL) were added 3-maleimidopropionic NHS ester (27 mg, 0.10 mmol) and Et₃N (38 µL, 0.27 mmol). After stirring at room temperature for 28 h, the crude mixture was concentrated in vacuo and purified by flash column chromatography over silicagel (0% → 15% MeOH in DCM) to give XL05 as a clear oil (27 mg, 0.056 mmol, 62%). LCMS (ESI+) calculated for C₂₄H₃₄N₃O₇+ (M+H+) 476.54 found 476.46.

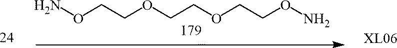

### Example 42. Synthesis of XL06

To a vial containing 24 (17.2 mg, 88 wt% by ¹⁻H-qNMR, 18.4 µmol, 1.00 equiv.) was added a solution of 179 in dry DMF (60 µL). To the resulting colorless solution was added triethylamine (40.6 µL, 15.8 equiv., 291 µmol), generating a yellow solution immediately. The reaction mixture was left at room temperature for circa 28 hours and was then conc. in vacuo until most of the Et₃N had evaporated. The residue was then diluted with DCM (1 mL) and purified directly by silica gel chromatography (1st column: DCM → 20% MeOH in DCM, 2_{nd} column: DCM → 20% MeOH in DCM). The desired product (XL06) was obtained as a colorless oil (4.3 mg, 18.4 µmol, 26% yield). LCMS (ESI+) calculated for C₃₄H₆₂N₇O₁₉S+ (M+H+) 904.38. found 904.52.

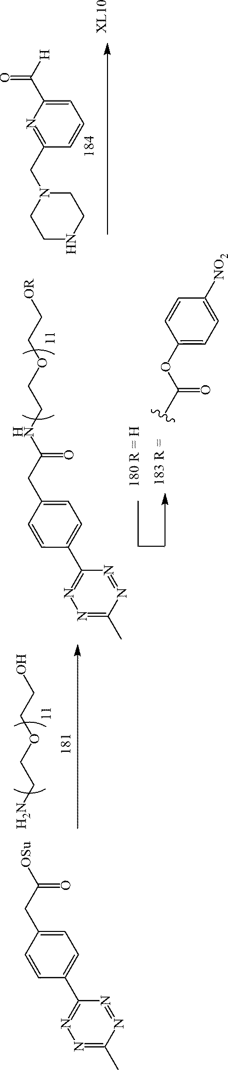

### Example 43. Synthesis of 182

To a solution of 180 (methyltetrazine-NHS ester, 19 mg, 0.058 mmol) in DCM (0.8 mL) were added 181 (33.6 mg, 0.061 mmol) and Et₃N (24 µL, 0.17 mmol). After stirring at room temperature for 2.5 h, the mixture was concentrated in vacuo and purified by flash column chromatography over silicagel (0 → 15% MeOH in DCM) which gave the desired compound 182 in 93% yield (41 mg, 0.054 mmol). LCMS (ESI+) calculated for C₃₅H₆₀N₅O₁₃+ (M+H+) 758.88 found 758.64.

### Example 44. Synthesis of 183

To a solution of 182 (41 mg, 0.054 mmol) in DCM (3 mL) were added 4-nitrophenyl chloroformate (16 mg, 0.081 mmol) and Et₃N (23 µL, 0.16 mmol). After stirring at room temperature for 21 h, the mixture was concentrated in vacuo and purified by flash column chromatography over silicagel (gradient: A. 0% → 20% EtOAc in DCM (till p-nitrophenol was eluded), followed by gradient B. 0% → 13% MeOH in DCM) which gave the desired compound 183 in 76% yield (37.9 mg, 0.041 mmol). LCMS (ESI+) calculated for C₄₂H₆₃N₆O₁₇+ (M+H+) 923.98 found 923.61.

### Example 45. Synthesis of XL10

To a solution of 184 (5.6 mg, 0.023 mmol), prepared according to MacDonald et al., Nat. Chem. Biol. 2015, 11, 326-334, incorporated by reference, in anhydrous DMF (0.1 mL) were added 183 (14.3 mg, 0.015 mmol) dissolved in anhydrous DMF (0.3 ml) and Et₃N (7 µL, 0.046 mmol). After stirring at room temperature for 2 h, the mixture was concentrated in vacuo and purified by flash column chromatography over silicagel (0 → 15% MeOH in DCM) which gave the desired compound XL10 in 50% yield (7.5 mg, 0.0076 mmol). LCMS (ESI+) calculated for C₄₇H₇₃N₈O₁₅+ (M+H+) 990.13 found 990.66.

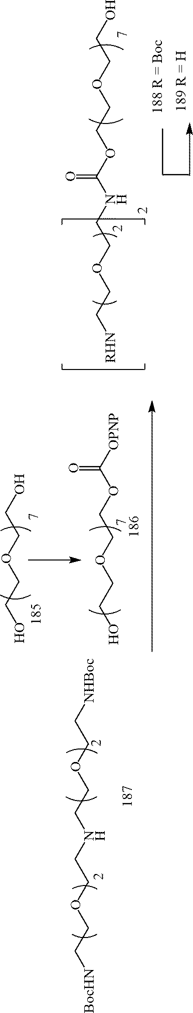

### Example 46. Synthesis of 186

To a solution of octa-ethylene glycol 185 in DCM (10 mL) was added triethylamine (1.0 mL, 7.24 mmol, 2.5 equiv.) followed by dropwise addition of a 4-nitrophenyl chloroformate (0.58 g, 2.90 mmol, 1 equiv.) solution in DCM (5 mL) in 28 minutes. After stirring the mixture for 90 minutes, it was concentrated in vacuo. The residue was purified by silicagel column chromatography (75% → 0% EtOAc in DCM followed by 0% → 7% MeOH in DCM). The product 186 was obtained in 38% yield as a colorless oil (584.6 mg, 1.09 mmol). LCMS (ESI+) calculated for C₂₃H₃₈NO₁₃+(M+H+) 536.23, found 536.93. ¹H-NMR (400 MHz, CDCI₃): δ (ppm) 8.28 (d, J = 12.0 Hz, 2H), 7.40 (d, J = 12.0 Hz, 2H), 4.47 - 4.42 (m, 2H), 3.84 - 3.79 (m, 2H), 3.75 - 3.63 (m, 26H), 3.63 - 3.59 (m, 2H), 2.70 -2.55 (bs, 1H).

### Example 47. Synthesis of 188

To a solution of 187 (BocNH—PEG₂)₂NH, 202 mg, 0.42 mmol) in DCM (1 mL) was added part (0.5 mL, 0.54 mmol 1.3 equiv.) of a prepared stock solution of 186 (584 mg in DCM (1 mL)) followed by triethylamine (176 µL, 1.26 mmol, 3 equiv.) and HOBt (57 mg, 0.42 mmol, 1 equiv.). After stirring the mixture for 8 days, it was concentrated in vacuo. The residue was taken up in a mixture of acetonitrile (4.2 mL) and 0.1N NaOH(_{aq}) (4.2 mL, 1 equiv.) and additional amount of solid NaOH (91.5 mg). After stirring the mixture for another 21.5 hours the mixture was extracted with DCM (3x 40 mL). The combined organic layers were concentrated in vacuo and the residue was purified by silicagel column chromatography (0% → 15% MeOH in DCM). Product 188 was obtained in 87% yield as a pale yellow oil (320.4 mg, 0.37 mmol). LCMS (ESI+) calculated for C₃₉H₇₈N₃o₁₈+(M+H+) 876.53, found 876.54.

¹H-NMR (400 MHz, CDCl₃): δ (ppm) 5.15 - 5.02 (bs, 2H), 4.25 - 4.19 (m, 2H), 3.76 - 3.46 (m, 50H), 3.35 - 3.26 (m, 4H), 2.79 - 2.69 (br. s, 1H), 1.44 (s, 18H).

### Example 48. Synthesis of 189

188 (320 mg, 0.37 mmol) was dissolved in DCM (1 mL). Then 4 M HCI in dioxane (456 µL, 1.83 mmol, 5 equiv.) was added. After stirring the mixture for 3.5 hours, additional 4 M HCI in dioxane (450 µL, 1.80 mmol, 4.9 equiv.) was added. After stirring the mixture for another 3.5 hours, additional 4 M HCI in dioxane (450 µL, 1.80 mmol, 4.9 equiv.) was added. After stirring the mixture for 16.5 hours the mixture was concentrated in vacuo. Product 189 was obtained in quantitative yield as a white sticky solid. This was used directly in the next step. ¹H-NMR (400 MHz, DMSO-d6): δ (ppm) 8.07 - 7.81 (bs, 6H), 4.15 - 4.06 (m, 2H), 3.75 - 3.66 (m, 2H), 3.65 - 3.48 (m, 48H), 3.03 - 2.92 (m, 4H).

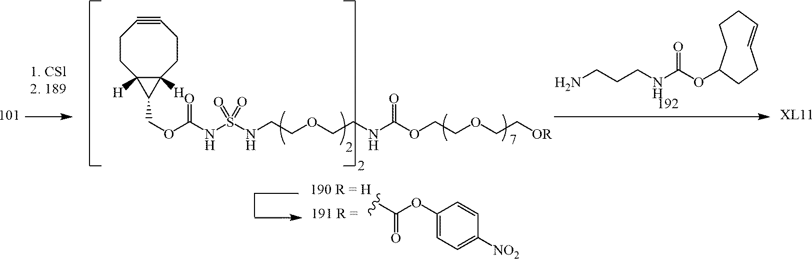

### Example 49. Synthesis of 190

To a solution of BCN-OH (164 mg, 1.10 mmol, 3 equiv.) in DCM (3 mL) was added CSI (76 µL, 0.88 mmol, 2.4 equiv.). After stirring for 15 minutes triethylamine (255 µL, 5.50 mmol, 5 equiv.) was added. A solution of 189 was prepared by adding DCM (3 mL) and triethylamine (508 µL, 11.0 mmol, 10 equiv.). This stock solution was added to the original reaction mixture after 6 minutes. After stirring the mixture for 21.5 hours, it was concentrated in vacuo. The residue was purified by silicagel column chromatography (0% → 10% MeOH in DCM). Product 190 was obtained in 39% yield as pale yellow oil (165.0 mg, 139 µmol). LCMS (ESI+) calculated for C₄₃H₇₂N₅O₁₈S₂+(M+H+) 1186.54, found 1186.65.

¹H-NMR (400 MHz, CDCl₃): δ (ppm) 6.09 - 5.87 (m, 2H), 4.31 - 4.19 (m, 6H), 3.76 - 3.50 (m, 50H), 3.40 - 3.29 (m, 4H), 2.38 - 2.16 (m, 12H), 1.66 - 1.47 (m, 4H), 1.40 (quintet, J = 8.0 Hz, 2H), 1.04 - 0.94 (m, 4H).

### Example 50. Synthesis of 191

To a solution of 190 (101 mg, 0.085 mmol) in DCM (2.0 mL) were added bis(4-nitrophenyl) carbonate (39 mg, 0.127 mmol) and Et₃N (36 uL, 0.25 mmol). After stirring at room temperature for 42 h, the crude mixture was concentrated in vacuo and purified by flash column chromatography over silicagel (A. 0% → 25% EtOAc in DCM (till p-nitrophenol was eluded), followed by gradient B. 0% → 12% MeOH in DCM) to give 191 as a clear oil (49 mg, 0.036 mmol, 42%). LCMS (ESI+) calculated for C₅₈H₉iN₆O₂₆S₂+ (M+H+) 1352.50 found 1352.78.

### Example 51. Synthesis of XL 11

To a solution of 191 (7 mg, 0.0059 mmol) in anhydrous DMF (130 µL) were added Et₃N (2.2 uL, 0.015 mmol) and TCO-amine hydrochloride (Broadpharm) (1.8 mg, 0.0068 mmol). After stirring at room temperature for 19 h, the crude mixture was purified by flash column chromatography over silicagel (0% → 15% MeOH in DCM) to give XL11 as a clear oil (1.5 mg, 0.001 mmol, 17%). LCMS (ESI+) calculated for C₆₄H₁₁₁N₈O₂₅S₂+ (M+NH₄+) 1456.73 found 1456.81.

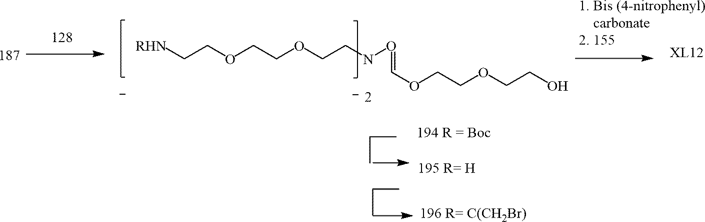

### Example 52. Synthesis of 194

To a solution of available 187 (638 mg, 1.33 mmol) in DCM (8.0 mL) were added 128 (470 mg, 1.73 mmol), Et₃N (556.0 µL, 4.0 mmol), and 1-hydroxybenzotriazole (179.0 mg, 1.33 mmol). After stirring for 41 h at ambient temperature, the mixture was concentrated in vacuo and redissolved in MeCN (10 mL) followed by the addition of aqueous 0.1 M NaOH solution (10 mL) and solid NaOH pellets (100.0 mg). After 1.5 h, DCM (20 mL) was added and the desired compound was extracted four times. The organic layers were concentrated in vacuo and the residue was purified by flash column chromatography over silicagel (0% → 12% MeOH in DCM) to give 194 as a clear yellow oil (733 mg, 1.19 mmol, 90%). ¹H NMR (400 MHz, CDCl₃) δ (ppm) 4.29 - 4.23 (m, 2H), 3.77 - 3.68 (m, 4H), 3.65 - 3.56 (m, 14H), 3.56 - 3.49 (m, 8H), 3.37 - 3.24 (m, 4H), 1.45 (s, 18H). LCMS (ESI+) calculated for C₂₇H₅₄N₃O₁₂+ (M+H+) 612.73 found 612.55.

### Example 53. Synthesis of 195

To a solution of 194 (31.8 mg, 0.052 mmol) in DCM (1.0 mL) was added 4.0 M HCI in dioxane (0.4 mL). After stirring for 2.5 h at ambient temperature, the reaction mixture was concentrated in vacuo and in between redissolved in DCM (2 mL) and concentrated. Compound 195 was obtained as a clear oil in quantitative yield. LCMS (ESI+) calculated for C₁₇H₃₈N₃O₈+(M+H+) 412.50 found 412.45

### Example 54. Synthesis of 196

To a cold solution (0° C.) of 195 (21.4 mg, 0.052 mmol) in DCM (1.0 mL) were added Et₃N (36 µL, 0.26 mmol) and 2-bromoacetyl bromide (10.5 µL, 0.12 mmol). After stirring for 10 min on ice, the ice bath was removed and aqueous 0.1 M NaOH solution (0.8 mL) was added. After stirring at room temperature for 20 min, the water layer was extracted with DCM (2x 5 mL). The organic layers were combined and concentrated in vacuo. The crude brown oil was purified by flash column chromatography over silicagel (0% → 18% MeOH in DCM) to give 196 as a clear oil (6.9 mg, 0.011 mmol, 20%). LCMS (ESI+) calculated for C₂₁H₄oBr₂N₃O₁₀+ (M+H+) 654.36 found 654.29.

### Example 55. Synthesis of XL12

To a solution of 196 (6.9 mg, 0.011 mmol) in DCM (0.8 mL) were added bis(4-nitrophenyl) carbonate (3.8 mg, 0.012 mmol) and Et₃N (5 µL, 0.03 mmol). After stirring at room temperature for 18 h, 155 (BCN-PEG₂-NH₂, 3.3 mg, 0.01 mmol) dissolved in DCM (0.5 mL) was added. After stirring for an additional of 2 h, the mixture was concentrated in vacuo and purified by flash column chromatography over silica gel (gradient: A. 0% → 30% EtOAc in DCM (till p-nitrophenol was eluded), followed by gradient B. 0% → 20% MeOH in DCM) to give XL12 as a clear oil (1.0 mg, 0.001 mmol, 9%). LCMS (ESI+) calculated for C₃₉H₆₆Br₂N₅O₁₅+ (M+H+) 1004.77 found 1004.51.

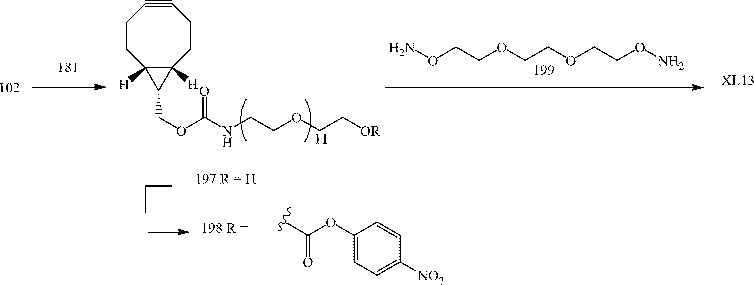

### Example 56. Synthesis of 197

To a solution of 102 (204 mg, 0.647 mmol) in DCM (20 mL) were added 181 (496 mg, 0.909 mmol) and Et₃N (350 µL, 2.27 mmol). After stirring at room temperature for 19 h, solvent was reduced in vacuo and the residue was purified by flash column chromatography over silicagel (2 → 20% MeOH in DCM) which gave the desired compound 197 as a yellow oil in 87% yield (410 mg, 0.567 mmol). LCMS (ESI+) calculated for C₃₅H₆₃NO₁₄Na+ (M+Na+) 744.86 found 744.43.

### Example 57. Synthesis of 198

To a solution of 197 (410 mg, 0.567 mmol) and 4-nitrophenyl chloroformate (172 mg, 0.853 mmol) in DCM (6 mL) was added Et₃N (260 µL, 1.88 mmol). After stirring at room temperature for 18 h, solvent was reduced in vacuo and the residue was purified by flash column chromatography over silicagel (0 → 7% MeOH in DCM) which gave the desired compound 198 as a clear oil in 70% yield (350 mg, 0.394 mmol). LCMS (ESI+) calculated for C₄₂H₆₆N₂O₁₈Na+ (M+Na+) 909.96 found 909.61.

### Example 58. Synthesis of XL13

To a solution of 198 (44.2 mg, 0.05 mmol) in DCM (5 mL) were added 199(bis-aminooxy-PEG₂, 33.3 mg, 0.18 mmol) and Et₃N (11 µL, 0.07 mmol). After stirring at room temperature for 67 h, the mixture was concentrated in vacuo and purified by RP HPLC (Column Xbridge prep C18 5 um OBD, 30x100 mm, 5% → 90% MeCN in H₂O (both containing 1% acetic acid)). The product XL13 was obtained as a clear oil (8.1 mg, 0.0087 µmol, 17%). LCMS (ESI+) calculated for C₄₂H₇₈N₃O₁₉+ (M+H+) 929.08 found 928.79.

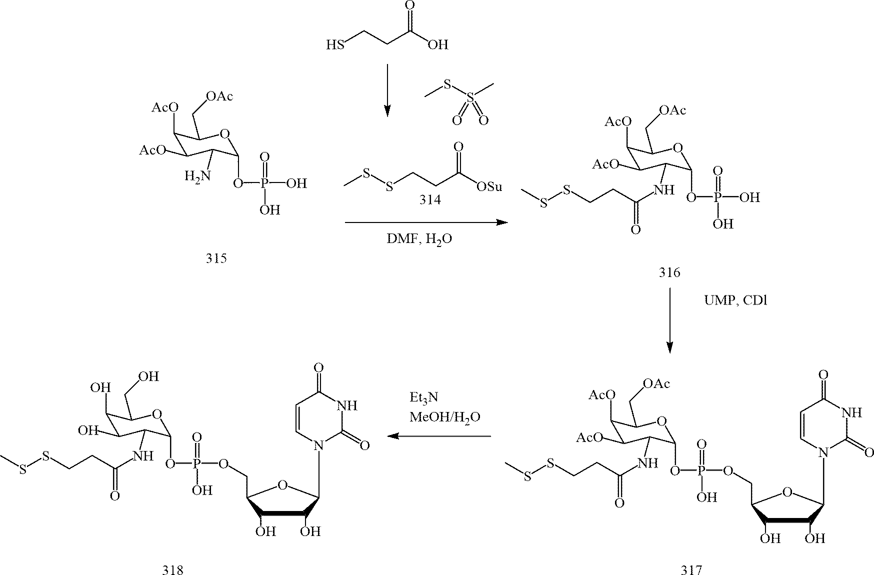

### Example 60. Synthesis of 314

A solution of 3-mercaptopropanoic acid (200 mg, 1.9 mmol) in water (6 mL) was cooled to 0° C., followed by the addition of methyl methanethiosulfonate (263 mg, 2.1 mmol) in ethanol (3 mL). The reaction was stirred overnight and warmed to room temperature. Subsequently, the reaction was quenched by saturated aqueous NaCl (10 mL) and Et₂O (20 mL). The water layer was extracted with Et₂O (3 x 20 mL), and the combined organic layers were dried over Na₂SO₄, filtrated and concentrated to yield the crude disulfide product (266 mg, 1.7 mmol, 93%). ¹H-NMR (400 MHz, CDCl₃): δ 7.00 (bs, 1H), 2.96-2.92 (m, 2H), 2.94-2.80 (m, 2H), 2.43 (s, 3H).

The crude disulfide derived from 3-mercaptopropanoic acid (266 mg, 1.7 mmol) was dissolved in CH₂Cl₂ (20 mL) followed by the addition of EDC.HCl (480 mg, 2.2 mmol) and N-hydroxy succinimide (270 mg, 2.1 mmol). The reaction was stirred for 90 minutes and quenched with water (20 mL). The organic layer was washed with saturated aqueous NaHCO₃ (2 x 20 mL). The organic layer was dried over Na₂SO₄, filtrated and concentrated to give crude 314 (346 mg, 1.4 mmol, 81%). ¹H-NMR (400 MHz, CDCl₃): δ 3.12-3.07 (m, 2H), 3.02-2.99 (m, 2H), 2.87 (bs, 4H), 2.44 (s, 3H).

### Example 61. Synthesis of 316

To a solution of 315 (prepared according WO2015057063 example 40, incorporated by reference) (420 mg, 1.14 mmol) in CH₂Cl₂/DMF (5 mL each) were added crude 314 (425 mg, 1.71 mmol) and Et₃N (236 µL, 1.71 mmol). The reaction mixture was stirred overnight followed by concentration under reduced pressure. Flash chromatography (1:0-6:4 MeCN:MeOH) afforded 316 (358 mg, 0.7 mmol, 60%). ¹H-NMR (400 MHz, CD₃OD): δ 5.46-5.45 (m, 1H), 5.33-5.27 (m, 1H), 5.15-5.11 (m, 1H), 4.43-4.41 (m, 1H), 4.17-4.06 (m, 2H), 3.97-3.88 (m, 1H), 2.89-2.83 (m, 2H), 2.69-2.53 (m, 2H), 2.32 (s, 3H), 2.04 (s, 3H), 1.91 (s, 3H), 1.86 (s, 3H).

### Example 62. Synthesis of UDP GalNProSSMe (318)

To a solution of UMP.NBus (632 mg, 1.12 mmol) in DMF (5 mL) CDI (234 mg, 1.4 mmol) was added and stirred for 30 minutes. Methanol (25 µL, 0.6 mmol) is added and after 15 minutes the reaction is placed under high vacuum for 15 minutes. Subsequently, 316 (358 mg, 0.7 mmol) and NMI.HCl (333 mg, 2.8 mmol) are dissolved in DMF (2 mL) and added to the reaction mixture. After stirring overnight, the reaction mixture is concentrated under reduced pressure to give crude 317. The crude product 317 is dissolved in MeOH:H₂O:Et₃N (7:3:3, 10 mL) and stirred overnight followed by the addition of additional MeOH:H₂O:Et₃N (7:3:3, 5 mL). After 48 h, total reaction time the reaction mixture was concentrated under reduced pressure. The crude product was purified via anion exchange column (Q HITRAP, 3 x 5 mL, 1 x 20 mL column) in two portions. First binding on the column was achieved via loading with buffer A (10 mM NaHCO₃) and the column was rinsed with 50 mL buffer A. Next a gradient to 70% B (250 mM NaHCO₃) was performed to elute UDP GaINProSSMe 318 (355 mg, 0.5 mmol, 72%). 1H-NMR (400 MHz, D₂O): δ 7.86-7.84 (m, 1H), 5.86-5.85 (m, 1H), 5.44 (bs, 1H), 4.26-4.22 (m, 2H), 4.17-4.08 (m, 6H), 3.92 (m, 1H), 3.84-3.83 (m, 1H), 3.66-3.64 (m, 2H), 2.88 (t, J = 7.2 Hz, 2H), 2.68 (t, J = 7.2 Hz, 2H), 2.31 (s, 3H).

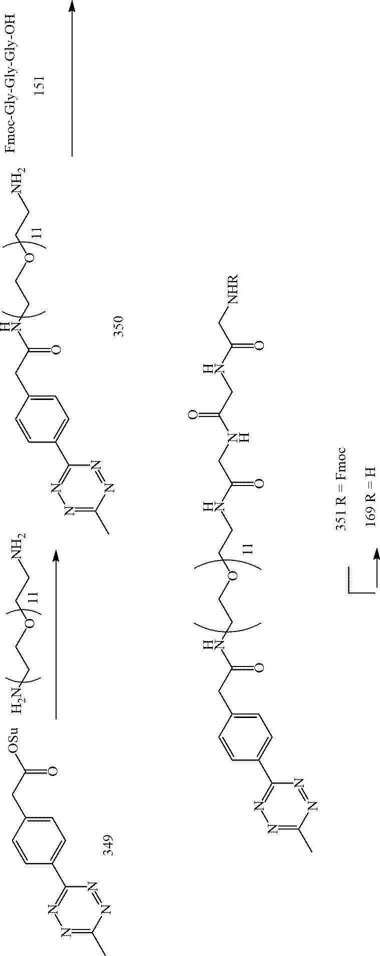

### Example 63. Synthesis of 350

To a solution of methyltetrazine-NHS ester 349 (19 mg, 0.057 mmol) in DCM (400 µL) was added amino-PEG₁₁-amine (47 mg, 0.086 mmol) dissolved in DCM (800 µL). After stirring at room temperature for 20 min, the mixture was concentrated in vacuo and purified by flash column chromatography over silicagel (0 → 50% MeOH (0.7 M NH₃) in DCM) which gave the desired compound 350 as a pink oil (17 mg, 0.022 mmol, 39%). LCMS (ESI+) calculated for C₃₅H₆₁N₆O₁₂+ (M+H+) 757.89 found 757.46.

### Example 64. Synthesis of 351

To a stirred solution of 151 (Fmoc—Gly—Gly—Gly—OH, 10 mg, 0.022 mmol) in anhydrous DMF (500 µL) were added DIPEA (11 µL, 0.067 mmol) and HATU (8.5 mg, 0.022 mmol). After 10 min, 350 (17 mg, 0.022 mmol) dissolved in anhydrous DMF (500 µL) was added. After stirring at room temperature for 18.5 h, the mixture was concentrated in vacuo and purified by flash column chromatography over silicagel (0 → 17% MeOH in DCM) which gave the desired compound 351 as a pink oil (26 mg, 0.022 mmol, quant.). LCMS (ESI+) calculated for C₅₆H₈₃N₁₀O₁₇₊ (M+NH₄+) 1168.32 found 1168.67

### Example 65. Synthesis of 169

To a solution of 351 (26 mg, 0.022 mmol) in anhydrous DMF (500 µL) was added diethylamine (12 µL, 0.11 mmol). After stirring at room temperature for 1.5 h, the crude mixture was purified by RP HPLC (Column Xbridge prep C18 5 µm OBD, 30x100 mm, 5% → 90% MeCN in H₂O (both containing 1% acetic acid)). The product 169 was obtained as a clear pink oil (10.9 mg, 0.011 mmol, 53%). LCMS (ESI+) calculated for C₄₁H₇₀N₉O₁₅+ (M+H+) 929.05 found 929.61.

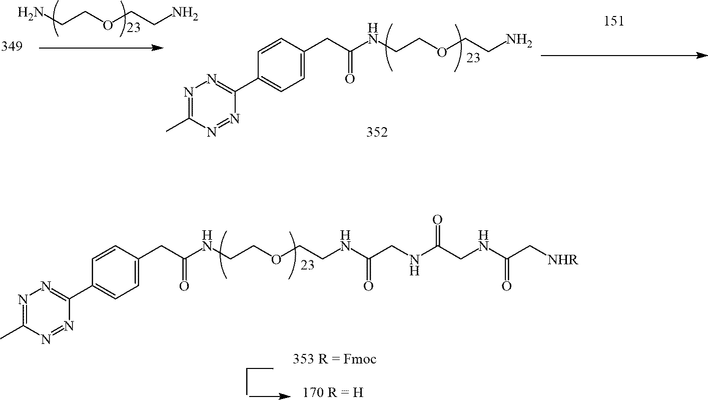

### Example 66. Synthesis of 352

To a solution of 349 (methyltetrazine-NHS ester, 10.3 mg, 0.031 mmol) in DCM (200 µL) was added amino-PEG₂₃-amine (50 mg, 0.046 mmol) dissolved in DCM (200 µL). After stirring at room temperature for 50 min, the mixture was concentrated in vacuo and purified by flash column chromatography over silicagel (0 → 60% MeOH (0.7 M NH₃) in DCM) which gave the desired compound 352 as a pink oil (17.7 mg, 0.013 mmol, 44%). LCMS (ESI+) calculated for C₅₉H₁₀₉N₆O₂₄+ (M+H+) 1286.52 found 1286.72.

### Example 67. Synthesis of 353

To a stirred solution of 151 (5.7 mg, 0.013 mmol) in anhydrous DMF (500 µL) were added DIPEA (7 µL, 0.04 mmol) and HATU (5.3 mg, 0.013 mmol). After 10 min, 352 (17.7 mg, 0.013 mmol) dissolved in anhydrous DMF (500 µL) was added. After stirring at room temperature for 6 h, the mixture was concentrated in vacuo and purified by flash column chromatography over silicagel (0 → 18% MeOH in DCM) which gave the desired compound 353 as a pink oil (21 mg, 0.012 mmol, 91%). LCMS (ESI+) calculated for C₈₀H₁₃₁N₁₀O₂₉+ (M/2+NH₄+) 857.45 found 857.08

### Example 68. Synthesis of 170

To a solution of 353 (21 mg, 0.012 mmol) in anhydrous DMF (500 µL) was added diethylamine (6.7 µL, 0.06 mmol). After stirring at room temperature for 4 h, the crude mixture was purified by RP HPLC (Column Xbridge prep C18 5 µm OBD, 30x100 mm, 5% → 90% MeCN in H₂O (both containing 1% acetic acid)). The product 170 was obtained as a pink oil (11.6 mg, 0.008 mmol, 66%). LCMS (ESI+) calculated for C₆₅H₁₁₈N₉O₂₇+ (M+H+) 1457.68 found 1457.92.

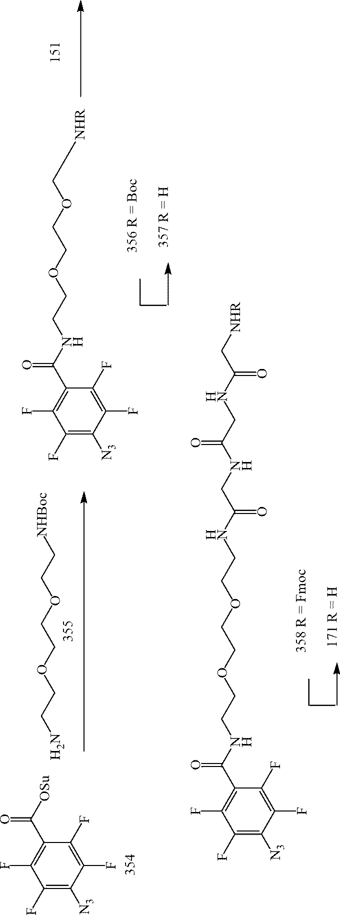

### Example 69. Synthesis of 356

To a solution of 354 (tetrafluorophenylazide-NHS ester, 40 mg, 0.12 mmol) in DCM (1 mL) were added 355 (Boc—NH—PEG₂—NH₂, 33 mg, 0.13 mmol) and Et₃N (50 µL, 0.36 mmol). After stirring in the dark at room temperature for 30 min, the mixture was concentrated in vacuo and purified by flash column chromatography over silicagel (0 → 7% MeOH in DCM) which gave the desired compound 356 as a clear oil (47 mg, 0.10 mmol, 84%). LCMS (ESI+) calculated for C₁₈H₂₄F₄N₅O₅+ (M+H+) 466.41 found 466.23.

### Example 70. Synthesis of 357

To a solution of 356 (47 mg, 0.10 mmol) in DCM (2 mL) was added 4.0 M HCI in dioxane (300 µL). After stirring in the dark at room temperature for 17.5 h, the mixture was concentrated and 357 was obtained as a white solid in quantitative yield (36 mg, 0.10 mmol). LCMS (ESI+) calculated for C₁₃H₁₆F₄N₅O₃₊ (M+H+) 366.29 found 366.20.

### Example 71. Synthesis of 358

To a stirred solution of 151 (Fmoc—Gly—Gly—Gly—OH, 42 mg, 0.10 mmol) in anhydrous DMF (600 µL) were added DIPEA (50 µL, 0.30 mmol) and HATU (39 mg, 0.10 mmol). After 15 min in the dark, 357 (36 mg, 0.10 mmol) dissolved in anhydrous DMF (500 µL) was added. After stirring in the dark at room temperature for 41 h, the mixture was concentrated in vacuo and purified by flash column chromatography over silicagel (0 → 20% MeOH in DCM) which gave the desired compound 358 as a clear oil (36 mg, 0.047 mmol, 47%). LCMS (ESI+) calculated for C₃₄H₃₅F₄N₈O₈+ (M+H+) 759.68 found 759.38.

### Example 72. Synthesis of 171

To a solution of 358 (36 mg, 0.047 mmol) in anhydrous DMF (750 µL) was added diethylamine (24 µL, 0.24 mmol). After stirring in the dark at room temperature for 55 min, the crude mixture was purified by RP HPLC (Column Xbridge prep C18 5 µm OBD, 30x100 mm, 5% → 90% MeCN in H₂O (both containing 1% acetic acid)). The product 171 was obtained as a clear oil (18.7 mg, 0.034 mmol, 74%). LCMS (ESI+) calculated for C₁₉H₂₅F₄N₈O₆+ (M+H+) 537.45 found 537.29.

### Example 73. Synthesis of BCN-LPETGG (172)

To a solution of 102 (10 mg, 0.031 mmol) in anhydrous DMF (500 µL) were added peptide 167(H-LPETGG-OH, 18 mg, 0.031 mmol) and Et₃N (13 µL, 0.095 mmol). After stirring at room temperature for 93 h, the crude mixture was purified by RP HPLC (Column Xbridge prep C18 5 µm OBD, 30x100 mm, 5% → 90% MeCN in H₂O (both containing 1% acetic acid)). The product 172 was obtained as a clear oil (16.8 mg, 0.022 mmol, 72%). LCMS (ESI+) calculated for C₃₅H₅₃N₆O₁₂+ (M+H+) 749.83 found 749.39.

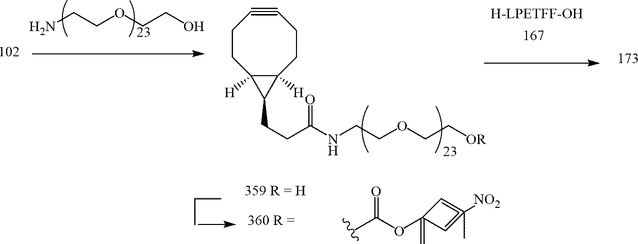

### Example 74. Synthesis of 359

To a solution of 102 (56 mg, 0.17 mmol) in DCM (8 mL) were added amino-PEG₂₄-alcohol (214 mg, 0.199 mmol) and Et₃N (80 µL, 0.53 mmol). After stirring at room temperature for 20 h, solvent was reduced in vacuo and the residue was purified by flash silica gel column chromatography (2 → 30% MeOH in DCM) which gave the desired compound 359 as a yellow oil in 95% yield (210 mg, 0.168 mmol). LCMS (ESI+) calculated for C₅₉H₁₁₁NO₂₆Na+ (M+Na+) 1273.50 found 1273.07.

### Example 75. Synthesis of 360

To a solution of 359 (170 mg, 0.136 mmol) and 4-nitrophenyl chloroformate (44 mg, 0.22 mmol) in DCM (7 mL) was added Et₃N (63 µL, 0.40 mmol). After stirring at room temperature for 41 h, solvent was reduced and the residue was purified by flash silica gel column chromatography (0 → 10% MeOH in DCM) which gave the desired compound 360 as a clear oil in 67% yield (129 mg, 0.091 mmol). LCMS (ESI+) calculated for C₆₆H₁₁₄N₂O₃₀Na+ (M+Na+) 1438.59 found 1438.13.

### Example 76. Synthesis of 173

To a solution of 360 (16 mg, 0.011 mmol) in anhydrous DMF (800 µL) were added 167 (peptide H-LPETGG-OH, 6.5 mg, 0.011 mmol) and Et₃N (5 µL, 0.04 mmol). After stirring at room temperature for 95 h, the crude mixture was purified by RP HPLC (Column Xbridge prep C18 5 µm OBD, 30x100 mm, 5% → 90% MeCN in H₂O (both containing 1% acetic acid)). The product 173 was obtained as a clear oil (12.6 mg, 0.0068 mmol, 62%). LCMS (ESI+) calculated for C₈₄H₁₅₃N₈O₃₇+ (M/2+NH₄+) 942.55 found 924.26.

### Example 77. Synthesis of 174

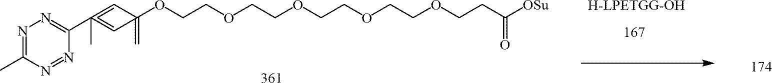

To a solution of 361 (methyltetrazine-PEGs-NHS ester, 6.1 mg, 0.011 mmol) in anhydrous DMF (230 µL) were added peptide H-LPETGG-OH (6.5 mg, 0.011 mmol) and Et₃N (4 µL, 0.028 mmol). After stirring at room temperature for 22 h, the crude mixture was purified by RP HPLC (Column Xbridge prep C18 5 µm OBD, 30x100 mm, 5% → 90% MeCN in H₂O (both containing 1% acetic acid)). The product 174 was obtained as a clear pink oil (9.9 mg, 0.01 mmol, 91%). LCMS (ESI+) calculated for C₄₄H₇₀N₁₁O₁₆+ (M+NH₄+) 1009.09 found 1009.61.

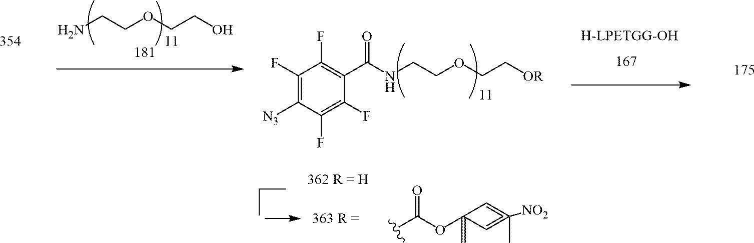

### Example 78. Synthesis of 362

To a solution of 354 (31 mg, 0.093 mmol) in DCM (1 mL) were added 181 (56 mg, 0.10 mmol) and Et₃N (40 µL, 0.28 mmol). After stirring in the dark at room temperature for 25 min, the mixture was concentrated in vacuo and purified by flash column chromatography over silicagel (0 → 15% MeOH in DCM) which gave the desired compound 362 as a clear oil (55 mg, 0.072 mmol, 77%). LCMS (ESI+) calculated for C₃₁H₅₁F₄N₄O₁₃+ (M+H+) 763.75 found 763.08.

### Example 79. Synthesis of 363

To a solution of 362 (55 mg, 0.072 mmol) in DCM (2 mL) were added 4-nitrophenyl chloroformate (13 mg, 0.064 mmol) and Et₃N (30 µL, 0.21 mmol). After stirring in the dark at room temperature for 21 h, the mixture was concentrated in vacuo and purified by RP HPLC (Column Xbridge prep C18 5 µm OBD, 30x100 mm, 5% → 90% MeCN (1% AcOH) in water (1% AcOH). The product 363 was obtained as a yellow oil (13.3 mg, 0.014 mmol, 20%). LCMS (ESI+) calculated for C₃₈H₅₄F₄N₅O₁₇+ (M+H+) 928.85 found 928.57.

### Example 80. Synthesis of 175

To a solution of 363 (13.3 mg, 0.014 mmol) in anhydrous DMF (300 µL) were added 167 (peptide H-LPETGG-OH, 8.2 mg, 0.014 mmol) and Et₃N (6 µL, 0.043 mmol). After 26 h in the dark, the crude mixture was purified by RP HPLC (Column Xbridge prep C18 5 µm OBD, 30x100 mm, 5% → 90% MeCN in H₂O (both containing 1% acetic acid)). The product 175 was obtained as a clear oil (11.4 mg, 0.0084 mmol, 59%). LCMS (ESI+) calculated for C₅₆H₈₉F₄N₁₀O₂₄+ (M+H+) 1362.35 found 1362.81.

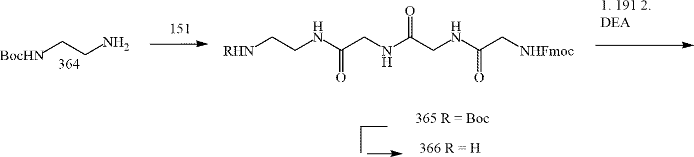

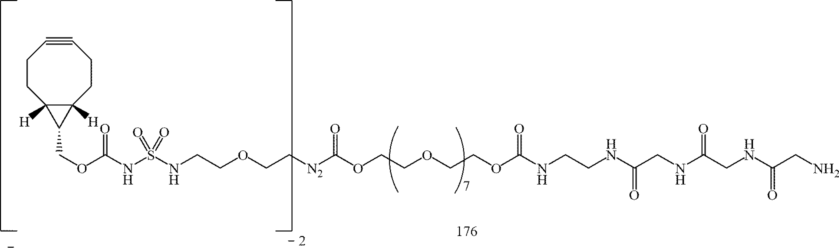

### Example 81. Synthesis of 365

To a stirred solution of 151 (Fmoc—Gly—Gly—Gly—OH, 20 mg, 0.049 mmol) in anhydrous DMF (350 µL) were added DIPEA (25 µL, 0.15 mmol) and HATU (18 mg, 0.049 mmol). After 10 min, compound 364 (/V-Boc-ethylenediamine, 7.8 mg, 0.049 mmol) dissolved in anhydrous was added. After stirring at room temperature for 45 min, the mixture was concentrated in vacuo and purified by flash column chromatography over silicagel (0 → 30% MeOH in DCM) which gave the desired compound 365 as a clear oil (12.4 mg, 0.022 mmol, 46%). LCMS (ESI+) calculated for C₂₈H₃₆N₅O₇+ (M+H+) 554.61 found 554.46.

### Example 82. Synthesis of 366

To a stirred solution of 365 (12.4 mg, 0.022 mmol) in DCM (0.7 mL) was added 4.0 M HCI in dioxane (400 µL). After stirring at room temperature for 1 h, the mixture was concentrated and 366 was obtained as a white solid (11 mg, 0.022 mmol, quant.). LCMS (ESI+) calculated for C₂₃H₂₈N₅O₇+ (M+H+) 545.50 found 454.33.

### Example 83. Synthesis of 176

To a solution of 191 (8 mg, 0.0059 mmol) in anhydrous DMF (300 µL) were added Et₃N (2.5 µL, 0.017 mmol) and stock of 366 in anhydrous DMF (110 µL, 3.0 mg, 0.0059 mmol). After stirring at room temperature for 18 h, diethylamine (2 uL) was added. After an additional of 2 h, the mixture was purified by RP HPLC (Column Xbridge prep C18 5 µm OBD, 30x100 mm, 5% → 90% MeCN in H₂O (both containing 1% acetic acid)). The product 176 was obtained as a clear oil (1.3 mg, 0.0009 mmol, 15%). LCMS (ESI+) calculated for C₆₀H₁₀₃N₁₀O₂₆S₂+ (M+H+) 1444.64 found 1444.75.

### Example 84. Anti-4-1BB PF31

Anti1BB scFv was designed with a C-terminal sortase A recognition sequence followed by a His tag (amino acid sequence being identified by SEQ ID NO: 4). Anti1BB scFv was transiently expressed in HEK293 cells followed by IMAC purification by Absolute Antibody Ltd (Oxford, United Kingdom). Mass spectral analysis showed one major product (observed mass 28013 Da, expected mass 28018 Da).

### Example 85. Cloning of SYR—(G4S)₃—IL15 (PF18) into pET32a Expression Vector

The SYR-(G4S)₃-IL15 (PF18) (amino acid sequence being identified by SEQ ID NO: 5) was designed with an N-terminal (M)SYR sequence, where the methionine will be cleaved after expression leaving an N-terminal serine, and a flexible (G4S)₃ spacer between the SYR sequence and IL15. The codon-optimized DNA sequence was inserted into a pET32A expression vector between Ndel and Xhol, thereby removing the sequence encoding the thioredoxin fusion protein, and was obtained from Genscript, Piscataway, USA.

### Example 86. E. Coli Expression of SYR-(G4S)₃-IL15 (PF18) and Inclusion Body Isolation

Expression of SYR-(G4S)₃-IL15 (PF18) starts with the transformation of the plasmid (pET32a-SYR-(G4S)3-IL15) into BL21 cells (Novagen). Transformed cells were plated on LB-agar with ampicillin and incubated overnight at 37° C. A single colony was picked and used to inoculate 50 mL of TB medium + ampicillin followed by incubated overnight at 37° C. Next, the overnight culture was used to inoculation 1000 mL TB medium + ampicillin. The culture was incubated at 37° C. at 160 RPM and, when OD600 reached 1.5, induced with 1 mM IPTG (1 mL of 1 M stock solution). After >16 hour induction at 37° C. at 160 RPM, the culture was pelleted by centrifugation (5000 xg - 5 min). The cell pellet gained from 1000 mL culture was lysed in 60 mL BugBuster™ with 1500 units of Benzonase and incubated on roller bank for 30 min at room temperature. After lysis the insoluble fraction was separated from the soluble fraction by centrifugation (15 minutes, 15000 x g). Half of the insoluble fraction was dissolved in 30 mL BugBuster™ with lysozyme (final concentration: 200 µg/mL) and incubated on the roller bank for 10 min. Next the solution was diluted with 6 volumes of 1:10 diluted BugBuster™ and centrifuged 15 min, 15000 x g . The pellet was resuspended in 200 mL of 1:10 diluted BugBuster™ by using the homogenizer and centrifuged at 10 min, 12000 x g . The last step was repeated 3 times.

### Example 87. Refolding of SYR-(G₄S)3- IL15 (PF18) from Isolated Inclusion Bodies

The purified inclusion bodies containing SYR-(G4S)3- IL15 (PF18), were dissolved and denatured in 30 mL 5 M guanidine with 40 mM Cysteamine and 20 mM Tris pH 8.0. The suspension was centrifuged at 16.000 x g for 5 min to pellet the remaining cell debris. The supernatant was diluted to 1 mg/mL with 5 M guanidine with 40 mM Cysteamine and 20 mM Tris pH 8.0, and incubated for 2 hours at RT on a rollerbank. The 1 mg/mL solution is added dropwise to 10 volumes of refolding buffer (50 mM Tris, 10.53 mM NaCl, 0.44 mM KCI, 2.2 mM MgCl₂, 2.2 mM CaCI₂, 0.055% PEG-4000, 0.55 M L-arginine, 4 mM cysteamine, 4 mM cystamine, at pH 8.0) in a cold room at 4° C., stirring required. Leave solution at least 24 hours at 4° C. Dialyze the solution to 10 mM NaCl and 20 mM Tris pH 8.0, 1x overnight and 2 x4 hours, using a Spectrum™ SpectrafPor™ 3 RC Dialysis Membrane Tubing 3500 Dalton MWCO. Refolded SYR-(G4S)3- IL15 (PF18) was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 M NaCl, pH 8.0) on a gradient of 30 mL from buffer A to buffer B. Mass spectrometry analysis showed a weight of 14122 Da (expected mass: 14122 Da) corresponding to PF18. The purified SYR-(G4S)3- IL15 (PF18) was buffer exchanged to PBS using HiPrep™ 26/10 Desalting column (Cytiva) on a AKTA Purifier-10 (GE Healthcare).

### Example 88. Cloning of SYR-(G4S)3-IL15Ra-linker-IL15 (PF26) Into pET32a Expression Vector

The SYR-(G4S)3-IL15Ra-linker-IL15 (PF26) (amino acid sequence being identified by SEQ ID NO: 6) was designed with an N-terminal (M)SYR sequence, where the methionine will be cleaved after expression leaving an N-terminal serine, and a flexible (G₄S)₃ spacer between the SYR sequence and IL15Ra-linker-IL15. The codon-optimized DNA sequence was inserted into a pET32A expression vector between Ndel and Xhol, thereby removing the sequence encoding the thioredoxin fusion protein, and was obtained from Genscript, Piscataway, USA.

### Example 89. E. Coli Expression of SYR-(G4S)3-IL15Ra-linker-IL15 (PF26) and Inclusion Body isolation

Expression of SYR-(G4S)3-IL15Ra-linker-IL15 (PF26) starts with the transformation of the plasmid (pET32a- SYR-(G4S)3-IL15Ra-linker-IL15) into BL21 cells (Novagen). Next step was the inoculation of 1000 mL culture (TB medium + ampicillin) with BL21 cells. When OD600 reached 1.5 , cultures were induced with 1 mM IPTG (1 mL of 1 M stock solution). After >16 hour induction at 37° C. at 160 RPM, the culture was pelleted by centrifugation (5000 xg - 5 min). The cell pellet gained from 1000 mL culture was lysed in 60 mL BugBuster™ with 1500 units of Benzonase and incubated on roller bank for 30 min at room temperature. After lysis the insoluble fraction was separated from the soluble fraction by centrifugation (15 minutes, 15000 x g). Half of the insoluble fraction was dissolved in 30 mL BugBuster™ with lysozyme (final concentration: 200 µg/mL) and incubated on the roller bank for 10 min. Next the solution was diluted with 6 volumes of 1:10 diluted BugBuster™ and centrifuged 15 min, 15000 x g . The pellet was resuspended in 200 mL of 1:10 diluted BugBuster™ by using the homogenizer and centrifuged at 10 min, 12000 x g . The last step was repeated 3 times.

### Example 90. Refolding of SYR-(G4S)3-IL15Ra-linker-IL15 (PF26) From Isolated Inclusion Bodies

The purified inclusion bodies containing SYR-(G4S)3-IL15Ra-linker-IL15 (PF26), were dissolved and denatured in 30 mL 5 M guanidine with 40 mM Cysteamine and 20 mM Tris pH 8.0. The suspension was centrifuged at 16.000 x g for 5 min to pellet the remaining cell debris. The supernatant was diluted to 1 mg/mL with 5 M guanidine with 40 mM Cysteamine and 20 mM Tris pH 8.0, and incubated for 2 hours at RT on a rollerbank. The 1 mg/mL solution is added dropwise to 10 volumes of refolding buffer (50 mM Tris, 10.53 mM NaCl, 0.44 mM KCI, 2.2 mM MgCl₂, 2.2 mM CaCl₂, 0.055% PEG-4000, 0.55 M L-arginine, 4 mM cysteamine, 4 mM cystamine, at pH 8.0) in a cold room at 4° C., stirring required. Leave solution at least 24 hours at 4° C. Dialyze the solution to 10 mM NaCl and 20 mM Tris pH 8.0, 1x overnight and 2 x4 hours using a Spectrum™ Spectra/Por™ 3 RC Dialysis Membrane Tubing 3500 Dalton MWCO. Refolded SYR-(G4S)3-IL15Ra-linker-IL15 (PF26) was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 M NaCl, pH 8.0) on a gradient of 30 mL from buffer A to buffer B. Mass spectrometry analysis showed a weight of 24146 Da (expected mass: 24146 Da) corresponding to PF26. The purified SYR-(G4S)3-IL15Ra-linker-IL15 (PF26) was buffer exchanged to PBS using HiPrep™ 26/10 Desalting column from cytiva on a AKTA Purifier-10 (GE Healthcare).

### Example 91. Humanized OKT3 200

Humanized OKT3 (hOKT3) with C-terminal sortase A recognition sequence (C-terminal tag being identified by SEQ ID NO: 1) was obtained from Absolute Antibody Ltd (Oxford, United Kingdom). Mass spectral analysis showed one major product (observed mass 28836 Da).

### Example 92. C-terminal Sortagging of Compound GGG-PEG₂-BCN (157) to hOKT3 200 Using Sortase A to Obtain hOKT3-PEG₂-BCN 201

A bioconjugate according to the invention was prepared by C-terminal sortagging using sortase A (identified by SEQ ID NO: 2). To a solution of hOKT3 200 (500 µL, 500 µg, 35 µM in PBS pH 7.4) was added sortase A (58 µL, 384 µg, 302 µM in TBS pH 7.5 + 10% glycerol), GGG-PEG₂-BCN (157, 28 µL, 50 mM in DMSO), CaCl₂ (69 µL, 100 mM in MQ) and TBS pH 7.5 (39 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27829 Da), corresponding to 201. The sample was dialyzed against PBS pH 7.4 and concentrated by spinfiltration (Amicon Ultra-0.5, Ultracel-10 Membrane, Millipore) to obtain hOKT3-PEG2-BCN 201 (60 µL, 169 µg, 101 µM in PBS pH 7.4).

### Example 93. C-terminal Sortagging of Compound GGG-PEG₂-BCN (157) to hOKT3 200 Using sortase A Pentamutant to Obtain hOKT3-PEG₂-BCN 201

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₂-BCN (157, 2 µL, 20 mM in DMSO:MQ=2:3), CaCl₂ (2 µL, 100 mM in MQ) and TBS pH 7.5 (1.2 µL). The reaction was incubated at 37° C. overnight. Mass spectral analysis showed one major product (observed mass 27829 Da), corresponding to hOKT3-PEG₂-BCN 201.

### Example 94. C-terminal Sortagging of Compound GGG-PEG₁₁-BCN (161) to hOKT3 200 using sortase A to obtain hOKT3-PEG₁₁-BCN 202

A bioconjugate according to the invention was prepared by C-terminal sortagging using sortase A (identified by SEQ ID NO: 2). To a solution of hOKT3 200 (14.3 µL, 14 µg, 35 µM in PBS pH 7.4) was added sortase A (0.9 µL, 12 µg, 582 µM in TBS pH 7.5 + 10% glycerol), GGG-PEG₁₁-BCN (161, 2 µL, 20 mM in MQ), CaCl₂ (2 µL, 100 mM in MQ) and TBS pH 7.5 (0.9 µL). The reaction was incubated at 37° C. overnight. Mass spectral analysis showed one major product (observed mass 21951 Da, approximately 85%), corresponding to sortase A, a minor product (observed masses 28227 Da, approximately 5%), corresponding to hOKT3-PEG₁₁-BCN 202, and two other minor products (observed masses 28051 Da and 28325 Da, each approximately 5%).

### Example 95. C-terminal Sortagging of Compound GGG-PEG₁₁-BCN (161) to hOKT3 200 Using Sortase a Pentamutant to Obtain hOKT3-PEG₁₁-BCN 202

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₁₁-BCN (161.2 µL, 20 mM in MQ), CaCl₂ (2 µL, 100 mM in MQ) and TBS pH 7.5 (1.2 µL). The reaction was incubated at 37° C. overnight. Mass spectral analysis showed one major product (observed mass 28225 Da, approximately 60%), corresponding to hOKT3-PEG₁₁-BCN 202, and one minor product (observed mass 28326 Da, approximately 40%).

### Example 96. C-terminal Sortagging of Compound GGG-PEG₂₃-BCN (163) to hOKT3 200 Using Sortase A to Obtain hOKT3-PEG₂₃-BCN 203

A bioconjugate according to the invention was prepared by C-terminal sortagging using sortase A (identified by SEQ ID NO: 2). To a solution of hOKT3 200 (14.3 µL, 14 µg, 35 µM in PBS pH 7.4) was added sortase A (0.9 µL, 12 µg, 582 µM in TBS pH 7.5 + 10% glycerol), GGG-PEG₂₃-BCN (163, 2 µL, 20 mM in MQ), CaCl₂ (2 µL, 100 mM in MQ) and TBS pH 7.5 (0.9 µL). The reaction was incubated at 37° C. overnight. Mass spectral analysis showed one major product (observed mass 21951 Da, approximately 70%), corresponding to sortase A, and one minor product (observed mass 28755 Da, approximately 30%), corresponding to hOKT3-PEG₂₃-BCN 203.

### Example 97. C-terminal Sortagging of Compound GGG-PEG₂₃-BCN (163) to hOKT3 200 Using Sortase A Pentamutant to Obtain hOKT3-PEG₂₃-BCN 203

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₂₃-BCN (163, 2 µL, 20 mM in MQ), CaCl₂ (2 µL, 100 mM in MQ) and TBS pH 7.5 (1.2 µL). The reaction was incubated at 37° C. overnight. Mass spectral analysis showed one major product (observed mass 28754 Da), corresponding to hOKT3-PEG₂₃-BCN 203.

### Example 98. C-terminal Sortagging of Compound GGG-PEG₄-tetrazine (154) to hOKT3 200 Using Sortase A to Obtain hOKT3-PEG₄-tetrazine 204

A bioconjugate according to the invention was prepared by C-terminal sortagging using sortase A (identified by SEQ ID NO: 2). To a solution of hOKT3 200 (500 µL, 500 µg, 35 µM in PBS pH 7.4) was added sortase A (58 µL, 384 µg, 302 µM in TBS pH 7.5 + 10% glycerol), GGG-PEG₄-tetrazine (154, 35 µL, 40 mM in MQ), CaCl₂ (69 µL, 100 mM in MQ) and TBS pH 7.5 (32 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27868 Da), corresponding to 104. The sample was dialyzed against PBS pH 7.4 and concentrated by spinfiltration (Amicon Ultra-0.5, Ultracel-10 Membrane, Millipore) to obtain hOKT3-PEG₄-tetrazine 204 (70 µL, 277 µg, 143 µM in PBS pH 7.4).

### Example 99. C-terminal Sortagging of Compound GGG-PEG₄-tetrazine (154) to hOKT3 200 Using Sortase A Pentamutant to Obtain hOKT3-PEG₄-tetrazine 204

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₄-tetrazine (154, 2 µL, 20 mM in MQ), CaCl₂ (2 µL, 100 mM in MQ) and TBS pH 7.5 (1.2 µL). The reaction was incubated at 37° C. overnight. Mass spectral analysis showed one major product (observed mass 27868 Da), corresponding to hOKT3-PEG₄-tetrazine 204.

### Example 100. C-terminal Sortagging of GGG-PEG₁₁-tetrazine (169) to hOKT3 200 with Sortase A to Obtain hOKT3-PEG₁₁-tetrazine PF01

A bioconjugate according to the invention was prepared by C-terminal sortagging with sortase A (identified by SEQ ID NO: 2). To a solution of hOKT3 200 (1908 µL, 5 mg, 91 µM in PBS pH 7.4) was added sortase A (81 µL, 948 µg, 533 µM in TBS pH 7.5 + 10% glycerol), GGG-PEG₁₁-tetrazine (169, 347 µL, 20 mM in MQ), CaCl₂ (347 µL, 100 mM in MQ) and TBS pH 7.5 (789 µL). The reaction was incubated at 37° C. overnight. Mass spectral analysis showed one major product (observed mass 28258 Da), corresponding to hOKT3-PEG₁₁-tetrazine PF01. The reaction was purified on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and buffer exchanged to PBS pH 6.5 using a HiPrep 26/10 desalting column (GE Healthcare). Addition dialysis was performed to PBS pH 6.5 for 3 days at 4° C. to remove residual 169.

### Example 101. C-terminal Sortagging of GGG-PEG₂₃-tetrazine (170) to hOKT3 200 with Sortase A to Obtain hOKT3-PEG₂₃-tetrazine PF02

A bioconjugate according to the invention was prepared by C-terminal sortagging with sortase A (identified by SEQ ID NO: 2). To a solution of hOKT3 200 (1908 µL, 5 mg, 91 µM in PBS pH 7.4) was added sortase A (81 µL, 948 µg, 533 µM in TBS pH 7.5 + 10% glycerol), GGG-PEG₂₃-tetrazine (170, 347 µL, 20 mM in MQ), CaCl₂ (347 µL, 100 mM in MQ) and TBS pH 7.5 (789 µL). The reaction was incubated at 37° C. overnight. Mass spectral analysis showed one major product (observed mass 28787 Da), corresponding to hOKT3-PEG₂₃-tetrazine PF02. The reaction was purified on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was dialyzed to PBS pH 6.5 followed by purification on a Superdex75 10/300 GL column (GE Healthcare) on an AKTA Purifier-10 (GE Healthcare) using PBS pH 6.5 as mobile phase.

### Example 102. C-terminal Sortagging of GGG-PEG₂-arylazide (171) to hOKT3 200 with Sortase A to Obtain hOKT3-PEG₂-arylazide PF03

A bioconjugate according to the invention was prepared by C-terminal sortagging with sortase A (identified by SEQ ID NO: 2). To a solution of hOKT3 200 (2092 µL, 5 mg, 83 µM in PBS pH 7.4) was added sortase A (95 µL, 950 µg, 456 µM in TBS pH 7.5 + 10% glycerol), GGG-PEG₂-arylazide (171, 347 µL, 20 mM in MQ), CaCl₂ (347 µL, 100 mM in MQ) and TBS pH 7.5 (591 µL). The reaction was incubated at 37° C. overnight. Mass spectral analysis showed one major product (observed mass 27865 Da), corresponding to hOKT3-PEG₂-arylazide PF03. The reaction was purified on a His-trap excel 1 mL column (GE Healthcare) on an AKTA Purifier-10 (GE Healthcare). The column was equilibrated with buffer A (20 mM Tris, 200 mM NaCl, 20 mM Imidazole, pH 7.5) and the sample was loaded with 1 mL/min. The flowthrough purified on a Superdex75 10/300 GL column (GE Healthcare) on an AKTA Purifier-10 (GE Healthcare) using PBS pH 7.4 as mobile phase.

### Example 103. C-terminal Sortagging of Compound GGG-PEG₂₃-BCN (163) anti-4-1BB PF31 With Sortase A to Obtain Anti-4-1BB PF07

A bioconjugate according to the invention was prepared by C-terminal sortagging with sortase A (identified by SEQ ID NO: 2). To a solution of anti-4-1BB-PF31 (665 µL, 2 mg, 107 µM in PBS pH 7.4) was added sortase A (100 µL, 1 mg, 357 µM in TBS pH 7.5 + 10% glycerol), GGG-PEG₂₃-BCN (163, 140 µL, 20 mM in MQ), CaCl₂ (140 µL, 100 mM in MQ) and TBS pH 7.5 (355 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and after concentration purified on a Superdex75 10/300 column (Cytiva). Mass spectral analysis showed one major product (observed mass 28478 Da) corresponding to anti-4-1BB-BCN PF07.

### Example 104. C-Terminal Sortagging of GGG-PEG₁₁-tetrazine (169) in Anti-4-1BB PF31 with Sortase A to Obtain Anti-4-1BB-PEG₁₁-tetrazine PF08

To a solution containing protein PF31 (1151 µL, 93 µM in TBS pH 7.5) was added TBS pH 7.5 (512 µL), CaCl₂ (214 µL, 100 mM) and GGG-PEG₁₁-tetrazine (169, 220 µL, 20 mM in MQ) and Sortase A (50 µL, 533 µM in TBS pH 7.5). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (Observed mass 27989 Da) corresponding to 4-1BB-tetrazine PF08.

### Example 105. C-terminal Sortagging of Compound GGG-PEG₂-arylazide (171) Anti-4-1BB-PF31 with Sortase A to Obtain Anti-4-1BB PF09

A bioconjugate according to the invention was prepared by C-terminal sortagging with sortase A (identified by SEQ ID NO: 2). To a solution of anti-4-1BB-PF31 (665 µL, 2 mg, 107 µM in PBS pH 7.4) was added sortase A (100 µL, 1 mg, 357 µM in TBS pH 7.5 + 10% glycerol), GGG-PEG₂-arylazide (171, 140 µL, 20 mM in MQ), CaCl₂ (140 µL, 100 mM in MQ) and TBS pH 7.5 (355 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27592 Da) corresponding to anti-4-1BB-azide PF09.

### Example 106. N-Terminal Sortagging of BCN-LPETGG (172) in GGG-IL15Rα-IL15 (208) with Sortase A to Obtain BCN-IL15Rα-IL15 (PF10)

To a solution containing protein 208 (465 µL, 133 µM in TBS pH 7.5) was added TBS pH 7.5 (1400 µL), CaCl₂ (124 µL, 100 mM), 172 (371 µL, 5 mM in DMSO) and Sortase A (115 µL, 537 µM in TBS pH 7.5) and incubated 3 hours at 37° C. After incubation, Sortase A was removed from the solution using Ni-NTA beads (300 µL Beads= 600 µL). The solution was incubated 1 hour with Ni-NTA beads on a roller bank, whereafter the solution was centrifuged (5 min, 7.000 xg). The supernatant, which contained the product PF10, was collected by separation of the supernatant from the pellet. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 23582 Da (expected mass: 23579 Da) corresponding to PF10.

### Example 107. N-Terminal Sortagging of BCN-PEG₂₄-LPETGG (173) in GGG-IL15Rα-IL15 (208) with sortase A to obtain BCN-PEG₂₄-IL15Rα-IL15 (PF11)

To a solution containing protein 208 (465 µL, 133 µM in TBS pH 7.5) was added TBS pH 7.5 (1400 µL), CaCl₂ (124 µL, 100 mM) and 173 (371 µL, 5 mM in DMSO) and Sortase A (115 µL, 537 µM in TBS pH 7.5) and incubated 3 hours at 37° C. After incubation, Sortase A was removed from the solution using Ni-NTA beads (300 µL Beads= 600 µL). The solution was incubated 1 hour with Ni-NTA beads on a roller bank, whereafter the solution was centrifuged (5 min, 7.000 xg). The supernatant, which contained the product PF11, was collected by separation of the supernatant from the pellet. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 24682 Da (expected mass: 24680 Da) corresponding to PF11.

### Example 108. N-Terminal Sortaggingof Tetrazine-PEG₃-LPETGG (174) in GGG-IL15Rα-IL15 (208) with Sortase A to Obtain Tetrazine-PEG₃-IL15Rα-IL15 (PF12)

To a solution containing protein 208 (465 µL, 133 µM in TBS pH 7.5) was added TBS pH 7.5 (1400 µL), CaCl₂ (124 µL, 100 mM) and 174 (371 µL, 5 mM in DMSO) and Sortase A (115 µL, 537 µM in TBS pH 7.5) and incubated 3 hours at 37° C. After incubation, Sortase A was removed from the solution using Ni-NTA beads (300 µL Beads= 600 µL slurry). The solution was incubated 1 hour with Ni-NTA beads on a roller bank, whereafter the solution was centrifuged (5 min, 7.000 xg). The supernatant, which contained the product PF12, was collected by separation of the supernatant from the pellet. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 23824 Da (expected mass: 23822 Da) corresponding to PF12.

Example 109. N-Terminal sortagging of Arylazide-PEG₁₁-LPETGG (175) in GGG-IL15Rα-IL15 (208) with sortase A to obtain Arylazide-PEG₁₁-GGG-IL15Rα-IL15 (PF13)

To a solution containing protein 208 (2000 µL, 140 µM in TBS pH 7.5) was added TBS pH 7.5 (2686 µL), CaCl₂ (559 µL, 100 mM) and 175 (83 µL, 50 mM in DMSO) and Sortase A (260 µL, 537 µM in TBS pH 7.5) and incubated 3 hours at 37° C. (shielded from light). After incubation, Sortase A was removed from the solution using Ni-NTA beads (500 µL Beads=1mL slurry). The solution was incubated ON at 4° C. with Ni-NTA beads on a roller bank, whereafter the solution was centrifuged (5 min, 7.000 xg). The supernatant, which contained the product PF13, was collected by separation of the supernatant from the pellet. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 24193 Da (expected mass: 24193 Da) corresponding to PF13.

Example 110. N-Terminal oxime ligation of BCN-PEG₁₂-aminooxy (XL13) to SYR-(G₄S)₃-IL15Rα-IL15 (PF26) to obtain BCN-PEG₁₂-SYR-(G₄S)3-IL15Rα-IL15 (PF14)

Prior to labeling of PF26, the N-terminal serine was oxidated using Sodium periodate. To a solution containing protein PF26 (700 µL, 70 µM in PBS pH 7.4) was added PBS pH 7.4 (286 µL), NalO₄ (0.98 µL, 100 mM in MQ) and L-methionine (5 µL, 100 mM in MQ) and incubated 5 minutes at 4° C. Mass spectrometry analysis showed a weight of 24114 & 24130 Da corresponding to the expected masses of 24114 (aldehyde) and 24132 Da (hydrate). Using a PD-10 desalting column the excess NalO₄ and L-methionine were removed. The oxidated PF26 was concentrated to a concentration of 50 µM using Amicon spin filter 0.5, MWCO 10 kDa (Merck-Millipore). To a solution containing oxidized PF26 (416 µL, 50 µM in PBS pH 7.4) was added, XL13 (41.6 µL, 50 mM in DMSO). After ON incubation at 37° C. the reaction mixture was purified using PD-10 desalting columns packed with Sephadex G-25 resin (Cytiva) and eluted using PBS. Mass spectrometry analysis showed a weight of 25024 Da (expected mass: 25042 Da) corresponding to PF14.

### Example 111. N-terminal BCN Functionalization of IL15Rα-IL15 PF26 by SPANC to Obtain BCN-IL15Rα-IL15 PF15

To IL15Ra-IL15 PF26 (2.9 mg, 50 µM in PBS) was added 2 eq NalO₄ (4.8 µL of 50 mM stock in PBS) and 10 eq L-Methionine (12.5 µL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the elute (2.6 mg, 50 µM in PBS) was added 160 eq N-methylhydroxylamine.HCI (340 µL of 50 mM stock in PBS) and 160 eq p-Anisidine (340 µL of 50 mM stock in PBS). The reaction mixture was incubated for 3 hours at 25° C. Mass spectral analysis showed one single peak (observed mass 24143 Da) corresponding to N-methyl-imine-oxide-IL15. The reaction mixture was purified using PD-10 desalting columns packed with Sephadex G-25 resin (Cytiva) and eluted using PBS. To the elute (2.47 mg, 50 µM in PBS) was added 25 eq Bis-BCN-PEG₁₁ (105) (51 µL, 50 mM in DMSO) and 150 µL DMF. The reaction was incubated overnight at room temperature. The reaction was purified using a Superdex75 10/300 column (Cytiva). Mass spectral analysis showed one major peak corresponding to BCN-IL15Rα-IL15 PF15 (observed mass 25041 Da).

### Example 112. N-Terminalincorporation of Maleimide-PEG2-BCN (XL05) in SYR-(G₄S)₃-IL15Rα-IL15 (PF26) Using Strain-Promoted Alkyne-Nitrone Cycloaddition to Obtain Maleimide- PEG2-SYR-(G₄S)₃-IL15Ra-IL15 (PF16)

To IL15Ra-IL15 PF26 (2560 µL, 50 µM in PBS) was added 2 eq NalO₄ (5.12 µL of 50 mM stock in PBS) and 10 eq L-Methionine (12.8 µL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the concentrated elute (2450 µL, 50 µM in PBS) was added 160 eq N-methylhydroxylamine.HCI (196 µL of 100 mM stock in PBS) and 160 eq p-Anisidine (196 µL of 100 mM stock in PBS). The reaction mixture was incubated for 3 hours at 25° C. Mass spectral analysis showed one single peak (observed mass 24143 Da) corresponding to N-methyl-imine-oxide-IL15Rα-IL15. The reaction mixture was purified using PD-10 desalting columns packed with Sephadex G-25 resin (Cytiva) and eluted using PBS. To the concentrated elute (1134 µL, 50 µM in PBS) was added 25 eq maleimide-PEG₂-BCN (XL05) (7.1 µL, 200 mM in DMF) and 106 µL DMF. The reaction was incubated o/n at RT. The reaction mixture was purified using PD-10 desalting columns packed with Sephadex G-25 resin (Cytiva) and eluted using PBS. Mass spectral analysis showed the desired maleimide-BCN-SYR-(G₄S)₃-IL15Ra-IL15 (PF16) (observed mass 24618 Da, expected mass: 24617 Da).

### Example 113. Diazotransfer to SYR-(G₄S)₃-IL15Rα-IL15 PF26 to Obtain Azido-IL15Rα-IL15 PF17

To a solution of SYR-(G₄S)₃-IL15Rα-IL15 PF26 (3289 µL, 5 mg, 63 µM in 0.1 M triethanolamine pH 8.0) was added triethanolamine pH 8.0 (461 µL, 0.1 M in MQ) and Imidazole-1-sulfonyl azide hydrochloride (commercially available from Fluorochem Ltd, 417 µL, 50 mM solution dissolved in 50 mM NaOH in MQ, 100 equiv.). The reaction was incubated overnight at 37° C. followed by purification on a Superdex75 10/300 GL column (GE Healthcare) on an AKTA Purifier-10 (GE Healthcare) using PBS pH 7.4 as mobile phase. Mass spectral analysis showed one major product (observed mass 24171 Da), corresponding to azido-IL15Rα-IL15 PF17, and a minor byproduct (observed mass 24412 Da).

### Example 114. N-terminal Diazotransfer Reaction of IL15 PF18 to Obtain azido-IL 15 PF19

To IL15 PF18 (5 mg, 50 µM in 0.1 M TEA buffer pH 8.0) imidazole-1-sulfonylazide hydrochloride (708 µL, 50 mM in 50 mM NaOH) was added and incubated overnight at 37° C. The reaction was purified using a HiPrep™ 26/10 Desalting column (Cytiva). Mass spectral analysis showed one main peak (observed mass 14147 Da) corresponding to azido-IL15 PF19.

### Example 115. N-Terminal Incorporation of tetrazine-PEG₁₂-2PCA (XL10) in SYR-(G₄S)₃-IL15 (PF18) using 2PCA to Obtain Tetrazine-PEG₁₂-SYR-(G₄S)₃-IL15 (PF21)

To SYR-(G₄S)₃-IL15 (PF18) (1052 µL, 50 µM in PBS) was added 20 eq. Tetrazine-PEG12-2PCA (XL10) (112 µL of 50 mM stock in DMSO) and 4359 µL PBS. The reaction was incubated overnight at 37° C. Using spinfiltration (Amicon Ultra-0.5, Ultracel-10 Membrane, Millipore) the sample was concentrated <1 mL and loaded on to a Superdex 75 10/300 GL column (GE Healthcare) on an AKTA Purifier-10 (GE Healthcare) using PBS pH 7.4 as mobile phase and a flow of 0.5 mL/min. Mass spectral analysis showed a weight of 24121 Da corresponding to the start material SYR-(G₄S)₃-IL15 (PF18) (Expected mass: 14121 Da) and the a mass of 15093 Da corresponding to the product PF21 (Expected mass: 15094 Da).

### Example 116. Conjugation of tri-BCN (150) to hOKT3-PEG₂-arylazide PF03 to Obtain bis-BCN-hOKT3 PF22

To a solution of hOKT3-PEG₂-arylazide PF03 (87 µL, 1 mg, 411 µM in PBS pH 7.4) was added PBS pH 7.4 (559 µL), DMF (49 µL) and compound 150 (22 µL, 40 mM solution in DMF, 25 equiv.). The reaction was incubated overnight at RT. Mass spectral analysis showed one major product (observed mass 29171 Da), corresponding to bis-BCN-hOKT3 PF22. The reaction was purified on a Superdex75 10/300 GL column (GE Healthcare) on an AKTA Purifier-10 (GE Healthcare) using PBS pH 7.4 as mobile phase.

### Example 117. C-terminal Sortagging of GGG-bis-BCN 176 to hOKT3 200 With Sortase A to Obtain bis-BCN-hOKT3 PF23

A bioconjugate according to the invention was prepared by C-terminal sortagging with sortase A (identified by SEQ ID NO: 2). To a solution of hOKT3 200 (272 µL, 0.7 mg, 83 µM in PBS pH 7.4) was added sortase A (25 µL, 250 µg, 456 µM in TBS pH 7.5 + 10% glycerol), GGG-bis-BCN (176, 45 µL, 20 mM in DMSO), CaCl₂ (45 µL, 100 mM in MQ) and TBS pH 7.5 (64 µL). The reaction was incubated at 37° C. overnight. Mass spectral analysis showed one major product (observed mass 28772 Da), corresponding to bis-BCN-hOKT3 PF23. The reaction was purified on a Superdex75 10/300 GL column (GE Healthcare) on an AKTA Purifier-10 (GE Healthcare) using PBS pH 7.4 as mobile phase.

### Example 118. N-Terminal Incorporation of Tri-BCN (150) in SYR-(G₄S)₃-IL15Rα-IL15 (PF26) Using Strain-Promoted Alkyne-Nitrone Cycloaddition to Obtain Bis-BCN- SYR-(G₄S)₃-IL15Rα-IL15 (PF27)

To IL15Rα-IL15 PF26 (3840 µL, 50 µM in PBS) was added 2 eq NalO₄ (7.7 µL of 50 mM stock in PBS) and 10 eq L-Methionine (19.2 µL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the concentrated elute (1800 µL, 50 µM in PBS) was added 160 eq N-methylhydroxylamine.HCl (320 µL of 90 mM stock in PBS) and 160 eq p-Anisidine (288 µL of 100 mM stock in PBS). The reaction mixture was incubated for 3 hours at 25° C. Mass spectral analysis showed one single peak (observed mass 24143 Da) corresponding to N-methyl-imine-oxide-IL15Rα-IL15. The reaction mixture was purified using PD-10 desalting columns packed with Sephadex G-25 resin (Cytiva) and eluted using PBS. To the concentrated elute (3100 µL, 60 µM in PBS) was added 25 eq tri-BCN (150) (116 µL, 40 mM in DMSO), 256 µL DMF and PBS pH 7.4 (248 µL). The reaction was incubated o/n at RT. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectral analysis showed the desired Bis-BCN-IL15Rα-IL15 PF27 (observed mass 25448 Da, expected mass 25447). RP-HPLC showed a labeling efficiency of 60%.

### Example 119. N-Terminal Incorporation of Bis-Maleimide-PEG₆-BCN (XL01) in SYR-(G₄S)₃-IL15Rα-IL15 (PF26) Using Strain-Promoted Alkyne-Nitrone Cycloaddition To Obtain Bis-Maleimide-PEG₆-SYR-(G₄S)₃-IL15Rα-IL15 (PF28)

To SYR-(G₄S)₃-IL15Rα-IL15 PF26 (2560 µL, 50 µM in PBS) was added 2 eq NalO₄ (5.12 µL of 50 mM stock in PBS) and 10 eq L-methionine (12.8 µL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the concentrated elute (2450 µL, 50 µM in PBS) was added 160 eq N-methylhydroxylamine.HCI (196 µL of 100 mM stock in PBS) and 160 eq p-anisidine (196 µL of 100 mM stock in PBS). The reaction mixture was incubated for 3 hours at 25° C. Mass spectral analysis showed one single peak (observed mass 24143 Da) corresponding to N-methyl-imine-oxide-IL15Rα-IL15. The reaction mixture was purified using PD-10 desalting columns packed with Sephadex G-25 resin (Cytiva) and eluted using PBS. To the concentrated elute (1134 µL, 50 µM in PBS) was added 25 eq bis-Maleimide-PEG₆-BCN (XL01) (28.5 µL, 50 mM in DMSO) and 86.5 µL DMF. The reaction was incubated o/n at RT. The reaction mixture was purified using PD-10 desalting columns packed with Sephadex G-25 resin (Cytiva) and eluted using PBS. Additional washing was performed using spinfiltration (Amicon Ultra-0.5, Ultracel-10 Membrane, Millipore), 6x with 400 µL PBS, to remove remaining Bis-Maleimide-PEG₂-BCN (XL01). Mass spectral analysis showed the desired Bis-maleimide-BCN-SYR-(G₄S)₃-IL15Rα-IL15 (PF28) (observed mass 25145 Da, Expected mass 25144 Da).

### Example 120. N-Terminal Incorporation of Tri-BCN (150) in N₃-SYR-(G₄S)₃-IL15 (PF19) Using Strain-Promoted Alkyne-Azide Cycloaddition to Obtain Bis-BCN-SYR-(G₄S)₃-IL15 (PF29)

To N₃-IL15 PF19 (706 µL, 50 µM in PBS) was added 4 eq tri-BCN (150) (3.5 µL of 40 mM stock in DMF) and 67 µL DMF. The reaction was incubated o/n at RT. Mass spectral analysis confirmed the formation of bis-BCN-SYR-(G₄S)₃-IL15 PF29 (observed mass 15453 Da, expected mass 15453 Da). The reaction mixture was purified using PD-10 desalting columns packed with Sephadex G-25 resin (Cytiva) and eluted using PBS. Additional washing was performed using spin-filtration (Amicon Ultra-0.5, Ultracel-10 Membrane, Millipore), 6x with 400 µL PBS, to remove remaining tri-BCN (150).

### Example 121. Enzymatic Deglycosylation of Trastuzumab With PNGase F

Trastuzumab (Herzuma) (20 mg, 12.5 mg/mL in PBS pH 7.4) was incubated with PNGase F (16 µL, 8000 units) at 37° C. Mass spectral analysis of a sample after IdeS treatment showed one major Fc/2 product (observed mass 23787 Da) corresponding to the expected product.

### Example 122. Enzymatic Deglycosylation of Rituximab With PNGase F

Rituximab (6 mg, 10 mg/mL in PBS pH 7.4) was incubated with PNGase F (6 µL, 3000 units) at 37° C. Mass spectral analysis of a sample after IdeS treatment showed one major Fc/2 product (observed mass 23754 Da) corresponding to the expected product.

### Example 123. Enzymatic Remodeling of Trastuzumab to Trastuzumab-(GalNAz)₂ (trast-v1b)

Trastuzumab (5 mg, 22.7 mg/mL) was incubated with EndoSH, described in PCT/EP2017/052792 (1% w/w), for 1 hour at room temperature followed by the addition of β(1,4)-Gal-T1(Y289L), (2% w/w) and UDP-GaINAz, (15 eq compared to IgG) in 10 mM MnCI2 and TBS for 16 hours at 30° C. After addition of the components the final concentration of trastuzumab is 19.6 mg/ml. The functionalized IgG was purified using a protA column (5 mL, MabSelect Sure, Cytiva). After loading of the reaction mixture, the column was washed with TBS. The IgG was eluted with 0.1 M NaOAc pH 3.5 and neutralized with 2.5 M Tris-HCI pH 7.2. After three times dialysis to PBS the functionalized trastuzumab was concentrated to 17.2 mg/mL using a Vivaspin Turbo 4 ultrafiltration unit (Sartorius). Mass spectral analysis of a sample after IdeS treatment showed one major Fc/2 product (observed mass 24380 Da) corresponding to the expected product trast-v1b.

### Example 124. Enzymatic Remodeling of Trastuzumab to trastuzumab-(GalNAz)₂ (trast-v2)

Trastuzumab (5 mg, 22.7 mg/mL) was incubated with β(1,4)-Gal-T1(Y289L), (2% w/w) and UDP-GalNAz, (20 eq compared to IgG) in 10 mM MnCl₂ and TBS for 16 hours at 30° C. After addition of the components the final concentration of trastuzumab is 19 mg/ml. The functionalized IgG was three times dialysed to PBS and concentrated to 19.45 mg/mL using a Vivaspin Turbo 4 ultrafiltration unit (Sartorius). Mass spectral analysis of a sample after IdeS treatment showed two major Fc/2 products (observed mass 25718 Da, approximately 50% of total Fc/2) corresponding to G0F with 2 × GalNAz and a minor product (observed mass 25636 Da, approximately 50% of total Fc/2) for G1F with 1 × GalNAz.

### Example 125. MTGase-catalyzed Incorpation of azido-PEG₃-amine Onto Deglycosylated Trastuzumab to Give Bis-Azido-Trastuzumab Trast-v3

To a solution of deglycosylated trastuzumab (806 µL, 10 mg, 12.4 mg/mL in PBS pH 7.4) was added PBS pH 7.4 (3544 µL), azido-PEG₃-amine (commercially available from BroadPharm, 500 µL, 10 mM solution in MQ, 75 equiv. compared to IgG) and recombinant microbial transglutaminase (commercially available from Zedira, 150 µL, 15 U, 0.1 U/µL). The reaction was incubated overnight at 37° C. Mass spectral analysis of an IdeS-digested sample showed one major product (observed mass 23988 Da), corresponding to bis-azido-trastuzumab trast-v3. The reaction was purified using a protA column (5 mL, MabSelect Sure, GE Healthcare) on an AKTA Explorer-100 (GE Healthcare) followed by dialysis to PBS pH 7.4.

### Example 126. MTGase-Catalyzed Incorpation of azido-Peg₃-Amine Onto Deglycosylated Rituximab to Give Bis-Azido-Rituximab rit-v3

To a solution of deglycosylated rituximab (90 µL, 1.8 mg, 20.2 mg/mL in PBS pH 7.4) was added PBS pH 7.4 (693 µL), azido-PEG₃-amine (commercially available from BroadPharm, 90 µL, 10 mM solution in MQ, 75 equiv. compared to IgG) and recombinant microbial transglutaminase (commercially available from Zedira, 27 µL, 2.7 U, 0.1 U/µL). The reaction was incubated overnight at 37° C. Mass spectral analysis of an IdeS-digested sample showed one major product (observed mass 23956 Da), corresponding to bis-azido-rituximab rit-v3. The reaction was buffer exchange to PBS pH 7.4 using centrifugal filters (Amicon Ultra-0.5 mL MWCO 10 kDa, Merck Millipore).

### Example 127. Enzymatic Remodeling of Trastuzumab to trastuzumab-(GalNProSSMe)₂ (trast-v5a)

Trastuzumab (5 mg, 22.7 mg/mL) was incubated with EndoSH, described in PCT/EP2017/052792 (1% w/w), for 1 hour followed by the addition TnGalNAcT (expressed in CHO), (10% w/w) and UDP-GalProSSMe, (318, 40 eq compared to IgG) in 10 mM MnCl₂ and TBS for 16 hours at 30° C. After addition of the components the final concentration of trastuzumab is 12.5 mg/ml. The functionalized IgG was purified using a protA column (5 mL, MabSelect Sure, Cytiva). After loading of the reaction mixture the column was washed with TBS. The IgG was eluted with 0.1 M NaOAc pH 3.5 and neutralized with 2.5 M Tris-HCI pH 7.2. After three times dialysis to PBS the functionalized trastuzumab was concentrated to 17.4 mg/mL using a Vivaspin Turbo 4 ultrafiltration unit (Sartorius). Mass spectral analysis of a sample after IdeS treatment showed one major Fc/2 product (observed mass 24430 Da) corresponding to the expected product (trast-v5a).

### Example 128. Enzymatic Remodeling of Trastuzumab to trastuzumab-(GalNAc-Lev)₂ (trast-v8)

Trastuzumab (5 mg, 22.7 mg/mL) was incubated with EndoSH, described in PCT/EP2017/052792 (1% w/w), for 1 hour followed by the addition of β(1,4)-Gal-T1(Y289L), (10% w/w) and UDP-GaINAc-Lev (11 g, x = 1) prepared according example 9-17 in WO2014/065661A1), (75 eq compared to IgG) in 10 mM MnCl₂ and TBS for 16 hours at 30° C. After addition of the components the final concentration of trastuzumab is 14.4 mg/ml. The functionalized IgG was purified using a protA column (5 mL, MabSelect Sure, Cytiva). After loading of the reaction mixture, the column was washed with TBS. The IgG was eluted with 0.1 M NaOAc pH 3.5 and neutralized with 2.5 M Tris-HCI pH 7.2. After three times dialysis to PBS the functionalized trastuzumab was concentrated to 10.6 mg/mL using a Vivaspin Turbo 4 ultrafiltration unit (Sartorius). Mass spectral analysis of a sample after IdeS treatment showed one major Fc/2 product (observed mass 24393 Da) corresponding to the expected product (trast-v8).

### Example 129. Enzymatic Remodeling of Trastuzumab to trastuzumab-(GalNAc-alkyne)₂ (trast-v9)

Trastuzumab (5 mg, 22.7 mg/mL) was incubated with EndoSH, described in PCT/EP2017/052792 (1% w/w), for 1 hour followed by the addition of β(1,4)-Gal-T1(Y289L), (2% w/w) and UDP-GaINAc-Alkyne, (11f, x = 1) prepared according example 9-16 in WO2014/065661A1), (15 eq compared to IgG) in 10 mM MnCl₂ and TBS for 16 hours at 30° C. After addition of the components the final concentration of trastuzumab is 19.6 mg/ml. The functionalized IgG was purified using a protA column (5 mL, MabSelect Sure, Cytiva). After loading of the reaction mixture the column was washed with TBS. The IgG was eluted with 0.1 M NaOAc pH 3.5 and neutralized with 2.5 M Tris-HCI pH 7.2. After three times dialysis to PBS the functionalized trastuzumab was concentrated to 12.1 mg/mL using a Vivaspin Turbo 4 ultrafiltration unit (Sartorius). Mass spectral analysis of a sample after IdeS treatment showed one major Fc/2 product (observed mass 24379 Da) corresponding to the expected product trast-v9.

### Example 130. Conjugation of Trastuzumab(6-N₃-GalNAc)₂205 with 201 to Obtain Conjugate 206

A bioconjugate according to the invention was prepared by conjugation of BCN-modified hOKT3 201 to azide-modified trastuzumab 205. To a solution of trastuzumab-(6-N₃-GalNAc)2 prepared according to WO2016170186 (205, 2 µL, 75 µg, 250 µM in PBS pH 7.4) was added hOKT3-PEG₂-BCN 201 (9.9 µL, 28 µg, 101 µM in PBS pH 7.4). The reaction was incubated at rt overnight. Mass spectral analysis of the Fabricator™-digested sample showed two major products (observed masses 24368 Da and 52196 Da, each approximately 50%), corresponding to the azido-modified Fc/2-fragment and conjugate 206, respectively.

### Example 131. Cloning of His₆-SSGENLYFQ-GGG-IL15Rα-IL15 into pET32a Expression Vector

The IL15Rα-IL15 fusion protein 207 was designed with an N-terminal His-tag (HHHHHH), TEV protease recognition sequence (SSGENLYFQ) and an N-terminal sortase A recognition sequence (GGG). A pET32A-vector containing a DNA sequence encoding His₆-SSGENLYFQ-GGG-IL15RaIL15 (SEQ ID NO: 3) between base pairs 158 and 692, thereby removing the thioredoxin coding sequence, was obtained from Genscript.

### Example 132. E. Coli Expression of His₆-SSGENLYFQ-GGG-IL15Rα-IL15 (207) and Inclusion Body Isolation

Expression of His₆-SSGENLYFQ-GGG-IL15Rα-IL15 207 starts with the transformation of the plasmid (pET32a-IL15Rα-IL15) into BL21 cells (Novagen). Next step was the inoculation of 500 mL culture (LB medium + ampicillin) with BL21 cells. When OD600 reached 0.7, cultures were induced with 1 mM IPTG (500 µL of 1 M stock solution). After 4 hour induction at 37° C., the culture was pelleted by centrifugation. The cell pellet gained from 500 mL culture was lysed in 25 mL BugBuster™ with 625 units of benzonase and incubated on roller bank for 20 min at room temperature. After lysis the insoluble fraction was separated from the soluble fraction by centrifugation (20 minutes, 12000 x g, 4° C.). The insoluble fraction was dissolved in 25 mL BugBuster™ with lysozyme (final concentration: 200 µg/mL) and incubated on the roller bank for 5 min. Next the solution was diluted with 6 volumes of 1:10 diluted BugBuster™ and centrifuged 15 min, 9000 × g at 4° C. The pellet was resuspended in 250 mL of 1:10 diluted BugBuster™ by using the homogenizer and centrifuged at 15 min, 9000 x g at 4° C. The last step was repeated 3 times.

### Example 133. Refolding of His₆-SSGENLYFQ-GGG-IL15Rα-IL15 207 from Isolated Inclusion Bodies

The purified inclusion bodies containing His₆-SSGENLYFQ-GGG-IL15Rα-IL15 207, were sulfonated o/n at 4° C. in 25 mL denaturing buffer (5 M guanidine, 0.3 M sodium sulphite) and 2.5 mL 50 mM disodium 2-nitro-5-sulfobenzonate. The solution was diluted with 10 volumes of cold Milli-Q and centrifuged (10 min at 8000 x g). The pellet was solved in 125 mL cold Milli-Q using a homogenizer and centrifuged (10 min at 8000 x g). The last step was repeated 3 times. The purified His₆-SSGENLYFQ-GGG-IL15Rα-IL15 207 was denatured in 5 M guanidine and diluted to a concentration of 1 mg/mL of protein. Using a syringe with a diameter of 0.8 mm, the denatured protein was added dropwise to 10 volumes refolding buffer (50 mM Tris, 10.53 mM NaCl, 0.44 mM KCI, 2.2 mM MgCl₂, 2.2 mM CaCl₂, 0.055% PEG-4000, 0.55 M L-arginine, 8 mM cysteamine, 4 mM cystamine, at pH 8.0) on ice and was incubate 48 hours at 4° C. (stirring not required). The refolded His₆-SSGENLYFQ-GGG-IL15Rα-IL15 207 was loaded on a 20 mL HisTrap excel column (GE health care) on an AKTA Purifier-10 (GE Healthcare). The column was first washed with buffer A (5 mM Tris buffer, 20 mM imidazole, 500 mM NaCl, pH 7.5). Retained protein was eluted with buffer B (20 mM Tris buffer, 500 mM imidazole, 500 mM NaCl, pH 7.5) on a gradient of 25 mL from buffer A to buffer B. Fractions were analysed by SDS-PAGE on polyacrylamide gels (16%). The fractions that contained purified target protein were combined and the buffer was exchanged against TBS (20 mM Tris pH 7.5 and 150 mM NaCl₂) by dialysis performed overnight at 4° C. The purified protein was concentrated to at least 2 mg/mL using Amicon Ultra-0.5, MWCO 3 kDa (Merck-Millipore). Mass spectral analysis showed a weight of 25044 Da (expected: 25044 Da). The product was stored at -80° C. prior to further use.

### Example 134. TEV Cleavage of His₆-SSGENLYFQ-GGG-IL15Rα-IL15 207 to Obtain GGG-IL15Rα-IL15 208

To a solution of His₆-SSGENLYFQ-GGG-IL15Rα-IL15 (207, 330 µL, 2.3 mg/mL in TBS pH 7.5), was added TEV protease (50.5 µL, 10 Units/µL in 50 mM Tris-HCI, 250 mM NaCl, 1 mM TCEP, 1 mM EDTA, 50% glycerol, pH 7.5, New England Biolabs). The reaction was incubated for 1 hour at 30° C. After TEV cleavage, the solution was purified using size exclusion chromatography. The reaction mixture was loaded on to a Superdex 75 10/300 GL column (GE Healthcare) on an AKTA Purifier-10 (GE Healthcare) using TBS pH 7.5 as mobile phase and a flow of 0.5 mL/min. GGG-IL15Rα-IL15 208 was eluted at a retention time of 12 mL. The purified protein was concentrated to at least 2 mg/mL using an Amicon Ultra-0.5, MWCO 3 kDa (Merck Millipore). The product was analysed with mass spectrometry (observed mass: 22965 Da, expected mass: 22964 Da), corresponding to GGG-IL15Rα-IL15 208. The product was stored at -80° C. prior to further use.

### Example 135. Incorporation of BCN-PEG₁₂-LPETGG (168) in GGG-IL15Rα-IL15 208 Using Sortase A to Obtain BCN-PEG₁₂-IL15Rα-IL15 (209)

To a solution of GGG-IL15Rα-IL15 (208, 219 µL, 91.4 µM in TBS pH 7.5) was added TBS pH 7.5 (321 µL), CaCl₂ (40.0 µL, 100 mM) and BCN-PEG₁₂-LPETGG (168, 120 µL, 5 mM in DMSO) and incubated 1 hour at 37° C. After incorporation of 168 was complete, sortase A was removed from the solution using the same volume of Ni-NTA beads as reaction volume (800 µL). The solution was incubated for 1 hour in a spinning wheel/or table shaker, afterwards the solution was centrifuged (2 min, 13000 rpm) and the supernatant was discarded. BCN-PEG₁₂-IL15Rα-IL15 (209) was collected from the beads by incubating the beads 5 min with 800 µL washing buffer (40 mM imidazole, 20 mM Tris, 0.5 M NaCI) in a table shaker at 800 rpm. The beads were centrifuged (2 min, 13000 x rpm), the supernatant containing 209 was separated and the buffer was exchanged to TBS by dialysis o/n at 4° C. Finally, the solution was concentrated to 0.5-1 mg/mL using Amicon spin filter 0.5, MWCO 3 kDa (Merck-Millipore). Mass spectrometry analysis showed a weight of 24155 Da (expected mass: 24152) corresponding to BCN-PEG₁₂-IL15Rα-IL15 (209).

### Example 136. Conjugation of BCN-PEG₁₂-IL15Ra-IL15 (209) to Trastuzumab(6-N₃-GaINAc)₂205 to Obtain Conjugate 210

A bioconjugate according to the invention was prepared by conjugation of 209 to azide-modified trastuzumab (205, trastuzumab(6-N₃-GaINAc)₂, prepared according to WO2016170186) in a 2:1 molar ratio. Thus, to a solution of BCN-PEG₁₂-IL15Rα-IL15 (209, 20 µL, 20 µM in TBS pH 7.4) wasadded trastuzumab(6-N₃-GaINAc)₂ (205, 1.2 µL, 82 µM in PBS pH 7.4) and incubated o/n at 37° C. Mass spectral analysis of the IdeS-digested sample showed a mass of 48526 Da (expected mass: 48518 Da) corresponding to the Fc/2-fragment of conjugate 210.

### Example 137. Intramolecular Cross-linking of Trastuzumab-(azide)₂ with Bivalent Linker 105 to Give 211

To a solution of trastuzumab-(6-azidoGaINAc)₂ (7.5 µL, 150 µg, 17.56 mg/mL in PBS pH 7.4; also referred to as trast-v1a), prepared according to WO2016170186, was added compound 105 (2.5 µL, 0.8 mM solution in DMF, 2 equiv. compared to IgG). The reaction was incubated for 1 day at RT followed by buffer exchange to PBS pH 7.4 using centrifugal filters (Amicon Ultra-0.5 mL MWCO 10 kDa, Merck-Millipore). Mass spectral analysis of the IdeS digested sample showed one major product (calculated mass 49625 Da, observed mass 49626 Da), corresponding to intramolecularly cross-linked trastuzumab derivative 211. HPLC-SEC showed <4% aggregation, hence excluding intermolecular cross-linking.

### Example 138. Intramolecular Cross-linking of Trastuzumab-(azide)₂ with Bivalent Linker 107 to Give 212

To a solution of trastuzumab-(6-azido-GaINAc)₂ (7.5 µL, 150 µg, 17.56 mg/mL in PBS pH 7.4) was added compound 107 (2.5 µL, 4 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck-Millipore). Mass spectral analysis of the IdeS digested sample showed the product (calculated mass 50153 Da, observed mass 50158 Da), corresponding to intramolecularly cross-linked trastuzumab derivative 212. HPLC-SEC showed <4% aggregation, hence excluding intermolecular cross-linking.

### Example 139. Intramolecular Cross-Linking of Trastuzumab-(azide)₂ with Bivalent Linker 117 to Give 213

To a solution of trastuzumab-(6-azidoGaINAc)₂ (7.5 µL, 150 µg, 17.56 mg/mL in PBS pH 7.4) was added compound 117 (2.5 µL, 0.8 mM solution in DMF, 2 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49580 Da, observed mass 49626 Da), corresponding to intramolecularly cross-linked trastuzumab derivative 213. HPLC-SEC showed <4% aggregation, hence excluding intermolecular cross-linking.

### Example 140. Intramolecular Cross-Linking of Trastuzumab-(azide)₂ with Bivalent Linker 118 to Give 214

To a solution of trastuzumab-(6-azidoGaINAc)₂ (7.5 µL, 150 µg, 17.56 mg/mL in PBS pH 7.4) was added compound 118 (2.5 µL, 4 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed the product (calculated mass 49358 Da, observed mass 49361 Da), corresponding to intramolecularly cross-linked trastuzumab derivative 214. HPLC-SEC showed <4% aggregation, hence excluding intermolecular cross-linking.

### Example 141. Intramolecular Cross-Linking of Trastuzumab-(azide)₂ with Bivalent Linker 124 to Give 215

To a solution of trastuzumab-(6-azidoGaINAc)₂ (7.5 µL, 150 µg, 17.56 mg/mL in PBS pH 7.4) was added compound 124 (2.5 µL, 4 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed the product (calculated mass 49406 Da, observed mass 49409 Da), corresponding to intramolecularly cross-linked trastuzumab derivative 215. HPLC-SEC showed <4% aggregation, hence excluding intermolecular cross-linking.

### Example 142. Intramolecular Cross-linking of Trastuzumab-(azide)₂ with Bivalent Linker 125 to Give 216

To a solution of trastuzumab-(6-azidoGaINAc)₂ (7.5 µL, 150 µg, 17.56 mg/mL in PBS pH 7.4) was added compound 125 (2.5 µL, 0.8 mM solution in DMF, 2 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49184 Da, observed mass 49184 Da), corresponding to intramolecularly cross-linked trastuzumab derivative 216. HPLC-SEC showed <4% aggregation, hence excluding intermolecular cross-linking.

### Example 143. Intramolecular Cross-Linking of Trastuzumab-(azide)₂ with Bivalent Linker 145 to Give 217

To a solution of trastuzumab-(6-azidoGaINAc)₂ (320 µL, 2 mg, 5.56 mg/mL in PBS pH 7.4) was added compound 145 (80 µL, 1.66 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49796 Da, observed mass 49807 Da), corresponding to intramolecularly cross-linked trastuzumab derivative 217. HPLC-SEC showed <4% aggregation, hence excluding intermolecular cross-linking.

### Example 144. Intramolecular Cross-linking of Trastuzumab Derivative 217 (Containing Single BCN) with Tetrazine-Modified Anti-CD3 Immune Cell Engager 204 to Give T Cell Engager 221 with 2:1 Molecular Format

To a solution of 217 (8 µL, 141 µg, 17.7 mg/mL in PBS pH 7.4) was added hOKT3-PEG₄-tetrazine (204, 13.15 µL, 280 µg, 21.45 mg/mL in PBS pH 7.4, 2 equiv. compared to IgG). Mass spectral analysis of the IdeS-digested sample showed one major product (calculated mass 77664 Da, observed mass 77647 Da), corresponding to the conjugated Fc-PEG₄-hOKT3 (221).

### Example 145. Intramolecular Cross-linking of Bis-azido-rituximab Rit-v1a With Trivalent Linker 145 to give BCN-rituximab rit-v1a-145

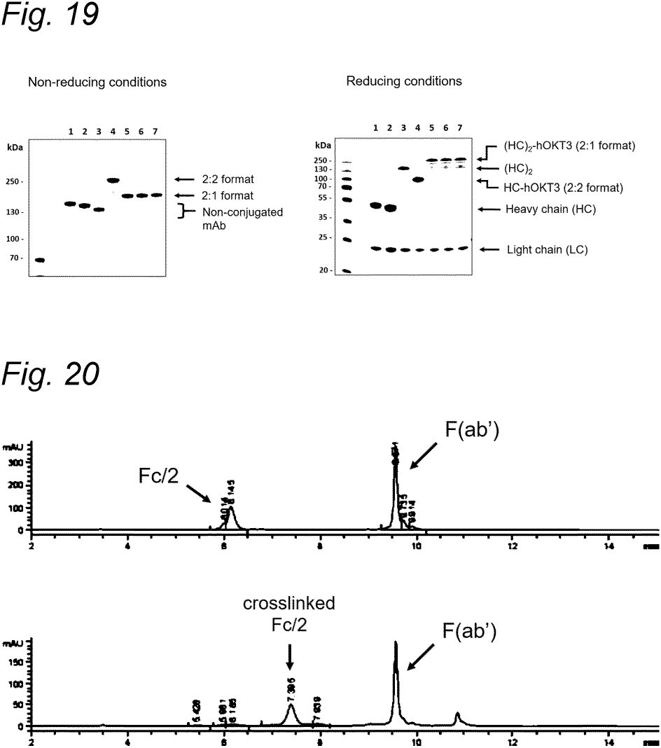

To a solution of bis-azido-rituximab rit-v1a (494 µL, 30 mg, 60.7 mg/mL in PBS pH 7.4), prepared according to WO2016170186, was added PBS pH 7.4 (2506 µL), propylene glycol (2980 µL) and trivalent linker 145 (20 µL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Reducing SDS-PAGE showed one major HC product, corresponding to the crosslinked heavy chain (See FIG. 19, right panel, lane 3), indicating formation of rit-v1a-145. Furthermore, non-reducing SDS-PAGE showed one major band around the same height as rit-v1a (See FIG. 19, left panel, lane 3), demonstrating that only intramolecular cross-linking occurred.

### Example 146. Intramolecular Cross-linking of bis-azido-B12 B12-v1a With Trivalent Linker 145 to Give BCN-B12 B12-v1a-145

To a solution of bis-azido-B12 B12-v1a (415 µL, 4 mg, 9.6 mg/mL in PBS pH 7.4), prepared according to WO2016170186, was added propylene glycol (412 µL) and trivalent linker 145 (2.7 µL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. RP-HPLC analysis of an IdeS-digested sample shows formation of B12-v1a-145. (See FIG. 20).

### Example 147. Intramolecular Crosslinking Trastuzumab-GaINProSSMe Trast-v5a With bis-maleimide-BCN XL01

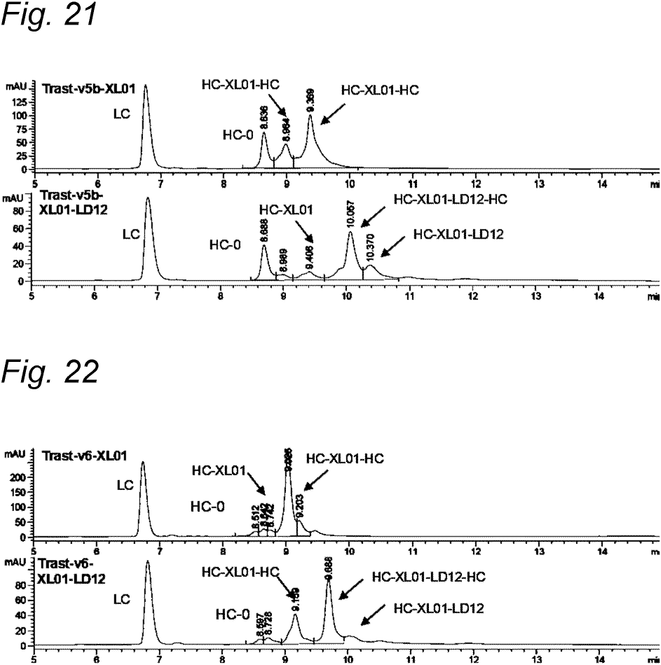

Trastuzumab-GaINProSSMe (trast-v5a) (1.2 mg, 10 mg/mL in PBS + 10 mM EDTA, trast-v5a) was incubated with TCEP (7.8 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 100 µL. Subsequent DHA (6.5 µL, 10 mM in MQ:DMSO (9:1)) was added and the reaction was incubated for 3 hours at room temperature. To a part of the reaction mixture (82 µL, 0.8 mg) was added bis-maleimide-BCN XL01 (8 µL, 2 mM in DMF) followed by incubation for 1 hour at room temperature. The conjugate was spinfiltered to PBS using centrifugal filters (Amicon Ultra-0.5 mL MWCO 10 kDa, Merck Millipore). RP-HPLC analysis of DTT treated sample showed the conversion into the conjugate trast-v5b-XL01 (see FIG. 21).

### Example 148. Intramolecular Crosslinking Trastuzumab-S239C Mutant Trast-v6 With bis-maleimide-BCN XL01

Trastuzumab S239C mutant (transient expressed in CHO by Evitria, heavy chain mutation S239C) (2 mg, 10 mg/mL in PBS + 10 mM EDTA, trast-v6) was incubated with TCEP (13 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 200 µL. Subsequent DHA (13 µL, 10 mM in MQ:DMSO (9:1)) was added and the reaction was incubated for 3 hours at room temperature. To a part of the reaction mixture (176 µL, 1.5 mg) was added bis-maleimide-BCN XL01 (15 µL, 2 mM in DMF) followed by incubation for 1 hour at room temperature. The conjugate was spin-filtered to PBS using centrifugal filters (Amicon Ultra-0.5 mL MWCO 10 kDa, Merck Millipore). RP-HPLC analysis of DTT treated sample showed 71% conversion into the conjugate trast-v6-XL01 (see FIG. 22).

### Example 149. Intramolecular Crosslinking Trastuzumab Trast-v7 With bis-maleimide-BCN XL01

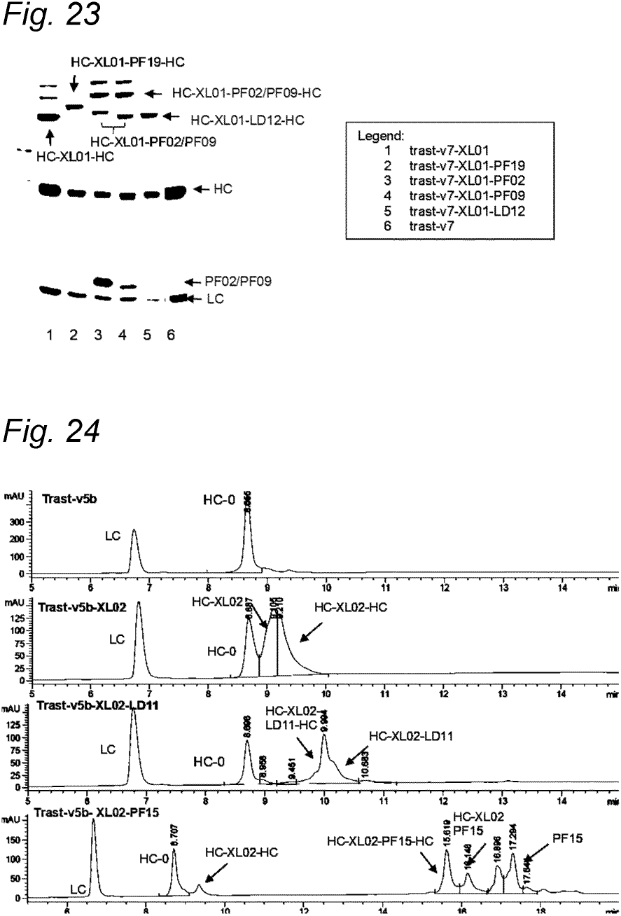

Trastuzumab (1 mg, 10 mg/mL in PBS + 10 mM EDTA, trast-v7) was incubated with TCEP (6.5 µL, 10 mM in MQ) for 2 hours at 37° C. To the reaction mixture was added bis-maleimide-BCN XL01 (10 µL, 2 mM in DMF) followed by incubation for 2 hours at room temperature. The conjugate was spinfiltered to PBS using centrifugal filters (Amicon Ultra-0.5 mL MWCO 10 kDa, Merck Millipore). SDS-page gel analysis under reducing conditions showed the formation of the conjugate trast-v7-XL01 (see FIG. 23).

### Example 150. Intramolecular Crosslinking Trastuzumab GaINProSSMe Trast-v5a With Bis-maleimide-azide XL02

Trastuzumab GaINProSSMe (1.5 mg, 10 mg/mL in PBS + 10 mM EDTA, trast-v5a) was incubated with TCEP (9.3 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 150 µL. Subsequent DHA (9.3 µL, 10 mM in DMSO) was added and the reaction was incubated for 3 hours at room temperature. To a portion of the reaction (100 µL, 1 mg antibody) was added bis-maleimide azide XL02 (10 µL, 4 mM in DMF) followed by incubation for 1 hour at room temperature. The conjugate was spinfiltered to PBS using centrifugal filters (Amicon Ultra-0.5 mL MWCO 10 kDa, Merck Millipore) and subsequent analyzed on RP-HPLC and SDS-page gel (see FIG. 24)and 24. RP-HPLC analysis of DTT treated conjugate, showed the conversion into the conjugate trast-v5b-XL02

### Example 151. Intramolecular Crosslinking Trastuzumab S239C Mutant Trast-v6 With Bis-maleimide-azide XL02

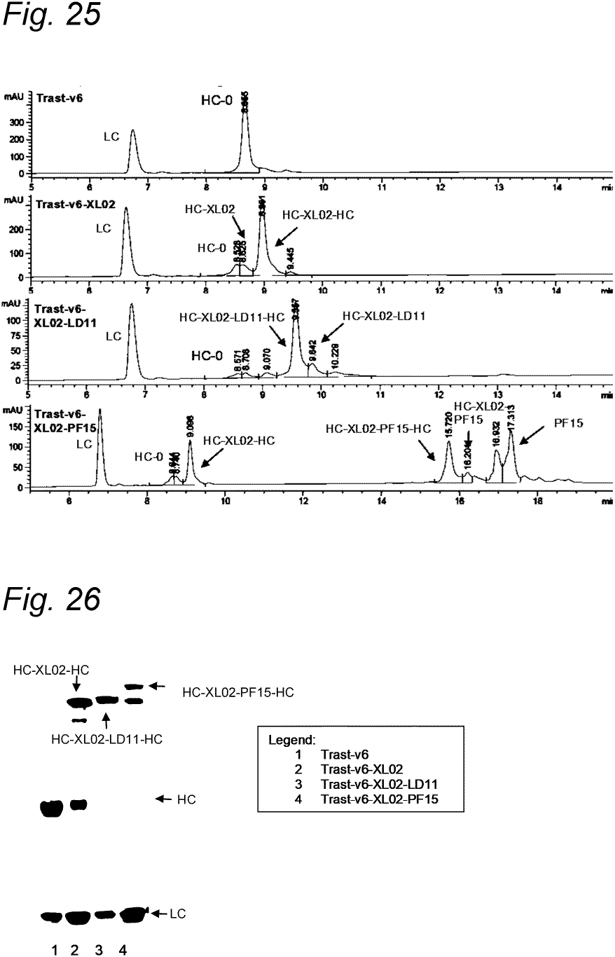

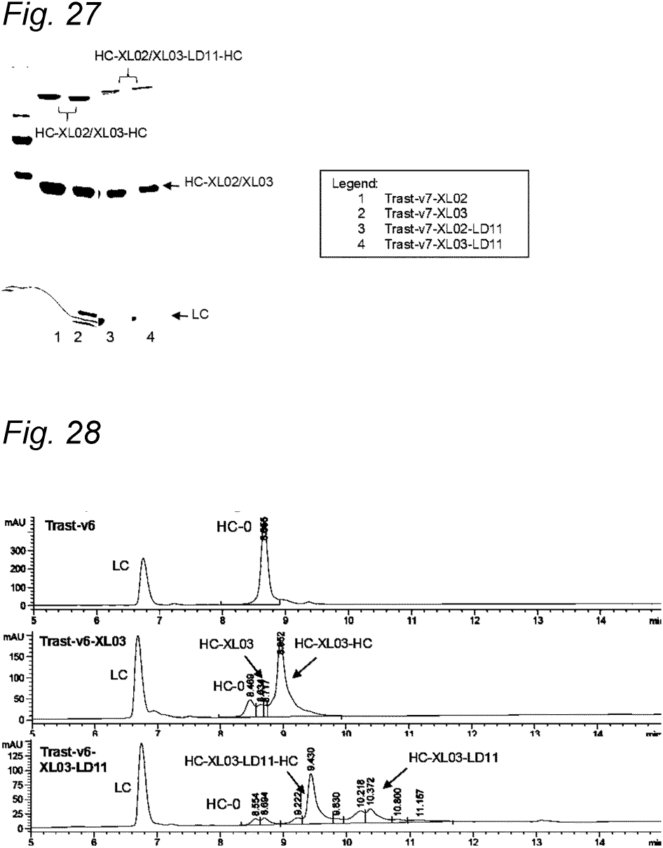

Trastuzumab S239C mutant (transient expressed in CHO by Evitria, heavy chain mutation S239C) (2 mg, 10 mg/mL in PBS + 10 mM EDTA, trast-v6) was incubated with TCEP (13 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and subsequent diluted to 200 µL. Subsequent DHA (13 µL, 10 mM in DMSO) was added and the reaction was incubated for 3 hours at room temperature. To a portion of the reaction (62 µL, 660 µg antibody) was added bis-maleimide azide XL02 (6.6 µL, 4 mM in DMF) followed by incubation for 1 hour at room temperature. The conjugate was spinfiltered to PBS using centrifugal filters (Amicon Ultra-0.5 mL MWCO 10 kDa, Merck Millipore) and subsequent analyzed on RP-HPLC and SDS-page gel (see FIGS. 25 and 26). RP-HPLC analysis of DTT treated conjugate, showed 74% conversion into the conjugate trast- v6-XL02.

### Example 152. Intramolecular Crosslinking Trastuzumab S239C Mutant Trast-v6 With C-Lock-Azide XL03

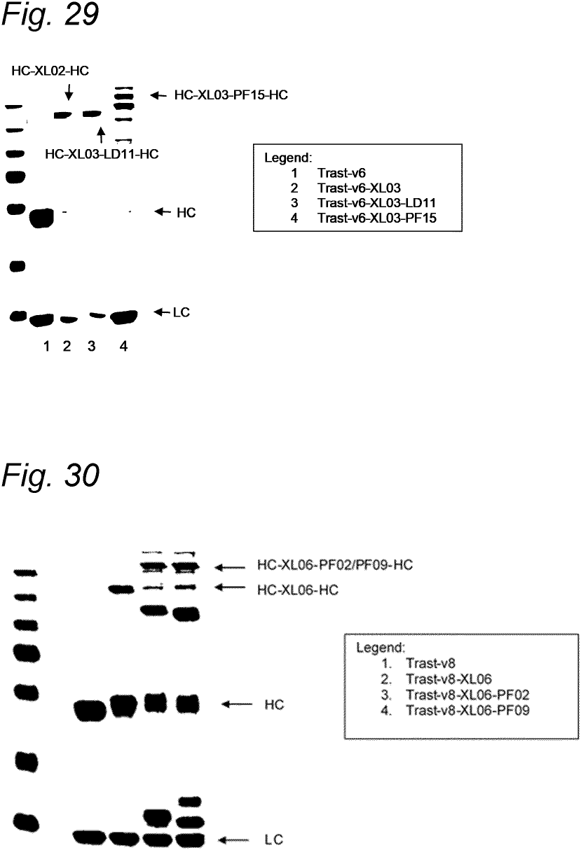

Trastuzumab S239C mutant (transient expressed in CHO by Evitria, heavy chain mutation S239C) (2 mg, 10 mg/mL in PBS + 10 mM EDTA, trast-v6) was incubated with TCEP (13 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and subsequent diluted to 200 µL. Subsequent DHA (13 µL, 10 mM in DMSO) was added and the reaction was incubated for 3 hours at room temperature. To a portion of the reaction (62 µL, 660 µg antibody) was added C— lock azide XL03 (6.6 µL, 2.7 mM in DMF) followed by incubation overnight at 37° C. The conjugate was spinfiltered to PBS using centrifugal filters (Amicon Ultra-0.5 mL MWCO 10 kDa, Merck Millipore) and subsequent analyzed on RP-HPLC and SDS-page gel (see FIGS. 28 and 29). RP-HPLC analysis of DTT treated conjugate, showed 78% conversion into the conjugate trast-v6-XL03.

### Example 153. Intramolecular Crosslinking Trastuzumab S239C Mutant Trast-v6 With maleimide-BCN (XL05)₂

Trastuzumab S239C mutant (transient expressed in CHO by Evitria, heavy chain mutation S239C) (1 mg, 10 mg/mL in PBS + 10 mM EDTA, trast-v6) was incubated with TCEP (6.5 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 100 µL. Subsequent DHA (6.5 µL, 10 mM in MQ:DMSO (9:1)) was added and the reaction was incubated for 3 hours at room temperature. Next maleimide-BCN XL05 (10 µL, 2.7 mM in DMF) was added followed by incubation for 1 hour at room temperature. The conjugate was spinfiltered to PBS using centrifugal filters (Amicon Ultra-0.5 mL MWCO 10 kDa, Merck Millipore). Mass spectral analysis of a sample after IdeS/EndoSH treatment showed one major Fc/2 product (observed mass 24627 Da) corresponding to the expected product trast-v6-(XL05)₂.

### Example 154. Intramolecular Crosslinking trastuzumab-GaINProSSMe Trast-v5a With maleimide-BCN XL05

Trastuzumab-GaINProSSMe (trast-v5a) (1 mg, 10 mg/mL in PBS + 10 mM EDTA, trast-v5a) was incubated with TCEP (6.5 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 100 µL. Subsequent DHA (6.5 µL, 10 mM in MQ:DMSO (9:1)) was added and the reaction was incubated for 3 hours at room temperature. Next maleimide-BCN XL05 (10 µL, 2.7 mM in DMF) was added followed by incubation for 1 hour at room temperature. The conjugate was spinfiltered to PBS using centrifugal filters (Amicon Ultra-0.5 mL MWCO 10 kDa, Merck Millipore). Mass spectral analysis of a sample after IdeS treatment showed one major Fc/2 product (observed mass 24861 Da) corresponding to the expected product trast-v5b-(XL05)₂.

### Example 155. Conjugation of Bis-Hydroxylamine-BCN XL06 to Trast-v8 via Oxime Ligation

Trast-v8 was spin-filtered to 0.1 M Sodium Citrate pH 4.5 using a Vivaspin Turbo 4 ultrafiltration unit (Sartorius) and concentrated to 16.45 mg/mL. Trast-v8 (1 mg, 8.1 mg/mL in 0.1 M Sodium Citrate pH 4.5) was incubated with bis-hydroxylamine-BCN XL06 (50 µL, 200 eq in DMF) and p-anisidine (26.7 µL, 200 eq in 0.1 M Sodium Citrate pH 4.5) overnight at room temperature. SDS-page gel analysis showed the formation of trast-v8-XL06 (see FIG. 30). The reaction was spin-filtered to PBS and concentrated using a Vivaspin Turbo 4 ultrafiltration unit (Sartorius) to 16.85 mg/mL.

### Example 156. Intramolecular Cross-linking of Bis-azido-trastuzumab Trast-v1a With bis-BCN-TCO XL11 to give TCO-trastuzumab trast-v1a-XL11

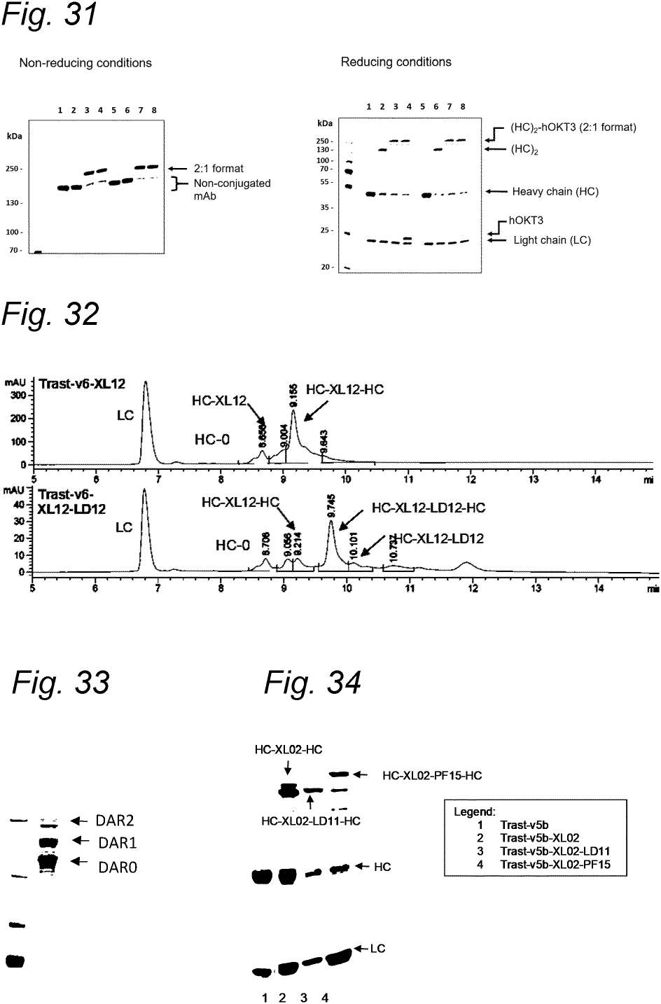

To a solution of bis-azido-trastuzumab trast-v1a (36 µL, 2 mg, 56.1 mg/mL in PBS pH 7.4) was added PBS pH 7.4 (164 µL), propylene glycol (195 µL) and bis-BCN-TCO XL11 (5.3 µL, 10 mM solution in DMF, 4.0 equiv. compared to IgG). The reaction was incubated overnight at rt followed by buffer exchange to PBS pH 7.4 using centrifugal filters (Amicon Ultra-0.5 mL MWCO 10 kDa, Merck Millipore). Reducing SDS-PAGE showed two major HC products, corresponding to the nonconjugated heavy chain and the crosslinked heavy chain (See FIG. 31, right panel, lane 2), indicating partial conversion into trast-v1a-XL11. Furthermore, non-reducing SDS-PAGE showed one major band at the height of trast-v1a (See FIG. 31, left panel, lane 2), indicating that only intramolecular crosslinking occurred.

### Example 157. Intramolecular Cross-linking of Bis-azido-rituximab Rit-v1a With bis-BCN-TCO XL11 to give TCO-rituximab rit-v1a-XL11

To a solution of bis-azido-rituximab rit-v1a (37 µL, 2 mg, 54.5 mg/mL in PBS pH 7.4) was added PBS pH 7.4 (163 µL), propylene glycol (195 µL) and bis-BCN-TCO XL11 (5.3 µL, 10 mM solution in DMF, 4.0 equiv. compared to IgG). The reaction was incubated overnight at rt followed by buffer exchange to PBS pH 7.4 using centrifugal filters (Amicon Ultra-0.5 mL MWCO 10 kDa, Merck Millipore). Reducing SDS-PAGE showed two major HC products, corresponding to the nonconjugated heavy chain and the crosslinked heavy chain (See FIG. 31, right panel, lane 6), indicating partial conversion into rit-v1a-XL11. Furthermore, non-reducing SDS-PAGE showed one major band at the height of rit-v1a (See FIG. 31, left panel, lane 2), indicating that only intramolecular crosslinking occurred.

### Example 158. Intramolecular Crosslinking trastuzumab-S239C Mutant Trast-v6 With bis-bromoacetamide-BCN XL12

Trastuzumab S239C mutant (transient expressed in CHO by Evitria, heavy chain mutation S239C) (1 mg, 10 mg/mL in PBS + 10 mM EDTA, trast-v6) was incubated with TCEP (6.5 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 200 µL. Subsequent DHA (6.5 µL, 10 mM in MQ:DMSO (9:1)) was added and the reaction was incubated for 3 hours at room temperature. To the reaction mixture were added bis-bromoacetamide-BCN XL12 (5.3 µL, 10 mM in DMF), borate buffer (4 µL, 1 M, pH 8.5), PBS (100 µL) and DMF (15 µL) followed by incubation for 3 hours at 37° C. The conjugate was spinfiltered to PBS using centrifugal filters (Amicon Ultra-0.5 mL MWCO 10 kDa, Merck Millipore). RP-HPLC analysis of DTT treated sample showed the conversion into the conjugate trast-v6-XL12 (see FIG. 32).

### Example 159. Conjugation of Trast-v1b With anti-4-1BB-BCN PF07 to Give Conjugate trast-v1b-(PF07)₂ (P:A ratio 2:1)

To a solution of trast-v1b (4.36 µL, 75 µg, 17.2 mg/mL in PBS pH 7.4) was added anti-4-1BB-BCN (PF07, 12.9 µL, 4.4 mg/mL in PBS pH 7.4, 4 eq compared to IgG). The reaction was incubated for 16 hours at room temperature. Mass spectral analysis of the IdeS digested sample showed one major product (mass 52861 Da), corresponding to conjugate trast-v1b-(PF07)₂.

### Example 160. Conjugation of Trast-v1b With BCN-IL15Rα-IL15 PF15 to Give Conjugate trast-v1b-(PF15)₂ (P:A ratio 2:1)

To a solution of trast-v1b (4.36 µL, 75 µg, 17.2 mg/mL in PBS pH 7.4) was added BCN-IL15Rα-IL15 (PF15, 13.0 µL, 6.7 mg/mL in PBS pH 7.4, 5 equiv. BCN-labelled IL15Rα-IL15 compared to IgG). The reaction was incubated for 16 hours at room temperature. Mass spectral analysis of the IdeS digested sample showed one major product (observed mass 49419 Da), corresponding to conjugate trast-v1b-(PF15)₂.

### Example 161. Conjugation of Trast-v2 With BCN-IL15Rα-IL15 PF15 to Give Conjugate trast-v2-(PF15)₂ (P:A ratio 2:1)

To a solution of trast-v2 (3.9 µL, 75 µg, 19.5 mg/mL in PBS pH 7.4) was added BCN-IL15Rα-IL15 PF15 (13 µL, 6.7 mg/mL in PBS pH 7.4, 5 eq. BCN-labelled compared to IgG). The reaction was incubated for 16 hours at room temperature. Native gel analysis confirmed the formation of trast-v2-(PF15)₂, see FIG. 33.

### Example 162. Conjugation of BCN-IL15Rα-IL15 PF15 to trast-v6-XL02 via SPAAC (P:A Ratio 1:1)

Trast-v6-XL02 (0.1 mg, 10 mg/mL in PBS) was incubated with BCN-IL15Rα-IL15 PF15 (12.4 µL, 6.7 mg/mL, 3 eq. BCN-labelled IL15Rα-IL15 compared to IgG) overnight at room temperature. RP-HPLC analysis showed the formation of trast-v6-XL02-PF15 (see FIG. 25) and SDS-page gel analysis confirmed this conclusion (see FIG. 26).

### Example 163. Conjugation of BCN-IL15Rα-IL15 PF15 to trast-v5b-XL02 via SPAAC (P:A Ratio 1:1)

Trast-v5b-XL02 (0.1 mg, 10 mg/mL in PBS) was incubated with BCN-IL15Rα-IL15 PF15 (12.4 µL, 6.7 mg/mL, 3 eq. BCN-labelled IL15Rα-IL15 compared to IgG) overnight at room temperature. RP-HPLC analysis showed the formation of trast-v5b-XL02-PF15 (see FIG. 24) and SDS-page gel analysis confirmed this conclusion (see FIG. 34).

### Example 164. Conjugation of BCN-IL15Rα-IL15 PF15 to trast-v6-XL03 via SPAAC (P:A Ratio 1:1)

Trast-v6-XL03 (0.1 mg, 10 mg/mL in PBS) was incubated with BCN-IL15Rα-IL15 PF15 (12.4 µL, 6.7 mg/mL, 3 eq. BCN-labelled IL15Rα-IL15 compared to IgG) overnight at room temperature. SDS-page gel analysis showed the formation of trast-v6-XL03-PF15 (see FIG. 29).

### Example 165. Conjugation of Trast-v3 With BCN-IL15Rα-IL15 PF15 to Give Conjugate trast-v3-(PF15)₂ (P:A ratio 2:1)

To a solution of trast-v3 (3.85 µL, 75 µg, 19.5 mg/mL in PBS pH 7.4) was added BCN-IL15Rα-IL15 (PF15, 13.0 µL, 6.7 mg/mL in PBS pH 7.4, 5 eq. BCN labeled IL15Rα-IL15 compared to IgG). The reaction was incubated for 16 hours at room temperature. Mass spectral analysis of the IdeS digested sample showed one major product (observed mass 49030 Da), corresponding to trast-v3-(PF15)₂.

### Example 166. Conjugation of Trast-v3 With anti-4-1BB-BCN PF07 to Give Conjugate trast-v3-(PF07)₂ (P:A ratio 2:1)

To a solution of trast-v3 (3.85 µL, 75 µg, 19.5 mg/mL in PBS pH 7.4) was added anti-4-1BB-BCN (PF07, 10.5 µL, 6.8 mg/mL in PBS pH 7.4, 5 eq. compared to IgG). The reaction was incubated for 16 hours at room temperature. Mass spectral analysis of the IdeS digested sample showed one major product (observed mass 52468 Da), corresponding to trast-v3-(PF07)₂.

### Example 167. Conjugation of Rit-v3 With BCN- IL15Rα-IL15 PF15 to Give Conjugate rit-v3-(PF15)₂ (P:A ratio 2:1)

To a solution of rit-v3 (4.70 µL, 75 µg, 13.0 mg/mL in PBS pH 7.4) was added BCN-IL15Rα-IL15 (PF15,13.0 µL, 6.7 mg/mL in PBS pH 7.4, 5 eq. BCN-labelled compared to IgG). The reaction was incubated for 16 hours at room temperature. Mass spectral analysis of the IdeS digested sample showed one major product (observed mass 48999 Da) corresponding to rit-v3-(PF15)₂.

### Example 168. Conjugation of azido-IL 15 PF19 to trast-v6-(XL05)₂ via SPAAC (P:A ratio 2:1)

Trast-v6-(XL05)₂ (0.1 mg, 16 mg/mL in PBS) was incubated with azido-IL15 PF19 (5.6 µL, 7.2 mg/mL) overnight at room temperature. Mass spectral analysis of a sample after IdeS/EndoSH treatment showed one major Fc/2 product (observed mass 38775 Da) corresponding to the expected product trast-v6-(XL05-PF19)₂.

### Example 169. Conjugation of hOKT3-tetrazine PF02 to trast-v6-(XL05)₂ via SPAAC (P:A ratio 2:1)

Trast-v6-(XL05)₂ (0.1 mg, 16 mg/mL in PBS) was incubated with hOKT3-tetrazine PF02 (8.6 µL, 7.7 mg/mL) overnight at room temperature. Mass spectral analysis of a sample after IdeS/EndoSH treatment showed one major Fc/2 product (observed mass 53399 Da) corresponding to the expected product trast-v6-(XL05-PF02)₂.

### Example 170. Conjugation of Anti-4-188-azide PF09 to trast-v6-(XL05)₂ via SPAAC (P:A ratio 2:1)

Trast-v6-(XL05)₂ (0.1 mg, 16 mg/mL in PBS) was incubated with anti-4-1BB-azide PF09 (9.9 µL, 6.2 mg/mL) overnight at room temperature. Mass spectral analysis of a sample after IdeS/EndoSH treatment showed one major Fc/2 product (observed mass 52220 Da) corresponding to the expected product trast-v6-(XL05-PF09)₂.

### Example 171. Conjugation of azido-IL15 PF19 to trast-v5b-(XL05)₂ via SPAAC (P:A ratio 2:1)

Trast-v5b-(XL05)₂ (0.1 mg, 12.7 mg/mL in PBS) was incubated with azido-IL15 PF19 (5.6 µL, 7.2 mg/mL) overnight at room temperature. Mass spectral analysis of a sample after IdeS treatment showed one major Fc/2 product (observed mass 39009 Da) corresponding to the expected product trast-v5b-(XL05-PF19)₂.

### Example 172. Conjugation of hOKT3-tetrazine PF02 to trast-v5b-XL05 via SPAAC (P:A Ratio 2:1)

Trast-v5b-(XL05)₂ (0.1 mg, 12.7 mg/mL in PBS) was incubated with hOKT3-tetrazine PF02 (8.6 µL, 7.7 mg/mL) overnight at room temperature. Mass spectral analysis of a sample after IdeS treatment showed one major Fc/2 product (observed mass 52220 Da) corresponding to the expected product trast-v5b-(XL05-PF02)₂.

### Example 173. Conjugation of Anti-4-188-azide PF09 to trast-v5b-(XL05)₂ via SPAAC (P:A ratio 2:1)

Trast-v5b-(XL05)₂ (0.1 mg, 12.7 mg/mL in PBS) was incubated with anti-4-1BB-azide PF09 (9.9 µL, 6.2 mg/mL) overnight at room temperature. Mass spectral analysis of a sample after IdeS treatment showed one major Fc/2 product (observed mass 52455 Da) corresponding to the expected product trast-v5b-(XL05-PF09)₂.

### Example 174. Conjugation of azido-IL15 PF19 to Trast-v9 via CuAAC (P:A Ratio 2:1)

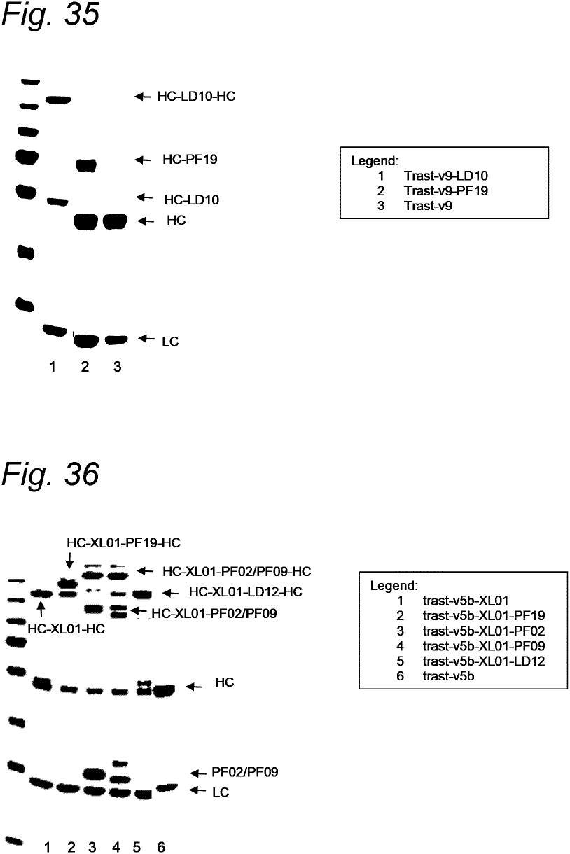

A solution was prepared with trast-v9 (0.2 mg, 16.5 µL 12.1 mg/mL) and azido-IL15 (PF19, 11 µL 7.2 mg/mL). In a separate vail a premix was prepared containing copper sulfate (71 µL, 15 mM), THTPA ligand (13 µL, 160 mM) amino guanidine (53 µL, 100 mM) and sodium ascorbate (40 µL, 400 mM). The premix was capped, vortexed and allowed to stand for 10 min. The premix (4.2 µL) was added to the antibody solution and the reaction was incubated for 2 hours followed by the addition of PBS + 1 mM EDTA (300 µL). The diluted solution was spinfiltered with PBS using centrifugal filters (Amicon Ultra-0.5 mL MWCO 10 kDa, Merck Millipore). Analysis on SDS-page gel showed the formation of the expected product trast-v9-(PF19)₂, see FIG. 35)

### Example 175. Conjugation of azido-IL15 PF19 to trast-v5b-XL01 via SPAAC (P:A Ratio 1:1) Trast-

v5b-XL01 (0.1 mg, 12.9 mg/mL in PBS) was incubated with azido-IL15 PF19 (5.6 µL, 7.2 mg/mL) overnight at room temperature. Analysis on SDS-page gel showed the formation of the expected product trast-v5b-XL01-PF19 (see FIG. 36).

### Example 176. Conjugation of hOkt3-tetrazine PF02 to trast-v5b-XL01 via SPAAC (P:A Ratio 1:1)

Trast-v5b-XL01 (0.1 mg, 12.9 mg/mL in PBS) was incubated with hOKT3-tetrazine PF02 (8.6 µL, 7.7 mg/mL) overnight at room temperature. Analysis on SDS-page gel showed the formation of the expected product trast-v5b-XL01-PF02 (see FIG. 36).

### Example 177. Conjugation of Anti-4-188-azide PF09 to trast-v5b-XL01 via SPAAC (P:A Ratio 1:1)

Trast-v5b-XL01 (0.1 mg, 12.9 mg/mL in PBS) was incubated with anti-4-1BB-azide PF09 (9.9 µL, 6.2 mg/mL) overnight at room temperature. Analysis on SDS-page gel showed the formation of the expected product trast-v5b-XL01-PF09 (see FIG. 36).

### Example 178. Conjugation of hOKT3-BCN 201 to Deglycosylated Trastuzumab via SPOCQ (P:A ratio 2:1)

Deglycosylated trastuzumab (4.0 µL, 0.075 mg, 18.6 mg/mL in PBS 5.5) was incubated with hOKT3-BCN (201, 6.56 µL, 4 eq., 11.0 mg/mL in PBS 5.5) and mushroom tyrosinase (1.5 µL, 10 mg/mL in phosphate buffer pH 6.0, Sigma Aldrich T3824) for 16 hours at room temperature. See also Dutch patent application no. 2026947, incorporated by reference herein. Mass spectral analysis of a sample after IdeS treatment showed one major Fc/2 product (observed mass 51824 Da) corresponding to the expected product trast-v4-(201)₂.

### Example 179. Intramolecular Crosslinking of bis-BCN-IL15Rα-IL15 PF27 to Trast-v3 via SPAAC (P:A ratio 1:1)

Trast-v3 (2.57 µL, 0.05 mg, 19.5 mg/mL in PBS) was incubated with bis-BCN-IL15Ra-IL15 (PF27, 5.6 µL, 3 eq. bis-BCN labelled IL15Rα-IL15, 7.6 mg/mL in PBS) for 16 hours at room temperature. Mass spectral analysis of a sample after IdeS treatment showed one major Fc/2 product (observed mass 73432 Da) corresponding to the expected product trast-v3-PF27.

### Example 180. Intramolecular Crosslinking of hOKT3-bis-BCN PF22 to Trast-v3 via SPAAC (P:A ratio 1:1)

Trast-v3 (2.57 µL, 0.05 mg, 19.5 mg/mL in PBS) was incubated with hOKT3-bis-BCN PF22 (5.15 µL, 3 eq., 5.7 mg/mL in PBS) for 16 hours at room temperature. Mass spectral analysis of a sample after IdeS treatment showed one major Fc/2 product (observed mass 77150 Da) corresponding to the expected product trast-v3-PF22.

### Example 181. Conjugation of hOKT3-BCN 201 to Trast-v3 via SPAAC (P:A Ratio 2:1)

Trast-v3 (2.57 µL, 0.05 mg, 19.5 mg/mL in PBS) was incubated with hOKT3-BCN (201,1.87 µL, 3 eq., 15.5 mg/mL in PBS) and 5 µL PBS for 16 hours at room temperature. Mass spectral analysis of a sample after IdeS treatment showed one major Fc/2 product (observed mass 51811 Da) corresponding to the expected product trast-v3-(201)₂.

### Example 182. Conjugation of Azido-IL15 PF19 to trast-v6-XL01 via SPAAC (P:A Ratio 1:1) Trast-

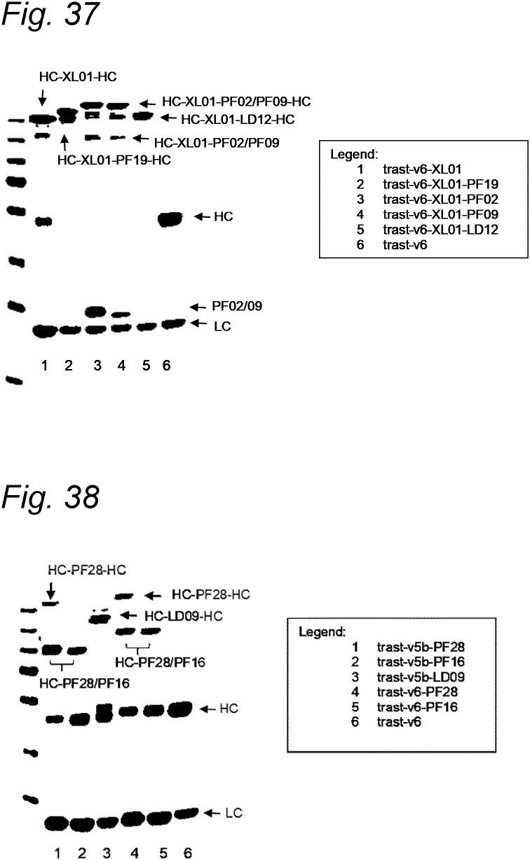

v6-XL01 (0.1 mg, 21.7 mg/mL in PBS) was incubated with azido-IL15 PF19 (5.6 µL, 7.2 mg/mL) overnight at room temperature. Analysis on SDS-page gel showed the formation of the expected product trast-v6-XL01-PF19 (see FIG. 37).

### Example 183. Conjugation of hOkt3-tetrazine PF02 to trast-v6-XL01 via SPAAC (P:A Ratio 1:1)

Trast-v6-XL01 (0.1 mg, 21.7 mg/mL in PBS) was incubated with hOKT3-tetrazine PF02 (8.6 µL, 7.7 mg/mL) overnight at room temperature. Analysis on SDS-page gel showed the formation of the expected product trast-v6-XL01-PF02 (see FIG. 37).

### Example 184. Conjugation of Anti-4-188-azide PF09 to trast-v6-XL01 via SPAAC (P:A Ratio 1:1)

Trast-v6-XL01 (0.1 mg, 21.7 mg/mL in PBS) was incubated with anti-4-1BB-azide PF09 (9.9 µL, 6.2 mg/mL) overnight at room temperature. Analysis on SDS-page gel showed the formation of the expected product trast-v6-XL01-PF09 (see FIG. 37).

### Example 185. Conjugation of azido-IL15 PF19 to trast-v7-XL01 via SPAAC (P:A Ratio 1:1) Trast-

v7-XL01 (0.1 mg, 20.8 mg/mL in PBS) was incubated with IL15 PF19 (5.6 µL, 7.2 mg/mL) overnight at room temperature. Analysis on SDS-page gel showed the formation of the expected product trast-v6-XL01-PF19 (see FIG. 23).

### Example 186. Conjugation of hOKT3-tetrazine PF02 to trast-v7-XL01 via SPAAC (P:A Ratio 1:1)

Trast-v7-XL01 (0.1 mg, 20.8 mg/mL in PBS) was incubated with hOKT3-tetrazine PF02 (8.6 µL, 7.7 mg/mL) overnight at room temperature. Analysis on SDS-page gel showed the formation of the expected product trast-v7-XL01-PF02 (see FIG. 23).

### Example 187. Conjugation of Anti-4-188-azide PF09 to trast-v7-XL01 via SPAAC (P:A Ratio 1:1)

Trast-v7-XL01 (0.1 mg, 20.8 mg/mL in PBS) was incubated with anti-4-1BB-azide PF09 (9.9 µL, 6.2 mg/mL) overnight at room temperature. Analysis on SDS-page gel showed the formation of the expected product trast-v7-XL01-PF09 (see FIG. 23).

### Example 188. Conjugation of trastuzumab-GaINProSSMe Trast-v5a With maleimide-IL15Rα-IL15 PF16 (P:A ratio 2:1)

Trastuzumab-GaINProSSMe (trast-v5a) (1.2 mg, 10 mg/mL in PBS + 10 mM EDTA, trast-v5a) was incubated with TCEP (7.8 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 120 µL. Subsequent DHA (7.8 µL, 10 mM in MQ:DMSO (9:1)) was added and the reaction was incubated for 3 hours at room temperature. To a part of the reaction mixture (0.1 mg, 10 µL) maleimide-IL15Rα-IL15 PF16 (6.6 µL 10 mg/mL) was added followed by incubation for 3 hours at room temperature. The conjugate was diluted with PBS to 1 mg/mL and subsequent analysis on SDS-page gel confirmed the formation of the conjugate trast-v5b-(PF16)₂ (see FIG. 38).

### Example 189. Conjugation of trastuzumab-GaINProSSMe Trast-v5a With bis-maleimide-IL15Rα-IL15 PF28 (P:A ratio 1:1)

Trastuzumab-GaINProSSMe (trast-v5a) (1.2 mg, 10 mg/mL in PBS + 10 mM EDTA, trast-v5a) was incubated with TCEP (7.8 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 120 µL. Subsequent DHA (7.8 µL, 10 mM in MQ:DMSO (9:1)) was added and the reaction was incubated for 3 hours at room temperature. To a part of the reaction mixture (0.1 mg, 10 µL) bis-maleimide- IL15Rα-IL15 PF28 (9.4 µL 7.1 mg/mL) was added followed by incubation for 3 hours at room temperature. The conjugate was diluted with PBS to 1 mg/mL and subsequent analysis on SDS-page gel confirmed the formation of the conjugate trast-v5b-PF28 (see FIG. 38).

### Example 190. Conjugation of trastuzumab-S239C Mutant Trast-v6 With maleimide-IL15Rα-IL15 PF16 (P:A ratio 2:1)

Trastuzumab S239C mutant (transient expressed in CHO by Evitria, heavy chain mutation S239C) (2 mg, 10 mg/mL in PBS + 10 mM EDTA, trast-v6) was incubated with TCEP (13 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 120 µL. Subsequent DHA (13 µL, 10 mM in MQ:DMSO (9:1)) was added and the reaction was incubated for 3 hours at room temperature. To a part of the reaction mixture (0.1 mg, 11 µL) maleimide-IL15Rα-IL15 PF16 (6.6 µL 10 mg/mL) was added followed by incubation for 3 hours at room temperature. The conjugate was diluted with PBS to 1 mg/mL and subsequent analysis on SDS-page gel confirmed the formation of the conjugate trast-v6-(PF16)₂ (see FIG. 38).

### Example 191. Conjugation oftrastuzumab-S239C Mutant Trast-v6 With bis-maleimide-IL15Rα-IL15 PF28 (P:A ratio 1:1)

Trastuzumab S239C mutant (transient expressed in CHO by Evitria, heavy chain mutation S239C) (2 mg, 10 mg/mL in PBS + 10 mM EDTA, trast-v6) was incubated with TCEP (13 µL, 10 mM in MQ) for 2 hours at 37° C. The reduced antibody was spinfiltered with PBS + 10 mM EDTA using centrifugal filters (Amicon Ultra-0.5 mL MWCO 10 kDa, Merck Millipore) and diluted to 120 µL. Subsequent DHA (13 µL, 10 mM in MQ:DMSO (9:1)) was added and the reaction was incubated for 3 hours at room temperature. To a part of the reaction mixture (0.1 mg, 11 µL) bis-maleimide- IL15 PF28 (9.4 µL 7.2 mg/mL) was added followed by incubation for 3 hours at room temperature. The conjugate was diluted with PBS to 1 mg/mL and subsequent analysis on SDS-page gel confirmed the formation of the conjugate trast-v6-PF28 (see FIG. 38).

### Example 192. Conjugation of hOkt3-tetrazine PF02 to trast-v6-XL12 via SPAAC (P:A Ratio 1:1)

Trast-v6-XL12 (0.1 mg, 15.9 mg/mL in PBS) was incubated with hOKT3-tetrazine PF02 (8.6 µL, 7.7 mg/mL) overnight at room temperature. Analysis on SDS-page gel showed the formation of the expected product trast-v6-XL12-PF02 (see FIG. 39).

### Example 193. Conjugation of hOkt3-tetrazine PF02 to trast-v8-XL06 via SPAAC (P:A Ratio 2:1)

To a solution of trast-v8-XL06 (4.45 µL, 75 µg, 16.85 mg/mL in PBS pH 7.4) was added hOkt3-tetrazine PF02 (8.90 µL, 6.2 mg/mL in PBS, 4 eq compared to IgG). The reaction was incubated for 16 hours at room temperature. Analysis on SDS-page gel showed the formation of the expected product trast-v8-XL06-PF02 (see FIG. 30).

### Example 194. Conjugation of Anti-4-188-azide PF09 to trast-v8-XL06 via SPAAC (P:A Ratio 2:1)

To a solution of trast-v8-XL06 (4.45 µL, 75 µg, 16.85 mg/mL in PBS pH 7.4) was added anti-4-1BB-zide PF09 (7.49 µL, 7.7 mg/mL in PBS, 4 eq compared to IgG). The reaction was incubated for 16 hours at room temperature. Analysis on SDS-page gel showed the formation of the expected product trast-v8-XL06-PF09 (see FIG. 30).

### Example 195. Conjugation of hOKT3-PEG₂-BCN 201 to bis-azido-rituximab rit-v1a to give T cell engager rit-v1a-(201)₂ with 2:2 molecular format

To a solution of rit-v1a (99 µL, 6.0 mg, 405 µM in PBS pH 7.4) was added hOKT3-PEG₂-BCN 201 (240 µL, 4.4 mg, 666 µM in PBS pH 7.4, 4 equiv. compared to IgG). The reaction was incubated overnight at 37° C. followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to two hOKT3 scFvs (See FIG. 19, left panel, lane 4), thereby confirming formation of rit-v1a-(201)₂. Furthermore, reducing SDS-PAGE showed one major HC product, corresponding to the heavy chain conjugated to hOKT3-PEG₂-BCN 201 (See FIG. 19, right panel, lane 4).

### Example 196. Conjugation of hOKT3-PEG₄-tetrazine 204 to BCN-rituximab rit-v1a-145 to give T Cell Engager rit-v1a-145-204 with 2:1 Molecular Format

To a solution of rit-v1a-145 (287 µL, 6.6 mg, 154 µM in PBS pH 7.4) was added hOKT3-PEG₄-tetrazine 204 (247 µL, 1.9 mg, 269 µM in PBS pH 6.5, 1.5 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (See FIG. 19, left panel, lane 5), thereby confirming formation of rit-v1a-145-204. Furthermore, reducing SDS-PAGE confirms one major HC product, corresponding to two heavy chains conjugated to a single hOKT3 (See FIG. 19, right panel, lane 5).

### Example 197. Conjugation of hOKT3-PEG₁₁-tetrazine PF01 to BCN-Rituximab rit-v1a-145 to Give T Cell Engager rit-v1a-145-PF01 with 2:1 Molecular Format

To a solution of rit-v1a-145 (247 µL, 6.3 mg, 171 µM in PBS pH 7.4) was added hOKT3-PEG₁₁-tetrazine PF01 (304 µL, 2.0 mg, 230 µM in PBS pH 6.5, 1.7 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (See FIG. 19, left panel, lane 6), thereby confirming formation of rit-v1a-145-PF01. Furthermore, reducing SDS-PAGE confirms one major HC product, corresponding to two heavy chains conjugated to a single hOKT3 (See FIG. 19, right panel, lane 6).

### Example 198. Conjugation of hOKT3-PEG₁₁-tetrazine PF01 to BCN-B12 B12-v1a-145 to Give T Cell Engager B12-v1a-145-PF01 with 2:1 Molecular Format

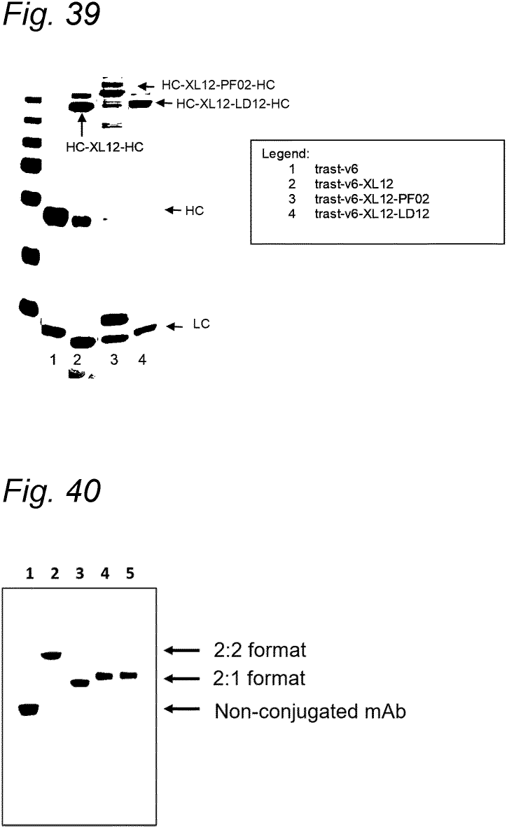

To a solution of B12-v1a-145 (38 µL, 1.0 mg, 178 µM in PBS pH 7.4) was added hOKT3-PEG₁₁-tetrazine PF01 (44 µL, 0.3 mg, 230 µM in PBS pH 6.5, 1.5 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (see FIG. 40, lane 4), thereby confirming formation of B12-v1a-145-PF01.

### Example 199. Conjugation of hOKT3-PEG₄-tetrazine 204 to TCO-trastuzumab trast-v1a-XL11 to Give T Cell Engager trast-v1a-XL11-204 with 2:1 Molecular Format

To a solution of TCO-trastuzumab trast-v1a-XL11 (5.7 µL, 100 µg, 117 µM in PBS pH 7.4) was added hOKT3-PEG₄-tetrazine 204 (5 µL, 38 µg, 269 µM in PBS pH 6.5, 2.0 equiv. compared to IgG). The reaction was incubated overnight at rt. Non-reducing SDS-PAGE analysis showed two major products corresponding to the non-conjugated antibody and the antibody conjugated to a single hOKT3 (See FIG. 31, left panel, lane 3), thereby confirming formation of trast-v1a-XL11-204. Furthermore, reducing SDS-PAGE confirms that OKT3 is conjugated to the crosslinked heavy chains containing the TCO reactive handle (See FIG. 31, right panel, lane 3).

### Example 200. Conjugation of hOKT3-PEG₄-tetrazine 204 to TCO-rituximab rit-v1a-XL11 to Give T Cell engager rit-v1a-XL11-204 with 2:1 Molecular Format

To a solution of TCO-rituximab rit-v1a-XL11 (56.3 µL, 100 µg, 106 µM in PBS pH 7.4) was added hOKT3-PEG₄-tetrazine 204 (5 µL, 38 µg, 269 µM in PBS pH 6.5, 2.0 equiv. compared to IgG). The reaction was incubated overnight at rt. Non-reducing SDS-PAGE analysis showed two major products corresponding to the non-conjugated antibody and the antibody conjugated to a single hOKT3 (See FIG. 31, left panel, lane 7), thereby confirming formation of rit-v1a-XL11-204. Furthermore, reducing SDS-PAGE confirms that OKT3 is conjugated to the crosslinked heavy chains containing the TCO reactive handle (See FIG. 31, right panel, lane 7).

### Example 201. Conjugation of hOKT3-PEG₂₃-tetrazine PF02 to BCN-rituximab rit-v1a-145 to Give T Cell Engager rit-v1a-145-PF02 with 2:1 Molecular Format

To a solution of rit-v1a-145 (247 µL, 6.3 mg, 171 µM in PBS pH 7.4) was added hOKT3-PEG₂₃-tetrazine PF02 (262 µL, 2.0 mg, 267 µM in PBS pH 6.5, 1.7 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (See FIG. 19, left panel, lane 7), thereby confirming formation of rit-v1a-145-PF02. Furthermore, reducing SDS-PAGE confirms one major HC product, corresponding to two heavy chains conjugated to a single hOKT3 (See FIG. 19, right panel, lane 7).

### Example 202. Conjugation of hOKT3-PEG₂-arylazide PF03 to BCN-trastuzumab trast-v1a-145 to Give T Cell Engager trast-v1a-145-PF03 with 2:1 Molecular Format

To a solution of trast-v1a-145 (2.9 µL, 150 µg, 347 µM in PBS pH 7.4) was added hOKT3-PEG₂-arylazide PF03 (4.9 µL, 56 µg, 411 µM in PBS pH 7.4, 2.0 equiv. compared to IgG). The reaction was incubated overnight at rt. Mass spectral analysis of the reduced sample showed one major heavy chain product (observed mass 128388 Da), corresponding to trast-v1a-145-PF03.

### Example 203. Conjugation of hOKT3-PEG₂-arylazide PF03 to BCN-rituximab rit-v1a-145 to Give T Cell engager rit-v1a-145-PF03 with 2:1 Molecular Format

To a solution of rit-v1a-145 (30 µL, 1.5 mg, 337 µM in PBS pH 7.4) was added hOKT3-PEG₂-arylazide PF03 (49 µL, 0.6 mg, 411 µM in PBS pH 7.4, 2.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Mass spectral analysis of the reduced sample showed one major heavy chain product (observed mass 128211 Da), corresponding to rit-v1a-145-PF03.

### Example 204. Conjugation bis-BCN-hOKT3 PF22 to Bis-azido-trastuzumab Trast-v1a to Give T Cell Engager trast-v1a-PF22 with 2:1 Molecular Format

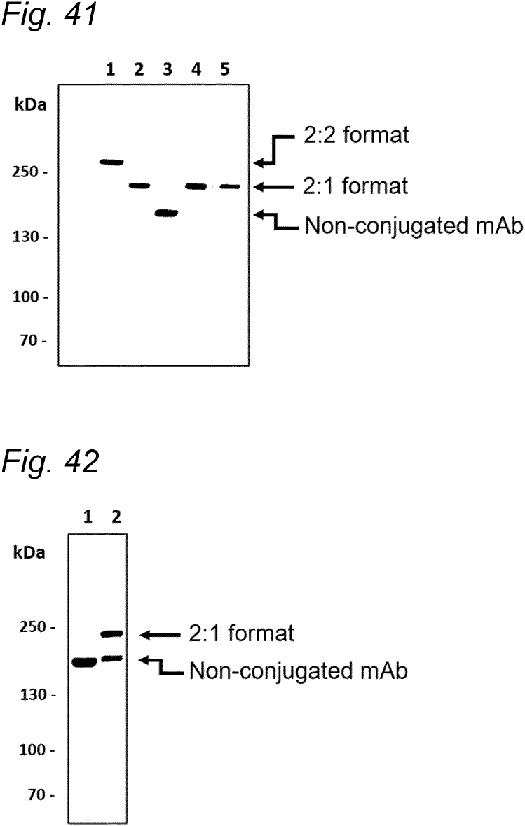

To a solution of trast-v1a (1.8 µL, 100 µg, 374 µM in PBS pH 7.4) was added PBS pH 7.4 (4.5 µL) and bis-BCN-hOKT3 PF22 (13.7 µL, 78 µg, 194 µM in PBS pH 7.4, 4.0 equiv. compared to IgG). The reaction was incubated overnight at rt. Non-reducing SDS-PAGE analysis showed one major product consisting of an antibody conjugated to a single hOKT3 (See FIG. 41, lane 5), thereby confirming formation of trast-v1a-PF22.

### Example 205. Conjugation of bis-BCN-hOKT3 PF22 to Bis-azido-rituximab Rit-v1a to Give T Cell Engager rit-v1a-145-PF22 with 2:1 Molecular Format

To a solution of rit-v1a (1.8 µL, 100 µg, 363 µM in PBS pH 7.4) was added PBS pH 7.4 (7.9 µL) and bis-BCN-hOKT3 PF22 (10.3 µL, 58 µg, 194 µM in PBS pH 7.4, 3.0 equiv. compared to IgG). The reaction was incubated overnight at rt. Non-reducing SDS-PAGE analysis showed one major product consisting of an antibody conjugated to a single hOKT3 (See FIG. 41, lane 4), thereby confirming formation of rit-v1a-PF22.

### Example 206. Conjugation of bis-BCN-hOKT3 PF23 to Bis-azido-trastuzumab Trast-v1a to Give T Cell Engager trast-v1a-PF23 with 2:1 Molecular Format

To a solution of trast-v1a (1.8 µL, 100 µg, 374 µM in PBS pH 7.4) was added PBS pH 7.4 (9.9 µL) and bis-BCN-hOKT3 PF23 (8.4 µL, 58 µg, 239 µM in PBS pH 7.4, 3.0 equiv. compared to IgG). The reaction was incubated overnight at 37° C. Non-reducing SDS-PAGE analysis showed two major products consisting of non-conjugated trastuzumab and trastuzumab conjugated to bis-BCN-hOKT3 PF23 (See FIG. 42, lane 2), thereby confirming partial formation of trast-v1a-PF23.

### Example 207. Conjugation of bis-BCN-hOKT3 PF23 to Bis-azido-rituximab Rit-v1a to Give T Cell Engager rit-v1a-PF23 with 2:1 Molecular Format

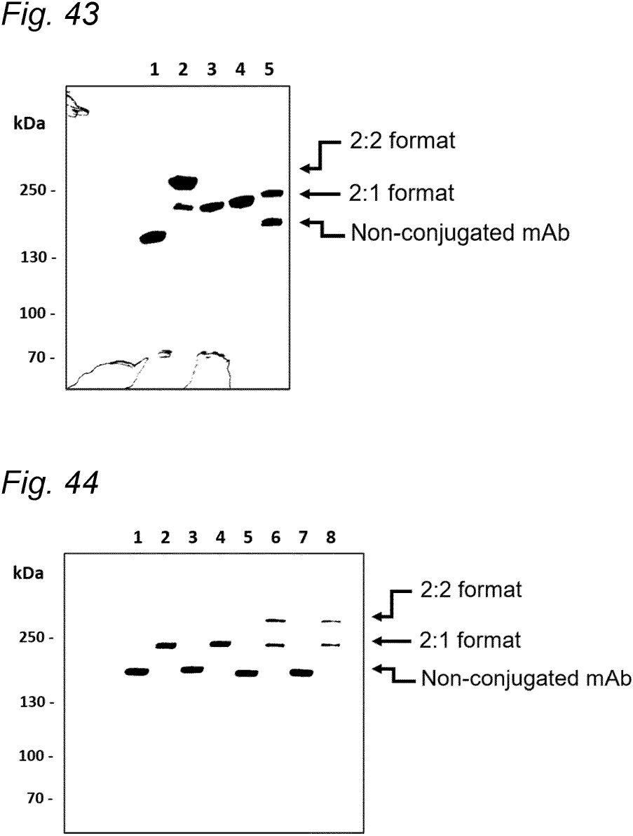

To a solution of rit-v1a (1.8 µL, 100 µg, 363 µM in PBS pH 7.4) was added PBS pH 7.4 (13.6 µL) and bis-BCN-hOKT3 PF23 (4.3 µL, 30 µg, 239 µM in PBS pH 7.4, 1.5 equiv. compared to IgG). The reaction was incubated overnight at 37° C. Non-reducing SDS-PAGE analysis showed two major products consisting of non-conjugated rituximab and rituximab conjugated once to bis-BCN-hOKT3 PF23 (See FIG. 43, lane 5), thereby confirming partial formation of rit-v1a-PF23.

### Example 208. Conjugation of 4-1BB-PEG₂₃-BCN PF07 to bis-azido-trastuzumab trast-v1a to Give T Cell Engager trast-v1a-(PF07)2 with 2:2 Molecular Format

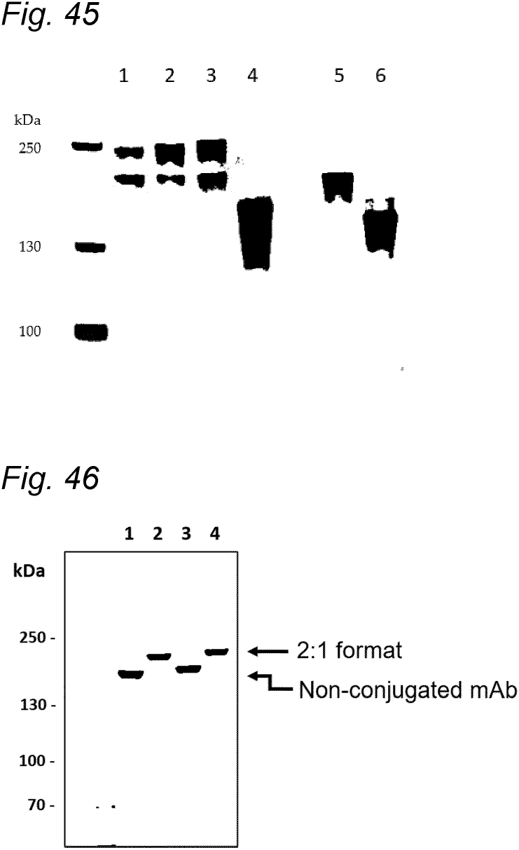

To a solution of trast-v1a (1.8 µL, 100 µg, 374 µM in PBS pH 7.4) was added 4-1BB-PEG₂₃-BCN PF07 (11.2 µL, 76 µg, 239 µM in PBS pH 7.4, 4.0 equiv. compared to IgG). The reaction was incubated overnight at 37° C. Non-reducing SDS-PAGE analysis showed two major products consisting of trastuzumab conjugated once and twice to 4-1BB-PEG₂₃-BCN PF07 (See FIG. 44, lane 8), thereby confirming partial formation of trast-v1a-(PF07)₂.

### Example 209. Conjugation of 4-1BB-PEG₂₃-BCN PF07 to bis-azido-rituximab rit-v1a to Give T Cell Engager rit-v1a-(PF07)₂ with 2:2 Molecular Format

To a solution of rit-v1a (1.8 µL, 100 µg, 363 µM in PBS pH 7.4) was added 4-1BB-PEG₂₃-BCN PF07 (11.2 µL, 76 µg, 239 µM in PBS pH 7.4, 4.0 equiv. compared to IgG). The reaction was incubated overnight at 37° C. Non-reducing SDS-PAGE analysis showed two major products consisting of rituximab conjugated once and twice to 4-1BB-PEG₂₃-BCN PF07 (See FIG. 44, lane 6), thereby confirming partial formation of rit-v1a-(PF07)₂. Furthermore, mass spectral analysis of the reduced sample showed two major heavy chain products (observed masses of 49640 and 78117 Da, each approximately 50% of total heavy chain), corresponding to the non-conjugated heavy chain and the heavy chain conjugated to 4-1BB-PEG₂₃-BCN PF07.

### Example 210. Conjugation of 4-1BB-PEG₁₁-tetrazine PF08 to BCN-rituximab rit-v1a-145 to Give T Cell Engager rit-v1a-145-PF08 with 2:1 Molecular Format

To a solution of rit-v1a-145 (35 µL, 0.9 mg, 170 µM in PBS pH 7.4) was added 4-1BB-PEG₁₁-tetrazine PF08 (40 µL, 248 µg, 222 µM in PBS pH 7.4, 1.5 equiv. compared to IgG). The reaction was incubated overnight at rt. Non-reducing SDS-PAGE analysis showed one major product consisting of rituximab conjugated to 4-1BB-PEG₂₃-BCN PF08 (See FIG. 40, lane 3), thereby confirming partial formation of rit-v1a-145-PF08.

### Example 211. Conjugation of 4-1BB-PEG₁₁-tetrazine PF08 to BCN-B12 B12-v1a-145 to Give T Cell Engager B12-v1a-145-PF08 with 2:1 Molecular Format

To a solution of B12-v1a-145 (34 µL, 0.9 mg, 178 µM in PBS pH 7.4) was added 4-1BB-PEG₁₁-tetrazine PF08 (40 µL, 248 µg, 222 µM in PBS pH 7.4, 1.5 equiv. compared to IgG). The reaction was incubated overnight at rt. Non-reducing SDS-PAGE analysis showed one major product consisting of B12 conjugated to 4-1BB-PEG₂₃-BCN PF08 (See FIG. 40, lane 5), thereby confirming partial formation of B12-v1a-145-PF08.

### Example 212. Conjugation of 4-1BB-PEG₂-arylazide PF09 to BCN-trastuzumab trast-v1a-145 to Give T Cell Engager trast-v1a-145-PF09 with 2:1 Molecular Format

To a solution of trast-v1a-145 (1.9 µL, 100 µg, 347 µM in PBS pH 7.4) was added 4-1BB-PEG₂-arylazide PF09 (5.9 µL, 37 µg, 225 µM in PBS pH 7.4, 2.0 equiv. compared to IgG). The reaction was incubated overnight at rt. Non-reducing SDS-PAGE analysis showed one major product consisting of trastuzumab conjugated to a single 4-1BB-PEG₂-arylazide PF09 (See FIG. 44, lane 4), thereby confirming formation of trast-v1a-145-PF09.

### Example 213. Conjugation of 4-1BB-PEG₂-arylazide PF09 to BCN-rituximab rit-v1a-145 to Give T Cell Engager rit-v1a-145-PF09 with 2:1 Molecular Format

To a solution of rit-v1a-145 (2.0 µL, 100 µg, 337 µM in PBS pH 7.4) was added 4-1BB-PEG₂-arylazide PF09 (5.9 µL, 37 µg, 225 µM in PBS pH 7.4, 2.0 equiv. compared to IgG). The reaction was incubated overnight at rt. Non-reducing SDS-PAGE analysis showed one major product consisting of rituximab conjugated to a single 4-1BB-PEG₂-arylazide PF09 (See FIG. 44, lane 2), thereby confirming formation of rit-v1a-145-PF09.

### Example 214. Conjugation of BCN-GGG-IL15Rα-IL15 (PF10) to Bis-azido-trastuzumab Trast-v1a to Give T Cell Engager trast-v1a-(PF10)₂ with 2:2 Molecular Format

Trast-v1a (11.5 µL, 0.305 mg, 27.7 mg/mL in PBS) was incubated with PF10 (35 µL, 4 eq., 5.9 mg/mL in PBS) for 16 h at 37° C. Analysis on non-reducing SDS-page gel confirmed the formation of Trast-v1a-PF10 and Trast-v1a-(PF10)₂. (See FIG. 45, lane 1)

### Example 215. Conjugation of BCN-PEG₂₄-GGG-IL15Rα-IL15 (PF11) to bis-azido-trastuzumab trast-v1a to Give T Cell Engager trast-v1a-(PF11)₂ with 2:2 Molecular Format

Trast-v1a (12 µL, 0.332 mg, 27.7 mg/mL in PBS) was incubated with PF11 (35 µL, 4 eq., 6.1 mg/mL in PBS) for 16 h at 37° C. Analysis on non-reducing SDS-PAGE confirmed the formation of trast-v1a-PF11 and trast-v1a-(PF11)₂ (see FIG. 45, lane 3).

### Example 216. Conjugation of Tetrazine-PEG₃-GGG-IL15Rα-IL15 (PF12) to BCN-trastuzumab trast-v1a-145 to Give T Cell Engager trast-v1a-145-PF12 with 2:1 Molecular Format

Trast-v1a-145 (75 µL, 1.575 mg, 21 mg/mL in PBS) was incubated with PF12 (80 µL, 2 eq., 6.5 mg/mL in PBS) for 16 h at 37° C. Analysis on non-reducing SDS-PAGE confirmed the formation of Trast-v1a-145-PF12 (see FIG. 45, lane 5).

### Example 217. Conjugation of Arylazide-PEG11-GGG-IL15Rα-IL15 (PF13) to BCN-trastuzumab trast-v1a-145 to Give T Cell Engager trast-v1a-145-PF13 with 2:1 Molecular Format

Trast-v1a-145 (280 µL, 5.2 mg, 18.6 mg/mL in PBS) was incubated with PF13 (477 µL, 1.5 eq., 2.6 mg/mL in PBS) for 16 h at 37° C. Mass spectral analysis of a sample after IdeS treatment showed one major product of 73991 Da, corresponding to the crosslinked Fc-fragment conjugated to PF13 (expected mass: 73989 Da), thereby confirming formation of trast-v1a-145-PF13.

### Example 218. Conjugation of Arylazide-PEG11-GGG-IL15Rα-IL15 (PF13) to BCN-Rituximab Rit-v1a-145to give T Cell Engager Rit-v1a-145-PF13 with 2:1 Molecular Format

Rit-v1a-145 (0.5 µL, 0.025 mg, 50.6 mg/mL in PBS) was incubated with PF13 (6.6 µL, 4 eq., 2.6 mg/mL in PBS) for 16 h at RT. Mass spectral analysis of a sample after IdeS treatment showed one major product of 73927 Da, corresponding to the crosslinked Fc-fragment conjugated to PF13 (expected mass: 73925 Da), thereby confirming formation of rit-v1a-145-PF13.

### Example 219. Conjugation of BCN-PEG₁₂-SYR-(G₄S)₃-IL15Rα-IL15 (PF14) to bis-azido-trastuzumab trast-v1a to Give T Cell Engager trast-v1a-(PF14)₂ with 2:2 Molecular Format Trast-

v1a (5.2 µL, 0.156 mg, 30 mg/mL in PBS) was incubated with PF14 (50 µL, 4 eq., 3.2 mg/mL in PBS) for 16 h at 37° C. Mass spectral analysis of a sample after IdeS treatment showed one major product of 49387 Da, corresponding to the Fc-fragment conjugated to PF14 (expected mass:49387), thereby confirming formation of trast-v1a-(PF14)₂.

### Example 220. Conjugation of BCN-SYR-(G₄S)₃-IL15Rα-IL15 (PF15) to bis-azido-trastuzumab trast-v1a to Give T Cell Engager trast-v1a-(PF15)₂ with 2:2 Molecular Format

Trast-v1a (0.8 µL, 0.045 mg, 56.1 mg/mL in PBS) was incubated with PF15 (6.9 µL, 4 eq., 6.2 mg/mL in PBS) for 16 h at RT. Mass spectral analysis of a sample after IdeS treatment showed one major product of 49403 Da, corresponding to the Fc-fragment conjugated to PF15 (Expected mass: 49405 Da), thereby confirming formation of trast-v1a-(PF15)₂.

### Example 221. Conjugation of BCN-SYR-(G₄S)₃-IL15Rα-IL15 (PF15) to bis-azido-Rituximab rit-v1a to Give T Cell Engager rit-v1a-(PF15)₂ with 2:2 Molecular Format

Rit-v1a (0.8 µL, 0.044 mg, 54.6 mg/mL in PBS) was incubated with PF15 (6.7 µL, 4 eq.,6.2 mg/mL in PBS) for 16 h at RT. Mass spectral analysis of a sample after IdeS treatment showed one major product of 49374 Da, corresponding to the Fc-fragment conjugated to PF15 (expected mass: 49373 Da), thereby confirming formation of rit-v1a-(PF15)₂.

### Example 222. Conjugation of bis-BCN-SYR-(G₄S)₃-IL15Rα-IL15 (PF27) to bis-azido-trastuzumab trast-v1a to Give T Cell Engager trast-v1a-145-PF27 with 2:1 Molecular Format

Trast-v1a (1.78 µL, 0.099 mg, 56.1 mg/mL in PBS) was incubated with PF27 (18.4 µL, 4 eq., 7.62 mg/mL in PBS) and with 2.87 µL PBS for 16 h at 37° C. Mass spectral analysis of a sample after IdeS treatment showed one major product of 74193 Da, corresponding to the crosslinked Fc-fragment conjugated to PF27 (expected mass: 74178 Da), thereby confirming formation of trast-v1a-145-PF27.

### Example 223. Conjugation of bis-BCN-SYR-(G₄S)₃-IL15Rα-IL15 (PF27) to bis-azido-Rituximab Rit-v1a to Give T Cell Engager Rit-v1a-145-PF27 with 2:1 Molecular Format

Rit-v1a (1 µL, 0.055 mg, 54.6 mg/mL in PBS) was incubated with PF27 (8.9 µL, 4 eq.,6.2 mg/mL in PBS) and with 1.6 µL PBS for 16 h at 37° C. Mass spectral analysis of a sample after IdeS treatment showed one major product of 74118 Da, corresponding to the crosslinked Fc-fragment conjugated to PF27 (Expected mass: 74114 Da), thereby confirming formation of rit-v1a-145-PF27.

### Example 224. Conjugation of azido-IL15Rα-IL15 PF17 to BCN-trastuzumab Trast-v1a-145 to Give T Cell Engager trast-v1a-145-PF17 with 2:1 Molecular Format

To a solution of trast-v1a-145 (29 µL, 1.5 mg, 347 µM in PBS pH 7.4) was added azido-IL15Rα-IL15 PF17 (97 µL, 1.1 mg, 411 µM in PBS pH 7.4, 4.0 equiv. compared to IgG). The reaction was incubated overnight at 37° C. Non-reducing SDS-PAGE analysis showed one major product consisting of trastuzumab conjugated to a single azido-IL15Rα-IL15 PF17 (See FIG. 46, lane 4), thereby confirming formation of trast-v1a-145-PF17.

### Example 225. Conjugation of azido-IL15Rα-IL15 PF17 to BCN-rituximab Rit-v1a-145 to Give T Cell Engager rit-v1a-145-PF17 with 2:1 Molecular Format

To a solution of rit-v1a-145 (3 µL, 150 µg, 337 µM in PBS pH 7.4) was added azido-IL15Rα-IL15 PF17 (9.7 µL, 111 µg, 411 µM in PBS pH 7.4, 4.0 equiv. compared to IgG). The reaction was incubated overnight at 37° C. Non-reducing SDS-PAGE analysis showed one major product consisting of rituximab conjugated to a single azido-IL15Rα-IL15 PF17 (See FIG. 46, lane 2), thereby confirming formation of rit-v1a-145-PF17.

### Example 226. Conjugation of azido-IL15 PF19 to BCN-trastuzumab Tras-v1a-145 to Give T Cell Engager tras-v1a-145-PF19 with 2:1 Molecular Format

Trast-v1a-145 (4.0 µL, 0.075 mg, 18.6 mg/mL in PBS) was incubated with PF19 (4.6 µL, 5 eq., 7.7 mg/mL in PBS) for 16 h at RT. Mass spectral analysis of a sample after IdeS treatment showed one major product of 63941 Da, corresponding to the crosslinked Fc-fragment conjugated to PF19 (Expected mass: 63936 Da), thereby confirming formation of trast-v1a-145-PF19.

### Example 227. Conjugation of azido-IL15 PF19 to BCN-rituximab Rit-v1a-145 to Give T Cell Engager rit-v1a-145-PF19 with 2:1 Molecular Format

Rit-v1a-145 (2.0 µL, 0.112 mg, 50.6 mg/mL in PBS) was incubated with PF19 (5.1 µL, 4 eq., 7.7 mg/mL in PBS) for 16 h at RT. Mass spectral analysis of a sample after IdeS treatment showed one major product of 63882 Da, corresponding to the crosslinked Fc-fragment conjugated to PF19 (Expected mass: 63879 Da), thereby confirming formation of rit-v1a-145-PF19.

### Example 228. Conjugation of bis-BCN-SYR-(G₄S)₃-IL15 (PF29) to bis-azido-trastuzumab tras-v1a to Give T Cell Engager Tras-v1a-PF29 with 2:1 Molecular Format

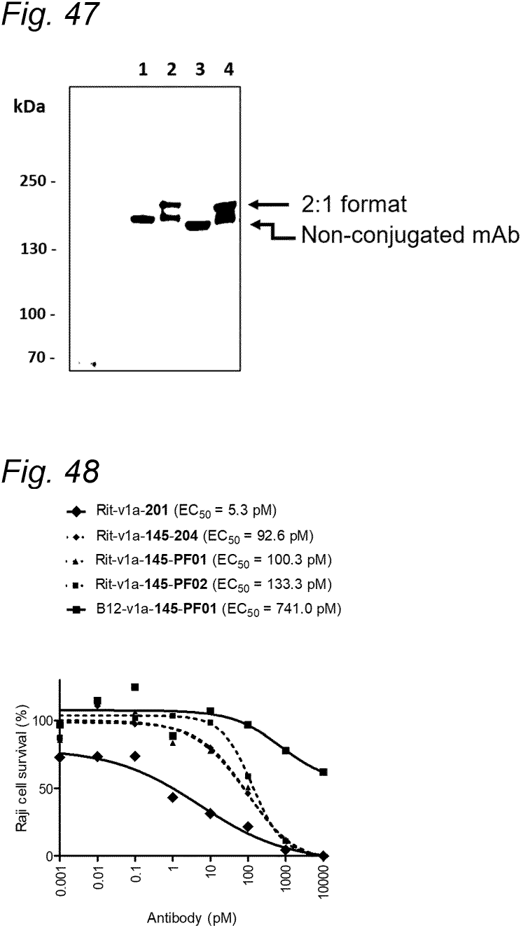

Trast-v1a (1 µL, 0.056 mg, 56.1 mg/mL in PBS) was incubated with PF29 (11 µL, 4 eq., 3.6 mg/mL in PBS) for 16 h at 37° C. Non-reducing SDS-PAGE analysis showed two major products corresponding to non-conjugated trastuzumab and trastuzumab conjugated to a single bis-BCN-SYR-(G₄S)₃-IL15 PF29 (See FIG. 47, lane 2), thereby confirming partial conversion into Tras-v1a-PF29.

### Example 229. Conjugation of bis-BCN-SYR-(G₄S)₃-IL15 (PF29) to bis-azido-rituximab Rit-v1a to Give T Cell Engager Rit-v1a-PF29 with 2:1 Molecular Format

Rit-v1a (1 µL, 0.055 mg, 54.6 mg/mL in PBS) was incubated with PF29 (11 µL, 4 eq.,3.6 mg/mL in PBS) for 16 h at 37° C. Non-reducing SDS-PAGE analysis showed two major products corresponding to non-conjugated rituximab and rituximab conjugated to a single bis-BCN-SYR-(G₄S)₃-IL15 PF29 (See FIG. 47, lane 4), thereby confirming partial conversion into rit-v1a-PF29.

### Example 230. Conjugation of tetrazine-PEG₁₂-SYR-(G₄S)₃-IL15 (PF21) to BCN-trastuzumab trast-v1a-145 to Give T Cell Engager trast-v1a-145-PF21 with 2:1 Molecular Format

Trast-v1a (2 µL, 0.042 mg, 21 mg/mL in PBS) was incubated with PF21 (10 µL, 6.7 eq., 2.9 mg/mL in PBS) for 16 h at 37° C. Mass spectral analysis of a sample after IdeS treatment showed one major product of 64865 Da, corresponding to the crosslinked Fc-fragment conjugated to PF21 (Expected mass: 64863 Da), thereby confirming formation of trast-v1a-145-PF21.

### Example 231. BCN-PEG₁₁-BCN (105) Functionalization of Tyrosine Residue in SYR-(G₄-S)₃-IL15 (PF18) Using Mushroom Tyrosinase to Obtain BCN-PEG₁₁-IL15 (PF20)

To a solution containing protein SYR-(G₄S)₃-IL15 (PF18) (334 µL, 269 µM in PBS pH 7.4) was added PBS pH 7.4 (103.9 µL), BCN-PEG11-BCN (105) (10 eq., 18 µL, 50 mM in DMSO) and mTyrosinase (443.7 µL, 203 µM in PBS pH 7.4) and incubated 4 hours at RT. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 15031 Da (expected mass: 15033 Da) corresponding to PF20.

### Example 232. Conjugation of BCN-PEG₁₁-IL15 (PF20) to bis-azido-trastuzumab trast-v1a to Give T Cell Engager trast-v1a-(PF20)₂ with 2:2 Molecular Format

Trast-v1a (1.5 µL, 0.084 mg, 56.1 mg/mL in PBS) was incubated with PF20 (7.3 µL, 4 eq., 6.2 mg/mL in PBS) for 16 h at RT. Mass spectral analysis of a sample after DTT treatment showed two major products, corresponding to the heavy chain conjugated to PF20 (observed mass: 64764 Da; expected mass: 64758 Da; approximately 20% of total heavy chain peaks) and the unconjugated heavy chain (49725 Da; approximately 80% of total heavy chain peaks), thereby confirming partial formation of trast-v1a-(PF20)₂.

### Example 233. Conjugation of BCN-PEG₁₁-IL15 (PF20) to bis-azido-rituximab rit-v1a to Give T Cell Engager rit-v1a-(PF20)₂ with 2:2 Molecular Format

Rit-v1a (1.5 µL, 0.082 mg, 54.6 mg/mL in PBS) was incubated with PF20 (7.1 µL, 4 eq.,6.2 mg/mL in PBS) for 16 h at RT. Mass spectral analysis of a sample after DTT treatment showed two major products, corresponding to the heavy chain conjugated to PF20 (observed mass: 64671 Da; expected mass: 64669 Da; approximately 10% of total heavy chain peaks) and the unconjugated heavy chain (49636 Da; approximately 90% of total heavy chain peaks). thereby confirming partial formation of rit-v1a-(PF20)₂.

### Example 234. CD3 Binding Analysis

Specific binding to CD3 was assessed using Jurkat E6.1 cells, which express CD3 on the cell surface, and MOLT-4 cells, which do not express CD3 on the cell surface. Both cell lines were cultured in RPMI 1640 supplemented with 1% pen/strep and 10% fetal bovine serum at a concentration of 2 x 10⁵ to 1 x 10⁶ cells/ml. Cells were washed in fresh medium before the experiment and 100,000 cells per well were seeded in a 96-wells plate (duplicate wells). The dilution series of 6 antibodies were made in phosphate-buffered saline (PBS). The antibodies were diluted 10 times in the cell suspension and incubated at 4° C. in the dark for 30 minutes. After incubation, the cells were washed twice in cold PBS / 0.5% BSA, and incubated with anti-HIS-PE (only for 200) or anti-IgG1-PE (for all other compounds) at 4° C., in the dark for 30 minutes. After the second incubation step, the cells were washed twice. 7AAD was added as a live-dead staining. Detection of the fluorescence in the Yellow-B channel (anti-IgG1-PE and anti-HIS-PE) and the Red-B channel (7AAD) was done with the Guava 5HT flow cytometer. Median fluorescence intensity in the Yellow-B channel (anti-IgG1-PE and anti-HIS-PE) in life cells was determined with Kaluza software. All bispecifics, but not the negative control rituximab, show concentration-dependent binding to the CD3 positive Jurkat E6.1 cell line (Table 1). In contrast, no binding was observed to the CD3 negative MOLT-4 cell line (Table 2).

TABLE 1

Analysis of antibody binding to CD3-positive cells (Jurkat E6.1) by FACS. The median fluorescence intensity of a duplicate is shown for each concentration tested.

| | | | | | | |
|---|---|---|---|---|---|---|
| Concentration (nM) | Rit-v1a-201 | Rit-v1a-145-204 | Rit-v1a-145-PF01 | Rit-v1a-145-PF02 | Rituximab | 200 |
| 0.32 | 104.79 | 77.80 | 80.30 | 58.79 | 58.28 | 63.63 |
| 1 | 90.00 | 82.15 | 88.23 | 71.52 | 56.55 | 66.22 |
| 3.16 | 159.28 | 112.67 | 116.72 | 114.55 | 55.37 | 83.74 |
| 10 | 160.91 | 113.22 | 142.62 | 168.91 | 60.83 | 109.70 |
| 31.6 | 202.99 | 165.47 | 221.00 | 229.84 | 58.71 | 154.42 |
| 100 | 248.66 | 200.74 | 252.20 | 278.91 | 55.49 | 177.16 |
| 316 | 294.49 | 263.83 | 256.79 | 291.09 | 54.99 | 223.52 |
| 1000 | 420.46 | 315.13 | 366.89 | 355.26 | 66.36 | 416.61 |

TABLE 2

Analysis of antibody binding to CD3 negative cells (MOLT-4) by FACS. The median fluorescence intensity is shown for each concentration tested.

| | | | | | | |
|---|---|---|---|---|---|---|
| Concentration (nM) | Rit-v1a-201 | Rit-v1a-145-204 | Rit-v1a-145- | Rit-v1a-145-PF02 | Rituximab | 200 |
| 1 | 75.76 | 78.38 | 79.32 | 73.79 | 78.96 | 79.40 |
| 10 | 72.18 | 75.93 | 81.42 | 72.70 | 75.92 | 76.59 |
| 100 | 61.51 | 62.37 | 79.01 | 70.44 | 68.42 | 70.49 |
| 1000 | 62.96 | 73.90 | 59.89 | 61.81 | 67.39 | 61.37 |

### Example 235. FcRn Binding Analysis

Binding to the FcRn receptor was determined at pH 7.4 and pH 6.0 using a Biacore T200 (serial no. 1909913) using single-cycle kinetics and running Biacore T200 Evaluation Software V 2.0.1. A CM5 chip was coupled with FcRn in sodium acetate pH 5.5 using standard amine chemistry. Serial dilution of bispecifics and controls were measured in PBS pH 7.4 with 0.05% tween-20 (9 points; 2-fold dilution series; 8000 nM Top conc.) and in PBS pH 6.0 with 0.05% tween-20 (3 points; 2-fold dilution series; 4000 nM Top conc.). A flow rate of 30 µl/min was used and an association time of 40 seconds and dissociation time of 75 seconds. Steady state analysis was used to analyze samples. FcRn binding was observed for all bispecifics at pH 6.0, with no binding observed at pH 7.4 (Table 3).

TABLE 3

Binding of different bispecifics, intermediates and control antibodies to FcRn at pH 6.0 or pH 7.4 as determined by Biacore.

| Antibody | pH 6.0 | K_{D} (M) 1.74E-06 | R_{MAX} (RU) 67 | Chi² (RU²) 0.783 |
|---|---|---|---|---|
| Irrelevant IgG1 WT | | | | |
| | 7.4 | - | - | - |
| Rituximab | 6.0 | 1.57E-06 | 96.4 | 2.82 |
| | 7.4 | - | - | - |
| Rit-v1a-201 | 6.0 | 2.16E-06 | 149.6 | 8.53 |
| | 7.4 | - | - | - |
| Rit-v1a-145-204 | 6.0 | 1.91E-06 | 122.9 | 5.36 |
| | 7.4 | - | - | - |
| Rit-v1a-145-PF01 | 6.0 | 1.90E-06 | 114.6 | 4.02 |
| | 7.4 | - | - | - |
| Rit-v1a-145-PF02 | 6.0 | 2.05E-06 | 123.5 | 5.47 |
| | 7.4 | - | - | - |
| Rit-v1a-145 | 6.0 | 1.89E-06 | 89.8 | 2.01 |
| | 7.4 | - | - | - |

### Example 236. Effect of Bispecifics on Raji-B Tumor Cell Killing With Human PBMCs.

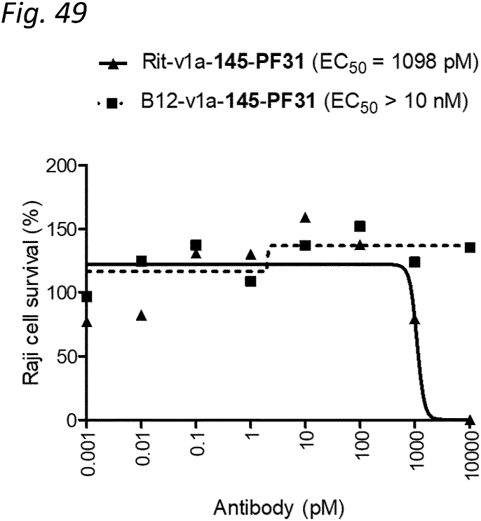

Duplicate wells were plated with Raji-B cells (5e4 cells) and human PBMCs (5e5) (1:10 cell ratio) into 96 well plates. Serial dilution of bispecifics (1:10 dilution; 8 points; 10 nM Top conc.) were added to wells and incubated for 24 hours at 37° C. in tissue culture incubator. Samples were stained with CD19, CD20 antibodies and propidium iodide was added prior to acquisition of BD Fortessa Cell Analyzer. Live RajiB cells were quantitated based on PI-/CD19+/CD20+ staining via flow cytometry analysis. The percentage of live RajiB cells was calculated relative to untreated cells. Target-dependent cell killing was demonstrated both for bispecifics based on hOKT3 200 (FIG. 48) and for bispecifics based on anti-4-1BB PF31 (FIG. 49).

### Example 237. Effect of Bispecifics on Cytokine Secretion in a Co-culture of Raji-B Tumor Cells And human PBMCs.

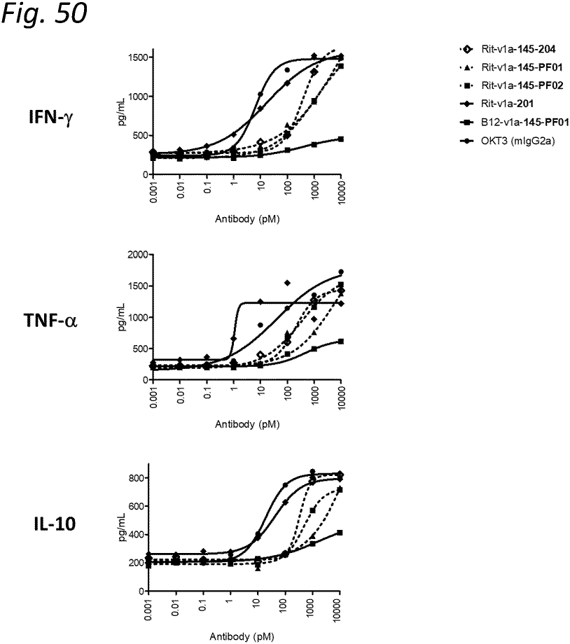

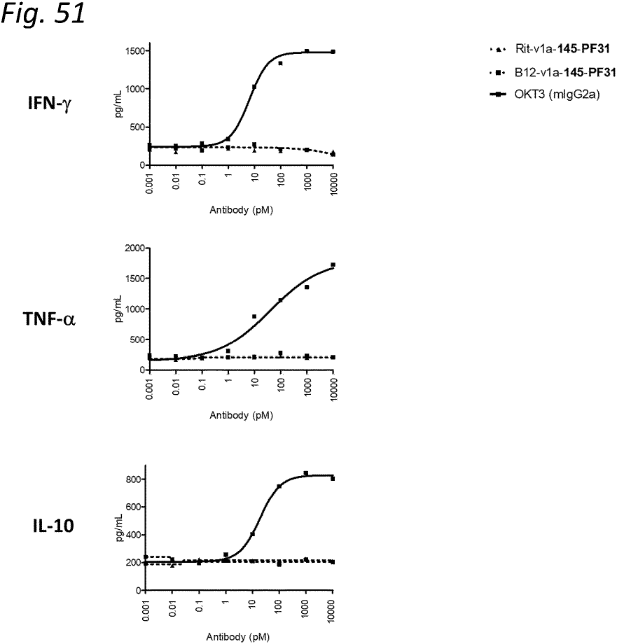

Duplicate wells were plated with Raji-B cells (5e4 cells) and human PBMCs (5e5) (1:10 cell ratio) into 96 well plates. Serial dilution of bispecifics (1:10 dilution; 8 points; 10 nM Top conc.) were added to wells and incubated for 24 hours at 37° C. in tissue culture incubator. Cytokine analysis was conducted on the supernatant for TNF-α, IFN-y and IL-10 (Kit: HCYTOMAG-60K-05, Merck Millipore). FIG. 50 shows cytokine levels for bispecifics based on hOKT3 200 and FIG. 51 shows cytokine levels for bispecifics based on anti-4-1BB PF31.

### Sequence List

Sequence identification of C-terminal sortase A recognition sequence (SEQ. ID NO: 1):

GGGGSGGGGSLPETGGHHHHHHHHHH

Sequence identification of sortase A (SEQ. ID NO: 2):

TGSHHHHHHGSKPHIDNYLHDKDKDEKIEQYDKNVKEQASKDKKQQAKPQ
IPKDKSKVAGYIEIPDADIKEPVYPGPATPEQLNRGVSFAEENESLDDQN
ISIAGHTFIDRPNYQFTNLKAAKKGSMVYFKVGNETRKYKMTSIRDVKPT
DVGVLDEQKGKDKQLTLITCDDYNEKTGVWEKRKIFVATEVK

Sequence identification of His6-TEVsite-GGG-IL15Rα-IL15 (SEQ. ID NO: 3):

MGSSHHHHHHSSGENLYFQGGGITCPPPMSVEHADIWVKSYSLYSRERYI
CNSGFKRKAGTSSLTECVLNKATNVAHWTTPSLKCIRDPALVHQRPAPPS
GGSGGGGSGGGSGGGGSLQNWVNVISDLKKIEDLIQSMHIDATLYTESDV
HPSCKVTAMKCFLLELQVISLESGDASIHDTVENLIILANNSLSSNGNVT
ESGCKECEELEEKNIKEFLQSFVHIVQMFINTS

Sequence identification of anti-4-1BB PF31 (SEQ. ID NO: 4):

DIVMTQSPPTLSLSPGERVTLSCRASQSISDYLHWYQQKPGQSPRLLIKY
ASQSISGIPARFSGSGSGTDFTLTISSLEPEDFAVYYCQDGHSFPPTFGG
GTKVEIKGGGGSGGGGSGGGGSGGGGSQVQLVQSGAEVKKPGASVKVSCK
ASGYTFSSYWMHWVRQAPGQRLEWMGEINPGNGHTNYSQKFQGRVTITVD
KSASTAYMELSSLRSEDTAVYYCARSFTTARAFAYWGQGTLVTVSSGGGG
SGGGGSLPETGGHHHHHH

Sequence identification of SYR-(G₄S)₃-IL15 (PF18) (SEQ. ID NO: 5):

SYRGGGGSGGGGSGGGGSNWVNVISDLKKIEDLIQSMHIDATLYTESDVH
PSCKVTAMKCFLLELQVISLESGDASIHDTVENLIILANNSLSSNGNVTE
SGCKECEELEEKNIKEFLQSFVHIVQMFINTS

Sequence identification of SYR-(G₄S)₃-IL15Rα-linker-IL15 (PF26) (SEQ. ID NO: 6):

SYRGGGGSGGGGSGGGGSITCPPPMSVEHADIWVKSYSLYSRERYICNSG
FKRKAGTSSLTECVLNKATNVAHWTTPSLKCIRDPALVHQRPAPPSGGSG
GGGSGGGSGGGGSLQNWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTS

1. A process for preparing a multispecific antibody construct, comprising conjugating a functionalized antibody Ab(F)ₓ containing x reactive moieties F, wherein x is an integer in the range 1 - 10, and an immune cell-engaging polypeptide containing one or two reactive moieties Q, wherein the antibody is specific for a tumour cell and the immune cell-engaging polypeptide is specific for an immune cell, wherein the reaction forms a covalent linkage between the functionalized antibody and the immune cell-engaging polypeptide by reaction of Q with F.

2. The process according to claim 1, wherein the antibody is specific for an extracellular receptor on a tumour cell selected from the group consisting of CD30, nectin-4, folate receptor alpha, CEACAM5, CD37, TF, ENPP3, CD203c, EGFR, CD138/syndecan-1, Axl, DKL-1, IL13R, HER3, CD166, LIV-1, c-Met, CD25, PTK7, CD71, FLT3, GD3, ASCT2, IGF-1R, CD123, CD74, guanyl cyclase C, CD205, ROR1, ROR2, CD46, CD228, CD70, Globo H, Lewis Y, MUC1, CA-IX, PSMA, CanAg, EphA2, Cripto, av-integrin, CD56, SLITRK6, 5T4, c-KIT, FGFR2, Notch3, CS1, gpNMB, TIM-1, CD19, CD20, Cadherin-6, P-cadherin, C4.4a, DPEP3, MFI2, CD48a, LRRC15, PRLR, DLL3, CD324, RNF43, ADAM-9, AMHRII, CD13, CD38, CD45, claudin, Gal-3BP, GFRA1, MICA/B, RON, TM4SF, TWEAKR, TROP-2, BCMA, B7-H3, BMPR1B, E16, STEAP1, MUC16, MPF, NaPi2b, Sema 5b, PSCA hlg, ETBR, MSG783, STEAP2, TrpM4, CRIPTO, CD21, CD79b, FcRH2, HER2, NCA, MDP, IL20Rα, Brevican, EphB2R, ASLG659, PSCA, GEDA, BAFF-R, CD22, CD79a, CXCR5, HLA-DOB, P2X5, CD72, LY64, FcRH1, FcRH5, TENB2, PMEL17, TMEFF, GDNF-Ra1, Ly6E, TMEM46, Ly6G6D, LGR5, RET, LY6K, GPR19, GPR54, ASPHD1, Tyrosinase, TMEM118, GPR172A, CD33, CLL-1, CLEC12A, MOSPD2, EpCAM, CD133, TAG72, FAP, PD-L1 and SSTR2.

3. The process according to claim 1, wherein the immune cell-engaging polypeptide is selected from the group consisting of Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides.

4. The process according to claim 1, wherein:
(i) the immune cell-engaging polypeptide is specific for a cellular receptor on a T cell selected from the group consisting of CD3, CD28, CD137, CD134, CD27, Vγ9Vδ2 and ICOS; or
(ii) the immune cell-engaging polypeptide is specific for a cellular receptor on a NK cell selected from the group consisting of CD16, CD56, CD335, CD336, CD337, CD28, NKG2A, NKG2D, KIR, DNAM-1 and CD161; or
(iii) the immune cell-engaging polypeptide is specific for a cellular receptor on a monocyte or a macrophage selected from CD64; or
(iv) the immune cell-engaging polypeptide is specific for a cellular receptor on a granulocyte selected from CD89; or
(v) the immune cell-engaging polypeptide is an antibody specific for IL-2 or IL-15.

5. The process according to claim 4, wherein the immune cell-engaging polypeptide is selected from the group consisting of OKT3, UCHT1, BMA031, VHH 6H4, IL-2, IL-15, IL-15/IL-15R complex, IL-15/IL-15R fusion, mAb602, Nara1 or TCB2, an antibody specific for IL-2 and an antibody specific for IL-15m.

6. The process according to claim 1, wherein the immune cell-engaging polypeptide contains one reactive moiety Q.

7. The process according to claim 1, wherein Q is selectively introduced onto the immune cell-engaging polypeptide by chemical or enzymatic modification.

8. The process according to claim 1, wherein:
(i) the multispecific antibody construct is bispecific, and the functionalized antibody and the cell-engaging polypeptide are both monospecific; or
(ii) the multispecific antibody construct is trispecific, the functionalized antibody is bispecific,
(iii) and the immune cell-engaging polypeptide is monospecific.

9. The process according to claim 1, wherein the conjugation is preceded by reacting a linker compound, comprising one or two reactive moieties Q and one reactive moiety Q¹ with an immune cell engaging polypeptide, containing a reactive moiety F² reactive towards Q¹, to afford a linker-polypeptide construct (Q)yL-polypeptide, wherein L is a linker and y = 1 or 2, which is subjected to the conjugation.

10. The process according to claim 1, wherein the conjugation comprises:
(i) reacting a linker compound comprising a reactive moiety Q with Ab(F)ₓ, to afford a modified antibody Ab(Z-L-Q¹)x, wherein L is a linker and Z is a connecting group formed by the reaction of Q with F; or
(ii) reacting a linker compound comprising two reactive moieties Q with Ab(F)ₓ, to afford a modified antibody having structure: 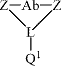
wherein L is a linker and Z is a connecting group formed by the reaction of Q with F.

11. The process according to claim 1, wherein x = 1, 2, 4 or 8.

12. The process according to claim 1, wherein the immune cell-engaging polypeptide comprises one reactive moiety Q, and the conjugation comprises reaction of ximmune cell-engaging polypeptides with Ab(F)ₓ, or wherein the immune cell-engaging polypeptide comprises two reactive moieties Q, and the conjugation involves reaction of x / 2 immune cell-engaging polypeptides with Ab(F)ₓ.

13. The process according to claim 1, wherein Q¹ comprises a cyclooctyne moiety.

14. The process according to claim 13, wherein Q¹ is selected from bicyclononyne (BCN), azadibenzocyclooctyne (DIBAC/DBCO), dibenzocyclooctyne (DIBO) or sulfonylated dibenzocyclooctyne (s-DIBO).

15. The process according to claim 1, wherein F is present on the Fc fragment of the antibody.

16. The process according to claim 1, wherein F is present on a native glycan of the antibody.

17. A multispecific antibody construct obtainable by the process according to claim 1.

18. The multispecific antibody construct according to claim 17, which has structure (13a) or (13b): 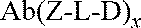 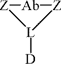
wherein:
Ab is an antibody specific for a tumour cell;
Z is a connecting group;
L is a linker;
D is an immune cell-engaging polypeptide specific for an immune cell;
x is an integer in the range of 1 - 10.

19. The multispecific antibody construct according to claim 18, wherein linker L is selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂oo arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR₃, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

20. The multispecific antibody construct according to claim 18, wherein Z contains a succinimide, a triazole, a cyclohexene, a cyclohexadiene, an isoxazoline, an isoxazolidine, a pyrazoline, or a piperazine.

21. The multispecific antibody construct according to claim 18, wherein Z is according to any one of structures (Za) to (Zk): 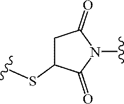 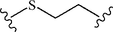 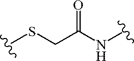 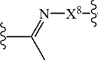 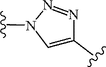 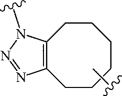 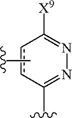 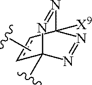 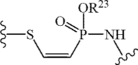
wherein,
X⁸ is O or NH;
X⁹ is selected from H, C₁₋₁₂ alkyl and pyridyl;
R²³ is C₁₋₁₂alkyl;
in structure (Zg) and (Zh), the 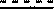
bond represents either a single or a double bond, and may be connected via either side of this bond to linkers L;
the wavy lines indicate the connection to linkers L.

22. A method of treating cancer comprising administering to a subject a multispecific antibody construct according to claim 17.


SEQUENCE LISTING (6 sequences)

SEQ ID NO: 1 - protein, 26 aa, synthetic construct
  REGION 1..26: note = Sequence identification of C-terminal sortase A recognition sequence
GGGGSGGGGSLPETGGHHHHHHHHHH

SEQ ID NO: 2 - protein, 192 aa, synthetic construct
  REGION 1..192: note = Sequence identification of sortase A
TGSHHHHHHGSKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 3 - protein, 233 aa, synthetic construct
  REGION 1..233: note = Sequence identification of His6-TEVsite-GGG-IL15R -IL15
MGSSHHHHHHSSGENLYFQGGGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 268 aa, synthetic construct
  REGION 1..268: note = Sequence identification of anti-4-1BB PF31
DIVMTQSPPTLSLSPGERVTLSCRASQSISDYLHWYQQKPGQSPRLLIKYASQSISGIPARFSGSGSGTDFTLTISSLEPEDFAVYYCQDGHSFPPTFGGGTKVEIKGGGGSGGGGSGGGGSGGGGSQVQLVQSGAEVKKPGASVKVSCKASGYTFSSYWMHWVRQAPGQRLEWMGEINPGNGHTNYSQKFQGRVTITVDKSASTAYMELSSLRSEDTAVYYCARSFTTARAFAYWGQGTLVTVSSGGGGSGGGGSLPETGGHHHHHH

SEQ ID NO: 5 - protein, 132 aa, synthetic construct
  REGION 1..132: note = Sequence identification of SYR-(G4S)3-IL15 (PF18)
SYRGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 229 aa, synthetic construct
  REGION 1..229: note = Sequence identification of SYR-(G4S)3-IL15R?-linker-IL15 (PF26)
SYRGGGGSGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS